SEQUENCE LISTING (38 sequences)

SEQ ID NO: 1 - RNA, 3491 nt, Homo sapiens
  source 1..3491: mol_type = mRNA
aattgtggctatagccgcctcgatcgctgtctccccagcctcgccgcggccgctccgggacgcgcccgcccgccgcccggctctccccccctttgggctgctgctgctgctgctgtgactgctgctgcgagaggaggaggaggaggaggaagcagcgggggggggagcggggggtggggggggagaccaagaagtacagttgggagcgagggagcttcacccccggggcggtggttgtttcttttttctttctttcttttttcttttcctttttttttttttttctaattcctgaggggtggttgctgcttttgctacatgacttgccagcgcccgagcctgcggtccaactgcgctgctgccggagcgctcagtgccgccgctgccgcccgcgccccccgcgccccgttcggcacccaccggtcgccgccgcccgccgcgccgctgtcccgctcccgcgccgccgccgccgtttccccccgacgactgggtgatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggacgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaacatccctgggaccagactgtaagtgaacgttttacacacatttgcattgtaaatgataattaaaaaaataagtccaggtattttttattaagcccccccctcccatttctgtacgtttgttcagtctctagggttgtttattattctaacaaggtgtggagtgtcagcgaggtgcaatgtggggagaatacattgtagaatataaggtttggaagtcaaattatagtagaatgtgtatctaaatagtgactgctttgccatttcattcaaacctgacaagtctatctctaagagccgccagatttccatgtgtgcagtattataagttatcatggaactatatggtggacgcagaccttgagaacaacctaaattatggggagaattttaaaatgttaaactgtaatttgtatttaaaaagcattcgtagtaaaggtgcccaagaaattattttggccatttattgttttgtccttttctttaaagaactgtttttttttcttttgtttacttttagaccaaagattgggttctagaaaatgcacttggtatactaagtattaaaacaaacaaaaaggaaagttgtttcagttggcaacactgcccattcaattgaatcagaaggggacaaaattaacgattgccttcagtttgtgttgtgtatattttgatgtatgtggtcactaacaggtcacttttattttttctaaatgtagtgaaatgttaatacctattgtacttataggtaaaccttgcaaatatgtaacctgtgttgcgcaaatgccgcataaatttgagtgattgttaatgttgtcttaaaatttcttgattgtgatactgtggtcatatgcccgtgtttgtcacttacaaaaatgtttactatgaacacacagaaataaaaaataggctaaattcatatatatcttgatacttttgtctcttttattaagtagagctaattttttaaagaccaatcaacttatagggaattcaaaggctttttcagccaaactaaaatttaaactgctcctttaatttgaactgactctaaaaatgaaaatagtatttttccctttgtgaacaaattttacaaggagcagcctatttaataaacactagctttaaacaaagtataggcttttcagctgatacctgtaagtttctgtggatatacagcaaaaagagatataatttaattttctgtgcatagctctttaccctgtgtttatttccaaatccattaatagaatgccatttatatattttgtttcaggtatattgttaatagagcttggcaaattataaataaatatatgtatatggttagatagaagtgactataatgcacacatatgtaatatatatagacacacagagcccttcagttcaggtacaatttgcgctatgaatgctgcaaacatttttgtttaaatatttgtatttatactttctaagtcagcatttatttttgtggctgtttacccacaatgaaagagttctaataaagatgtgctgaagttgcaatata

SEQ ID NO: 2 - protein, 489 aa, Homo sapiens [sequence omitted]

SEQ ID NO: 3 - RNA, 3018 nt, Homo sapiens
  source 1..3018: mol_type = mRNA
agcattagacaccggagggggcacctgggaccaacttcgcgaagcgggaagcccggcgggggggtggggggagctaaagacctgcggcctcagcccctccaaagaacagggagatgacggggaaagcaggggaagcgctgagcaagcccaaatccgagacagtggccaagagtacctcggggggcgccccggccaggtgcactgggttcggcatccaggagatcctgggcttgaacaaggagcccccgagctcccacccgcgggcagcgctcgacggcctggcccccgggcacttgctggcggcgcgctcagtgctcagccccgcgggggtgggcggcatggggcttctggggcccggggggctccctggcttctacacgcagcccaccttcctggaagtgctgtccgacccgcagagcgtccacttgcagccattgggcagagcatcggggccgctggacaccagccagacggccagctcggattctgaagatgtttcctccagcgatcgaaaaatgtccaaatctgctttaaaccagaccaagaaacggaagaagcggcgacacaggacaatctttacctcctaccagctagaggagctggagaaggcattcaacgaagcccactacccagacgtctatgcccgggagatgctggccatgaaaacggagctgccggaagacaggatacaggtctggttccagaaccgtcgagccaagtggaggaagcgggagaagtgctggggccggagcagtgtcatggcggagtatgggctctacggggccatggtgcggcactccatccccctgcccgagtccatcctcaagtcagccaaggatggcatcatggactcctgtgccccgtggctactggggatgcacaaaaagtcgctggaggcagcagccgagtcggggaggaagcccgagggggaacgccaggccctgcccaagctcgacaagatggagcaggacgagcggggccccgacgctcaggcggccatctcccaggaggaactgagggagaacagcattgcggtgctccgggccaaagctcaggagcacagcaccaaagtgctggggactgtgtctgggccggacagcctggcccggagtaccgagaagccagaggaggaggaggccatggatgaagacaggccggcggagaggctcagtccaccgcagctggaggacatggcttaggtcaaggcgcgctcagatgccggagccccaagactctgctctcctcgggccctgtggtgctgggagatgctctctgaggcaaggcccagacctggcctctgccatcctccctgttccccacaggtcctccatcacccctggtggctgcaggcaccgctgggttctgactctggaccatgctgagacatccctcatctagtcttgacctctccagcatcccagcctcagaagccttcttgctgcccacaacgtcccctcaagccccttctctcaatccctttgcaacgctcactggttttggccaccccttgctctctgttctcttgctttaaagagtcctccttcccagctctacattctgctctgcccatgcctaaagcccattgctgcaaatgcattgtgaattgcctgccatggctgtgacacagacggaggactggaactgcaactccagcgtcctcagcaccccactcctcagtaaaagtcttctcccaactcagcctgttccttcctgcagacctggctgggcctgggcttccacagtgtgaagacactgtgcaggcccaggcaggcccagcctctgccccacccatcagtggagtccagatggcaggctacagttgggaagtctcagcctgggcccctcagccacccttggtctcatgccccgccatggtcaccctcaggaacccaccctctccacacccagcctgtcccactggctcctcccagcacaggcacctatgtggcatgtggtgatgtacgctgcgtgccatgagtccatgtcctatgcctcacaaatgctgtggttcactgcactgttcaggagtccaaaccttctaccctgggtcctcgggcccctgagcctgtgtcctgaagaatctcgactctcgtgatatgctgcttgtgaccttgacttgccatgaaggcacctgcccccacagctccttgcaaactatgagttcacatgtgccctgccttgaacaacccaatctggctggtaaatgacatctacgaagccacaggcctggcctcaactgagaagccaagcctggagtaggcctggggctatgcatacgcattaagggcctgctggagaaaccttcactccagacagacagggggctaaggggctagggctgagcacctctcagagcaagcctttgacctgttcagaaggatgggaaaaaggctggaaggaaagcaaggcagaaggccaggagtaagcccaagctaagctgcaggcacacacgccttcatgccagtgcatgccaggaagcacgttcactagactaaaacacggtaaccatgatggaataaggagctggggcttctttctgctggctgatgggtggacctgtgtatggtgacctctccggcctggccttttccattcgcatgaacaatgggagcacatagccctgttgacctgcccgatccagattcccaggccaggctcccgaccccatccacctatccaccaccccacccacctttgcagggaatacctcgttagagaatggcctgagacagtgcgttgcaactttttaattccatatcaaaagttgatttcttcttcattccatttatggaacttccctccccctagtccggttcttcttgtctttgtccctgttggtttgaagtgttagactatagcccctggtgtgtaaataatgtacatagagtgagaagaaagaaacttgatattgaggtgtttgaaatatggaactgtaataacttctaggtattcgaaacctgtgatttctgtgccattttctgtaaagatacaagtaagaataaaattgacttaaaaata

SEQ ID NO: 4 - protein, 361 aa, Homo sapiens
MTGKAGEALSKPKSETVAKSTSGGAPARCTGFGIQEILGLNKEPPSSHPRAALDGLAPGHLLAARSVLSPAGVGGMGLLGPGGLPGFYTQPTFLEVLSDPQSVHLQPLGRASGPLDTSQTASSDSEDVSSSDRKMSKSALNQTKKRKKRRHRTIFTSYQLEELEKAFNEAHYPDVYAREMLAMKTELPEDRIQVWFQNRRAKWRKREKCWGRSSVMAEYGLYGAMVRHSIPLPESILKSAKDGIMDSCAPWLLGMHKKSLEAAAESGRKPEGERQALPKLDKMEQDERGPDAQAAISQEELRENSIAVLRAKAQEHSTKVLGTVSGPDSLARSTEKPEEEEAMDEDRPAERLSPPQLEDMA

SEQ ID NO: 5 - RNA, 2638 nt, Homo sapiens
  source 1..2638: mol_type = mRNA
gtgtgtcacagtacagttaaaggggtgaagatctaaacgccaaaagagaagttaatcacaataagtgaggtttgggataaaaagttgggcttgcccctttcaaagtcccagaaagctgggaggtagatggagagggggccattgggaagtttttttggtgtagggagaggagtagaagataaagggtaagcagagtgttgggttctgggggtcttgtgaagttccttaaggaaggagggagtgtggccctgcagccctcccaaactgctccagcctatgctctccggcaccaggaagttccaaggttcccttcccctggtctccaaacttcagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaa

SEQ ID NO: 6 - protein, 422 aa, Homo sapiens [sequence omitted]

SEQ ID NO: 7 - RNA, 1706 nt, Homo sapiens
  source 1..1706: mol_type = mRNA
ctcagcagccagcccggcccgcccgcgcccgtccgcagccgcccgccagacgcgcccagtatgagggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggcggcacgggctccggcatgggcacgttgctcatcagcaaggtgcgtgaggagtatcccgaccgcatcatgaacaccttcagcgtcgtgccctcacccaaggtgtcagacacggtggtggagccctacaacgccacgctgtccatccaccagctggtggagaacacggatgagacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcacagcccggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaagctgctcgcagctggagtgagaggcaggtggcggccggggccgaagccagcagtgtctaaacccccggagccatcttgctgccgacaccctgctttcccctcgccctagggctcccttgccgccctcctgcagtatttatggcctcgtcctccccacctaggccacgtgtgagctgctcctgtctctgtcttattgcagctccaggcctgacgttttacggttttgttttttactggtttgtgtttatattttcggggatacttaataaatctattgctgtcagataccctt

SEQ ID NO: 8 - protein, 450 aa, Homo sapiens
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 9 - RNA, 1519 nt, Homo sapiens
  source 1..1519: mol_type = mRNA
ctgcactctccgacagctactgcgctaaaagcgctccttccctgagcttcgggaaagagttcatcttcctgcaaaggagtctcaggctttcccagaggacttgaaaggccttcctcgaaccagccacaccaaactctgctgcagaaggtttccttctctttttcaacttcatgttgagaaaatgactttctcttgagcatctcattttcccctaaatttgggcaagtgaagagatatcagcctggtcatccagtagaacagaaggccgagtcccgcactcccccactgtaaactatttgattgcacgtgagttgctttgtttatgacttatttgctcagaagaggcacgttgggaagcggctcgagagaccagcccacgcgcaggtcctgagcgggcgggcgtgcgaggtcggcgcctcgctgcttggggccggggatgaagtcctgcaagcccagcggcccgccggcgggagcgcgcgttgcacccccgtgcgcgggcggcaccgagtgcgcgggcacgtgcgccggggccgggcggctggagagcgcggcgcgcaggcgcctggcggccaacgcgcgcgagcgccgccgcatgcaggggctcaacactgccttcgaccgcttacgcagggtggttccccagtggggccaggataaaaagctgtccaagtacgagaccctgcagatggccctgagctacatcatggctctgacccggatcctggccgaggccgagcgattcggctcggagcgggactgggtgggtctccactgtgagcacttcggccgcgaccactacctcccgttcccgggcgcgaagctgccgggcgagagcgagctgtacagccagagactcttcggcttccagcccgagcccttccagatggccacctagggcgcgcgcctccgcgggggtgggtgtccggcagccgctccgagcctcggccctgccccaagtagcccagaagcctccggcggcccaggattctaaggatgcaatcctcgaggaaaattagtcgattctcagattacctttattcgcatcatcagacctatggacgcaatcatttaattgcctttcttttcccctcctcctttgtattttgtagatttcattaatggatcttgtgaatgggttgattgctgtgaaaataatgccccctttccccttttctgggctactttgagggaaaacaatcttaagaaaaataggattaagctattctgttccagtcctcagagaaataatcactttcttaaactttgtgagtttgtcctgttcgggtgaagttacagtatccattacttgtgtttgctcacaacagagctaccttcctgttgtgtaaatgcgtttttgctttagtgcattgtgtgtgcaagcatgaagtagaaacactttttttttctgggtacagtacatgggtatcggtgctctgtatttttttaaactgtgtacacattattaaaatatacattttataaaatataaataaaaacgtggatttgtttttcatgccaa

SEQ ID NO: 10 - protein, 152 aa, Homo sapiens
MKSCKPSGPPAGARVAPPCAGGTECAGTCAGAGRLESAARRRLAANARERRRMQGLNTAFDRLRRVVPQWGQDKKLSKYETLQMALSYIMALTRILAEAERFGSERDWVGLHCEHFGRDHYLPFPGAKLPGESELYSQRLFGFQPEPFQMAT

SEQ ID NO: 11 - RNA, 3144 nt, Homo sapiens
  source 1..3144: mol_type = mRNA
agtttcgggtgccgaggtctgcagctagcggcaagcggagtcaggcatccgttcagactgacagcagaggcggcgaaggagcgcgtagccgagatcaggcgtacagagtccggaggcggcggcgggtgagctcaacttcgcacagcccttcccagctccagccccggctggcccggcacttctcggagggtcccggcagccgggaccagtgagtgcctctacggaccagcgccccggcgggcgggaagatgatgatgatgtccctgaacagcaagcaggcgtttagcatgccgcacggcggcagcctgcacgtggagcccaagtactcggcactgcacagcacctcgccgggctcctcggctcccatcgcgccctcggccagctcccccagcagctcgagcaacgctggtggtggcggcggcggcggcggcggcggcggcggcggcggaggccgaagcagcagctccagcagcagtggcagcagcggcggcgggggctcggaggctatgcggagagcctgtcttccaaccccaccgagcaatatattcggcgggctggatgagagtctgctggcccgcgccgaggctctggcagccgtggacatcgtctcccagagcaagagccaccaccaccatccaccccaccacagccccttcaaaccggacgccacctaccacactatgaataccatcccgtgcacgtcggccgcctcttcttcatcggtgcccatctcgcacccttccgcgttggcgggcacgcaccaccaccaccaccatcaccaccaccaccaccaccaaccgcaccaggcgctggagggcgagctgctggagcacctgagtcccgggctggccctgggcgctatggcgggccccgacggcgctgtggtgtccacgccggctcacgcgccgcacatggccaccatgaaccccatgcaccaagcagcgctcagcatggcccacgcgcacgggctgccgtcgcacatgggctgcatgagcgacgtggacgccgacccgcgggacctggaggcattcgccgagcgcttcaagcagcgacgcatcaagctgggggtgacccaggcagatgtgggctccgcgctggccaacctcaagatccccggcgtgggctcgcttagccagagcaccatctgcaggttcgagtccctcacactgtcccacaataatatgatcgcgctcaaacccatcctgcaggcatggctcgaggaggccgagaagtcccaccgcgagaagctcaccaagcctgaactcttcaatggcgcggagaagaagcgcaagcgcacgtccatcgctgcgccagagaagcgctcgctcgaagcctactttgccattcagcctcggccctcctctgaaaagatcgccgccatcgcggagaagctggacctgaagaaaaacgtggtgcgcgtctggttctgcaaccagaggcagaaacagaaaagaatgaaatattccgccggcatttagaagactcttggcctctccagagacgcccctttcctcgtccgctcttttctctcctctcttctgcctcttttcacttttggcgactagaaacaattccagtaaatgtgaatctcgacaaatcgaggactgaagagggagcgaacgagcgaacaactgagcccaagccggtgagaatgtgaaacagtttctcaaaggaaagaataacaaaagatggtatttgtctgttgtagcaaagttgtccctttgaaccccacctcggcttcttcagaggaagtgtggagatggctgtttgcaggaaggcagacgagacagtgtttaaaaagtccacaagaatgatcaagtaagatttgtttttattcttacagacatcacccgtgttcaagtttaaaagtacactttgcaactatttttcagaaatagaaattgattcaggactaaaactttaaactagagttgatgcttaatgtgatagagacatctctaaagtattttgaattttaaaaaaagatggcagattttctgcatttacactgtatattatatatatatttttattgtggttcttacccccttttccttctctgaagtgttaatgcttaagaaaagagttgcgcctgctgtgttcactgatcttgaaagctattattagattattgcagaacaaccctctgtaaattattaatttatctctctagcaacttaattttgtgcacattctaattaattaaacttcttccgtctaaaaaaagtgggggaaatgtatagctagtaacgttcaaaaaattttgtttgatgagtttaccgaatttttacagctttcctcctatactgtgttccttttgacccatttgtatattctcacttgaatgaagattgtttttttctttgtttttactggtagtgttctgatttgtgagtcgacactcagtaatggatgtcttaatcgtgtagacctgattcactgtctgaagtattgtttacttcgttacatatttaatggggattcccacattgtccccatgacacatgagcgctctcacttacccttacacacacacacacacacacacacacctctaacagaagggaagaagcagttggaagcatgaccgatgcaccattttctagttttaggtgcatttgccacttggtgtttgcccttcagattttagatttcaccaaggtatttcagtcttccagttttcaattgctttgttggctacatgttaatatttataggaatacttcagtttttccttttggaggtttgtttgtagaaaaactaatttgaactataagaaagacagtgcactgcttgtaaattcacattgtttggaaaaattcttttggaacaaaaaattaggtacatgataactggtaccttatctactgtaaatatttcattaaaaatgatgcacacatagatatattcttacaaattttgctgtattgctgttctctttgaggctctccaaagtcttgagttctgtatatggcctggtttcttgtttttattaatagatggtttatttactatggtaatgtattaatttatttttggtgttgttcgattgtctttcattgaagagataattttaatgttttattggcaacgtatgctgctttttcattaaaatatgctattaaaattaaatggcttttaaaa

SEQ ID NO: 12 - protein, 409 aa, Homo sapiens
MMMMSLNSKQAFSMPHGGSLHVEPKYSALHSTSPGSSAPIAPSASSPSSSSNAGGGGGGGGGGGGGGGRSSSSSSSGSSGGGGSEAMRRACLPTPPSNIFGGLDESLLARAEALAAVDIVSQSKSHHHHPPHHSPFKPDATYHTMNTIPCTSAASSSSVPISHPSALAGTHHHHHHHHHHHHQPHQALEGELLEHLSPGLALGAMAGPDGAVVSTPAHAPHMATMNPMHQAALSMAHAHGLPSHMGCMSDVDADPRDLEAFAERFKQRRIKLGVTQADVGSALANLKIPGVGSLSQSTICRFESLTLSHNNMIALKPILQAWLEEAEKSHREKLTKPELFNGAEKKRKRTSIAAPEKRSLEAYFAIQPRPSSEKIAAIAEKLDLKKNVVRVWFCNQRQKQKRMKYSAGI

SEQ ID NO: 13 - RNA, 16433 nt, Homo sapiens
  source 1..16433: mol_type = mRNA
agagcccttctggacagctcccgctcacccaaacagaagacgtcggcgccggagcgggctcggacatggcgaggctgcgagccggcccgagcggcggggcccggtgatccctccctccctccccgtcccctcccctctcccgcacgcacgccccgtccgcccccaccccgcccccaccccgggcgagcccgcccgcagcccggggcgcacacccgcacgcgcactcctctccactcactcccgcgcccgcccccactcccgcagccgagccccgccacgcgcgccttgcccgcccgccggccgcccccgccgcccccgccgcccccgggccctgatggactgaatgaaggctgcctacaccgcctatcgatgcctcaccaaagacctagaaggctgcgccatgaacccggagctgacaatggaaagtctgggcactttgcacgggccggccggcggcggcagtggcgggggcggcggcgggggcggcgggggcggcggcgggggcccgggccatgagcaggagctgctggccagccccagcccccaccacgcgggccgcggcgccgctggctcgctgcggggccctccgccgcctccaaccgcgcaccaggagctgggcacggcggcagcggcggcagcggcggcgtcgcgctcggccatggtcaccagcatggcctcgatcctggacggcggcgactaccggcccgagctctccatcccgctgcaccacgccatgagcatgtcctgcgactcgtctccgcctggcatgggcatgagcaacacctacaccacgctgacaccgctccagccgctgccacccatctccaccgtgtctgacaagttccaccaccctcacccgcaccaccatccgcaccaccaccaccaccaccaccaccagcgcctgtccggcaacgtcagcggcagcttcaccctcatgcgcgacgagcgcgggctcccggccatgaacaacctctacagtccctacaaggagatgcccggcatgagccagagcctgtccccgctggccgccacgccgctgggcaacgggctaggcggcctccacaacgcgcagcagagtctgcccaactacggtccgccgggccacgacaaaatgctcagccccaacttcgacgcgcaccacactgccatgctgacccgcggtgagcaacacctgtcccgcggcctgggcaccccacctgcggccatgatgtcgcacctgaacggcctgcaccacccgggccacactcagtctcacgggccggtgctggcacccagtcgcgagcggccaccctcgtcctcatcgggctcgcaggtggccacgtcgggccagctggaagaaatcaacaccaaagaggtggcccagcgcatcacagcggagctgaagcgctacagtatcccccaggcgatctttgcgcagagggtgctgtgccggtctcaggggactctctccgacctgctccggaatccaaaaccgtggagtaaactcaaatctggcagggagaccttccgcaggatgtggaagtggcttcaggagcccgagttccagcgcatgtccgccttacgcctggcagcgtgcaaacgcaaagagcaagaaccaaacaaagacaggaacaattcccagaagaagtcccgcctggtgttcactgacctccaacgccgaacactcttcgccatcttcaaggagaacaaacgcccgtcaaaggagatgcagatcaccatttcccagcagctgggcctggagctcacaaccgtcagcaacttcttcatgaacgcccggcgccgcagcctggagaagtggcaagacgatctgagcacagggggctcctcgtccacctccagcacgtgtaccaaagcatgatggaaggactctcacttgggcacaagtcacctccaaatgaggacaacagataccaaaagaaaacaaaggaaaaagacaccggattcctagctggggcccttcactggtgatttgaaagcacaattctcttgcaaagaaacttatattctagctgtaatcataggccaggtgttcttcttttgtttttaatggctatggagtccaagtgcaagctgaaaaattaatctcttagaaccagacactgttctctgagcatgctaagcatcccagaaacccaaatggggccttcctggagcgagttaattccagtatggtgtcaaccaagctcgggattgcttaaaatatcatccatcccacttcaggtcctgtcagcttcttgcagtcagagttcctatgagtaacaataggagtttggcctatgtaaggactctgagtttaggcttccaagatacaacaataagagaagaatctagcaacgagaatgacctcatttgctttccacatgcttagcctcattataccatgttatgtccaagttcacagccacaacatcagaatggtaattactgagcacaagttttaaatatggacgttaaaaaaaaaaatccaaggacctgtttttccaacccagacatcttttcattgaatgatttagaaagctttaagttgatccagcttacaattttttttttctttacctcctggaaatctcatatggtcttggatccgtcaaaaaaaccagtcagttcacttgcgctcaaagtatcaagcacaacaaagataaacagaagtgaggaaggttctgggttcactacatctggattttcaagacacctattgtgaagtcattagggaattgatgagaatatggcttcaagcacattttgcagtttgctacaaattctgttgtacataatgcagacgcacactcaggaggccaatttaactgttaacagtgcatggagcgaatgcagcattttaaaagatctaggtttttttaggtcattaatgtgtccttggttgatcagtcatctggtccctcctactgtgtgttatgaccaccacgtaatccattctcgctctttctgatttggggtttttcctcatccatcccattagtagggatgttttctgtgttttctagcaagaaaaaaaaatcaatcaatcaaacctgcatacatgttactcatgactgtcatctagtcctaaatctcttctgttgttgaatcatccttgcaaaacagctgaatacatctggagaaaacacagcacaccaaagaagcagaatactgcaaaccaaagacatttatgacttgtcattttctagcctaaaaatactgtgattacttttagaaatcagaaaacctctgcaactccgaatggcattcagctcttgcatttggcgcatcatcgggctgagcggaccagctacaccaaggacattagccaagccacccagaggggtggctttgccacaccagttgtcaccttcccatagcaagtggaagagcgcccacagaactctgggagattgcaaaggtcacaatgtgcatatttaccagtgaatggccccgggtggggccacgtgggggtgttcaaagcaagccaaacgctgcaatcattctttacagacacttgagactgacttttttatgaattacttagtcgaaaccaaagaaactttttctgcacctacttctgcaacaaacaaaactgtcccattaaaatgaataaataaatccgtaaatcaatggaaatcaccaccaataagaaggaagcacgccagaaaataaacgaaaacaaaaacagggagacacactgtgttcaaacagacctcttgggacattttttggaagcagattttaaagaaagggttgagacaaagatagaaataaggaagagcctcagtggctgctgcttcatttgacaactcacacggtaatcttaaagctgaagattgtctttaatttgtgcctatgcagtttttcaaaagaacacggaacagagcaacagaaacctcaacagctacaataccaaagatgaggatttctcacaccttttgtttcagttcattatctcctcttgcctggctaaaatactaatagcgccattgaactgtataaaggtaatcaattatgtttctctgagcaacaaaaggaaagggccatttatttgattttattgtttcatttcaattttgtcttatggttttttgccccaacatggaatctctcaaaagtttccatggactccaagtttaagatgttgggatattgaacagttctctctgctcagcagagggtagggaataacattatcacttgaatgttctttgcttaacccttagacttggttccttctatgttcagagtctcatcatcaggggaaggaaagggagtgagggtcagggataggggtcttggtgatgcatcctctcccgagccacagaaccaaagagtttatagaggaatttacagcctcgttttcatgtgattgctacatcctaacagggcttcatttgggggtggggggaaacatgtaaaaataattgccagtttctacttttctattagctttttaaaaatcagctgtaaagttgcatttctaaagaaagatatatataatatataaaatacatatatagatcaacttgacattggtgataaccaaaattattgctgtccaaattcatgtcttgttttggtccagtgcttcatttgctaagtattcggttcagaatttttctcatttctcatgccattccagagttaatttgccactgtggatgatttgaagtattcagatctctatggaagtttctgggacaggtttaaagtcaagatcaagcattttagcatttaacctgttgataaatggatccatggtgtacatgagttttatttgtattcggagtcatctctattctatccctcagcctcgattaaggtggtgagtgaagtgcatccaacagactcggcccagaactgggtcctgacagtggggtgctcatcttctgtaactgttgggaaggctcggtggtccattttcaccagttaaagaatatgaggccagcccagaaatctgttctccaggagctgccctgtcccatctgggtgtgccagaccccctcagtgagcaggtccaccaaagggacttctcacaggggaagcccaactcctgttgcaatgggttgatagatttcctcagggtggtaattaccaattcgtattttgacaagcctatgtgcaaccacagctggcactggggtgggcagtggtgttgggtgggatgggggagagtgtctcaatcctgaagagaaaatataaagcaggttttggggagacttctggagtcctgcccctagagagccccattgttgttctttgtgccccctcctcattccccctatgtgggtctccctatgcaggagctgtgagagaatgtgactctccacaatttttataattcatccttcctaggagattgttcattggctcttcccttgtgtccctttgtcccttgctcatactccatgtttcctttgtcaaaggactaagaaaagagcatatttcagcagaggagtgttcccatgtgggttgatttcaacttgggtatttctaaaagagtccttgtgacatgtgtccagtggaaatggttgctcttttccagactggattgaggaatggagcctgtttgatttggttagtgattctttgacatactaatctcagcgtttgggtctccagcatcctctgaagatgtctagactagtagaggctgcctttgtgacctgacattacaacattggtcaaaccagtcctctgataatcagaagaacatgtcataattgtttaaaaaaaaaaaaaaggcaagaatttctctccaaggagctttaataaatgtctcattccagataatgtcataccagagaaaagtgcttgcttttagaaaattatttacatacatatataaatatatatgtgtatctatacagttatgtatcaaaattttaagccctgcagaatttcaatttgttagaaatctaacagaaaaaaatttctatattgaaaggtaatagaatttaacccagtgagtttactcaaggatttttaaatttaagttaataatttcagagaaaataaccatttgggtgtggttatagtttagtatccattacctcaatccaaggaaaattccaggcattcctcaaccatcaggaaaaggtacagtgtgaaggaacagttctcagccaaatttcacattcttgaggcaacagaaatcaaaacactcagagccattgagtggaaaaacaatttactttattcctttacacaaataggcttgcattgtttttgttttaatgtgattttggtactagggatataattatttcattccaggaaataataaaaaaaaacagacagagccaatacatttctttttttaaaggaaacagcaacaacaataaaaactcagcaccaatatttaaaagcttttccaaaatgtaaaagaagtgtttagcttgcaccatgcataaaggtgcaggctagttgaaccaggaagcatggcacttcctctggagaaatccagaaagagttgcttctaagctcccttttccccctgcaggctcttggcaattgtaggctttagcaaatccagaataattttcaattcaagctaaaataaaatcaacatttggaatgtaaatctgatacacacacacttttctaagtcaaacaacatatttcaaaaccaaaaataaataccttttagataatcagttattttctttgtctatactgggcacccacctactagtgccagtaaattcaagttgaacagatttttaaaatcactattatctgggtatgggggaaacttccccacttttgaaaatgttggtagaattataggaatgtctgtttgattatcattaccaaagtgtcatgacagtatgcctttgtagtgaactcggattttcaggagtttgaatagttggatattttaaaatctaagaagaaaaggcctgtttccaatgttgttgaagaataatgaactctattaaaaagtggagaaaaagataatacatgtggtcaaggttgaccacaaggcccaggcacaactaccttggcgataatcttctagattcgtaacaggttagagctgactttttgtttttgttgttgctgatgctgtgtgattcagacttctcagcctaaccaggaagagtaagtggaaatggtagatgaagaaggggtagagctggtgtatctataactttctgatatttgtctgccaaacttgatatattagtaatttttttatctttagctaagatcaagtcacccctgaaacaacaggagattctagttttaaaataaggccacaaaaatccttacggaatgaagaatggcaccccagttggttgtataagtctcataagataatgatgttgattttaaatatggatgtctcaatgcctgttttctatcaatgatttgtttgtttccaaggtcggggagggaaagaggggagggtttatctgttttagaaagtctcagaatacttataaaatacagaagtagttattaaaatatataggacctcacataggtagatacagaacttaccattgaggctgatgggctgttgtgtgaatcacacaggaccttaaatgaggctcattattctcacacaccaaaatgactctgacagcctgaagcagttattgctagagcccaagctttccttggaggttttggagttaggttgattggaagtaaccagctaataccttttctagtggagaaaaagacattgctaccagcttgttcatcccatagaagtcttccactctgctccatttttagcagcaagcatttcatgtagcataaaccttggcagataagtgtgcctaaggtttatacagtctgtccgcttggatgtatacaaatttagatacatattttaacatgtgttctcatagatgactttataacaacacacattacctataggtgtctagactgtgtacatacaagtgtgtacagacaagcttcatacgtatatactgtaatccgttacaacaaataaattttaaatcatcgtttaacatgtatgtggtacttctacagtgtacattgttttcattatttattgtaacattgaaaaccacagtgcagggaaaacaaaagtatcccagcatcttcatcctgtacacttggaattaatttcatttgggcatatccaagataaactcaactttcaagaaatcttgtatattatttaatcatctgtgttaggatgacacctatgattgatgacttcggttgaatagctttattctggatttttcataactaaagctaaatccaaagacctgaaaaaggacaaaaagaaaaaaaaaaaaagaaaaaacaaagaaaaagaagaaaaaataataaagtcaagcgcaaactgatggggagacagtgggctctggtttccaggattgagacaatggtactgcggtcttggggagactgcgttagctagtggggagtggtgatttttttcatgcttgtcacatctaaatggtctttaacatgagaaagttttagaggttataatttcctgctttgtttttatttagactatcaaatgaagttatacatgttgtcagtcaaaaaatgaagacaccctctgccccaccccacagaatgctttttatcttgtctctttgggttatgacccaacaagctaagtaccattaatgtaattaacttatttaaattagttcctagtacataaatgtataggatttgggtaattatttaatcatccttccttagtttgattctactccttgtacttatttatcaaaacctagaccaatggtgcatcagagatgcaaaattctacttggaatactcttgaagtttagtttgctttataaagcagtgaaattctgttacagacagggaagaaatacaggttacaaaaagagaatttgggatattcttccctcttaaattaacttttaaaatagtctaagtaacaatttttaaattatttaacttaagttcgcagccccacctggtaccaggcgaacttcacctcttaattattgtggccctcggagccttcatattgtaacttatttatttaacttattcagcatctgtgaaaggtgcactgtatagtttatatttttaatttaaaacaacagagagcactgcagtttgtttgctgtcagaacaacagagcaaattttgtggacaagcaatgactattcagcctgaacctgtgcattcagaaaacataagctgagaccctgcttcaccagcctggatttcggggcttctatacagaaactggaaaaataaattttaaaaaaatcgtaaacaaaaagagagaaacccttacactagctgcttccaagaatgaactctgtgtgtatgtaaagcaacaaaacaaaaaaggaaaaaaacaaaaagcagaaaaaagaaaaaaaaaatgaaaaactttctatttctagtgagaaccaaagaaggctacctcactgactttttccatttgtaattttaatcgtgttgatgacaccaaagataccaaagatttctttctctgtgcggtctgcattttgcttgtgctcttttataatttgaacgattttctctgacatatggtatgtacagccacagctcagataccccaaagaaataattatctatgcgacggcggctgctaatttggaaagggatattttctgtgtttctcttatatgtttgctgtctgctcgacatgttcaagatgcgagttcagatgctgctgtaattggattccttaaattctgattacaaattgaggaaggaaactggttggaaatggccttcagtcctagccatggcctctatccccgctgggacctgtcacagtaaagactgccaattactgaaccacagaagctctgaccattgagtagttgagctggaagagaccttaggaatcatttagtccaagccccggtggcccagaggaatgaaatagttatccaaatcaaataactcttgagagtgaaagcccacacatgcctcctggttcctgccccagtgctccgcttattgtacagtgctacctctgcatgagagcggtcccacattgacaaataggatggtggcaatcctttagcaatgagcagggactggggtttatctcttaacattttcagctgtaaaattagtcacaagcattttcagtgtcccattagtacatagtcacatatggtcggttgcttcgtgaaggtggcctgtcttgaaatactagggctcatacgggatttttgccctaggaaaaacatgttgatcccaatgatgtgatcacttttgaacctttccattacaaagcattgtatagataactttttaattcagtaggaggagaaagttcattcttggcctgttggctttgattattatgggtactttaaagtcagtatttatcaagaaagggaacttgaccaccattggcacatgtgacatttaagctcttcagccttttcctttttagttgtaggtgtttacatttcatttctaagccaactctgtatttatgagagaagtttaagccttacatcatttgatactaaagggttatttgtggtaaatgaaaaatgaccccaaaattacagaggaatatgccagtttaagaaatggctacttaaagttgcttctctctttccttcttactcatgaaattaattggtcttcttcaagtttctttagattccattaaatgattaaatcactattaagagccattcatcaacgtgatttgtgtgttagccaatgaatctgtctcagcttttgaccaaatgggttttagacaaatgcaaagatctgcctctagtccatatggctctttttgagtgctagtattttgcatttcacataatgtagttattttgagcttttaaagagagcatttagacaaagaagcaaagagaggaagggaccaatcaactcatcagttccatgcatcaacaaagcatagctagtagaggaatataaatgacagattgacaaactgtaggaaacactgttactctctttctgaagttttcaagcaccatcctatgtgaaagttccctcctgtccaaacaagctcaaggcccatcttctccctatacaaggcaaacctgtaaggccttccttccaaagagtacattgctttggttttcttcctaaattcctattggaattagaactctcagaatccctgggagacagagcaaagatgacttaattcattgagcagcagagctccctataagtgaacatcaccttccccatctttcctactgccacacccatacgagagaggatctagaaagagcgatggcagcctgaacacagaaaacatccccacttggcagacctctcctcagcaatccccccagcctcatgcttcacttgcaaagtgtgacataaccacgggacgagtgccttgcttgaaccaaagcaacgatttagccagtctggacctctctgtgctttttttaattcttcctgtgaatacctcagcttcaactgggcctccatacagtcagttggtgggcttattgtactgtggtgctttgcaatgcaaccctgcaaagaacaagatttgtactaataccaaaggttctttctctatgtctcctcctctgcctccctcgttcttcccttttttctagttcttcacggttccaaagctttactatgaacctgggcatgttggcaatgcagaccgcgcaattccttaccgaattttctcagatatacctcatagacaatagtgtttagagtaatgttattatagcgtatgtaataaattattcactgtttcttttggtaactgtgatttaaaaaaagaaaaaagaaaaaaaagctttatacgttttaggttgtgcttttgtaatagatgaaaaaaggtgcgcttaaaaagaaaatgtatgtttttttccccctttggattttatttatgctggattggggaaagttgcagaatgagcccaaagtttacagtttcatattttgctgaagaaacaatctgtgttcatttgctctgttgaaaagaataattattttctacatttgtgccacttggtctgaacaattaattgttccgtgttaacagtgtagtattatgattagcaactgccaatcagtgctataattttatgcatgaggctaaaaatttagcagtgtgatgcattgtggtcttaatagcaacatttttcattttgaactagatcttcccctttggttcaatggactttatttatgcatgggcgcctattgtttgttagcagttgtggaacagttgtgtatacattaaactgtgaaaatgtacacagttcagcctcagacggtggtaatattggttttattgggagatgtgtcacctcgaaaataccctttacatctgttgggatctgaaaatgagtcacattgaattgggttccagctttataatgagaaacgttattcctaatttttgagttagccaatttgcattccacaaattgggatcctcataacccaaatatatcaccgtatgtgagagggatttgaaagcgagtattgaaaaactcacctttgcatatttaatttccaccaaaaggagttattttggctttatgctcatgaacttagacctaactggccatgtatatgtagatgcaaattcatctagctgtggccctctttgatctctgcttgggaatggctatttttgactatgcgtggtttcttctcgtattttgtgatcaggtcagctcccagtagaaactcaaatggcatcaatattactaactcttctctgcccacttctcttttgtccactctcctagacattcccaccaactgttccagtgatttgggcaaaaatacgcagccatttcccaaaacttcacatgtgcagctatcatggctgtccctccctagacttggaggtgactctcacttaatttttacctgcccaacaatgttccatctaccatctaaaaggtaatataagaagaagttttgaaacccactttaggaaaaccatcttctttaaatccttcaattatctgaggcctctatatgtcaaaactatttttcagttgcaggggattgggcaaacttgttctttcttatacttgggttcaaagacccattctccagtttcatatttcccaaaccaaaatgcttgacataaagccaaatcaactgccaagcacactttattttgcataggagtatgcagcctagggaaccttggttgaaaagcagcagtctgctatgcaaaatattggaaatcactgacagtgtagcattcatattatctgtcaatgagggtatattgggaacgtgctctcgtgaataataaaaagcaacatatttttatttggccttataaattaggttgtggtaatgtaaactttgatatatagtctttttatttttctcttattaatctgccaaagatgggaacagatacaagaatttttcaaattggcttttgtaagacaattgatgattgtaatagtgtttaatcttccagaaagctttatatgttgttccacaataaaattgatatttgtttcagcaaagttttcctgacactcacaaacccacaaactgttcctcttaatgcagatattgtagaatctacaaagttcaaatccatttttgatccaaagaaagtagaggagtatttgagacatgagtgtacccagccctttttttaatcacaggcaatgcatgggtctggctggttacactttgccaagaagacttgtcttatgaaacccaaggtatattttgttatgccattttatgtccttttcttttaacattgtggaaagtggtatgttgaatcaagtgtaagctgagttttccagacaactgaagtagctacatcatgaatgttattttgttattaaagggtttttactcagtgctttgtgccaatggatgtccttttccttggagacacataactacaaaattacctcagcttggcctggttttctctcctgccctcttggggaaacatgggcctggcctgggaaaaggcaggtcatgggctggaaggtaggttttggtactaggaagaaatctctgtatctgtcagctttaaagagaactgggccaaaaatctctaacctcactctctctggactccaacacttccctgcaatcctttggtcttgagcatgtgccagcatgaaggcagactccagttcatacatgaaaggcaagaaaaagaaaatagtaaccttgaatcttctgtgggccaccaggcactcacctttccccaccttgcacactatccagtcaaggctattgcagcccatctggtggctttacatgggacattaccaaaggcttcttcctccatcctggggttgcaaaggatccaggtcccctccatccagtggggctcttccacatcagaagtccccctcccaccatcctctgcatcctgtttagctatcccatctataccttttggagatgattatttagaaaacaaagaaaggtatggaatggggtttcctattgtttgctaggttatattttagcaattctcaattctttgatctggaaaaatacaagagggaaaaggagaccccactatctccctgtgctttgctcccatctcagggggcaggggcagtgcacattgcctatgctgttgatctgtcttgggcgacaggctgaatcacagctattgccccagccaaaaacatggcccatcaatgcctactttatctctgcttgaaaatcctattcaaaaagttgtagagtttgaggtttttatccccccatatcctttgctttggtccagtttggcctttagcataagagtcagctttatctctaggaaagttttttcagattatgacaaggaacctgccacctgggaagaaaagagtccgaagactagcaatcggataggtagtcataccattaacagatacttccttgaaggtagaatattatttcctttctttacagttttgtgttacacaagtccaagtggtgccagcaaacttcttaccgtgaaatgttgtaaaacacctggcatactgaaatttctgaaacaaaaacacaagctccacattgataacttgataaataaccactaaagtttagatgcagggactgagatgatacaggcaaaatcttggtgttggtttctcttttaattcgtatcttcgatcacctaacctttctcaatccaagagcagttcagtcttttctccccaagtctaggatgccaaagagcatcataggaaaagataattagggattgaccagcatttcaattagttctcttcttcatctttgcatttctcaaaagtgttctcctggaccagagggaaagagctggtccattttttttcattctttctattcaaatttttccacccagacaatactttattaacacagatactgtagatccttccttggtcagtgaattattacaagaggagctatccttccaccaaagtgagtgaaaacaagttccagtatcttttcttccatccagttttgttctcagaatccaagtcagtcctgggtcttttctcactttagaccctggcctcagatgtgtttattcttgctatttaaaaatacctttaaatttcacatgctggcctgcagaacttgcatcctttgttctatactgttgactgcttgatggtattgaaaggtgactataatgagggaagaaaggaggaggtaaagagagaagaatttgtcccagatctgtttaaagtttcaaaatttaaaaagggacccattaaattatgggaaaatggctatagagtgtgagcctccgttgaccatatgctcaaagaccgtactctgccacctgccttccaggtagctattctagaaactcagtcctttgtggaaacccaactaccttttaaaagtctctttccagattccaaaaggacaagagatcagagagtcacatatacgcctcttgttttattttcttgctttcacgggtattattgccaagaaaatcgtagggaaaaactttaaacttttcttttcagttgatccctttgacatcacctctcatgtttaaaatcaggaaaacacacccctaaaatttgcactctcttccgttttgaaaaagaaaacccacacacaaatgcacactattaccgtctttcaccctgcgctatatttccaaagtgtattataatccagatattgccccatctcaaacatgttaagtcagactgtgctgaaagactttccagggacggtcaacagggtatatgttcagtggctgccctgaaatcctggtggggatgaggatcacgcttcatcatcaaggggatgcccatcccctgataagctcccagtccttttggaagatttctttgaatgttaattgcattttcagttttgctcatttcccaccccaatgttttgtctgcaacatcgcttacactggattctttctatttttattcctatcattaaatggtagtgctgtaaattctgcaattaatgttaaataaactgctttaattcattga

SEQ ID NO: 14 - protein, 504 aa, Homo sapiens
MKAAYTAYRCLTKDLEGCAMNPELTMESLGTLHGPAGGGSGGGGGGGGGGGGGGPGHEQELLASPSPHHAGRGAAGSLRGPPPPPTAHQELGTAAAAAAAASRSAMVTSMASILDGGDYRPELSIPLHHAMSMSCDSSPPGMGMSNTYTTLTPLQPLPPISTVSDKFHHPHPHHHPHHHHHHHHQRLSGNVSGSFTLMRDERGLPAMNNLYSPYKEMPGMSQSLSPLAATPLGNGLGGLHNAQQSLPNYGPPGHDKMLSPNFDAHHTAMLTRGEQHLSRGLGTPPAAMMSHLNGLHHPGHTQSHGPVLAPSRERPPSSSSGSQVATSGQLEEINTKEVAQRITAELKRYSIPQAIFAQRVLCRSQGTLSDLLRNPKPWSKLKSGRETFRRMWKWLQEPEFQRMSALRLAACKRKEQEPNKDRNNSQKKSRLVFTDLQRRTLFAIFKENKRPSKEMQITISQQLGLELTTVSNFFMNARRRSLEKWQDDLSTGGSSSTSSTCTKA

SEQ ID NO: 15 - RNA, 7827 nt, Homo sapiens
  source 1..7827: mol_type = mRNA
agacagcgccccgcggcctgtccatgagcggcccatgcattatggaagacgcgcgctgggctcgctccctggtccttaaatgacagcaaatgaggtcctttctcagcctccagctgggctgtccccaagctgagctgaggctcttctcctccgatccccacctctgcccggacatccaccatggggacagccaccaggaggaagccacacctgctgctggtagctgctgtggcccttgtctcctcttcagcttggagttcagccctgggatcccaaaccaccttcgggcctgtctttgaagaccagcccctcagtgtgctattcccagaggagtccacggaggagcaggtgttgctggcatgccgcgcccgggccagccctccagccacctatcggtggaagatgaatggtaccgagatgaagctggagccaggttcccgtcaccagctggtggggggcaacctggtcatcatgaaccccaccaaggcacaggatgccggggtctaccagtgcctggcctccaacccagtgggcaccgttgtcagcagggaggccatcctccgcttcggctttctgcaggaattctccaaggaggagcgagacccagtgaaagctcatgaaggctggggggtgatgttgccctgtaacccacctgcccactacccaggcttgtcctaccgctggctcctcaacgagttccccaacttcatcccgacggacgggcgtcacttcgtgtcccagaccacagggaacctgtacattgcccgaaccaatgcctcagacctgggcaactactcctgtttggccaccagccacatggacttctccaccaagagcgtcttcagcaagtttgctcagctcaacctggctgctgaagatacccggctctttgcacccagcatcaaggcccggttcccagcagagacctatgcactggtggggcagcaggtcaccctggagtgcttcgcctttgggaaccctgtcccccggatcaagtggcgcaaagtggacggctccctgtccccgcagtggaccacagctgagcccaccctgcagatccccagcgtcagctttgaggatgagggcacctacgagtgtgaggcggagaactccaagggccgagacaccgtgcagggccgcatcatcgtgcaggctcagcctgagtggctaaaagtgatctcggacacagaggctgacattggctccaacctgcgttggggctgtgcagccgccggcaagccccggcctacagtgcgctggctgcggaacggggagcctctggcctcccagaaccgggtggaggtgttggctggggacctgcggttctccaagctgagcctggaagactcgggcatgtaccagtgtgtggcagagaataagcacggtaccatctacgccagcgccgagctagccgtgcaagcactcgcccctgacttcaggctgaatcccgtgaggcgtctgatccccgcggcccgcgggggagagatccttatcccctgccagccccgggcagctccaaaggccgtggtgctctggagcaaaggcacggagattttggtcaacagcagcagagtgactgtaactccagatggcaccttgatcataagaaacatcagccggtcagatgaaggcaaatacacctgctttgctgagaacttcatgggcaaagccaacagcactggaatcctatctgtgcgagatgcaaccaaaatcactctagccccctcaagtgccgacatcaacttgggtgacaacctgaccctacagtgccatgcctcccacgaccccaccatggacctcaccttcacctggaccctggacgacttccccatcgactttgataagcctggagggcactaccggagaactaatgtgaaggagaccattggggatctgaccatcctgaacgcccagctgcgccatggggggaagtacacgtgcatggcccagacggtggtggacagcgcgtccaaggaggccacagtcctggtccgaggtccgccaggtcccccaggaggtgtggtggtgagggacattggcgacaccaccatccagctcagctggagccgtggcttcgacaaccacagccccatcgctaagtacaccctgcaagctcgcactccacctgcagggaagtggaagcaggttcggaccaatcctgcaaacatcgagggcaatgccgagactgcacaggtgctgggcctcaccccctggatggactatgagttccgggtcatagccagcaacattctgggcactggggagcctagtgggccctccagcaaaatccggaccagggaagcagccccctcggtggcaccctcaggactcagcggaggaggtggagcccccggagagctcatcgtcaactggacgcccatgtcacgggagtaccagaacggagacggcttcggctacctgctgtccttccgcaggcagggcagcactcactggcagaccgcccgggtgcctggcgccgatgcccagtactttgtctacagcaacgagagcgtccggccctacacgccctttgaggtcaagatccgcagctacaaccgccgcggggatgggcccgagagcctcactgcactcgtgtactcagctgaggaagagcccagggtggcccctaccaaggtgtgggccaaaggggtctcatcctcagagatgaacgtgacctgggaacccgtgcagcaggacatgaatggtatcctcctggggtatgagatccgctactggaaagctggggacaaagaagcagctgcggaccgagtgaggacagcagggctggacaccagtgcccgagtcagcggcctgcatcccaacaccaagtaccatgtgaccgtgagggcctacaaccgggctggcactgggcctgccagcccttctgccaacgccacgaccatgaagccccctccgcggcgacctcctggcaacatctcctggactttctcaagctctagtcttagcattaagtgggaccctgtggtccctttccgaaatgagtctgcagtcaccggctataagatgctgtaccagaatgacttacacctgactcccacgctccacctcaccggcaagaactggatagaaatcccagtgcctgaagacattggccatgccctggtacaaattcggaccacagggcccggaggggatgggatccctgcagaagtccacatcgtgaggaatggaggcacaagcatgatggtggagaacatggcagtccgcccagcaccacaccctggcaccgtcatttcccactccgtggcgatgctgatcctcataggctccctggagctctgatcctggaacccctccctctgcgccgcagctggacgccacctccgacggacacagccagccccttcctgctgccaaggtggcctgacactgtgccagagagtggctggttttaaatacctactttaaacagtgccctttttgtaggaggtaggatattttatattctgccgcaggatagaacccacgcaaggattttctttaaattgagaggcaccaggcagtaacttccatgatgacactgacgcctatacctgagctctaggctgcctggagggaaggaacaggcccatgggaagaagggggttttaaaaacatgtcttcaactcagcagagatggccctctgggaccctatacggactccgccacttgagagcagtcctaggcccggcaggaacaccagacatgaacaggttgaagaactggagcgaagtgcacacctcaccatccttcagtctaaggaagaagggcaagccctgggaccaagagctctcccgccttctccctcgagcagcagcaaggaccctgacgctgtccccgataactccctaggggctcctgcctgcccaagcggctgagaaccagcgccccgatgcctgaggctgggagcctgagccccttcagctttgaggggggtgatactccaggctgtttggggtgggagccaaaaagagttgagaggccagggcccttggtggaaaggggcaccagccttggtctgagatagtcacaacccaggtgacgatgccctctcagccaacactgccaacctgaccctgtcatcccgattgacagcgccacttcaggtggctgggtgactaaagggcttgtcttggtggggtctcccacccctccaagacccattctgcacagtccctccagggtttgggcaggagatggccaatcatgcgcccacctctccagtgctgcctgcagtcagctcggcctccccgacctgcagccccagactctgctctcccagcactgactcactcctgcctgggaggggaatgcagcattcatgctgtgtgtcctggtattgggaggtttctgggaagggcagaggataaatgtggccctgcctgctcccaggtatacctaggaccacctggccagatccgctcccagacggccttggactgcttgcatttccccggagaaaaaggggttaataaatgggccatcctttcctgagctctgggtatactaccagtcacagaacgtcagagctggaagaagccttagagctcaacttcttcaagcccctcactttacagatgaggaaatggaggtggtccagagagggtctgggattcccaaggtcacacagcccagaagagatggggctgggttaagaactcgagtcttccacctttctgttcaaggctgtttgtctacccagaggaaggaggcactgctgaatggctatggcctggctaagaaggtgattagtcagtagggtgtgaaaattctacttcaaggggttcggattggtgatcatggggattggcatggctgggttcccgtccaaggtgtgggcagagcttctaccaaacttcaacatggagggctgacttgaagctccctgtccccctcactcttgccccaagaaaagaggccaaagcaagagcagattccctaggcaagagcagcagcacaactaggaaaccccaaagcccatgctccgacaggtggcccttcacagggggcagcgggacaggcatcttgaagggcatatgtcctcggaagctccgagcctgttttctgtagtttatagttagagctctattttgttatggttttttaaacttttaagtcctgctctattttcctgggcaggtttatgttgatgtttacccactacaattttttaaaaatataagctcacatgccttttccctgccacagccaaacccccactgcaccctacccacccacccctagcccaggtcagctttcctggagctggctaatgaaagcctcctcacctcttcccaacccttacaagcaagggtgctaggggctcagctatacgaccattctccctgacagggagtccaaacttggcctagcatccctcctggcccccctctggccacgacttggcctgtgcctggttctctatcagaaaggggatgctgaacaaaacctccttccaagttttatccaattcgttcctcattgcctcgggctgcgtcaggggaagcaggggacaggtgtccagttgctgggccgagggaggagctggtttggcataggacctaaccagtgaagctagaggctacagccactaaacttgcttcaggccaacgatagttactcacaagtaagtaccttaatgctaatgaggtccactaaaaaggggaggaaggcagacctcctgggagacccacgaagggtttttagccagggaaaactgagccccaggaaaacctaaccactgggcaggcagaatttgtttgagggatagaacgacaacaaaataaatgttcctgcagcctgagatttcaggtagagtactgactaaggtttaataagacaataggtgacctgaggacatgcaagcttgtaaaatgcaacagcctcctgctagagtgacttgtacatgagcttgcttgcagaagactagattagatgtttctcaggatcccctcctgcgcaggggttctctgattttcgtgttctctgcccagatgggctgggggagttgagagtgtgcttattttcactgcgatcatgagaccacagttctgggttatctcctctcatacatcaagccccagaggaggcggcaagaggaacagccacaaacaagtactttaccccacagcttagtggccagtaaacaccctggggactaggaaaaggaaccaactgtaggcacctctccagggcctagggagacaagtgtcctctcttctgcatacatttgggctccccttacagagccctttgccctggctctctggtccttgttgctctaacagtccagatgtacacccagcctcagggggaaggcagctctctccagacagagtctcagggcccagcaaggtcaggttatctgctttcattcagggcaacaaatgatacaaatggtgccagggagtggcaaggccatgggggtaggtgggggtgtctttttcttttcataaagtaacaacagacgagactgaggttaaacatcagaaaaaaacctctggaatgaccttcctcattccaggaggccctggaataaggaagaggcttctttctgagggagctttgaggaattttgacagctgttgacatgggatttgggaaaggtgaagctgtgactggaggggcaggagatggtccaagtgtccatccagagatgagactcttagaatcaaagtgttcagcccaggaagtcttggagatcccaccttctgtggccctgcaccttatgggaagccattaagggggctcatctaggaattctggttacagcccagtgctcatcccagcgtatgctgcctctttagggcagccccaagggccagccagcctgtactctgggcaagagcccaaaatggctaggaatgtttgactcccttaatctcttccccagctacagaggaatcttttctctgcctggtctcagaatgggactgccaactggctcattggtgggagacacagtatcctcaaacctgtggccactggcatgacagtggtgctctgtctccctgggtgacacccaccctaggcttcctcctggatgtgatggggattgccagagaggctcttagcataaaaggcattaggtgggcatttttctgtgtgcccccaaaaagctccatggaaacaggcacctggtagctgcggaacacccgtggacttgtgtatatggtcataggctttgggaagacaggacgtaaaggaaaatgagagaaacaaaatgggtcagatagctttggccacagccccaggcagcctttggggcctatgacacttagtgcccttagatgggatacatcttgcctcggccccaagactcctccaacttacccgtcccatccagggcctgcacagcttagagaggctcacagcttggcaaatgctagggcttcatcagaccactgacttgactcagtgtttgttaaaatggaaccactcccgttggcctactgtttctctcctgtacttcttgtaatgatagttatttattgactctggtagcaggcagttcttaaataaagatggtttctcaacctgttggggaagctggcacgagctgtgggtgttatcatcaggctaaagcatacctccttgtcctgttcttcactccagaggcttttatccaagaatttctttaccccccacacagtgaaatataagtaagttacaacatacagtctatattgcttcatccagtcccaggaggagggaaaaattaggccaagatcttaaggcaggtccaagcagagcacaggcaggcatttggctagaatcagttggctttacctaatacagtggcagtaaacaatgcttatgtgatggta

SEQ ID NO: 16 - protein, 1040 aa, Homo sapiens
MGTATRRKPHLLLVAAVALVSSSAWSSALGSQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVENMAVRPAPHPGTVISHSVAMLILIGSLEL

SEQ ID NO: 17 - RNA, 4189 nt, Homo sapiens
  source 1..4189: mol_type = mRNA
agtctccggccgagtcttctcgcagccgcaacccacctggggccagcccagagctgccagcgccgctcggctccctccctccctcccggcccttcggccgcggcggcgtgcgcctgccttttccgggggcgggggcctggcccgcgcgctcccctcccgcaggcgccacctcggacatccccgggattgctacttctctgccaacttcgccaactcgccagcacttggagaggcccggctcccctcccggcgccctctgaccgcccccgccccgcgcgctctccgaccaccgcctctcggatgaccaggttccaggggagctgagcgagtcgcctcccccgcccagcttcagccctggctgcagctgcagcgcgagccatgcgcccccagtgcaccccggcccggcccaccgccccggggccattctgctgaccgcccagccccgagccccgacagtggcaagttgcggctactgcagttgcaagctccggccaacccggaggagccccagcggggagcgcagtgctgcgccccccgcccccgcgcgccccgcagcagccgggcgttcactcatcctccctcccccaccgtccctcccttttctcctcaagtcctgaagttgagtttgagaggcgacacggcggcggcggccgcgctgctcccgctcctctgcctccccatggatatgcactgcaaagcagaccccttctccgcgatgcacccagggcacgggggtgtgaaccagctcgggggggtgtttgtgaacggccggcccctacccgacgtggtgaggcagcgcatcgtggagctggcccaccagggtgtgcggccctgtgacatctcccggcagctgcgggtcagccacggctgtgtcagcaaaatcctgggcaggtactacgagaccggcagcatcaagccgggtgtgatcggtggctccaagcccaaagtggcgacgcccaaagtggtggacaagattgctgaatacaaacgacagaacccgactatgttcgcctgggagattcgagaccggctcctggccgagggcatctgtgacaatgacacagtgcccagcgtctcttccatcaacagaatcatccggaccaaagttcagcagcctttccacccaacgccggatggggctgggacaggagtgaccgcccctggccacaccattgttcccagcacggcctcccctcctgtttccagcgcctccaatgacccagtgggatcctactccatcaatgggatcctggggattcctcgctccaatggtgagaagaggaaacgtgatgaagatgtgtctgagggctcagtccccaatggagattcccagagtggtgtggacagtttgcggaagcacttgcgagctgacaccttcacccagcagcagctggaagctttggatcgggtctttgagcgtccttcctaccctgacgtcttccaggcatcagagcacatcaaatcagaacaggggaacgagtactccctcccagccctgacccctgggcttgatgaagtcaagtcgagtctatctgcatccaccaaccctgagctgggcagcaacgtgtcaggcacacagacatacccagttgtgactggtcgtgacatggcgagcaccactctgcctggttacccccctcacgtgccccccactggccagggaagctaccccacctccaccctggcaggaatggtgcctgggagcgagttctccggcaacccgtacagccacccccagtacacggcctacaacgaggcttggagattcagcaaccccgccttactaagttccccttattattatagtgccgccccccggggctccgcccctgccgctgctgccgctgcctatgaccgccactagttaccgcggggaccacatcaagcttcaggccgacagcttcggcctccacatcgtccccgtctgaccccaccccggagggagggaggaccgacgcgacgcgatgcctcccggccaccgccccagcctcaccccatcccacgacccccgcaacccttcacatcacccccctcgaaggtcggacaggacgggtggagccgtgggcgggaccctcaggcccgggcccgccgcccccagccccgcctgccgcccctccccgcctgcctggactgcgcggcgccgtgagggggattcggcccagctcgtcccggcctccaccaagccagccccgaagcccgccagccaccctgccggactcgggcgcgacctgctggcgcgcgccggatgtttctgtgacacacaatcagcgcggaccgcagcgcggcccagccccgggcacccgcctcggacgctcgggcgccaggaggcttcgctggaggggctgggccaaggagattaagaagaaaacgactttctgcaggaggaagagcccgctgccgaatccctgggaaaaattcttttcccccagtgccagccggactgccctcgccttccgggtgtgccctgtcccagaagatggaatgggggtgtgggggtccggctctaggaacgggctttgggggcgtcaggtctttccaaggttgggacccaaggatcggggggcccagcagcccgcaccgatcgagccggactctcggctcttcactgctcctcctggcctgcctagttccccagggcccggcacctcctgctgcgagacccggctctcagccctgccttgcccctacctcagcgtctcttccacctgctggcctcccagtttcccctcctgccagtccttcgcctgtcccttgacgccctgcatcctcctccctgactcgcagccccatcggacgctctcccgggaccgccgcaggaccagtttccatagactgcggactggggtcttcctccagcagttacttgatgccccctcccccgacacagactctcaatctgccggtggtaagaaccggttctgagctggcgtctgagctgctgcggggtggaagtggggggctgcccactccactcctcccatcccctcccagcctcctcctccggcaggaactgaacagaaccacaaaaagtctacatttatttaatatgatggtctttgcaaaaaggaacaaaacaacacaaaagcccaccaggctgctgctttgtggaaagacggtgtgtgtcgtgtgaaggcgaaacccggtgtacataacccctccccctccgccccgccccgcccggccccgtagagtccctgtcgcccgccggccctgcctgtagatacgccccgctgtctgtgctgtgagagtcgccgctcgctgggggggaagggggggacacagctacacgcccattaaagcacagcacgtcctgggggaggggggcattttttatgttacaaaaaaaaattacgaaagaaaagaaatctctatgcaaaatgacgaacatggtcctgtggactcctctggcctgttttgttggctctttctctgtaattccgtgttttcgctttttcctccctgcccctctctccctctgcccctctctcctctccgcttctctccccctctgtctctgtctctctccgtctctgtcgctcttgtctgtctgtctctgctctttcctcggcctctctccccagacctggcccggccgccctgtctccgcaggctagatccgaggtggcagctccagcccccgggctcgccccctcgcgggcgtgccccgcgcgccccgggcggccgaaggccgggccgccccgtcccgccccgtagttgctctttcggtagtggcgatgcgccctgcatgtctcctcacccgtggatcgtgacgactcgaaataacagaaacaaagtcaataaagtgaaaataaataaaaatccttgaacaaatccgaaaaggcttggagtcctcgcccagatctctctcccctgcgagccctttttatttgagaaggaaaaagagaaaagagaatcgtttaagggaacccggcgcccagccaggctccagtggcccgaacggggcggcgagggcggcgagggcgccgaggtccggcccatcccagtcctgtggggctggccgggcagagaccccggacccaggcccaggcctaacctgctaaatgtccccggacggttctggtctcctcggccactttcagtgcgtcggttcgttttgattctttttcttttgtgcacataagaaataaataataataataaataaagaataaaattttgtatgtcactc

SEQ ID NO: 18 - RNA, 3469 nt, Homo sapiens
  source 1..3469: mol_type = mRNA
agcgggctgcgcagtgtccgggccgagccggtgcgccgcagactagggcgcctcgggccagggagcgcggaggagccatggccaccgctaacggggccgtggaaaacgggcagccggacaggaagccgccggccctgccgcgccccatccgcaacctggaggtcaagttcaccaagatatttatcaacaatgaatggcacgaatccaagagtgggaaaaagtttgctacatgtaacccttcaactcgggagcaaatatgtgaagtggaagaaggagataagcccgacgtggacaaggctgtggaggctgcacaggttgccttccagaggggctcgccatggcgccggctggatgccctgagtcgtgggcggctgctgcaccagctggctgacctggtggagagggaccgcgccaccttggccgccctggagacgatggatacagggaagccatttcttcatgcttttttcatcgacctggagggctgtattagaaccctcagatactttgcagggtgggcagacaaaatccagggcaagaccatccccacagatgacaacgtcgtgtgcttcaccaggcatgagcccattggtgtctgtggggccatcactccatggaacttccccctgctgatgctggtgtggaagctggcacccgccctctgctgtgggaacaccatggtcctgaagcctgcggagcagacacctctcaccgccctttatctcggctctctgatcaaagaggccgggttccctccaggagtggtgaacattgtgccaggattcgggcccacagtgggagcagcaatttcttctcaccctcagatcaacaagatcgccttcaccggctccacagaggttggaaaactggttaaagaagctgcgtcccggagcaatctgaagcgggtgacgctggagctgggggggaagaacccctgcatcgtgtgtgcggacgctgacttggacttggcagtggagtgtgcccatcagggagtgttcttcaaccaaggccagtgttgcacggcagcctccagggtgttcgtggaggagcaggtctactctgagtttgtcaggcggagcgtggagtatgccaagaaacggcccgtgggagaccccttcgatgtcaaaacagaacaggggcctcagattgatcaaaagcagttcgacaaaatcttagagctgatcgagagtgggaagaaggaaggggccaagctggaatgcgggggctcagccatggaagacaaggggctcttcatcaaacccactgtcttctcagaagtcacagacaacatgcggattgccaaagaggagattttcgggccagtgcaaccaatactgaagttcaaaagtatcgaagaagtgataaaaagagcgaatagcaccgactatggactcacagcagccgtgttcacaaaaaatctcgacaaagccctgaagttggcttctgccttagagtctggaacggtctggatcaactgctacaacgccctctatgcacaggctccatttggtggctttaaaatgtcaggaaatggcagagaactaggtgaatacgctttggccgaatacacagaagtgaaaactgtcaccatcaaacttggcgacaagaacccctgaaggaaaggcggggctccttcctcaaacatcggacggcggaatgtggcagatgaaatgtgctggaggaaaaaaatgacatttctgaccttcccgggacacattcttctggaggctttacatctactggagttgaatgattgctgttttcctctcactctcctgtttattcaccagactggggatgcctataggttgtctgtgaaatcgcagtcctgcctggggagggagctgttggccatttctgtgtttccctttaaaccagatcctggagacagtgagatactcagggcgttgttaacagggagtggtatttgaagtgtccagcagttgcttgaaatgctttgccgaatctgactccagtaagaatgtgggaaaaccccctgtgtgttctgcaagcagggctcttgcaccagcggtctcctcagggtggacctgcttacagagcaagccacgcctctttccgaggtgaaggtgggaccattccttgggaaaggattcacagtaaggttttttggtttttgttttttgttttcttgtttttaaaaaaaggatttcacagtgagaaagttttggttagtgcataccgtggaagggcgccagggtctttgtggattgcatgttgacattgaccgtgagattcggcttcaaaccaatactgcctttggaatatgacagaatcaatagcccagagagcttagtcaaagacgatatcacggtctaccttaaccaaggcactttcttaagcagaaaatattgttgaggttacctttgctgctaaagatccaatcttctaacgccacaacagcatagcaaatcctaggataattcacctcctcatttgacaaatcagagctgtaattcgctttaacaaattacgcatttctatcacgttcactaacagcttatgataagtctgtgtagtcttccttttctccagttctgttacccaatttagattagtaaagcgtacacaactggaaagactgctgtaataacacagccttgttatttttaagtcctattttgatattaatttctgattagttagtaaataacacctggattctatggaggacctcggtcttcatccaagtggcctgagtatttcactggcaggttgtgaatttttcttttcctctttggggatccaaatgatgatgtgcaatttcatgttttaacttgggaaactgaaagtgttcccatatagcttcaaaaacaaaaacaaatgtgttatccgacggatacttttatggttactaactagtactttcctaattgggaaagtagtgcttaagtttgcaaattaagttggggagggcaataataaaatgagggcccgtaacagaaccagtgtgtgtataacgaaaaccatgtataaaatgggcctatcacccttgtcagagatataaattaccacatttgccttcccttcatcagctaacacttatcacttatactaccaataacttgttaaatcaggatttggcttcatacactgaattttcagtattttatctcaagtagatatagacactaaccttgatagtgatacgttagagggttcctattcttccattgtacgataatgtctttaatatgaaatgctacattatttataattggtagagttattgtatctttttatagttgtaagtacacagaggtggtatatttaaacttctgtaatatactgtatttagaaatggaaatatatatagtgttaggtttcacttcttttaaggtttacccctgtggtgtggtttaaaaatctataggcctgggaattccgatcctagctgcagatcgcatcccacaatgcgagaatgataaaataaaattggatatttgagaaa

SEQ ID NO: 19 - DNA, 20 bp, synthetic construct
  source 1..20: mol_type = other DNA
cggaagctaggtgagtgtgg

SEQ ID NO: 20 - DNA, 43 bp, synthetic construct
  source 1..43: mol_type = other DNA
gagtaatacgactcactatagggggaagcccctttcccaatca

SEQ ID NO: 21 - DNA, 20 bp, synthetic construct
  source 1..20: mol_type = other DNA
gaggtccctctccattccct

SEQ ID NO: 22 - DNA, 43 bp, synthetic construct
  source 1..43: mol_type = other DNA
gagtaatacgactcactatagggtgcacccatcagacctccta

SEQ ID NO: 23 - DNA, 20 bp, synthetic construct
  source 1..20: mol_type = other DNA
gttgcacttgctggtcctct

SEQ ID NO: 24 - DNA, 43 bp, synthetic construct
  source 1..43: mol_type = other DNA
gagtaatacgactcactatagggttgagttttgggtgccctac

SEQ ID NO: 25 - DNA, 20 bp, synthetic construct
  source 1..20: mol_type = other DNA
gctgtctgtgctgtgagagt

SEQ ID NO: 26 - DNA, 43 bp, synthetic construct
  source 1..43: mol_type = other DNA
gagtaatacgactcactatagggccggggacatttagcaggtt

SEQ ID NO: 27 - DNA, 20 bp, synthetic construct
  source 1..20: mol_type = other DNA
cagaggctcaggcacaatga

SEQ ID NO: 28 - DNA, 43 bp, synthetic construct
  source 1..43: mol_type = other DNA
gagtaatacgactcactatagggtccgtgtctacccaggactt

SEQ ID NO: 29 - DNA, 20 bp, synthetic construct
  source 1..20: mol_type = other DNA
cccagcggttcttcatgagt

SEQ ID NO: 30 - DNA, 43 bp, synthetic construct
  source 1..43: mol_type = other DNA
gagtaatacgactcactataggggcacacttggttgcgttagt

SEQ ID NO: 31 - DNA, 21 bp, synthetic construct
  source 1..21: mol_type = other DNA
agccctctaactgaacgtgtg

SEQ ID NO: 32 - DNA, 50 bp, synthetic construct
  source 1..50: mol_type = other DNA
gagtaatacgactcactatagggtcttctgagtatctacattcaattgct

SEQ ID NO: 33 - DNA, 20 bp, synthetic construct
  source 1..20: mol_type = other DNA
accgagaaagggagagggaa

SEQ ID NO: 34 - DNA, 43 bp, synthetic construct
  source 1..43: mol_type = other DNA
gagtaatacgactcactatagggtcggccaatttctccaacca

SEQ ID NO: 35 - DNA, 20 bp, synthetic construct
  source 1..20: mol_type = other DNA
gtccgctatgaacctgtggt

SEQ ID NO: 36 - DNA, 43 bp, synthetic construct
  source 1..43: mol_type = other DNA
gagtaatacgactcactatagggatagtctcgcttgcccaagg

SEQ ID NO: 37 - DNA, 20 bp, synthetic construct
  source 1..20: mol_type = other DNA
aacaccgaaatgctggaggt

SEQ ID NO: 38 - DNA, 43 bp, synthetic construct
  source 1..43: mol_type = other DNA
gagtaatacgactcactataggggagaccacatgtacacgcca

(19) United States
(12) Patent Application Publication
(10) Pub. No.: US 2024/0352415 A1
(43) Pub. Date: Oct. 24, 2024

(54) METHODS FOR THE GENERATION OF HUMAN RETINAL GANGLION CELLS AND COMPOSITIONS, ASSAYS, DEVICES, AND KITS COMPRISING SAME

(71) Applicant: Albert Einstein College of Medicine, Bronx, NY (US)

(72) Inventor: Wei Liu, Pelham, NY (US)

(21) Appl. No.: 18/687,436

(22) PCT Filed: Aug. 29, 2022
(86) PCT No.: PCT/US2022/041843

§ 371 Date: Feb. 28, 2024

Related U.S. Application Data
(60) Provisional application No. 63/239,234, filed on Aug. 31, 2021.

(51) Int. Cl. C12N 5/079; C12N 5/071

(52) U.S. Cl. CPC C12N 5/0621; C12N 5/0697; C12N 2506/02; C12N 2506/45; C12N 2513/00; C12N 2533/90

(57) ABSTRACT

Provided herein are organoids having concentric zones of telencephalic and ocular progenitors and methods of producing these organoids. Also provided herein are retinal ganglion cells (RGCs) growing directional axons in these organoids and methods of isolating RGCs.

### CROSS-REFERENCE TO RELATED APPLICATIONS

This application claims the benefit of U.S. Provisional Application No. 63/239,234, filed on Aug. 31, 2021, the entire contents of which are incorporated herein in their entirety by this reference.

### STATEMENT OF GOVERNMENT SUPPORT

This invention was made with government support under grant numbers R01-EY022645 and R21EY029806 awarded by the National Institutes of Health. The government has certain rights in the invention.

### BACKGROUND OF THE INVENTION

The disclosures of all publications, patents, patent application publications and books referred to herein, are hereby incorporated by reference in their entirety into the subject application to more fully describe the art to which the subject invention pertains.

Glaucoma is a leading cause of incurable blindness, affecting millions of people worldwide. In glaucoma, retinal ganglion cells (RGCs) progressively degenerate in subjects having or not having high intraocular pressure. Since RGCs do not regenerate, damage to RGCs is permanent. Current treatments for glaucoma are mostly focused on lowering intraocular pressure. However, intraocular pressure control is often insufficient for RGC protection.

Human RGCs are increasingly important for therapeutic studies for glaucoma. They are advantageous over rodent models in numerous aspects that facilitate the study of the cellular and molecular features of the disease. In addition, rodent models often do not faithfully recapitulate glaucoma phenotypes due to the differences in anatomy and gene regulation between rodents and humans. Human pluripotent stem cells, which include both human embryonic stem cells (hESCs) and human induced pluripotent stem cells (hiPSCs), have remarkable capability to differentiate into any cell types and therefore serve as an unlimited resource for the generation of human RGCs. Human RGCs can be used in a drug delivery studies or platform and can potentially be used for cell replacement therapies for glaucoma.

Previous studies described the generation and isolation of retinal cells from human pluripotent stem cells in 2D and/or 3D cell cultures. In 3D cultures of retinal cells, also known as retinal organoids, all major retinal cells are generated and organized into a laminar structure (Kim et al., Proc Natl Acad Sci USA 116, 10824-10833 (2019); Lowe et al., Stem Cell Reports 6, 743-756 (2016); Meyer et al., Proc Natl Acad Sci USA 106, 16698-16703 (2009); Nakano et al., Cell Stem Cell 10, 771-785 (2012); Zhong et al., Nat Commun 5, 4047 (2014)). However, RGCs do not grow any directional axons in retinal organoids. When retinal organoids are cut into pieces or dissociated into single cells for adherent culture, RGCs grow neurites (Fligor et al., 2018).

Efforts have been made to purify human RGCs. Cell surface protein THY1 is routinely used for purifying rodent RGCs but is not sufficiently specific for isolating human RGCs in stem cell-derived cultures (Sluch et al., Stem Cells Transl Med 6, 1972-1986 (2017)). THY1.2 is engineered for isolating human RGCs, but the RGC isolation is not in a native condition (Sluch et al., 2017). Human RGCs that grow directional axons guided by intrinsic cues and human RGC isolation in a native condition have not been described.

There is an urgent need for effective methods for generating RGCs that grow directional axons and methods for isolating and/or purifying such cells.

### SUMMARY OF THE INVENTION

The present invention is based in part on the development of organoids comprising, among others, RGCs that grow directional long axons. Specifically, the organoids (“CONCEPT organoids”) have concentric zones of anterior ectodermal progenitors and that exhibit coordinated development of telencephalon and ocular tissues, e.g., telencephalic, optic stalk, optic disc, neuroretinal, and multi-lineage ocular progenitors. Additionally, a specific biomarker for developing human retinal ganglion cells (RGCs) is disclosed herein. Accordingly, methods of producing organoids having concentric zones of anterior ectodermal progenitors (“CONCEPT organoids”) and purifying RGCs from organoids. Such methods are valuable in developing and implementing drug discovery assays and cell replacement therapies. Additionally, the methods described herein and the organoids generated from these methods can be used to develop assays, devices, and kits for isolating RGCs, drug screening and/or delivery, and cell replacement therapies.

Certain aspects of the present invention provide an organoid comprising at least two concentric zones of telencephalic and ocular progenitors. In some embodiments, the organoid also comprises a retinal ganglion cell (RGC). The organoid can comprise FOXG1+ telencephalic cells, and in some embodiments, the organoid can comprise PAX6+ multi-lineage retinal cells. In some embodiments, the organoid also comprises VSX2+ neuroretinal cells and/or PAX2+ optic disc cells and/optic stalk cells. In some embodiment, the RGC expresses at least one surface marker selected from the group consisting of ATOH7, POU4F2, ONECUT2, and TUBB3. In some embodiments, the RGC comprises a TUBB3+ axon. In some embodiments, the axon grows in a long, directional manner. In some embodiment, the RGC expresses CNTN2 on the cell surface.

In other aspects, provided herein is a retinal ganglion cell (RGC) that is derived from the organoid of the present disclosure. In some embodiments, the RGC is CNTN2+.

In yet other aspects, a method is provided for isolating a retinal ganglion cell (RGC) from an organoid, the method comprising contacting a cell or cells from the organoid (e.g., partially or fully dissociated organoid) with an antibody that specifically binds to CNTN2, capturing an anti-CNTN2 antibody-bound RGC, and removing the antibody from the antibody-bound RGC, thereby isolating the RGC from the organoid. In some embodiments, the anti-CNTN2 antibody is conjugated to the surface of a paramagnetic bead. In some embodiments, the isolating comprises performing magnetic activated cell sorting (MACS). In some embodiments, the antibody comprises a label, and this label can be a fluorescent moiety. In some embodiments, the isolating comprises performing fluorescence activated cell sorting.

Other aspects provide a method of treating a subject having or suspected of having glaucoma, the method comprising: administering to the subject a composition comprising a therapeutic amount of an RGC described herein.

In yet other aspects, provided herein is a method for treating a subject having or suspected of having glaucoma, the method comprising: administering to the subject a composition comprising a therapeutic amount of RGCs isolated from an organoid using a method described herein (e.g., the methods described above). In some embodiments, the organoid comprises FOXG1+ telencephalic cells. In some embodiments, the organoid comprises PAX6+ multi-lineage ocular cells. In some embodiments, the organoid comprises VSX2+ neuroretinal cells. The RGC, in some embodiments, expresses at least one surface marker selected from the group consisting of ATOH7, POU4F2, ONECUT2, and TUBB3. In some embodiments, the RGC comprises a TUBB3+ axon, and this axon can grow in a long, directional manner in some embodiments. In some embodiments, the RGC expresses CNTN2 on the cell surface.

The invention disclosure describes:
a) The discovery of a telencephalic-eye organoid that is composed of concentric zones of anterior ectodermal progenitors (“CONCEPT organoids”), including FOXG1+ telencephalic progenitors, PAX2+ optic stalk progenitor cells, PAX2+ optic disc progenitor cells, VSX2+ neuroretinal progenitor cells, and PAX6+ multi-lineage retinal progenitors (FIG. 1-FIG. 4, FIG. 6-FIG. 12, FIG. 14-FIG. 18, FIG. 23-FIG. 25);
b) The CONCEPT organoids are generated via a method comprising adherent culture of low-density cysts, which are human pluripotent stem cell-derived hollow spheres with an epithelial structure, sequentially in two types of culture media (FIG. 1, FIG. 6). In some embodiments, the method comprises these sequential steps: i) inducing pluripotent stem cells to form a spherical epithelium called cyst via suspension culture of Matrigel-embedded stem cell sheets in a medium containing N2 B27 supplements; ii) plating individual cysts onto a culture surface at a low density for adherent growth in a medium containing N2 B27 supplements; iii) growing the adherent colonies subsequently in a medium containing KnockOut Serum Replacement;
c) The identification of a native, cell surface protein marker CNTN2 that is specific for developing human RGCs, which is demonstrated by the co-expression of CNTN2 with RGC markers ATOH7, POU4F2, ONECUT2, and TUBB3 (FIG. 2; FIG. 3A-FIG. 3C; FIG. 11; FIG. 12A-FIG. 12J);
d) Through the coordinated tissue development in CONCEPT organoids, the generation of POU4F2+ RGCs that grow directional long axons along a path that is defined by tissues reminiscent of the optic disc and/or optic stalk (FIG. 3; FIG. 4A-FIG. 4C; FIG. 7; FIG. 12A-FIG. 12J; FIG. 25);
e) The immuno-purification of developing human RGCs using magnetic activated cell sorting with an antibody against CNTN2 (FIG. 4D; FIG. 5; FIG. 12L-FIG. 12P).

### BRIEF DESCRIPTION OF THE DRAWINGS

FIG. 1A-FIG. 1L shows fluorescent images of CONCEPT organoids, which were generated via adherent culture of cysts that were passaged to 24-well plates at a low density on day 3-5. FIG. 1A-FIG. 1C show the specificity of telencephalon marker FOXG1, neuroretinal marker VSX2, and multi-lineage retinal marker PAX6, respectively, using immuno-stained sections of mouse embryos. FIG. 1D-FIG. 1L shows that FOXG1, VSX2, and PAX6 exhibit concentric patterns in CONCEPT organoids. Markers, stages of the samples, and scale bars are shown in the images.

FIG. 2A-FIG. 2J shows the results of a cluster analysis of CONCEPT organoids at day 24 using single-cell RNA sequencing. FIG. 2A-2E show the cluster analysis confirming that CONCEPT organoids were composed of FOXG1+ telencephalon cells and PAX6+ and/or VSX2+ retinal cells. FIG. 2F-2J shows cell cluster 11, which specifically expressed retinal ganglion cell (RGC) markers ATOH7, POU4F2, ONECUT2, and TUBB3, as well as cell surface protein CNTN2.

FIG. 3A-FIG. 3J shows that human RGCs in CONCEPT organoids grow directional long axons toward and then along a path defined by tissues reminiscent of the optic disc and optic stalk. FIG. 3A-3F show concentric patterns of immunostaining for RGC nuclear marker POU4F2 (red), RGC axon marker CNTN2+(green), and optic disc and optic stalk marker PAX2 (magenta) in CONCEPT organoids at day 25. Composite color images of three and two channels at low and high magnification are shown. POU4F2+ RGC nucleus (red) are mostly abundant adjacent to a PAX2+ tissue (magenta at moderate levels, bracketed areas in FIG. 3B, FIG. 3C). RGCs grow axons toward and then along a path that is defined by the adjacent PAX2+ cell population (FIG. 3A-FIG. 3E), reminiscent of RGC axon growth towards the optic disc in vivo. In areas where there is a gap in the PAX2+ cell population, RGC axons exit the circular path and grow centrifugally (regions between the two arrowheads in FIG. 3C, FIG. 3F), further indicating that the PAX2+ cell population play critical roles in RGCs axon growth. The PAX2^{High} cell population in the inner circle appears to be a border of the path for RGC axon growth. FIG. 3G-FIG. 3J show that the path for RGC axon growth is within regions that do not express optic stalk marker ALDH1A3, reminiscent of RGC axon growth that is confined in the optic stalk in vivo.

FIG. 4A-FIG. 4D show that CNTN2 and TUBB3 exhibit similar expression in CONCEPT organoids and RGCs can be efficiently purified using magnetic activated cell sorting (MACS) with an antibody against CNTN2. FIG. 4A-FIG. 4B are images taken at day 22 and day 26, respectively, that show POU4F2+ RGCs grow directional TUBB3+ long axons in CONCEPT organoids. FIG. 4B and FIG. 4C show CNTN2 and TUBB3 exhibit very similar expression patterns in CONCEPT organoids. FIG. 4D shows that isolated human RGCs using MACS via an CNTN2 antibody express RGC marker TUBB3.

FIG. 5A shows that isolated human RGCs using MACS via an CNTN2 antibody express both CNTN2 and POU4F2.

FIG. 5B shows that isolated human RGCs using MACS via an CNTN2 antibody express both TUBB3 and POU4F2.

FIG. 6A-FIG. 6W show generation of a telencephalon-eye organoid composed of concentric zones of anterior ectodermal progenitors (CONCEPT). (FIG. 6A) A scheme of the procedure. (FIG. 6B) A diagram of CONCEPT telencephalon-eye organoids showing concentric zones of the anterior ectodermal progenitors. A summary of FIG. 6-FIG. 8, FIG. 10, FIG. 12, FIG. 14, and FIG. 25. (FIG. 6C) Morphology of cysts at day 2 showing the epithelial structure as revealed by the TJ::GFP reporter at the apical surface. (FIG. 6D) Morphology of CONCEPT telencephalon-eye organoids at day 26 under the dark field of a stereomicroscope. Concentric zones of cell populations were visible. n=62 CONCEPT telencephalon-eye organoids in 64 adherent cell colonies. (FIG. 6E-FIG. 6G) Expression of the telencephalon marker Foxg1, retinal markers Vsx2 and Pax6 in mouse eyes at E10-10.5. (FIG. 6H-FIG. 6O) FOXG1+ telencephalic progenitors, VSX2+ and/or PAX6+ retinal progenitors formed concentric zones in CONCEPT telencephalon-eye organoids. N>5 experiments. (FIG. 6P-FIG. 6W) In CONCEPT telencephalon-eye organoids, morphogens FGF8, BMP4, and BMP7 were expressed starting at early stages and subsequently formed concentric gradients. N>5 experiments. Scale bars, 100 μm (FIGS. 6C, 6E, 6M, 6O, 6P, 6S, 6T), 200 μm (FIGS. 6I, 6K), 500 μm (FIGS. 6Q, 6U), 1 mm (FIGS. 6D, 6H, 6J, 6L, 6N, 6R, 6V, 6W).

FIG. 7A-FIG. 7L show retinal ganglion cells (RGCs) grow axons toward and along a path defined by adjacent PAX2+ cell populations in CONCEPT telencephalon-eye organoids. N>5 experiments. (FIG. 7A-FIG. 7D) POU4F2+ RGCs grew TUBB3+ axons toward and then along a path with a circular or a portion of circular shape. (FIG. 7E, FIG. 7F) In mice, Pax2 was expressed in central regions of the retina (arrow in FIG. 7E) and optic stalk (arrowhead in FIG. 7E) at E10.5 and in the optic disc regions and optic stalk at E13.5 (arrowhead and arrow in FIG. 7F, respectively). Tubb3+ axons from the initial RGCs grew toward the optic disc, exited the eye, and navigated within the optic stalk (FIG. 7G). (FIG. 7H-FIG. 7L) In CONCEPT organoids at day 26, TUBB3+ RGC axons grew toward and then along a path defined by an adjacent PAX2+VSX2+ cell population (arrowhead in FIG. 7H, brackets in FIGS. 7I, 7J); PAX2+VSX2− cell population set up an inner boundary of the path. (FIG. 7L) A diagram summarizing RGC axon growth, PAX2+ optic disc, and PAX2+ optic stalk in CONCEPT telencephalon-eye organoids. OS, optic stalk; OD, optic disc. Scale bars, 50 μm (FIGS. 7E, 7F, 7G), 100 μm (FIG. 7B), 200 μm (FIGS. 7A, 7H, 7I).

FIG. 8A-FIG. 8L show that CONCEPT telencephalon-eye organoids contain lens cells that undergo terminal differentiation. N>5 experiments. (FIG. 8A-FIG. 8H) In CONCEPT organoids, lens markers CRYAA and beta crystalline (shown as CRY B in FIG. 8B) were expressed at day 22 (FIGS. 8A, 8B) and day 39 (FIG. 8C, FIG. 8D). Lens cells were not stained by DAPI (FIG. 8E, FIG. 8F); they exhibited a crystal-like shape (FIG. 8G, FIG. 8H). (FIG. 8I, FIG. 8J) When CONCEPT organoids were detached using Dispase at around day 28 and subsequently grown as suspension cultures, crystal-like clusters with fused transparent spheres were found (FIG. 8I), and they continuously survived for months (FIG. 8J). (FIG. 8K-FIG. 8L) The crystal-like lens clusters were free of organelles and exhibited ball-and-socket structures (FIGS. 8K, 8L), as revealed by electron microscopy. Scale bars, 100 μm (FIGS. 8A, 8B, 8C, 8D, 8I), 200 μm (FIG. 8J), 5 μm (FIG. 8K), 200 nm (FIG. 8L).

FIG. 9A-FIG. 9P show that cell clustering analysis identifies telencephalic cells, ocular cells, and two PAX2+ cell populations that mimic the optic disc and optic stalk, respectively. See also FIG. 11, FIG. 12, FIG. 15-FIG. 21, FIG. 23, FIG. 24. CONCEPT telencephalon-eye organoids in one culture well at day 24 were used for single-cell RNA sequencing. Data analysis was performed using Seurat (v3.2.0). (FIG. 9A) Identification of 14 cell clusters. (FIG. 9B) Cell cycle phases as shown as cell cycle scores. (FIG. 9C) FOXG1 expression marked telencephalic cells. (FIG. 9D, FIG. 9E) The expression of PAX6 and/or VSX2 marked retinal cells. (FIG. 9F) PAX2+ cells were found in two major cell populations, which expressed VSX2 and FOXG1, respectively. PAX2+VSX2+ cells were assigned as the optic disc (OD), whereas PAX2+FOXG1+VSX2− cells were assigned as the optic stalk (OS). (FIG. 9G) The expression of the optic disc/stalk marker SEMASA. (FIG. 9H-FIG. 9L) The expression of major differentially expressed genes (DEGs) in cluster 2, the major cell population that mimic the optic disc. (FIG. 9M-FIG. 9P) The expression of major gene markers for PAX2+VSX2− optic stalk cells. OD, optic disc; OS, optic stalk.

FIG. 10A-FIG. 10Q show that inhibition of FGF signaling prior to RGC differentiation with FGFR and MEK inhibitors drastically decreases PAX2+ optic disc cells, RGCs, and directional RGC axon growth. (FIG. 10A) Single-cell RNA sequencing analysis indicate that PAX2, FGF9, and FGF8 are differentially expressed in cluster 2, the major component of PAX2+ optic disc cells. (FIG. 10B) PAX2 expression in CONCEPT organoids on day 25 revealed by in situ hybridization. Two PAX2+ concentric zones corresponding to the optic stalk (OS) and optic disc (OD) are labeled. (FIG. 10C) Dual-color immunohistochemistry indicates the co-localization of FGF8 and PAX2 in the optic disc zone in CONCEPT organoids on day 25. (FIG. 10D-FIG. 10E) TUBB3+ axons grew towards and then along the regions with high FGF8 (FIG. 10D) and FGF9 (FIG. 10E) in CONCEPT organoids on day 25. (FIG. 10F-FIG. 10Q) After inhibition of FGF signaling with MEK inhibitor PD 0325901 starting on day 15, retinal progenitors marked by VSX2 expression (FIG. 10G) and optic stalk cells marked by high-level PAX2 expression largely remained (FIGS. 10J, 10M); RGC somas and directional axon growth marked by CNTN2 expression (FIGS. 10J, 10P), however, were substantially reduced or nearly absent. After inhibition of FGF signaling with FGFR inhibitor PD 161570, VSX2 expression in retinal progenitors (FIG. 10H) and PAX2 expression in optic stalk cells at inner regions (FIG. 10K) were reduced; RGC somas and directional axon growth were drastically reduced or nearly absent (FIG. 10K, 10Q). N>3 experiments. OD, optic disc; OS, optic stalk. Scale bar, 250 μm (FIG. 10B), 100 μm (FIG. 10C-FIG. 10F).

FIG. 11A-FIG. 11H show identification of glycosylphosphatidylinositol (GPI)-anchored cell membrane protein CNTN2 as a specific marker for developing human RGCs. The dataset for FIG. 9 was used for plotting RGC markers. (FIG. 11A-FIG. 11H) Cluster 11 (see also FIG. 9A) differentially expressed RGC markers, including CNTN2.

FIG. 12A-FIG. 12P show that RGCs grow CNTN2+ axons toward and then along a defined path in CONCEPT telencephalon-eye organoids and can be isolated in one step via CNTN2 in a native condition. N>5 experiments. (FIG. 12A-FIG. 12F) PAX2+ cells formed two concentric zones at the inner and outer regions, mimicking the optic stalk (OS) and optic disc (OD), respectively (FIG. 12A-FIG. 12C; high magnifications in FIG. 12D, FIG. 12E). Outside of the two PAX2+ cell populations, POU4F2+ RGCs were located (FIG. 12B). POU4F2+ RGCs grew CNTN2+ axons toward and then along a path defined by the PAX2+ optic disc cells (FIG. 12A-FIG. 12E). RGCs a few hundreds of micrometers away from PAX2+ optic disc cells grew their axons centrifugally (arrow in FIG. 12C). At regions where there was a gap in PAX2+ optic disc cells, CNTN2+ RGC axons exited the circular path and grew centrifugally (diamond arrowhead in FIG. 12A, double arrowheads in FIG. 12B and FIG. 12F). The inner PAX2+ cell population, optic stalk cells, set up a boundary of the path for RGC axon growth. (FIG. 12G, FIG. 12H) Cells that defined the path for RGC axon outgrowth did not express ALDH1A3. In contrast, the cells that set up the boundaries of the path highly expressed ALDH1A3. (FIG. 12I, FIG. 12J) Cells that set up the inner boundary of the path for RGC axon growth were immuno-stained by an antibody that recognizes optic stalk markers VAX1 and VAX2. (FIG. 12K) In E13.5 mouse eye, Aldh1a3 was highly expressed in the peripheral retina (arrow in FIG. 12K) and a small region in the optic disc (arrowhead). Aldh1a3 expression was at low or nearly absent in the central retina. (FIG. 12L-FIG. 12P) One-step isolation of RGCs using MACS with an antibody against CNTN2. RGCs from floating retinal organoids at day 41 (FIG. 12L, 12M) and day 70 (FIG. 12N-FIG. 12O) were dissociated into single cells using Accutase and then isolated using MACS via a CNTN2 antibody. Isolated RGCs were grown for additional 10 days and then were used for immunostaining. Isolated RGCs expressed POU4F2 and grew TUBB3+ neurites in random directions (FIG. 12L). RGCs also expressed CNTN2 (FIG. 12M), ISL1 (FIG. 12N), RBPMS (FIG. 12N), and SNCG (FIG. 12O). Positive cells were counted (FIG. 12P). Scale bars, 200 μm (FIGS. 12A, 12G, 12I), 100 μm (FIG. 12D-FIGS. 12F, 12H, 12J, 12K), 50 μm (FIG. 12L).

FIG. 13A-FIG. 13E show electrophysiological features of RGCs. RGCs from retinal organoids on day 48 were isolated using MACS via a CNTN2 antibody and then grown on polymer coverslips in a chamber slide for 20-25 days before whole-cell patch clamp recordings. (FIG. 13A) Resting membrane potential of RGCs. Black triangle: average; black line: median; box: interquartile range, n=9. (FIG. 13B) RGCs can fire action potentials. Cells were patched in current-clamp mode. A steady current (Im) was injected to maintain the membrane potential at −70 mV and depolarizing current steps of 10 ms (left), 100 ms (middle) or is (right) were injected to elicit action potentials (Vm). (FIG. 13C) RGCs show functional voltage-gated currents. Cells were recorded in voltage-clamp (holding=−80 mV) and depolarizing voltage steps (200 ms, +10 mV steps up to 80 mV, Vm) were applied to record inward and outward voltage-gated currents (Im). Inset: zoom on inward currents. (FIG. 13D) Outward currents are primarily due to voltage-gated potassium channels. Left, representative example of a current-voltage experiment performed in presence of 20 mM Tetraethylammonium (TEA), a blocker of voltage-gated potassium channels. Inset: zoom on inward currents. Right, amplitude of potassium current as a function of membrane potential (mean±SEM; ncontrol=5, nTEA=5). (FIG. 13E) Inward currents result from activity of voltage-gated sodium channels. Left, representative example of a current-voltage experiment performed in presence of 1 μM Tetrodotoxin (TTX), a blocker of voltage-gated sodium channels. Inset: zoom on inward currents. Right, amplitude of sodium current as a function of membrane potential (mean±SEM; ncontrol=5, nTTX=3).

FIG. 14A-FIG. 14F show that reproducibility of CONCEPT telencephalon-eye organoids is demonstrated by consistent gene expression profiles of multiple organoids in whole culture wells. Related to FIG. 1, FIG. 6, and FIG. 12. (FIG. 14A-FIG. 14C) CONCEPT organoids on day 17 expressed FGF8 (FIG. 14A, n=5/5), BMP4 (FIG. 14B, n=4/4; the broken colony was not counted since its morphology is not quite clear), and BMP7 (FIG. 14C, n=4/4) in concentric zones. (FIG. 14D-FIG. 14F) CONCEPT organoids on day 25 expressed FGF8 (FIG. 14D, n=5/8), RGC marker CNTN2 (FIG. 14E, n=5/6), and telencephalon marker EMX2 (FIG. 14F, n=2/2) in concentric zones. Scale bars, 1 mm (FIG. 14A-FIG. 14F).

FIG. 15 shows that mesoderm, endoderm, and neural crest markers are not expressed in CONCEPT telencephalon-eye organoids at 24. Related to FIG. 9. The dataset for FIG. 9 was used for plotting. Expression profiles of mesoderm markers TBXT, GATA2, and HAND1, endoderm markers GATA1, GATA4, and SOX17, neural crest markers SNAI1, SOX10, FOXD3 are shown.

FIG. 16 shows the expression of top differentially expressed genes (DEGs) in cluster 0. Related to FIG. 9. The dataset for FIG. 9 was used for plotting. Cluster 0 was marked by DEGs that were lowly expressed.

FIG. 17A-FIG. 17L show that diencephalon markers and midbrain/hindbrain markers are rarely expressed in CONCEPT telencephalon-eye organoids at day 24. Related to FIG. 9. (FIG. 17A-FIG. 17F) Diencephalon markers GBX2, WNT3, and SOX14, and midbrain/hindbrain markers EN2, PAX7, and TFAP2B were rarely expressed. The dataset for FIG. 9 was used for plotting. (FIG. 17G-FIG. 17L) Expression of the diencephalon markers and midbrain/hindbrain markers in the E14.5 mouse brain. The images were downloaded from a public database (World Wide Web at gp3.mpg.de/) with permission. Image IDs were shown following gene symbols.

FIG. 18A-FIG. 18L show that DEGs for telencephalic clusters in CONCEPT telencephalon-eye organoids include both pallium and subpallium markers. Related to FIG. 9 and FIG. 19. DEGs for telencephalic clusters were used for plotting, and expression of their orthologs in E14.5 mouse brain was shown in FIG. 19. (FIG. 18A-FIG. 18D) Expression of the DEGs POU3F3, RGMA, EDNRB, and SOX13, whose orthologs are expressed in the pallium of E14.5 mouse brain (see also FIG. 19B-FIG. 19E). (FIG. 18E-FIG. 18L) Expression of the DEGs DLX2, RGS16, DLX1, DLL1, DLX6-AS1, NEFL, DCX, and RTN1, which orthologs are expressed in the subpallium of E14.5 mouse brain (see FIG. 19F-FIG. 19M).

FIG. 19A-FIG. 19M show mouse orthologs of the telencephalic DEGs are expressed in both the pallium and subpallium of the E14.5 mouse brain. Related to FIG. 9 and FIG. 18. The images were downloaded from a public database (World Wide Web at gp3.mpg.de/) with permission. Image IDs were shown following gene symbols. (FIG. 19A) Expression of Foxg1. (FIG. 19B-FIG. 19E) Expression of the genes Pou3f3, Rgma, Ednrb, and Sox3 in the pallium of E14.5 mouse brain. (FIG. 19F-FIG. 19M) Expression of the genes Dlx2, Rgs16, Dlx1, Dll1, Dlx6os1, Nefl, Dcx, and Rtn1 in the subpallium of E14.5 mouse brain.

FIG. 20 shows that DEGs in cluster 6 include retina pigment epithelium (RPE) markers. Related to FIG. 9. Top DEGs in cluster 6 include RPE markers PMEL, HSD17B2, DCT, and MITF.

FIG. 21A-FIG. 21T show that mouse orthologs of the markers for Pax2+ optic disc and Pax2+ optic stalk cell clusters in CONCEPT organoids are indeed expressed in the optic disc and optic stalk/nerve, respectively, in E14.5 mouse embryos. Related to FIG. 9. In situ hybridization images of the E14.5 mouse brain were downloaded from a public database (World Wide Web at gp3.mpg.de/) with permission. Image IDs were shown following gene symbols. (FIG. 21A-FIG. 21T) Expression of the gene markers in E14.5 brain is shown.

FIG. 22A-FIG. 22G show that PAX2+ cells, FOXG1+ cells, SIX3+ cells, and ATOH7+ cells are extremely rare in Gabriel et al.'s organoids (Gabriel et al. (2021) Cell Stem Cell, 28(10):1740-1757). Related to FIG. 9. The dataset deposited by Gabriel et al. (2021) (World Wide Web at github.com/Gpasquini/Brain_organoids_with_OpticCups) was used for plotting. (FIG. 22A-FIG. 22G) Cell clustering was reproduced (FIG. 22A, FIG. 22C). In Gabriel et al.'s organoids on day 30 and 60, PAX2+ cells were extremely rare (FIG. 22B, FIG. 22D). Instead of forming cell clusters, a few PAX2+ cells were scattered across datasets. Similarly, in Gabriel et al.'s organoids on day 30, FOXG1+ telencephalic progenitors, SIX3+ retinal progenitors, and ATOH7+ retinogenic progenitors were extremely rare; a few positive cells were scattered across the dataset. VSX2 was filtered out in the dataset of Gabriel et al.'s organoids on day 30 due to its extremely low expression.

FIG. 23A-FIG. 23E show expression signatures of cell cluster 2. Related to FIG. 9 and FIG. 10. (FIG. 23A) A list of top 10 DEGs and additional DEG markers SEMA5A, PAX2, COL13A1, FGF9, and FGF8 in cluster 2 were identified using Seurat analysis. (FIG. 23B-FIG. 23E) Expression of PAX2, SEMA5A, CYP1B1, and LEFTY2 in CONCEPT organoids at day 25 was revealed by in situ hybridization. The PAX2+ optic disc at the outer zone was marked (bracket in FIG. 23B).

FIG. 24 shows FGFR1, FGFR2, FGFR3, MAP2K1, and MAP2K2 are expressed in multiple types of cells in CONCEPT organoids. Related to FIG. 9. In RGCs, FGFR1 and MAP2K2 were expressed.

FIG. 25A-FIG. 25D show CONCEPT telencephalon-eye organoids are generated using hiPSC line AICS 0023. Related to FIG. 12. hiPSCs (AICS 0023) were used for the generation of CONCEPT telencephalon-eye organoids. (FIG. 25A-FIG. 25D) POU4F2+ RGCs grew CNTN2+ axons toward and then along a circular path defined by PAX2+ cell populations. Scale bar, 500 μm (FIG. 25A), 100 μm (FIG. 25B).

### DETAILED DESCRIPTION

The present disclosure is directed to compositions and methods related to human telencephalon-eye organoids having concentric zones of anterior ectodermal progenitors (“CONCEPT organoids”) and a cell surface biomarker specific for developing human RGC's from organoids. Such methods are valuable for developing and implementing drug discovery assays and cell replacement therapies. Additionally, the methods described herein and the organoids generated from these methods can be used to develop assays, devices, and kits for isolating retinal ganglion cells (RGCs). The present disclosure is the represents a significant advancement in the production RGCs that grow directional axons and the isolation of such RGCs in a native condition.

Accordingly, provided herein are CONCEPT organoids and RGCs produced or isolated using the methods described herein. Methods of isolating RGCs using a CNTN2 antibody-mediated magnetic activated cell sorting are also provided.

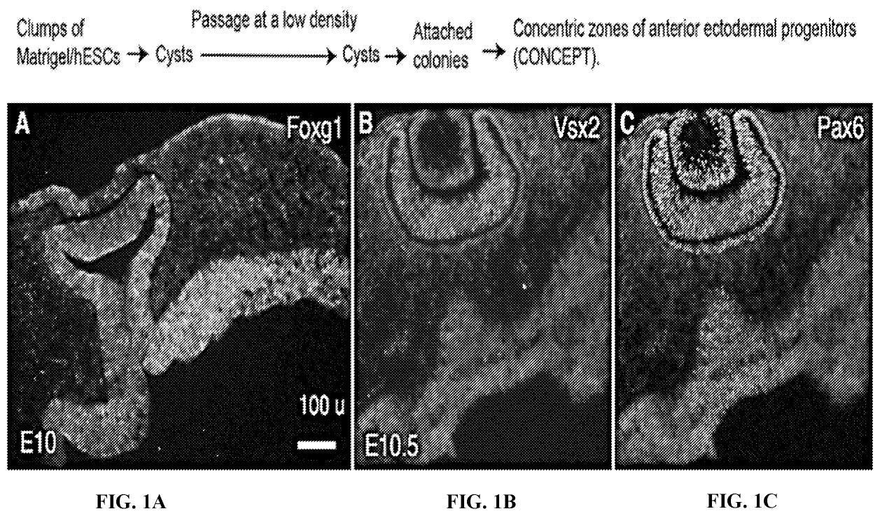

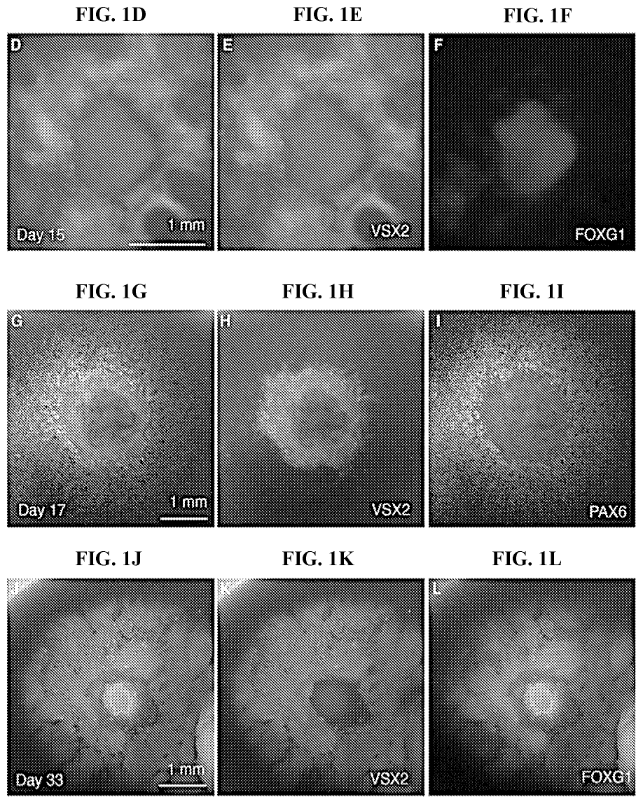

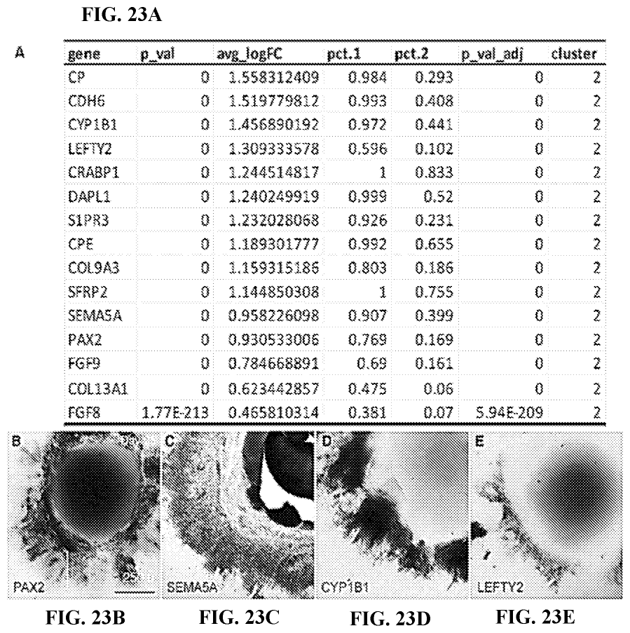

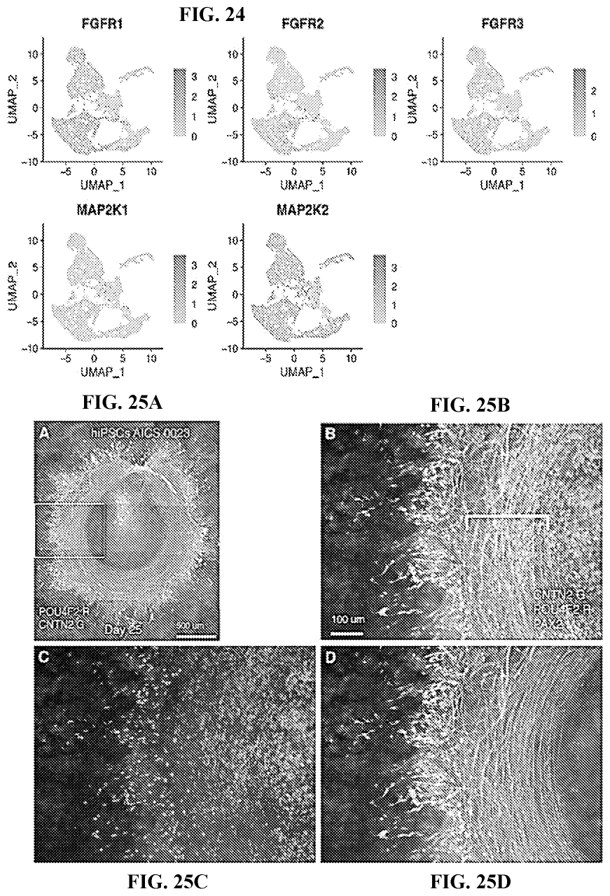

An exemplary method of producing CONCEPT organoids is as follows. The method works well for various types of pluripotent stem cells (see e.g., FIG. 1-FIG. 18, FIG. 23, FIG. 24 for examples using H1 hESCs; see FIG. 25 for examples using hiPSCs. Briefly, hESCs or iPSCs are passaged using ReLeSR three days before the experiment. On day 0, cells are detached using dispase and suspended in ice-cold Matrigel. After gelling at 37° C. for 20 minutes, the hESC/Matrigel clump is gently dispersed in a N2 B27 Medium for suspension culture in a low-adherent 24-well plate. Cysts with a single lumen form on day 1. At a time during day 3-5, cysts are passaged to 24-well plate at low densities. Cysts spontaneously attach to the culture surface and grow as attached colonies. Starting at a time during days 13-16, cell colonies are grown in a medium comprising KnockOut Serum Replacement. Coordinated tissue development in CONCEPT organoids results in concentric zones of anterior ectodermal progenitors, including FOXG1+ telencephalon progenitors, PAX2+ optic stalk progenitors, PAX2+ optic disc progenitors, VSX2+ neuroretinal progenitors, and PAX6+ multi-lineage ocular progenitors. PAX2+ optic disc and optic stalk cells provide guidance cues for RGC axon growth and pathfinding. To generate floating retinal organoids, CONCEPT organoids were detached using dispase and grown as a suspension culture.

Cell clustering analysis of CONCEPT organoids is as follows. CONCEPT organoids at day 24 were dissociated into single cells using papain enzyme. Dissociated single cells were then captured using the 10× Genomics platform for library preparation. 11,158 single cells were captured and sequenced at a depth of 27,842 reads and 2,967 genes per cell. Sequenced cells were filtered (nFeature_RNA >200 & nFeature_RNA <6000 & percent.mt<20) using Seurat (v3.2.0) (Stuart et al., 2019), resulting in 10218 cells with high quality. Cell clustering analysis grouped cells into 14 clusters. Cell cluster 11 were identified as RGCs since they specifically express RGC markers ATOH7, POU4F2, ONECUT2, and TUBB3. Importantly, cell surface protein CNTN2 was identified as a top marker for cluster 11 and therefore its expression in CONCEPT organoids was further characterized using immunostaining assays. After confirming the specific expression of CNTN2 in developing human RGCs, methods of RGC isolation using magnetic activated cell sorting with an antibody against CNTN2 was developed.

Isolation of developing human RGCs using magnetic activated cell sorting is as follows. First, CONCEPT organoids or floating retinal organoids described herein are dissociated into single cells using Accutase or papain. Next, the dissociated cells are bound to MagnaBind IgG beads that are previously coupled to a CNTN2 antibody. Then, RGCs bound to the beads are isolated using a magnetic stand. The isolated RGCs are released from the beads using papain enzyme or directly plated to the culture dishes or chamber slides for culture in the BrainPhys medium supplemented with N2 and B27 (ThermoFisher Scientific). Isolated RGCs were viable in culture for at least 20 days, and their cell identities were confirmed by the expression of multiple RGC markers.

Characterization of protein expression in CONCEPT organoids is performed using standard immunostaining protocols. Organoids were fixed in 4% PFA for 15-30 minutes at room temperature. Phosphate buffered saline (PBS) containing 0.1% tween-20 was used for permeabilization and washes. Target proteins in organoids were recognized by primary antibodies and then visualized by fluorescent secondary antibodies.

Isolation of lens cells is performed as follows. CONCEPT organoids at days 16-50 are detached and partially dissociated using enzymes such as Accutase, Papain, or Dispase so that lens cells are separated from other types of cells whereas lens cells themselves remain as clusters. The partially dissociated cultures are then grown in suspension. Crystal-like structures were manually picked for further culture or assays.

Big crystal-like lens cell clusters can be directly picked from the partially dissociated culture based on their crystal morphology. Alternatively, further growth of partially dissociated cultures in suspension generates additional crystal-like lens cell clusters, which can be picked based on their crystal morphology. CONCEPT organoids comprise multiple cell types, including lens cell. We provide a method to isolate lens cells from CONCEPT organoids. When CONCEPT organoids are detached and partially dissociated into small clusters using enzymes, lens cells are separated from other types of cells, display as crystal-like clusters, can be manually picked based on their crystal morphology, and survive in a suspension culture. Other dissociated cell clusters mostly die in the suspension culture. Remaining surviving cell clusters other than lens cells are morphologically different from lens cell clusters.

### Definitions

In order that the present description may be more readily understood, certain terms are first defined. Additional definitions are set forth throughout the detailed description.

The term “antigen-binding protein” encompasses any polypeptide that binds specifically to any one of the antigens described herein. In some embodiments, an antigen-binding protein comprises an antibody. The term “antibody” as used to herein includes whole antibodies and any antigen binding fragments (i.e., “antigen-binding portions”) or single chains thereof. An “antibody” refers, in some embodiments, to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (KD) of 10⁻⁵ to 10⁻¹ M or less. Any KD greater than about 10⁻⁴ M is generally considered to indicate nonspecific binding. As used herein, an antibody that “binds specifically” to an antigen refers to an antibody that binds to the antigen and substantially identical antigens with high affinity, which means having a KD of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE 2000 instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to an antigen” refers to an antibody that binds to a soluble or cell bound antigen with a K_{D} of 10⁻⁷ M or less, such as approximately less than 10⁻⁸ M, 10⁻⁹ M or 10⁻¹⁰ M or even lower.

“And/or” as used herein, for example, with option A and/or option B, encompasses the separate embodiments of (i) option A, (ii) option B, and (iii) option A plus option B.

A “biological sample” can be obtained from a subject. Subjects from which biological samples can be obtained can be healthy or asymptomatic individuals, individuals that have or are suspected of having a disease (e.g., a loss of RGC, e.g., glaucoma) or a pre-disposition to a disease, and/or individuals that are in need of therapy or suspected of needing therapy.

“Detect” refers to identifying the presence, absence or amount of the analyte to be detected.

A “detectable label” or “label” is meant that a composition that when linked to a molecule of interest renders the latter detectable, via spectroscopic, photochemical, biochemical, immunochemical, or chemical means. For example, useful non-limiting examples of the labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include a condition due to a loss of RGCs, e.g., glaucoma.

The terms “isolated,” “captured,” “purified,” or “biologically pure” refer to material that is free to varying degrees from components which normally accompany it as found in its native state. “Isolate” or “capture” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a cell, nucleic acid, or peptide of this invention is purified if it is substantially free of cellular material, viral material, or culture medium when produced by recombinant DNA techniques, or chemical precursors or other chemicals when chemically synthesized. Purity and homogeneity of cells are generally determined using a cell sorting technique, such as MACS or FACS). Purity and homogeneity of nucleic acids and proteins are typically determined using analytical chemistry techniques, for example, polyacrylamide gel electrophoresis or high-performance liquid chromatography (HPLC).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “organoid” is meant a two-dimensional (2D) or three-dimensional (3D) in vitro tissue culture that mimics some of the structural and/or functional properties of a particular organ. Organoids can be derived from stem or other progenitor cells and can more accurately mimic an organ than non-primate animal models. For 3D organoids, decellularized extracellular matrices or engineered matrices are used as scaffolds with the latter having the advantage of being optimized to support growth and maturation of the organoid. Organoid technology has been previously described for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165(7):1586-1597).

A “polypeptide” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. A “protein” may comprise one or more polypeptides.

The term “nucleic acid molecule,” as used herein, is intended to include DNA molecules and RNA molecules. A nucleic acid molecule may be single-stranded or double-stranded, and may be cDNA.

The nucleic acid compositions of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, cDNA, or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

As used herein, the term “percent sequence identity” refers to the degree of identity between any given query sequence and a subject sequence. A percent identity for any query nucleic acid or amino acid sequence, e.g., a transcription factor, relative to another subject nucleic acid or amino acid sequence can be determined as follows. The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The percent identity between two nucleotide sequences can be determined using the GAP program in the GCG software package (available on the World Wide Web at gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See World Wide Web at ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

As used herein, “administering” refers to the physical introduction of a composition comprising a therapeutic agent to a subject, using any of the various methods and delivery systems known to those skilled in the art. Examples of routes of administration include implantation, transplantation, grafting, and injection (intramuscular, subcutaneous, intravenous, parenterally, intraperitoneally, intrathecal, etc.) routes. The injection can be a bolus injection or can be a continuous infusion. Depending on the route of administration, the agent can be coated with or disposed in a selected material to protect it from natural conditions which may detrimentally affect its ability to perform its intended function. The cellular compositions of the present invention can be administered intraorbitally or in any other manner that safely and effectively delivers the composition to the retina. Administering can also be performed, for example, once, a plurality of times, and/or over one or more extended periods.

By “subject” is meant an animal, including, but not limited to, a human or non-human animal, such as a bovine, equine, canine, ovine, rodent (e.g., a mouse), or feline. The term “non-human animal” includes all vertebrates, e.g., mammals and non-mammals, such as non-human primates, sheep, dog, cow, chickens, amphibians, reptiles, etc. In preferred embodiments, the subject is a mammal (e.g., a human).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug or cellular composition includes a “prophylactically effective amount” or a “prophylactically effective dosage,” which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive either prophylactic or therapeutic treatment.

As used herein, the terms “ug” and “uM” are used interchangeably with “μg” and “μm”.

The articles “a” and “an” are used herein to refer to one or to more than one (i.e., to at least one) of the grammatical object of the article. By way of example, “an element” means one element or more than one element.

The amount of a biomarker (e.g., an RGC biomarker) in a subject is “significantly” higher or lower than the normal amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level, altered activity, changes in biomarker inhibition/blocking, changes in test agent binding, and the like.

The “amount” of a marker, e.g., level of an RGC biomarker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

The term “control” refers to any reference standard suitable to provide a comparison to the RGC biomarkers described herein in a test sample. In some embodiments, the control comprises obtaining a “control sample” from which antigen levels are detected and compared to the antigen levels from the test sample. Such a control sample may comprise any suitable sample, including but not limited to a sample from a control RGC (can be a stored sample or a previous sample measurement) with a known outcome; normal RGCs isolated from a subject, such as a normal subject, cultured primary cells/tissues isolated from a subject, such as a normal subject or a subject with altered RGCs (e.g., a subject having glaucoma), or a primary cells/tissues obtained from a depository. In another preferred embodiment, the control may comprise a reference standard level from any suitable source, including but not limited to housekeeping genes, an antigen level range from normal tissue (or other previously analyzed control sample), a previously determined antigen level range within a test sample from a group of subjects, or a set of subjects with a certain outcome or receiving a certain treatment. It will be understood by those of skill in the art that such control samples and reference standard marker levels can be used in combination as controls in the methods of the present invention.

The “normal” level of a marker is the level of the marker in cells of a subject, e.g., a human patient, not afflicted with a disease or disorder related to aberrant marker levels.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of glaucoma or the likelihood of recovery from glaucoma. In some embodiments, the use of statistical algorithms provides a prognosis of glaucoma in a subject.

The terms “response” or “responsiveness” refers to response to a therapy. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a subject will exhibit a favorable response is equivalent to evaluating the likelihood that the subject will not exhibit favorable response (i.e., will exhibit a lack of response or be non-responsive).

A suspension culture is a type of cell culture in which single cells or small aggregates of cells are allowed to function and multiply in an agitated growth medium, thus forming a suspension (i.e., without attaching to a surface).

### Eye Development and Certain Significance of the Present Studies

Our understanding of eye and brain development in humans is mostly deduced from animal studies. In mice, fate mapping of the anterior neural plate reveals that the eye field is located in rostral regions surrounded anteriorly and laterally by the telencephalic field and caudally and medially by the diencephalic field, indicating the proximity of their embryonic origins. Subsequent evagination of the eye field generates bilateral optic vesicles and optic stalks; the optic stalks connect the optic vesicles to the forebrain, forming a midline-periphery axis. The optic vesicle then invaginates ventrally, forming a groove called the optic fissure. The two opposing edges of the optic fissure subsequently fuse, resulting in the double-layered optic cup in which the inner and outer layers develop into the neuroretina and retina pigment epithelium (RPE), respectively. The posterior pole of the optic-cup forms the optic disc (also known as optic nerve head), which serves as the exit into the optic stalk. In the central neuroretina close to the nascent optic disc, retinal ganglion cells (RGCs) start to appear as the optic fissure nearly closes. Early RGC axons find their path toward the optic disc and then enter the optic stalk to reach their targets in the brain. Concentrically organized growth-promoting and growth-inhibitory guidance molecules around the optic disc regulate RGC axon growth and pathfinding through multiple mechanisms. The optic stalk later forms the optic nerve. Coinciding with optic-cup formation, the surface ectoderm facing the optic vesicle thickens to form the lens placode and later invaginates to form the lens vesicle. Collectively, the early development of telencephalic and ocular tissues is highly coordinated in mammals.

Early telencephalic and eye development is marked and regulated by a group of signal transduction molecules and tissue-specific transcription factors. In mice, Bmp4 and Bmp7 are expressed in the dorsomedial telencephalon, optic vesicles, and presumptive lens placodes. Bmp4 is required for lens induction, and Bmp7 is required for proper patterning of the optic fissure. Fgf8 is specifically expressed at the rostral forebrain at early stages, induces Foxg1 expression, and regulates telencephalic patterning in a dose-dependent manner. In chicks, Fgf8 and Fgf3 coordinate the initiation of retinal differentiation; FGF8 maintains Pax2 expression in the optic stalk. In mice, Foxg1 is specifically expressed in the presumptive telencephalon and is essential for the development of the cerebral hemispheres. Pax6 is specifically expressed in the eye field and is essential for the development of multiple retinal lineages such as the neuroretina, lens, and retinal pigment epithelium. Pax2 is expressed in the ventral optic stalk, optic vesicles, central neuroretina, and optic disc, and is essential for optic stalk and nerve development. Vax1 and Vax2 are expressed in the optic stalk and ventral retina and are jointly required for the optic stalk development. Vsx2 and Mitf are specifically expressed in the neuroretina and RPE, respectively, and are essential for retinal development. Aldh1a3 expression was low in the differentiating central retina but was high in the peripheral retina and the optic stalk. In humans, ALDH1A3 loss of function lead to bilateral anophthalmia and/or microphthalmia and hypoplasia of the optic nerve and optic chiasm. Despite these findings in vertebrates, the mechanisms underlying early development of the eye and telencephalon in humans is still largely unknown.

Human organoids become increasingly important for studying human tissue development, drug discovery, and regenerative medicine. Three-dimensional (3-D) retinal organoids further improved in follow-up studies. Retinal organoids derived from human embryonic stem cells (hESCs) display a stratified structure containing all major types of retinal cells. Although RGCs are differentiated in 3-D retinal organoids, there is no proper RGC axon outgrowth and pathfinding since the tissues that mimic the optic disc and optic stalk are missing. When retinal organoids are dissociated into single cells or cut into pieces for adherent culture, RGCs generate neurites. A variety of brain organoids have been described, mimicking brain development in numerous aspects. Although rudimentary ocular tissues are occasionally found in some brain organoids, ocular and brain tissues are not patterned along any defined axis. Assembloids in which retinal, thalamic, and cortical organoids are assembled together are recently reported. Despite these advances, RGC axon outgrowth and pathfinding directed by intrinsic cues within organoids, such as optic stalk and optic disc tissues, have not been reported.

It was hypothesized herein that coordinated specification of telencephalic and ocular tissues via morphogen gradients generates tissues that provide guidance cues for RGC axon growth and pathfinding. In support of this hypothesis, generated herein were self-formed human telencephalon-eye organoids that comprise concentric zones of anterior ectodermal progenitors (CONCEPT), including FOXG1+ telencephalon, PAX2+ optic stalk, PAX2+ optic disc, VSX2+ neuroretina, and PAX6+ multi-lineage tissues along the center-periphery axis. FGFs and BMPs were expressed starting at early stages and subsequently exhibited concentric gradients, indicating their involvement in coordinated cell specification. Early differentiated RGCs grew their axons toward and then along a path defined by adjacent PAX2+ cell populations. Single-cell RNA sequencing of CONCEPT organoids revealed telencephalic and ocular identities. Developed herein is a one-step method for isolating human RGCs via CNTN2 under a native condition. The studies of the present disclosure provide deeper insight into coordinated specification of telencephalic and ocular tissues for directional RGC axon growth in humans and establish tools for studying RGC-related diseases such as glaucoma.

The telencephalon and eye in mammals are originated from adjacent fields at the anterior neural plate. Morphogenesis of the embryonic fields leads to the formation of telencephalon, optic stalk, optic disc, and neuroretina along the midline-periphery axis. Retinal ganglion cells (RGCs) grow axons toward the optic disc and then along the optic stalk to reach the brain. How these telencephalic and ocular tissues are specified coordinately for directional RGC axon growth in humans are unclear. Demonstrated herein is the self-formation of human telencephalon-eye organoids comprising concentric zones of FOXG1+ telencephalon, PAX2+ optic-stalk, PAX2+ optic-disc, VSX2+ neuroretina, and PAX6+ multi-lineage tissues along the center-periphery axis. FGFs and BMPs were early expressed and subsequently exhibited concentric gradients, indicating their involvement in coordinated cell specification. Initially-differentiated RGCs grew axons towards and then along a path defined by adjacent PAX2+ cell populations. Non-neural ocular tissues also existed. Single-cell RNA sequencing confirmed telencephalic and ocular identities, revealed expression signatures of two PAX2+ cell populations that mimic the optic-disc and optic-stalk, respectively, and identified RGC-specific cell-surface protein CNTN2. PAX2+ optic-disc cells differentially expressed FGF8 and FGF9; inhibition of FGF signaling with FGFR and MEK inhibitors prior to RGC differentiation drastically decreased PAX2+ optic-disc cells, RGCs, and directional RGC axon growth. Using a CNTN2 antibody, isolated herein are RGCs that exhibited electrophysiological signatures of excitable cells. The findings of the present disclosure provide insight into the coordinated specification of early telencephalic and ocular tissues for directional RGC axon growth in humans and establish valuable tools for studying RGC-related diseases such as glaucoma.

Such findings are significant for the treatment of eye diseases, including but not limited to glaucoma. Retinal ganglion cells (RGCs) degenerate in glaucoma. Therapeutic studies for RGC regeneration need better understanding of RGC differentiation and axon growth in humans. Although RGCs are differentiated in retinal organoids, proper RGC axon growth is absent. Through coordinated cell specification mediated by morphogen gradients, generated herein are self-organized organoids comprising concentric zones of telencephalic and ocular tissues, including optic stalk and optic disc that provide cues for RGC axon growth and pathfinding. Single-cell RNA sequencing identified RGC-specific cell surface protein CNTN2, leading to one-step isolation of functional RGCs under a native condition. The present studies provide deeper insight into coordinated specification of telencephalic and ocular tissues for RGC axon growth in humans and establish tools for studying RGC-related diseases such as glaucoma.

### Biomarkers

A “biomarker” or “marker” is a particular protein, nucleic acid molecule, or other attribute that can be used to identify, isolate, or purify a particular cell. Some biomarkers have been associated with physiological states (e.g., disease states) or with risks of developing a disease or condition. In particular embodiments, a biomarker is an organic biomolecule that is differentially present in a test sample compared to a control. For example, a biomarker associated with a loss of a retinal ganglion cell (RGC) (e.g., glaucoma) is differentially present in a sample taken from a subject of one phenotypic status (e.g., having or at risk of developing a loss of an RGC, e.g., glaucoma) as compared with another phenotypic status (e.g., not having a loss of an RGC, e.g., glaucoma). Biomarkers can also be used to characterize a sample or the source of the sample. For example, an organoid can be characterized by the biomarkers it expresses or does not express. These biomarkers can be used to compare to the biomarker profile of a naturally occurring tissue or organ that the organoid is meant to model. Accordingly, if the biomarker profiles between the organoid and the tissue or organ it is meant to model are significantly different, the organoid may not be a suitable model for the tissue or organ. Conversely, similar or even identical biomarker profiles between an organoid and a tissue or organ is one indication that the organoid is a suitable model of the tissue or organ.

Biomarkers are often used in the detection and diagnosis of different phenotypic statuses (e.g., having/not having disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney, and odds ratio. Biomarkers, alone or in combination, can provide measures of relative risk that a subject belongs to one phenotypic status or another. Therefore, they are useful as markers for detecting and/or characterizing a disease.

Any suitable method can be used to detect markers expressed by specific cells, for example, cells in an organoid can have distinct biomarker patterns that are similar to or the same as the biomarker profile of the organ the organoid is designed to mimic. For example, retinal organoids will have cells that express one or more of FOXG1, VSX2, PAX6, TUBB3, ATOH7, POU4F2, ONECUT2, and CNTN2. In some embodiments, methods to detect markers further comprise steps to quantify the markers.

Cells can be harvested from an organoid. For example, RGCs can be isolated and purified from an organoid using the methods presented herein. In some embodiments, the methods can be used to detect 1, 2, 3, 4, 5, 6, 7, 8, or more markers. In some embodiments, a biomarker used to identify, isolate, or purify RGCs or other cell types from a retinal organoid is any one of the biomarkers listed in Table 1.

TABLE 1
SEQ ID NO: 1 Human Forkhead Box Protein G1 Nucleic Acid Sequence (NM_005249.5,
CDS region from 494-1963)

| | |
|---|---|
| 1 | aattgtggct atagccgcct cgatcgctgt ctccccagcc tcgccgcggc cgctccggga |
| 61 | cgcgcccgcc cgccgcccgg ctctcccccc ctttgggctg ctgctgctgc tgctgtgact |
| 121 | gctgctgcga gaggaggagg aggaggagga agcagcgggg gggggagcgg ggggtggggg |
| 181 | gggagaccaa gaagtacagt tgggagcgag ggagcttcac ccccggggcg gtggttgttt |
| 241 | cttttttctt tctttctttt ttcttttcct tttttttttt ttttctaatt cctgaggggt |
| 301 | ggttgctgct tttgctacat gacttgccag cgcccgagcc tgcggtccaa ctgcgctgct |
| 361 | gccggagcgc tcagtgccgc cgctgccgcc cgcgcccccc gcgccccgtt cggcacccac |
| 421 | cggtcgccgc cgcccgccgc gccgctgtcc cgctcccgcg ccgccgccgc cgtttccccc |
| 481 | cgacgactgg gtgatgctgg acatgggaga taggaaagag gtgaaaatga tccccaagtc |
| 541 | ctcgttcagc atcaacagcc tggtgcccga ggcggtccag aacgacaacc accacgcgag |
| 601 | ccacggccac cacaacagcc accaccccca gcaccaccac caccaccacc accatcacca |
| 661 | ccacccgccg ccgcccgccc cgcaaccgcc gccgccgccg cagcagcagc agccgccgcc |
| 721 | gccgccgccc ccggcaccgc agccccccca gacgcggggc gccccggccg ccgacgacga |
| 781 | caagggcccc cagcagctgc tgctcccgcc gccgccaccg ccaccaccgg ccgccgccct |
| 841 | ggacggggct aaagcggacg ggctgggcgg caagggcgag ccgggcggcg ggccggggga |
| 901 | gctggcgccc gtcgggccgg acgagaagga gaagggcgcc ggcgccgggg gggaggagaa |
| 961 | gaagggggcg ggcgagggcg gcaaggacgg ggaggggggc aaggagggcg agaagaagaa |
| 1021 | cggcaagtac gagaagccgc cgttcagcta caacgcgctc atcatgatgg ccatccggca |
| 1081 | gagccccgag aagcggctca cgctcaacgg catctacgag ttcatcatga agaacttccc |
| 1141 | ttactaccgc gagaacaagc agggctggca gaactccatc cgccacaatc tgtccctcaa |
| 1201 | caagtgcttc gtgaaggtgc cgcgccacta cgacgacccg ggcaagggca actactggat |
| 1261 | gctggacccg tcgagcgacg acgtgttcat cggcggcacc acgggcaagc tgcggcgccg |
| 1321 | ctccaccacc tcgcgggcca agctggcctt caagcgcggt gcgcgcctca cctccaccgg |
| 1381 | cctcaccttc atggaccgcg ccggctccct ctactggccc atgtcgccct tcctgtccct |
| 1441 | gcaccacccc cgcgccagca gcactttgag ttacaacggc accacgtcgg cctaccccag |
| 1501 | ccaccccatg ccctacagct ccgtgttgac tcagaactcg ctgggcaaca accactcctt |
| 1561 | ctccaccgcc aacggcctga gcgtggaccg gctggtcaac ggggagatcc cgtacgccac |
| 1621 | gcaccacctc acggccgccg cgctagccgc ctcggtgccc tgcggcctgt cggtgccctg |
| 1681 | ctctgggacc tactccctca acccctgctc cgtcaacctg ctcgcgggcc agaccagtta |
| 1741 | ctttttcccc cacgtcccgc acccgtcaat gacttcgcag agcagcacgt ccatgagcgc |
| 1801 | cagggccgcg tcctcctcca cgtcgccgca ggccccctcg accctgccct gtgagtcttt |
| 1861 | aagaccctct ttgccaagtt ttacgacggg actgtctggg ggactgtctg attatttcac |
| 1921 | acatcaaaat caggggtctt cttccaaccc tttaatacat taacatccct gggaccagac |
| 1981 | tgtaagtgaa cgttttacac acatttgcat tgtaaatgat aattaaaaaa ataagtccag |
| 2041 | gtatttttta ttaagccccc ccctcccatt tctgtacgtt tgttcagtct ctagggttgt |
| 2101 | ttattattct aacaaggtgt ggagtgtcag cgaggtgcaa tgtggggaga atacattgta |
| 2161 | gaatataagg tttggaagtc aaattatagt agaatgtgta tctaaatagt gactgctttg |
| 2221 | ccatttcatt caaacctgac aagtctatct ctaagagccg ccagatttcc atgtgtgcag |
| 2281 | tattataagt tatcatggaa ctatatggtg gacgcagacc ttgagaacaa cctaaattat |
| 2341 | ggggagaatt ttaaaatgtt aaactgtaat ttgtatttaa aaagcattcg tagtaaaggt |
| 2401 | gcccaagaaa ttattttggc catttattgt tttgtccttt tctttaaaga actgtttttt |
| 2461 | tttcttttgt ttacttttag accaaagatt gggttctaga aaatgcactt ggtatactaa |
| 2521 | gtattaaaac aaacaaaaag gaaagttgtt tcagttggca acactgccca ttcaattgaa |
| 2581 | tcagaagggg acaaaattaa cgattgcctt cagtttgtgt tgtgtatatt ttgatgtatg |
| 2641 | tggtcactaa caggtcactt ttattttttc taaatgtagt gaaatgttaa tacctattgt |
| 2701 | acttataggt aaaccttgca aatatgtaac ctgtgttgcg caaatgccgc ataaatttga |
| 2761 | gtgattgtta atgttgtctt aaaatttctt gattgtgata ctgtggtcat atgcccgtgt |
| 2821 | ttgtcactta caaaaatgtt tactatgaac acacagaaat aaaaaatagg ctaaattcat |
| 2881 | atatatcttg atacttttgt ctcttttatt aagtagagct aattttttaa agaccaatca |
| 2941 | acttataggg aattcaaagg ctttttcagc caaactaaaa tttaaactgc tcctttaatt |
| 3001 | tgaactgact ctaaaaatga aaatagtatt tttccctttg tgaacaaatt ttacaaggag |
| 3061 | cagcctattt aataaacact agctttaaac aaagtatagg cttttcagct gatacctgta |
| 3121 | agtttctgtg gatatacagc aaaaagagat ataatttaat tttctgtgca tagctcttta |
| 3181 | ccctgtgttt atttccaaat ccattaatag aatgccattt atatattttg tttcaggtat |
| 3241 | attgttaata gagcttggca aattataaat aaatatatgt atatggttag atagaagtga |
| 3301 | ctataatgca cacatatgta atatatatag acacacagag cccttcagtt caggtacaat |
| 3361 | ttgcgctatg aatgctgcaa acatttttgt ttaaatattt gtatttatac tttctaagtc |
| 3421 | agcatttatt tttgtggctg tttacccaca atgaaagagt tctaataaag atgtgctgaa |
| 3481 | gttgcaatat a |

SEQ ID NO: 2 Human Forkhead Box Protein G1 Amino Acid Sequence (NP_005240.3)

| | |
|---|---|
| 1 | mldmgdrkev kmipkssfsi nslvpeavqn dnhhashghh nshhpqhhhh hhhhhhhppp |
| 61 | papqpppppq qqqppppppp apqppqtrga paadddkgpq qlllpppppp ppaaaldgak |
| 121 | adglggkgep gggpgelapv gpdekekgag aggeekkgag eggkdgeggk egekkngkye |
| 181 | kppfsynali mmairqspek rltingiyef imknfpyyre nkqgwqnsir hnlslnkcfv |
| 241 | kvprhyddpg kgnywmldps sddvfiggtt gklrrrstts raklafkrga rltstgltfm |
| 301 | dragslywpm spflslhhpr asstlsyngt tsaypshpmp yssvltqnsl gnnhsfstan |
| 361 | glsvdrlvng eipyathhlt aaalaasvpc glsvpcsgty slnpcsvnll agqtsyffph |
| 421 | vphpsmtsqs stsmsaraas sstspqapst lpceslrpsl psfttglsgg lsdyfthqnq |
| 481 | gsssnplih |

SEQ ID NO: 3 Human Visual System Homeobox 2 Nucleic Acid Sequence
(NM_182894.3, CDS region from 114-1199)

| | |
|---|---|
| 1 | agcattagac accggagggg gcacctggga ccaacttcgc gaagcgggaa gcccggcggg |
| 61 | ggggtggggg gagctaaaga cctgcggcct cagcccctcc aaagaacagg gagatgacgg |
| 121 | ggaaagcagg ggaagcgctg agcaagccca aatccgagac agtggccaag agtacctcgg |
| 181 | ggggcgcccc ggccaggtgc actgggttcg gcatccagga gatcctgggc ttgaacaagg |
| 241 | agcccccgag ctcccacccg cgggcagcgc tcgacggcct ggcccccggg cacttgctgg |
| 301 | cggcgcgctc agtgctcagc cccgcggggg tgggcggcat ggggcttctg gggcccgggg |
| 361 | ggctccctgg cttctacacg cagcccacct tcctggaagt gctgtccgac ccgcagagcg |
| 421 | tccacttgca gccattgggc agagcatcgg ggccgctgga caccagccag acggccagct |
| 481 | cggattctga agatgtttcc tccagcgatc gaaaaatgtc caaatctgct ttaaaccaga |
| 541 | ccaagaaacg gaagaagcgg cgacacagga caatctttac ctcctaccag ctagaggagc |
| 601 | tggagaaggc attcaacgaa gcccactacc cagacgtcta tgcccgggag atgctggcca |
| 661 | tgaaaacgga gctgccggaa gacaggatac aggtctggtt ccagaaccgt cgagccaagt |
| 721 | ggaggaagcg ggagaagtgc tggggccgga gcagtgtcat ggcggagtat gggctctacg |
| 781 | gggccatggt gcggcactcc atccccctgc ccgagtccat cctcaagtca gccaaggatg |
| 841 | gcatcatgga ctcctgtgcc ccgtggctac tggggatgca caaaaagtcg ctggaggcag |
| 901 | cagccgagtc ggggaggaag cccgaggggg aacgccaggc cctgcccaag ctcgacaaga |
| 961 | tggagcagga cgagcggggc cccgacgctc aggcggccat ctcccaggag gaactgaggg |
| 1021 | agaacagcat tgcggtgctc cgggccaaag ctcaggagca cagcaccaaa gtgctgggga |
| 1081 | ctgtgtctgg gccggacagc ctggcccgga gtaccgagaa gccagaggag gaggaggcca |
| 1141 | tggatgaaga caggccggcg gagaggctca gtccaccgca gctggaggac atggcttagg |
| 1201 | tcaaggcgcg ctcagatgcc ggagccccaa gactctgctc tcctcgggcc ctgtggtgct |
| 1261 | gggagatgct ctctgaggca aggcccagac ctggcctctg ccatcctccc tgttccccac |
| 1321 | aggtcctcca tcacccctgg tggctgcagg caccgctggg ttctgactct ggaccatgct |
| 1381 | gagacatccc tcatctagtc ttgacctctc cagcatccca gcctcagaag ccttcttgct |
| 1441 | gcccacaacg tcccctcaag ccccttctct caatcccttt gcaacgctca ctggttttgg |
| 1501 | ccaccccttg ctctctgttc tcttgcttta aagagtcctc cttcccagct ctacattctg |
| 1561 | ctctgcccat gcctaaagcc cattgctgca aatgcattgt gaattgcctg ccatggctgt |
| 1621 | gacacagacg gaggactgga actgcaactc cagcgtcctc agcaccccac tcctcagtaa |
| 1681 | aagtcttctc ccaactcagc ctgttccttc ctgcagacct ggctgggcct gggcttccac |
| 1741 | agtgtgaaga cactgtgcag gcccaggcag gcccagcctc tgccccaccc atcagtggag |
| 1801 | tccagatggc aggctacagt tgggaagtct cagcctgggc ccctcagcca cccttggtct |
| 1861 | catgccccgc catggtcacc ctcaggaacc caccctctcc acacccagcc tgtcccactg |
| 1921 | gctcctccca gcacaggcac ctatgtggca tgtggtgatg tacgctgcgt gccatgagtc |
| 1981 | catgtcctat gcctcacaaa tgctgtggtt cactgcactg ttcaggagtc caaaccttct |
| 2041 | accctgggtc ctcgggcccc tgagcctgtg tcctgaagaa tctcgactct cgtgatatgc |
| 2101 | tgcttgtgac cttgacttgc catgaaggca cctgccccca cagctccttg caaactatga |
| 2161 | gttcacatgt gccctgcctt gaacaaccca atctggctgg taaatgacat ctacgaagcc |
| 2221 | acaggcctgg cctcaactga gaagccaagc ctggagtagg cctggggcta tgcatacgca |
| 2281 | ttaagggcct gctggagaaa ccttcactcc agacagacag ggggctaagg ggctagggct |
| 2341 | gagcacctct cagagcaagc ctttgacctg ttcagaagga tgggaaaaag gctggaagga |
| 2401 | aagcaaggca gaaggccagg agtaagccca agctaagctg caggcacaca cgccttcatg |
| 2461 | ccagtgcatg ccaggaagca cgttcactag actaaaacac ggtaaccatg atggaataag |
| 2521 | gagctggggc ttctttctgc tggctgatgg gtggacctgt gtatggtgac ctctccggcc |
| 2581 | tggccttttc cattcgcatg aacaatggga gcacatagcc ctgttgacct gcccgatcca |
| 2641 | gattcccagg ccaggctccc gaccccatcc acctatccac caccccaccc acctttgcag |
| 2701 | ggaatacctc gttagagaat ggcctgagac agtgcgttgc aactttttaa ttccatatca |
| 2761 | aaagttgatt tcttcttcat tccatttatg gaacttccct ccccctagtc cggttcttct |
| 2821 | tgtctttgtc cctgttggtt tgaagtgtta gactatagcc cctggtgtgt aaataatgta |
| 2881 | catagagtga gaagaaagaa acttgatatt gaggtgtttg aaatatggaa ctgtaataac |
| 2941 | ttctaggtat tcgaaacctg tgatttctgt gccattttct gtaaagatac aagtaagaat |
| 3001 | aaaattgact taaaaata |

SEQ ID NO: 4 Human Visual System Homeobox 2 Amino Acid Sequence (NP_878314.1)

| | |
|---|---|
| 1 | mtgkageals kpksetvaks tsggaparct gfgiqeilgl nkeppsshpr aaldglapgh |
| 61 | llaarsvlsp agvggmgllg pgglpgfytq ptflevlsdp qsvhlqplgr asgpldtsqt |
| 121 | assdsedvss sdrkmsksal nqtkkrkkrr hrtiftsyql eelekafnea hypdvyarem |
| 181 | lamktelped riqvwfqnrr akwrkrekcw grssvmaeyg lygamvrhsi plpesilksa |
| 241 | kdgimdscap wllgmhkksl eaaaesgrkp egergalpkl dkmeqdergp daqaaisqee |
| 301 | lrensiavlr akagehstkv lgtvsgpdsl arstekpeee eamdedrpae rlsppqledm |
| 361 | a |

SEQ ID NO: 5 Human Paired Box Protein Pax-6 Isoform A 6 Nucleic Acid Sequence
(NM_001368891.2, CDS region from 462-1730)

| | |
|---|---|
| 1 | gtgtgtcaca gtacagttaa aggggtgaag atctaaacgc caaaagagaa gttaatcaca |
| 61 | ataagtgagg tttgggataa aaagttgggc ttgccccttt caaagtccca gaaagctggg |
| 121 | aggtagatgg agagggggcc attgggaagt ttttttggtg tagggagagg agtagaagat |
| 181 | aaagggtaag cagagtgttg ggttctgggg gtcttgtgaa gttccttaag gaaggaggga |
| 241 | gtgtggccct gcagccctcc caaactgctc cagcctatgc tctccggcac caggaagttc |
| 301 | caaggttccc ttcccctggt ctccaaactt cagggggaag actttaacta ggggcgcgca |
| 361 | gatgtgtgag gccttttatt gtgagagtgg acagacatcc gagatttcag agccccatat |
| 421 | tcgagccccg tggaatcccg cggcccccag ccagagccag catgcagaac agtcacagcg |
| 481 | gagtgaatca gctcggtggt gtctttgtca acgggcggcc actgccggac tccacccggc |
| 541 | agaagattgt agagctagct cacagcgggg cccggccgtg cgacatttcc cgaattctgc |
| 601 | aggtgtccaa cggatgtgtg agtaaaattc tgggcaggta ttacgagact ggctccatca |
| 661 | gacccagggc aatcggtggt agtaaaccga gagtagcgac tccagaagtt gtaagcaaaa |
| 721 | tagcccagta taagcgggag tgcccgtcca tctttgcttg ggaaatccga gacagattac |
| 781 | tgtccgaggg ggtctgtacc aacgataaca taccaagcgt gtcatcaata aacagagttc |
| 841 | ttcgcaacct ggctagcgaa aagcaacaga tgggcgcaga cggcatgtat gataaactaa |
| 901 | ggatgttgaa cgggcagacc ggaagctggg gcacccgccc tggttggtat ccggggactt |
| 961 | cggtgccagg gcaacctacg caagatggct gccagcaaca ggaaggaggg ggagagaata |
| 1021 | ccaactccat cagttccaac ggagaagatt cagatgaggc tcaaatgcga cttcagctga |
| 1081 | agcggaagct gcaaagaaat agaacatcct ttacccaaga gcaaattgag gccctggaga |
| 1141 | aagagtttga gagaacccat tatccagatg tgtttgcccg agaaagacta gcagccaaaa |
| 1201 | tagatctacc tgaagcaaga atacaggtat ggttttctaa tcgaagggcc aaatggagaa |
| 1261 | gagaagaaaa actgaggaat cagagaagac aggccagcaa cacacctagt catattccta |
| 1321 | tcagcagtag tttcagcacc agtgtctacc aaccaattcc acaacccacc acaccggttt |
| 1381 | cctccttcac atctggctcc atgttgggcc gaacagacac agccctcaca aacacctaca |
| 1441 | gcgctctgcc gcctatgccc agcttcacca tggcaaataa cctgcctatg caacccccag |
| 1501 | tccccagcca gacctcctca tactcctgca tgctgcccac cagcccttcg gtgaatgggc |
| 1561 | ggagttatga tacctacacc cccccacata tgcagacaca catgaacagt cagccaatgg |
| 1621 | gcacctcggg caccacttca acaggactca tttcccctgg tgtgtcagtt ccagttcaag |
| 1681 | ttcccggaag tgaacctgat atgtctcaat actggccaag attacagtaa aaaaaaaaaa |
| 1741 | aaaaaaaaaa aggaaaggaa atattgtgtt aattcagtca gtgactatgg ggacacaaca |
| 1801 | gttgagcttt caggaaagaa agaaaaatgg ctgttagagc cgcttcagtt ctacaattgt |
| 1861 | gtcctgtatt gtaccactgg ggaaggaatg gacttgaaac aaggaccttt gtatacagaa |
| 1921 | ggcacgatat cagttggaac aaatcttcat tttggtatcc aaacttttat tcattttggt |
| 1981 | gtattatttg taaatgggca tttgtatgtt ataatgaaaa aaagaacaat gtagactgga |
| 2041 | tggatgtttg atctgtgttg gtcatgaagt tgtttttttt ttttttaaaa agaaaaccat |
| 2101 | gatcaacaag ctttgccacg aatttaagag ttttatcaag atatatcgaa tacttctacc |
| 2161 | catctgttca tagtttatgg actgatgttc caagtttgta tcattccttt gcatataatt |
| 2221 | aaacctggaa caacatgcac tagatttatg tcagaaatat ctgttggttt tccaaaggtt |
| 2281 | gttaacagat gaagtttatg tgcaaaaaag ggtaagatat aaattcaagg aagaaaaaaa |
| 2341 | gttgatagct aaaaggtaga gtgtgtcttc gatataatcc aatttgtttt atgtcaaaat |
| 2401 | gtaagtattt gtcttcccta gaaatcctca gaatgatttc tataataaag ttaatttcat |
| 2461 | ttatatttga caagaatata gatgttttat acacattttc atgcaatcat acgtttcttt |
| 2521 | tttggccagc aaaagttaat tgttcttaga tatagttgta ttactgttca cggtccaatc |
| 2581 | attttgtgca tctagagttc attcctaatc aattaaaagt gcttgcaaga gttttaaa |

SEQ ID NO: 6 Human Paired Box Protein Pax-6 Isoform A 6 Amino Acid Sequence
(NP_001355820.1)

| | |
|---|---|
| 1 | mqnshsgvnq lggvfvngrp lpdstrqkiv elahsgarpc disrilqvsn gcvskilgry |
| 61 | yetgsirpra iggskprvat pevvskiagy krecpsifaw eirdrllseg vctndnipsv |
| 121 | ssinrvlrnl asekqqmgad gmydklrmln gqtgswgtrp gwypgtsvpg qptqdgcqqq |
| 181 | egggentnsi ssngedsdea qmrlqlkrkl qrnrtsftqe qiealekefe rthypdvfar |
| 241 | erlaakidlp eariqvwfsn rrakwrreek lrnqrrqasn tpshipisss fstsvyqpip |
| 301 | qpttpvssft sgsmlgrtdt altntysalp pmpsftmann lpmqppvpsq tssyscmlpt |
| 361 | spsvngrsyd tytpphmqth mnsqpmgtsg ttstglispg vsvpvqvpgs epdmsqywpr |
| 421 | lq |

SEQ ID NO: 7 Human Tubulin Beta-3 Chain Isoform 1 Nucleic Acid Sequence
(NM_006086.4, CDS region from 61-1413)

| | |
|---|---|
| 1 | ctcagcagcc agcccggccc gcccgcgccc gtccgcagcc gcccgccaga cgcgcccagt |
| 61 | atgagggaga tcgtgcacat ccaggccggc cagtgcggca accagatcgg ggccaagttc |
| 121 | tgggaagtca tcagtgatga gcatggcatc gaccccagcg gcaactacgt gggcgactcg |
| 181 | gacttgcage tggagcggat cagcgtctac tacaacgagg cctcttctca caagtacgtg |
| 241 | cctcgagcca ttctggtgga cctggaaccc ggaaccatgg acagtgtccg ctcaggggcc |
| 301 | tttggacatc tcttcaggcc tgacaatttc atctttggtc agagtggggc cggcaacaac |
| 361 | tgggccaagg gtcactacac ggagggggcg gagctggtgg attcggtcct ggatgtggtg |
| 421 | cggaaggagt gtgaaaactg cgactgcctg cagggcttcc agctgaccca ctcgctgggg |
| 481 | ggcggcacgg gctccggcat gggcacgttg ctcatcagca aggtgcgtga ggagtatccc |
| 541 | gaccgcatca tgaacacctt cagcgtcgtg ccctcaccca aggtgtcaga cacggtggtg |
| 601 | gagccctaca acgccacgct gtccatccac cagctggtgg agaacacgga tgagacctac |
| 661 | tgcatcgaca acgaggcgct ctacgacatc tgcttccgca ccctcaagct ggccacgccc |
| 721 | acctacgggg acctcaacca cctggtatcg gccaccatga geggagtcac cacctccttg |
| 781 | cgcttcccgg gccagctcaa cgctgacctg cgcaagctgg ccgtcaacat ggtgcccttc |
| 841 | ccgcgcctgc acttcttcat gcccggcttc gcccccctca cagcccgggg cagccagcag |
| 901 | taccgggccc tgaccgtgcc cgagctcacc cagcagatgt tcgatgccaa gaacatgatg |
| 961 | gccgcctgcg acccgcgcca cggccgctac ctgacggtgg ccaccgtgtt ccggggccgc |
| 1021 | atgtccatga aggaggtgga cgagcagatg ctggccatcc agagcaagaa cagcagctac |
| 1081 | ttcgtggagt ggatccccaa caacgtgaag gtggccgtgt gtgacatccc gccccgcggc |
| 1141 | ctcaagatgt cctccacctt catcgggaac agcacggcca tccaggagct gttcaagcgc |
| 1201 | atctccgagc agttcacggc catgttccgg cgcaaggcct tcctgcactg gtacacgggc |
| 1261 | gagggcatgg acgagatgga gttcaccgag gccgagagca acatgaacga cctggtgtcc |
| 1321 | gagtaccagc agtaccagga cgccacggcc gaggaagagg gcgagatgta cgaagacgac |
| 1381 | gaggaggagt cggaggccca gggccccaag tgaagctgct cgcagctgga gtgagaggca |
| 1441 | ggtggcggcc ggggccgaag ccagcagtgt ctaaaccccc ggagccatct tgctgccgac |
| 1501 | accctgcttt cccctcgccc tagggctccc ttgccgccct cctgcagtat ttatggcctc |
| 1561 | gtcctcccca cctaggccac gtgtgagctg ctcctgtctc tgtcttattg cagctccagg |
| 1621 | cctgacgttt tacggttttg ttttttactg gtttgtgttt atattttcgg ggatacttaa |
| 1681 | taaatctatt gctgtcagat accctt |

SEQ ID NO: 8 Human Tubulin Beta-3 Chain Isoform 1 Amino Acid Sequence
(NP_006077.2)

| | |
|---|---|
| 1 | mreivhiqag qcgnqigakf wevisdehgi dpsgnyvgds dlqlerisvy yneasshkyv |
| 61 | prailvdlep gtmdsvrsga fghlfrpdnf ifgqsgagnn wakghytega elvdsvldvv |
| 121 | rkecencdcl qgfqlthslg ggtgsgmgtl liskvreeyp drimntfsvv pspkvsdtvv |
| 181 | epynatlsih qlventdety cidnealydi cfrtlklatp tygdlnhlvs atmsgvttsl |
| 241 | rfpgqlnadl rklavnmvpf prlhffmpgf apltargsqq yraltvpelt qqmfdaknmm |
| 301 | aacdprhgry ltvatvfrgr msmkevdeqm laiqsknssy fvewipnnvk vavcdipprg |
| 361 | lkmsstfign staigelfkr iseqftamfr rkaflhwytg egmdemefte aesnmndlvs |
| 421 | eyqqyqdata eeegemyedd eeeseaqgpk |

SEQ ID NO: 9 Human Protein Atonal Homolog 7 Nucleic Acid Sequence (NM_145178.4,
CDS region from 437-895)

| | |
|---|---|
| 1 | ctgcactctc cgacagctac tgcgctaaaa gcgctccttc cctgagcttc gggaaagagt |
| 61 | tcatcttcct gcaaaggagt ctcaggcttt cccagaggac ttgaaaggcc ttcctcgaac |
| 121 | cagccacacc aaactctgct gcagaaggtt tccttctctt tttcaacttc atgttgagaa |
| 181 | aatgactttc tcttgagcat ctcattttcc cctaaatttg ggcaagtgaa gagatatcag |
| 241 | cctggtcatc cagtagaaca gaaggccgag tcccgcactc ccccactgta aactatttga |
| 301 | ttgcacgtga gttgctttgt ttatgactta tttgctcaga agaggcacgt tgggaagcgg |
| 361 | ctcgagagac cagcccacgc gcaggtcctg agcgggcggg cgtgcgaggt cggcgcctcg |
| 421 | ctgcttgggg ccggggatga agtcctgcaa gcccagcggc ccgccggcgg gagcgcgcgt |
| 481 | tgcacccccg tgcgcgggcg gcaccgagtg cgcgggcacg tgcgccgggg ccgggcggct |
| 541 | ggagagcgcg gcgcgcaggc gcctggcggc caacgcgcgc gagcgccgcc gcatgcaggg |
| 601 | gctcaacact gccttcgacc gcttacgcag ggtggttccc cagtggggcc aggataaaaa |
| 661 | gctgtccaag tacgagaccc tgcagatggc cctgagctac atcatggctc tgacccggat |
| 721 | cctggccgag gccgagcgat tcggctcgga gcgggactgg gtgggtctcc actgtgagca |
| 781 | cttcggccgc gaccactacc tcccgttccc gggcgcgaag ctgccgggcg agagcgagct |
| 841 | gtacagccag agactcttcg gcttccagcc cgagcccttc cagatggcca cctagggcgc |
| 901 | gcgcctccgc gggggtgggt gtccggcagc cgctccgagc ctcggccctg ccccaagtag |
| 961 | cccagaagcc tccggcggcc caggattcta aggatgcaat cctcgaggaa aattagtcga |
| 1021 | ttctcagatt acctttattc gcatcatcag acctatggac gcaatcattt aattgccttt |
| 1081 | cttttcccct cctcctttgt attttgtaga tttcattaat ggatcttgtg aatgggttga |
| 1141 | ttgctgtgaa aataatgccc cctttcccct tttctgggct actttgaggg aaaacaatct |
| 1201 | taagaaaaat aggattaagc tattctgttc cagtcctcag agaaataatc actttcttaa |
| 1261 | actttgtgag tttgtcctgt tcgggtgaag ttacagtatc cattacttgt gtttgctcac |
| 1321 | aacagagcta ccttcctgtt gtgtaaatgc gtttttgctt tagtgcattg tgtgtgcaag |
| 1381 | catgaagtag aaacactttt tttttctggg tacagtacat gggtatcggt gctctgtatt |
| 1441 | tttttaaact gtgtacacat tattaaaata tacattttat aaaatataaa taaaaacgtg |
| 1501 | gatttgtttt tcatgccaa |

SEQ ID NO: 10 Human Protein Atonal Homolog 7 Amino Acid Sequence (NP_660161.1)

| | |
|---|---|
| 1 | mksckpsgpp agarvappca ggtecagtca gagrlesaar rrlaanarer rrmqglntaf |
| 61 | drlrrvvpqw gqdkklskye tlqmalsyim altrilaeae rfgserdwvg lhcehfgrdh |
| 121 | ylpfpgaklp geselysqrl fgfqpepfqm at |

SEQ ID NO: 11 Human POU Domain, Class 4, Transcription Factor 2 Nucleic Acid
Sequence (NM_004575.3, CDS region from 249-1478)

| | |
|---|---|
| 1 | agtttcgggt gccgaggtct gcagctagcg gcaagcggag tcaggcatcc gttcagactg |
| 61 | acagcagagg cggcgaagga gcgcgtagcc gagatcaggc gtacagagtc cggaggcggc |
| 121 | ggcgggtgag ctcaacttcg cacagccctt cccagctcca gccccggctg gcccggcact |
| 181 | tctcggaggg tcccggcagc cgggaccagt gagtgcctct acggaccagc gccccggcgg |
| 241 | gcgggaagat gatgatgatg tccctgaaca gcaagcaggc gtttagcatg ccgcacggcg |
| 301 | gcagcctgca cgtggagccc aagtactcgg cactgcacag cacctcgccg ggctcctcgg |
| 361 | ctcccatcgc gccctcggcc agctccccca gcagctcgag caacgctggt ggtggcggcg |
| 421 | gcggcggcgg cggcggcggc ggcggcggag gccgaagcag cagctccagc agcagtggca |
| 481 | gcagcggcgg cgggggctcg gaggctatgc ggagagcctg tcttccaacc ccaccgagca |
| 541 | atatattcgg cgggctggat gagagtctgc tggcccgcgc cgaggctctg gcagccgtgg |
| 601 | acatcgtctc ccagagcaag agccaccacc accatccacc ccaccacagc cccttcaaac |
| 661 | cggacgccac ctaccacact atgaatacca tcccgtgcac gtcggccgcc tcttcttcat |
| 721 | cggtgcccat ctcgcaccct tccgcgttgg cgggcacgca ccaccaccac caccatcacc |
| 781 | accaccacca ccaccaaccg caccaggcgc tggagggcga gctgctggag cacctgagtc |
| 841 | ccgggctggc cctgggcgct atggcgggcc ccgacggcgc tgtggtgtcc acgccggctc |
| 901 | acgcgccgca catggccacc atgaacccca tgcaccaagc agcgctcagc atggcccacg |
| 961 | cgcacgggct gccgtcgcac atgggctgca tgagcgacgt ggacgccgac ccgcgggacc |
| 1021 | tggaggcatt cgccgagcgc ttcaagcagc gacgcatcaa gctgggggtg acccaggcag |
| 1081 | atgtgggctc cgcgctggcc aacctcaaga tccccggcgt gggctcgctt agccagagca |
| 1141 | ccatctgcag gttcgagtcc ctcacactgt cccacaataa tatgatcgcg ctcaaaccca |
| 1201 | tcctgcaggc atggctcgag gaggccgaga agtcccaccg cgagaagctc accaagcctg |
| 1261 | aactcttcaa tggcgcggag aagaagcgca agcgcacgtc catcgctgcg ccagagaagc |
| 1321 | gctcgctcga agcctacttt gccattcagc ctcggccctc ctctgaaaag atcgccgcca |
| 1381 | tcgcggagaa gctggacctg aagaaaaacg tggtgcgcgt ctggttctgc aaccagaggc |
| 1441 | agaaacagaa aagaatgaaa tattccgccg gcatttagaa gactcttggc ctctccagag |
| 1501 | acgccccttt cctcgtccgc tcttttctct cctctcttct gcctcttttc acttttggcg |
| 1561 | actagaaaca attccagtaa atgtgaatct cgacaaatcg aggactgaag agggagcgaa |
| 1621 | cgagcgaaca actgagccca agccggtgag aatgtgaaac agtttctcaa aggaaagaat |
| 1681 | aacaaaagat ggtatttgtc tgttgtagca aagttgtccc tttgaacccc acctcggctt |
| 1741 | cttcagagga agtgtggaga tggctgtttg caggaaggca gacgagacag tgtttaaaaa |
| 1801 | gtccacaaga atgatcaagt aagatttgtt tttattctta cagacatcac ccgtgttcaa |
| 1861 | gtttaaaagt acactttgca actatttttc agaaatagaa attgattcag gactaaaact |
| 1921 | ttaaactaga gttgatgctt aatgtgatag agacatctct aaagtatttt gaattttaaa |
| 1981 | aaaagatggc agattttctg catttacact gtatattata tatatatttt tattgtggtt |
| 2041 | cttaccccct tttccttctc tgaagtgtta atgcttaaga aaagagttgc gcctgctgtg |
| 2101 | ttcactgatc ttgaaagcta ttattagatt attgcagaac aaccctctgt aaattattaa |
| 2161 | tttatctctc tagcaactta attttgtgca cattctaatt aattaaactt cttccgtcta |
| 2221 | aaaaaagtgg gggaaatgta tagctagtaa cgttcaaaaa attttgtttg atgagtttac |
| 2281 | cgaattttta cagctttcct cctatactgt gttccttttg acccatttgt atattctcac |
| 2341 | ttgaatgaag attgtttttt tctttgtttt tactggtagt gttctgattt gtgagtcgac |
| 2401 | actcagtaat ggatgtctta atcgtgtaga cctgattcac tgtctgaagt attgtttact |
| 2461 | tcgttacata tttaatgggg attcccacat tgtccccatg acacatgagc gctctcactt |
| 2521 | acccttacac acacacacac acacacacac acctctaaca gaagggaaga agcagttgga |
| 2581 | agcatgaccg atgcaccatt ttctagtttt aggtgcattt gccacttggt gtttgccctt |
| 2641 | cagattttag atttcaccaa ggtatttcag tcttccagtt ttcaattgct ttgttggcta |
| 2701 | catgttaata tttataggaa tacttcagtt tttccttttg gaggtttgtt tgtagaaaaa |
| 2761 | ctaatttgaa ctataagaaa gacagtgcac tgcttgtaaa ttcacattgt ttggaaaaat |
| 2821 | tcttttggaa caaaaaatta ggtacatgat aactggtacc ttatctactg taaatatttc |
| 2881 | attaaaaatg atgcacacat agatatattc ttacaaattt tgctgtattg ctgttctctt |
| 2941 | tgaggctctc caaagtcttg agttctgtat atggcctggt ttcttgtttt tattaataga |
| 3001 | tggtttattt actatggtaa tgtattaatt tatttttggt gttgttcgat tgtctttcat |
| 3061 | tgaagagata attttaatgt tttattggca acgtatgctg ctttttcatt aaaatatgct |
| 3121 | attaaaatta aatggctttt aaaa |

SEQ ID NO: 12 Human POU Domain, Class 4, Transcription Factor 2 Amino Acid Sequence
(NP_004566.2)

| | |
|---|---|
| 1 | mmmmslnskq afsmphggsl hvepkysalh stspgssapi apsasspsss snaggggggg |
| 61 | ggggggggrs ssssssgssg gggseamrra clptppsnif ggldesllar aealaavdiv |
| 121 | sqskshhhhp phhspfkpda tyhtmntipc tsaassssvp ishpsalagt hhhhhhhhhh |
| 181 | hhqphqaleg ellehlspgl algamagpdg avvstpahap hmatmnpmhq aalsmahahg |
| 241 | lpshmgcmsd vdadprdlea faerfkqrri klgvtqadvg salanlkipg vgslsqstic |
| 301 | rfesltlshn nmialkpilq awleeaeksh rekltkpelf ngaekkrkrt siaapekrsl |
| 361 | eayfaiqprp ssekiaaiae kldlkknvvr vwfcnqrqkq krmkysagi |

SEQ ID NO: 13 Human One Cut Domain Family Member 2 Nucleic Acid Sequence
(NM_004852.3, CDS region from 344-1858)

| | |
|---|---|
| 1 | agagcccttc tggacagctc ccgctcaccc aaacagaaga cgtcggcgcc ggagcgggct |
| 61 | cggacatggc gaggctgcga gccggcccga gcggcggggc ccggtgatcc ctccctccct |
| 121 | ccccgtcccc tcccctctcc cgcacgcacg ccccgtccgc ccccaccccg cccccacccc |
| 181 | gggcgagccc gcccgcagcc cggggcgcac acccgcacgc gcactcctct ccactcactc |
| 241 | ccgcgcccgc ccccactccc gcagccgagc cccgccacgc gcgccttgcc cgcccgccgg |
| 301 | ccgcccccgc cgcccccgcc gcccccgggc cctgatggac tgaatgaagg ctgcctacac |
| 361 | cgcctatcga tgcctcacca aagacctaga aggctgcgcc atgaacccgg agctgacaat |
| 421 | ggaaagtctg ggcactttgc acgggccggc cggcggcggc agtggcgggg gcggcggcgg |
| 481 | gggcggcggg ggcggcggcg ggggcccggg ccatgagcag gagctgctgg ccagccccag |
| 541 | cccccaccac gcgggccgcg gcgccgctgg ctcgctgcgg ggccctccgc cgcctccaac |
| 601 | cgcgcaccag gagctgggca cggcggcagc ggcggcagcg gcggcgtcgc gctcggccat |
| 661 | ggtcaccagc atggcctcga tcctggacgg cggcgactac cggcccgagc tctccatccc |
| 721 | gctgcaccac gccatgagca tgtcctgcga ctcgtctccg cctggcatgg gcatgagcaa |
| 781 | cacctacacc acgctgacac cgctccagcc gctgccaccc atctccaccg tgtctgacaa |
| 841 | gttccaccac cctcacccgc accaccatcc gcaccaccac caccaccacc accaccagcg |
| 901 | cctgtccggc aacgtcagcg gcagcttcac cctcatgcgc gacgagcgcg ggctcccggc |
| 961 | catgaacaac ctctacagtc cctacaagga gatgcccggc atgagccaga gcctgtcccc |
| 1021 | gctggccgcc acgccgctgg gcaacgggct aggcggcctc cacaacgcgc agcagagtct |
| 1081 | gcccaactac ggtccgccgg gccacgacaa aatgctcagc cccaacttcg acgcgcacca |
| 1141 | cactgccatg ctgacccgcg gtgagcaaca cctgtcccgc ggcctgggca ccccacctgc |
| 1201 | ggccatgatg tcgcacctga acggcctgca ccacccgggc cacactcagt ctcacgggcc |
| 1261 | ggtgctggca cccagtcgcg agcggccacc ctcgtcctca tcgggctcgc aggtggccac |
| 1321 | gtcgggccag ctggaagaaa tcaacaccaa agaggtggcc cagcgcatca cagcggagct |
| 1381 | gaagcgctac agtatccccc aggcgatctt tgcgcagagg gtgctgtgcc ggtctcaggg |
| 1441 | gactctctcc gacctgctcc ggaatccaaa accgtggagt aaactcaaat ctggcaggga |
| 1501 | gaccttccgc aggatgtgga agtggcttca ggagcccgag ttccagcgca tgtccgcctt |
| 1561 | acgcctggca gcgtgcaaac gcaaagagca agaaccaaac aaagacagga acaattccca |
| 1621 | gaagaagtcc cgcctggtgt tcactgacct ccaacgccga acactcttcg ccatcttcaa |
| 1681 | ggagaacaaa cgcccgtcaa aggagatgca gatcaccatt tcccagcagc tgggcctgga |
| 1741 | gctcacaacc gtcagcaact tcttcatgaa cgcccggcgc cgcagcctgg agaagtggca |
| 1801 | agacgatctg agcacagggg gctcctcgtc cacctccagc acgtgtacca aagcatgatg |
| 1861 | gaaggactct cacttgggca caagtcacct ccaaatgagg acaacagata ccaaaagaaa |
| 1921 | acaaaggaaa aagacaccgg attcctagct ggggcccttc actggtgatt tgaaagcaca |
| 1981 | attctcttgc aaagaaactt atattctagc tgtaatcata ggccaggtgt tcttcttttg |
| 2041 | tttttaatgg ctatggagtc caagtgcaag ctgaaaaatt aatctcttag aaccagacac |
| 2101 | tgttctctga gcatgctaag catcccagaa acccaaatgg ggccttcctg gagcgagtta |
| 2161 | attccagtat ggtgtcaacc aagctcggga ttgcttaaaa tatcatccat cccacttcag |
| 2221 | gtcctgtcag cttcttgcag tcagagttcc tatgagtaac aataggagtt tggcctatgt |
| 2281 | aaggactctg agtttaggct tccaagatac aacaataaga gaagaatcta gcaacgagaa |
| 2341 | tgacctcatt tgctttccac atgcttagcc tcattatacc atgttatgtc caagttcaca |
| 2401 | gccacaacat cagaatggta attactgagc acaagtttta aatatggacg ttaaaaaaaa |
| 2461 | aaatccaagg acctgttttt ccaacccaga catcttttca ttgaatgatt tagaaagctt |
| 2521 | taagttgatc cagcttacaa tttttttttt ctttacctcc tggaaatctc atatggtctt |
| 2581 | ggatccgtca aaaaaaccag tcagttcact tgcgctcaaa gtatcaagca caacaaagat |
| 2641 | aaacagaagt gaggaaggtt ctgggttcac tacatctgga ttttcaagac acctattgtg |
| 2701 | aagtcattag ggaattgatg agaatatggc ttcaagcaca ttttgcagtt tgctacaaat |
| 2761 | tctgttgtac ataatgcaga cgcacactca ggaggccaat ttaactgtta acagtgcatg |
| 2821 | gagcgaatgc agcattttaa aagatctagg tttttttagg tcattaatgt gtccttggtt |
| 2881 | gatcagtcat ctggtccctc ctactgtgtg ttatgaccac cacgtaatcc attctcgctc |
| 2941 | tttctgattt ggggtttttc ctcatccatc ccattagtag ggatgttttc tgtgttttct |
| 3001 | agcaagaaaa aaaaatcaat caatcaaacc tgcatacatg ttactcatga ctgtcatcta |
| 3061 | gtcctaaatc tcttctgttg ttgaatcatc cttgcaaaac agctgaatac atctggagaa |
| 3121 | aacacagcac accaaagaag cagaatactg caaaccaaag acatttatga cttgtcattt |
| 3181 | tctagcctaa aaatactgtg attactttta gaaatcagaa aacctctgca actccgaatg |
| 3241 | gcattcagct cttgcatttg gcgcatcatc gggctgagcg gaccagctac accaaggaca |
| 3301 | ttagccaagc cacccagagg ggtggctttg ccacaccagt tgtcaccttc ccatagcaag |
| 3361 | tggaagagcg cccacagaac tctgggagat tgcaaaggtc acaatgtgca tatttaccag |
| 3421 | tgaatggccc cgggtggggc cacgtggggg tgttcaaagc aagccaaacg ctgcaatcat |
| 3481 | tctttacaga cacttgagac tgactttttt atgaattact tagtcgaaac caaagaaact |
| 3541 | ttttctgcac ctacttctgc aacaaacaaa actgtcccat taaaatgaat aaataaatcc |
| 3601 | gtaaatcaat ggaaatcacc accaataaga aggaagcacg ccagaaaata aacgaaaaca |
| 3661 | aaaacaggga gacacactgt gttcaaacag acctcttggg acattttttg gaagcagatt |
| 3721 | ttaaagaaag ggttgagaca aagatagaaa taaggaagag cctcagtggc tgctgcttca |
| 3781 | tttgacaact cacacggtaa tcttaaagct gaagattgtc tttaatttgt gcctatgcag |
| 3841 | tttttcaaaa gaacacggaa cagagcaaca gaaacctcaa cagctacaat accaaagatg |
| 3901 | aggatttctc acaccttttg tttcagttca ttatctcctc ttgcctggct aaaatactaa |
| 3961 | tagcgccatt gaactgtata aaggtaatca attatgtttc tctgagcaac aaaaggaaag |
| 4021 | ggccatttat ttgattttat tgtttcattt caattttgtc ttatggtttt ttgccccaac |
| 4081 | atggaatctc tcaaaagttt ccatggactc caagtttaag atgttgggat attgaacagt |
| 4141 | tctctctgct cagcagaggg tagggaataa cattatcact tgaatgttct ttgcttaacc |
| 4201 | cttagacttg gttccttcta tgttcagagt ctcatcatca ggggaaggaa agggagtgag |
| 4261 | ggtcagggat aggggtcttg gtgatgcatc ctctcccgag ccacagaacc aaagagttta |
| 4321 | tagaggaatt tacagcctcg ttttcatgtg attgctacat cctaacaggg cttcatttgg |
| 4381 | gggtgggggg aaacatgtaa aaataattgc cagtttctac ttttctatta gctttttaaa |
| 4441 | aatcagctgt aaagttgcat ttctaaagaa agatatatat aatatataaa atacatatat |
| 4501 | agatcaactt gacattggtg ataaccaaaa ttattgctgt ccaaattcat gtcttgtttt |
| 4561 | ggtccagtgc ttcatttgct aagtattcgg ttcagaattt ttctcatttc tcatgccatt |
| 4621 | ccagagttaa tttgccactg tggatgattt gaagtattca gatctctatg gaagtttctg |
| 4681 | ggacaggttt aaagtcaaga tcaagcattt tagcatttaa cctgttgata aatggatcca |
| 4741 | tggtgtacat gagttttatt tgtattcgga gtcatctcta ttctatccct cagcctcgat |
| 4801 | taaggtggtg agtgaagtgc atccaacaga ctcggcccag aactgggtcc tgacagtggg |
| 4861 | gtgctcatct tctgtaactg ttgggaaggc tcggtggtcc attttcacca gttaaagaat |
| 4921 | atgaggccag cccagaaatc tgttctccag gagctgccct gtcccatctg ggtgtgccag |
| 4981 | accccctcag tgagcaggtc caccaaaggg acttctcaca ggggaagccc aactcctgtt |
| 5041 | gcaatgggtt gatagatttc ctcagggtgg taattaccaa ttcgtatttt gacaagccta |
| 5101 | tgtgcaacca cagctggcac tggggtgggc agtggtgttg ggtgggatgg gggagagtgt |
| 5161 | ctcaatcctg aagagaaaat ataaagcagg ttttggggag acttctggag tcctgcccct |
| 5221 | agagagcccc attgttgttc tttgtgcccc ctcctcattc cccctatgtg ggtctcccta |
| 5281 | tgcaggagct gtgagagaat gtgactctcc acaattttta taattcatcc ttcctaggag |
| 5341 | attgttcatt ggctcttccc ttgtgtccct ttgtcccttg ctcatactcc atgtttcctt |
| 5401 | tgtcaaagga ctaagaaaag agcatatttc agcagaggag tgttcccatg tgggttgatt |
| 5461 | tcaacttggg tatttctaaa agagtccttg tgacatgtgt ccagtggaaa tggttgctct |
| 5521 | tttccagact ggattgagga atggagcctg tttgatttgg ttagtgattc tttgacatac |
| 5581 | taatctcagc gtttgggtct ccagcatcct ctgaagatgt ctagactagt agaggctgcc |
| 5641 | tttgtgacct gacattacaa cattggtcaa accagtcctc tgataatcag aagaacatgt |
| 5701 | cataattgtt taaaaaaaaa aaaaaggcaa gaatttctct ccaaggagct ttaataaatg |
| 5761 | tctcattcca gataatgtca taccagagaa aagtgcttgc ttttagaaaa ttatttacat |
| 5821 | acatatataa atatatatgt gtatctatac agttatgtat caaaatttta agccctgcag |
| 5881 | aatttcaatt tgttagaaat ctaacagaaa aaaatttcta tattgaaagg taatagaatt |
| 5941 | taacccagtg agtttactca aggattttta aatttaagtt aataatttca gagaaaataa |
| 6001 | ccatttgggt gtggttatag tttagtatcc attacctcaa tccaaggaaa attccaggca |
| 6061 | ttcctcaacc atcaggaaaa ggtacagtgt gaaggaacag ttctcagcca aatttcacat |
| 6121 | tcttgaggca acagaaatca aaacactcag agccattgag tggaaaaaca atttacttta |
| 6181 | ttcctttaca caaataggct tgcattgttt ttgttttaat gtgattttgg tactagggat |
| 6241 | ataattattt cattccagga aataataaaa aaaaacagac agagccaata catttctttt |
| 6301 | tttaaaggaa acagcaacaa caataaaaac tcagcaccaa tatttaaaag cttttccaaa |
| 6361 | atgtaaaaga agtgtttagc ttgcaccatg cataaaggtg caggctagtt gaaccaggaa |
| 6421 | gcatggcact tcctctggag aaatccagaa agagttgctt ctaagctccc ttttccccct |
| 6481 | gcaggctctt ggcaattgta ggctttagca aatccagaat aattttcaat tcaagctaaa |
| 6541 | ataaaatcaa catttggaat gtaaatctga tacacacaca cttttctaag tcaaacaaca |
| 6601 | tatttcaaaa ccaaaaataa atacctttta gataatcagt tattttcttt gtctatactg |
| 6661 | ggcacccacc tactagtgcc agtaaattca agttgaacag atttttaaaa tcactattat |
| 6721 | ctgggtatgg gggaaacttc cccacttttg aaaatgttgg tagaattata ggaatgtctg |
| 6781 | tttgattatc attaccaaag tgtcatgaca gtatgccttt gtagtgaact cggattttca |
| 6841 | ggagtttgaa tagttggata ttttaaaatc taagaagaaa aggcctgttt ccaatgttgt |
| 6901 | tgaagaataa tgaactctat taaaaagtgg agaaaaagat aatacatgtg gtcaaggttg |
| 6961 | accacaaggc ccaggcacaa ctaccttggc gataatcttc tagattcgta acaggttaga |
| 7021 | gctgactttt tgtttttgtt gttgctgatg ctgtgtgatt cagacttctc agcctaacca |
| 7081 | ggaagagtaa gtggaaatgg tagatgaaga aggggtagag ctggtgtatc tataactttc |
| 7141 | tgatatttgt ctgccaaact tgatatatta gtaatttttt tatctttagc taagatcaag |
| 7201 | tcacccctga aacaacagga gattctagtt ttaaaataag gccacaaaaa tccttacgga |
| 7261 | atgaagaatg gcaccccagt tggttgtata agtctcataa gataatgatg ttgattttaa |
| 7321 | atatggatgt ctcaatgcct gttttctatc aatgatttgt ttgtttccaa ggtcggggag |
| 7381 | ggaaagaggg gagggtttat ctgttttaga aagtctcaga atacttataa aatacagaag |
| 7441 | tagttattaa aatatatagg acctcacata ggtagataca gaacttacca ttgaggctga |
| 7501 | tgggctgttg tgtgaatcac acaggacctt aaatgaggct cattattctc acacaccaaa |
| 7561 | atgactctga cagcctgaag cagttattgc tagagcccaa gctttccttg gaggttttgg |
| 7621 | agttaggttg attggaagta accagctaat accttttcta gtggagaaaa agacattgct |
| 7681 | accagcttgt tcatcccata gaagtcttcc actctgctcc atttttagca gcaagcattt |
| 7741 | catgtagcat aaaccttggc agataagtgt gcctaaggtt tatacagtct gtccgcttgg |
| 7801 | atgtatacaa atttagatac atattttaac atgtgttctc atagatgact ttataacaac |
| 7861 | acacattacc tataggtgtc tagactgtgt acatacaagt gtgtacagac aagcttcata |
| 7921 | cgtatatact gtaatccgtt acaacaaata aattttaaat catcgtttaa catgtatgtg |
| 7981 | gtacttctac agtgtacatt gttttcatta tttattgtaa cattgaaaac cacagtgcag |
| 8041 | ggaaaacaaa agtatcccag catcttcatc ctgtacactt ggaattaatt tcatttgggc |
| 8101 | atatccaaga taaactcaac tttcaagaaa tcttgtatat tatttaatca tctgtgttag |
| 8161 | gatgacacct atgattgatg acttcggttg aatagcttta ttctggattt ttcataacta |
| 8221 | aagctaaatc caaagacctg aaaaaggaca aaaagaaaaa aaaaaaaaga aaaaacaaag |
| 8281 | aaaaagaaga aaaaataata aagtcaagcg caaactgatg gggagacagt gggctctggt |
| 8341 | ttccaggatt gagacaatgg tactgcggtc ttggggagac tgcgttagct agtggggagt |
| 8401 | ggtgattttt ttcatgcttg tcacatctaa atggtcttta acatgagaaa gttttagagg |
| 8461 | ttataatttc ctgctttgtt tttatttaga ctatcaaatg aagttataca tgttgtcagt |
| 8521 | caaaaaatga agacaccctc tgccccaccc cacagaatgc tttttatctt gtctctttgg |
| 8581 | gttatgaccc aacaagctaa gtaccattaa tgtaattaac ttatttaaat tagttcctag |
| 8641 | tacataaatg tataggattt gggtaattat ttaatcatcc ttccttagtt tgattctact |
| 8701 | ccttgtactt atttatcaaa acctagacca atggtgcatc agagatgcaa aattctactt |
| 8761 | ggaatactct tgaagtttag tttgctttat aaagcagtga aattctgtta cagacaggga |
| 8821 | agaaatacag gttacaaaaa gagaatttgg gatattcttc cctcttaaat taacttttaa |
| 8881 | aatagtctaa gtaacaattt ttaaattatt taacttaagt tcgcagcccc acctggtacc |
| 8941 | aggcgaactt cacctcttaa ttattgtggc cctcggagcc ttcatattgt aacttattta |
| 9001 | tttaacttat tcagcatctg tgaaaggtgc actgtatagt ttatattttt aatttaaaac |
| 9061 | aacagagagc actgcagttt gtttgctgtc agaacaacag agcaaatttt gtggacaagc |
| 9121 | aatgactatt cagcctgaac ctgtgcattc agaaaacata agctgagacc ctgcttcacc |
| 9181 | agcctggatt tcggggcttc tatacagaaa ctggaaaaat aaattttaaa aaaatcgtaa |
| 9241 | acaaaaagag agaaaccctt acactagctg cttccaagaa tgaactctgt gtgtatgtaa |
| 9301 | agcaacaaaa caaaaaagga aaaaaacaaa aagcagaaaa aagaaaaaaa aaatgaaaaa |
| 9361 | ctttctattt ctagtgagaa ccaaagaagg ctacctcact gactttttcc atttgtaatt |
| 9421 | ttaatcgtgt tgatgacacc aaagatacca aagatttctt tctctgtgcg gtctgcattt |
| 9481 | tgcttgtgct cttttataat ttgaacgatt ttctctgaca tatggtatgt acagccacag |
| 9541 | ctcagatacc ccaaagaaat aattatctat gcgacggcgg ctgctaattt ggaaagggat |
| 9601 | attttctgtg tttctcttat atgtttgctg tctgctcgac atgttcaaga tgcgagttca |
| 9661 | gatgctgctg taattggatt ccttaaattc tgattacaaa ttgaggaagg aaactggttg |
| 9721 | gaaatggcct tcagtcctag ccatggcctc tatccccgct gggacctgtc acagtaaaga |
| 9781 | ctgccaatta ctgaaccaca gaagctctga ccattgagta gttgagctgg aagagacctt |
| 9841 | aggaatcatt tagtccaagc cccggtggcc cagaggaatg aaatagttat ccaaatcaaa |
| 9901 | taactcttga gagtgaaagc ccacacatgc ctcctggttc ctgccccagt gctccgctta |
| 9961 | ttgtacagtg ctacctctgc atgagagcgg tcccacattg acaaatagga tggtggcaat |
| 10021 | cctttagcaa tgagcaggga ctggggttta tctcttaaca ttttcagctg taaaattagt |
| 10081 | cacaagcatt ttcagtgtcc cattagtaca tagtcacata tggtcggttg cttcgtgaag |
| 10141 | gtggcctgtc ttgaaatact agggctcata cgggattttt gccctaggaa aaacatgttg |
| 10201 | atcccaatga tgtgatcact tttgaacctt tccattacaa agcattgtat agataacttt |
| 10261 | ttaattcagt aggaggagaa agttcattct tggcctgttg gctttgatta ttatgggtac |
| 10321 | tttaaagtca gtatttatca agaaagggaa cttgaccacc attggcacat gtgacattta |
| 10381 | agctcttcag ccttttcctt tttagttgta ggtgtttaca tttcatttct aagccaactc |
| 10441 | tgtatttatg agagaagttt aagccttaca tcatttgata ctaaagggtt atttgtggta |
| 10501 | aatgaaaaat gaccccaaaa ttacagagga atatgccagt ttaagaaatg gctacttaaa |
| 10561 | gttgcttctc tctttccttc ttactcatga aattaattgg tcttcttcaa gtttctttag |
| 10621 | attccattaa atgattaaat cactattaag agccattcat caacgtgatt tgtgtgttag |
| 10681 | ccaatgaatc tgtctcagct tttgaccaaa tgggttttag acaaatgcaa agatctgcct |
| 10741 | ctagtccata tggctctttt tgagtgctag tattttgcat ttcacataat gtagttattt |
| 10801 | tgagctttta aagagagcat ttagacaaag aagcaaagag aggaagggac caatcaactc |
| 10861 | atcagttcca tgcatcaaca aagcatagct agtagaggaa tataaatgac agattgacaa |
| 10921 | actgtaggaa acactgttac tctctttctg aagttttcaa gcaccatcct atgtgaaagt |
| 10981 | tccctcctgt ccaaacaagc tcaaggccca tcttctccct atacaaggca aacctgtaag |
| 11041 | gccttccttc caaagagtac attgctttgg ttttcttcct aaattcctat tggaattaga |
| 11101 | actctcagaa tccctgggag acagagcaaa gatgacttaa ttcattgagc agcagagctc |
| 11161 | cctataagtg aacatcacct tccccatctt tcctactgcc acacccatac gagagaggat |
| 11221 | ctagaaagag cgatggcagc ctgaacacag aaaacatccc cacttggcag acctctcctc |
| 11281 | agcaatcccc ccagcctcat gcttcacttg caaagtgtga cataaccacg ggacgagtgc |
| 11341 | cttgcttgaa ccaaagcaac gatttagcca gtctggacct ctctgtgctt tttttaattc |
| 11401 | ttcctgtgaa tacctcagct tcaactgggc ctccatacag tcagttggtg ggcttattgt |
| 11461 | actgtggtgc tttgcaatgc aaccctgcaa agaacaagat ttgtactaat accaaaggtt |
| 11521 | ctttctctat gtctcctcct ctgcctccct cgttcttccc ttttttctag ttcttcacgg |
| 11581 | ttccaaagct ttactatgaa cctgggcatg ttggcaatgc agaccgcgca attccttacc |
| 11641 | gaattttctc agatatacct catagacaat agtgtttaga gtaatgttat tatagcgtat |
| 11701 | gtaataaatt attcactgtt tcttttggta actgtgattt aaaaaaagaa aaaagaaaaa |
| 11761 | aaagctttat acgttttagg ttgtgctttt gtaatagatg aaaaaaggtg cgcttaaaaa |
| 11821 | gaaaatgtat gtttttttcc ccctttggat tttatttatg ctggattggg gaaagttgca |
| 11881 | gaatgagccc aaagtttaca gtttcatatt ttgctgaaga aacaatctgt gttcatttgc |
| 11941 | tctgttgaaa agaataatta ttttctacat ttgtgccact tggtctgaac aattaattgt |
| 12001 | tccgtgttaa cagtgtagta ttatgattag caactgccaa tcagtgctat aattttatgc |
| 12061 | atgaggctaa aaatttagca gtgtgatgca ttgtggtctt aatagcaaca tttttcattt |
| 12121 | tgaactagat cttccccttt ggttcaatgg actttattta tgcatgggcg cctattgttt |
| 12181 | gttagcagtt gtggaacagt tgtgtataca ttaaactgtg aaaatgtaca cagttcagcc |
| 12241 | tcagacggtg gtaatattgg ttttattggg agatgtgtca cctcgaaaat accctttaca |
| 12301 | tctgttggga tctgaaaatg agtcacattg aattgggttc cagctttata atgagaaacg |
| 12361 | ttattcctaa tttttgagtt agccaatttg cattccacaa attgggatcc tcataaccca |
| 12421 | aatatatcac cgtatgtgag agggatttga aagcgagtat tgaaaaactc acctttgcat |
| 12481 | atttaatttc caccaaaagg agttattttg gctttatgct catgaactta gacctaactg |
| 12541 | gccatgtata tgtagatgca aattcatcta gctgtggccc tctttgatct ctgcttggga |
| 12601 | atggctattt ttgactatgc gtggtttctt ctcgtatttt gtgatcaggt cagctcccag |
| 12661 | tagaaactca aatggcatca atattactaa ctcttctctg cccacttctc ttttgtccac |
| 12721 | tctcctagac attcccacca actgttccag tgatttgggc aaaaatacgc agccatttcc |
| 12781 | caaaacttca catgtgcagc tatcatggct gtccctccct agacttggag gtgactctca |
| 12841 | cttaattttt acctgcccaa caatgttcca tctaccatct aaaaggtaat ataagaagaa |
| 12901 | gttttgaaac ccactttagg aaaaccatct tctttaaatc cttcaattat ctgaggcctc |
| 12961 | tatatgtcaa aactattttt cagttgcagg ggattgggca aacttgttct ttcttatact |
| 13021 | tgggttcaaa gacccattct ccagtttcat atttcccaaa ccaaaatgct tgacataaag |
| 13081 | ccaaatcaac tgccaagcac actttatttt gcataggagt atgcagccta gggaaccttg |
| 13141 | gttgaaaagc agcagtctgc tatgcaaaat attggaaatc actgacagtg tagcattcat |
| 13201 | attatctgtc aatgagggta tattgggaac gtgctctcgt gaataataaa aagcaacata |
| 13261 | tttttatttg gccttataaa ttaggttgtg gtaatgtaaa ctttgatata tagtcttttt |
| 13321 | atttttctct tattaatctg ccaaagatgg gaacagatac aagaattttt caaattggct |
| 13381 | tttgtaagac aattgatgat tgtaatagtg tttaatcttc cagaaagctt tatatgttgt |
| 13441 | tccacaataa aattgatatt tgtttcagca aagttttcct gacactcaca aacccacaaa |
| 13501 | ctgttcctct taatgcagat attgtagaat ctacaaagtt caaatccatt tttgatccaa |
| 13561 | agaaagtaga ggagtatttg agacatgagt gtacccagcc ctttttttaa tcacaggcaa |
| 13621 | tgcatgggtc tggctggtta cactttgcca agaagacttg tcttatgaaa cccaaggtat |
| 13681 | attttgttat gccattttat gtccttttct tttaacattg tggaaagtgg tatgttgaat |
| 13741 | caagtgtaag ctgagttttc cagacaactg aagtagctac atcatgaatg ttattttgtt |
| 13801 | attaaagggt ttttactcag tgctttgtgc caatggatgt ccttttcctt ggagacacat |
| 13861 | aactacaaaa ttacctcagc ttggcctggt tttctctcct gccctcttgg ggaaacatgg |
| 13921 | gcctggcctg ggaaaaggca ggtcatgggc tggaaggtag gttttggtac taggaagaaa |
| 13981 | tctctgtatc tgtcagcttt aaagagaact gggccaaaaa tctctaacct cactctctct |
| 14041 | ggactccaac acttccctgc aatcctttgg tcttgagcat gtgccagcat gaaggcagac |
| 14101 | tccagttcat acatgaaagg caagaaaaag aaaatagtaa ccttgaatct tctgtgggcc |
| 14161 | accaggcact cacctttccc caccttgcac actatccagt caaggctatt gcagcccatc |
| 14221 | tggtggcttt acatgggaca ttaccaaagg cttcttcctc catcctgggg ttgcaaagga |
| 14281 | tccaggtccc ctccatccag tggggctctt ccacatcaga agtccccctc ccaccatcct |
| 14341 | ctgcatcctg tttagctatc ccatctatac cttttggaga tgattattta gaaaacaaag |
| 14401 | aaaggtatgg aatggggttt cctattgttt gctaggttat attttagcaa ttctcaattc |
| 14461 | tttgatctgg aaaaatacaa gagggaaaag gagaccccac tatctccctg tgctttgctc |
| 14521 | ccatctcagg gggcaggggc agtgcacatt gcctatgctg ttgatctgtc ttgggcgaca |
| 14581 | ggctgaatca cagctattgc cccagccaaa aacatggccc atcaatgcct actttatctc |
| 14641 | tgcttgaaaa tcctattcaa aaagttgtag agtttgaggt ttttatcccc ccatatcctt |
| 14701 | tgctttggtc cagtttggcc tttagcataa gagtcagctt tatctctagg aaagtttttt |
| 14761 | cagattatga caaggaacct gccacctggg aagaaaagag tccgaagact agcaatcgga |
| 14821 | taggtagtca taccattaac agatacttcc ttgaaggtag aatattattt cctttcttta |
| 14881 | cagttttgtg ttacacaagt ccaagtggtg ccagcaaact tcttaccgtg aaatgttgta |
| 14941 | aaacacctgg catactgaaa tttctgaaac aaaaacacaa gctccacatt gataacttga |
| 15001 | taaataacca ctaaagttta gatgcaggga ctgagatgat acaggcaaaa tcttggtgtt |
| 15061 | ggtttctctt ttaattcgta tcttcgatca cctaaccttt ctcaatccaa gagcagttca |
| 15121 | gtcttttctc cccaagtcta ggatgccaaa gagcatcata ggaaaagata attagggatt |
| 15181 | gaccagcatt tcaattagtt ctcttcttca tctttgcatt tctcaaaagt gttctcctgg |
| 15241 | accagaggga aagagctggt ccattttttt tcattctttc tattcaaatt tttccaccca |
| 15301 | gacaatactt tattaacaca gatactgtag atccttcctt ggtcagtgaa ttattacaag |
| 15361 | aggagctatc cttccaccaa agtgagtgaa aacaagttcc agtatctttt cttccatcca |
| 15421 | gttttgttct cagaatccaa gtcagtcctg ggtcttttct cactttagac cctggcctca |
| 15481 | gatgtgttta ttcttgctat ttaaaaatac ctttaaattt cacatgctgg cctgcagaac |
| 15541 | ttgcatcctt tgttctatac tgttgactgc ttgatggtat tgaaaggtga ctataatgag |
| 15601 | ggaagaaagg aggaggtaaa gagagaagaa tttgtcccag atctgtttaa agtttcaaaa |
| 15661 | tttaaaaagg gacccattaa attatgggaa aatggctata gagtgtgagc ctccgttgac |
| 15721 | catatgctca aagaccgtac tctgccacct gccttccagg tagctattct agaaactcag |
| 15781 | tcctttgtgg aaacccaact accttttaaa agtctctttc cagattccaa aaggacaaga |
| 15841 | gatcagagag tcacatatac gcctcttgtt ttattttctt gctttcacgg gtattattgc |
| 15901 | caagaaaatc gtagggaaaa actttaaact tttcttttca gttgatccct ttgacatcac |
| 15961 | ctctcatgtt taaaatcagg aaaacacacc cctaaaattt gcactctctt ccgttttgaa |
| 16021 | aaagaaaacc cacacacaaa tgcacactat taccgtcttt caccctgcgc tatatttcca |
| 16081 | aagtgtatta taatccagat attgccccat ctcaaacatg ttaagtcaga ctgtgctgaa |
| 16141 | agactttcca gggacggtca acagggtata tgttcagtgg ctgccctgaa atcctggtgg |
| 16201 | ggatgaggat cacgcttcat catcaagggg atgcccatcc cctgataagc tcccagtcct |
| 16261 | tttggaagat ttctttgaat gttaattgca ttttcagttt tgctcatttc ccaccccaat |
| 16321 | gttttgtctg caacatcgct tacactggat tctttctatt tttattccta tcattaaatg |
| 16381 | gtagtgctgt aaattctgca attaatgtta aataaactgc tttaattcat tga |

SEQ ID NO: 14 Human One Cut Domain Family Member 2 Amino Acid Sequence
(NP_004843.2)

| | |
|---|---|
| 1 | mkaaytayrc ltkdlegcam npeltmeslg tlhgpagggs gggggggggg ggggpghege |
| 61 | llaspsphha grgaagslrg ppppptahqe lgtaaaaaaa asrsamvtsm asildggdyr |
| 121 | pelsiplhha msmscdsspp gmgmsntytt ltplqplppi stvsdkfhhp hphhhphhhh |
| 181 | hhhhqrlsgn vsgsftlmrd erglpamnnl yspykempgm sqslsplaat plgnglgglh |
| 241 | naqqslpnyg ppghdkmlsp nfdahhtaml trgeqhlsrg lgtppaamms hlnglhhpgh |
| 301 | tqshgpvlap srerppssss gsqvatsgql eeintkevaq ritaelkrys ipqaifaqrv |
| 361 | lcrsqgtlsd llrnpkpwsk lksgretfrr mwkwlqepef qrmsalrlaa ckrkeqepnk |
| 421 | drnnsqkksr lvftdlqrrt lfaifkenkr pskemqitis qqlglelttv snffmnarrr |
| 481 | slekwqddls tggssstsst ctka |

SEQ ID NO: 15 Human Contactin-2 Precursor Nucleic Acid Sequence (NM_001346083.2,
CDS region from 181-3303)

| | |
|---|---|
| 1 | agacagcgcc ccgcggcctg tccatgagcg gcccatgcat tatggaagac gcgcgctggg |
| 61 | ctcgctccct ggtccttaaa tgacagcaaa tgaggtcctt tctcagcctc cagctgggct |
| 121 | gtccccaagc tgagctgagg ctcttctcct ccgatcccca cctctgcccg gacatccacc |
| 181 | atggggacag ccaccaggag gaagccacac ctgctgctgg tagctgctgt ggcccttgtc |
| 241 | tcctcttcag cttggagttc agccctggga tcccaaacca ccttcgggcc tgtctttgaa |
| 301 | gaccagcccc tcagtgtgct attcccagag gagtccacgg aggagcaggt gttgctggca |
| 361 | tgccgcgccc gggccagccc tccagccacc tatcggtgga agatgaatgg taccgagatg |
| 421 | aagctggagc caggttcccg tcaccagctg gtggggggca acctggtcat catgaacccc |
| 481 | accaaggcac aggatgccgg ggtctaccag tgcctggcct ccaacccagt gggcaccgtt |
| 541 | gtcagcaggg aggccatcct ccgcttcggc tttctgcagg aattctccaa ggaggagcga |
| 601 | gacccagtga aagctcatga aggctggggg gtgatgttgc cctgtaaccc acctgcccac |
| 661 | tacccaggct tgtcctaccg ctggctcctc aacgagttcc ccaacttcat cccgacggac |
| 721 | gggcgtcact tcgtgtccca gaccacaggg aacctgtaca ttgcccgaac caatgcctca |
| 781 | gacctgggca actactcctg tttggccacc agccacatgg acttctccac caagagcgtc |
| 841 | ttcagcaagt ttgctcagct caacctggct gctgaagata cccggctctt tgcacccagc |
| 901 | atcaaggccc ggttcccagc agagacctat gcactggtgg ggcagcaggt caccctggag |
| 961 | tgcttcgcct ttgggaaccc tgtcccccgg atcaagtggc gcaaagtgga cggctccctg |
| 1021 | tccccgcagt ggaccacagc tgagcccacc ctgcagatcc ccagcgtcag ctttgaggat |
| 1081 | gagggcacct acgagtgtga ggcggagaac tccaagggcc gagacaccgt gcagggccgc |
| 1141 | atcatcgtgc aggctcagcc tgagtggcta aaagtgatct cggacacaga ggctgacatt |
| 1201 | ggctccaacc tgcgttgggg ctgtgcagcc gccggcaagc cccggcctac agtgcgctgg |
| 1261 | ctgcggaacg gggagcctct ggcctcccag aaccgggtgg aggtgttggc tggggacctg |
| 1321 | cggttctcca agctgagcct ggaagactcg ggcatgtacc agtgtgtggc agagaataag |
| 1381 | cacggtacca tctacgccag cgccgagcta gccgtgcaag cactcgcccc tgacttcagg |
| 1441 | ctgaatcccg tgaggcgtct gatccccgcg gcccgcgggg gagagatcct tatcccctgc |
| 1501 | cagccccggg cagctccaaa ggccgtggtg ctctggagca aaggcacgga gattttggtc |
| 1561 | aacagcagca gagtgactgt aactccagat ggcaccttga tcataagaaa catcagccgg |
| 1621 | tcagatgaag gcaaatacac ctgctttgct gagaacttca tgggcaaagc caacagcact |
| 1681 | ggaatcctat ctgtgcgaga tgcaaccaaa atcactctag ccccctcaag tgccgacatc |
| 1741 | aacttgggtg acaacctgac cctacagtgc catgcctccc acgaccccac catggacctc |
| 1801 | accttcacct ggaccctgga cgacttcccc atcgactttg ataagcctgg agggcactac |
| 1861 | cggagaacta atgtgaagga gaccattggg gatctgacca tcctgaacgc ccagctgcgc |
| 1921 | catgggggga agtacacgtg catggcccag acggtggtgg acagcgcgtc caaggaggcc |
| 1981 | acagtcctgg tccgaggtcc gccaggtccc ccaggaggtg tggtggtgag ggacattggc |
| 2041 | gacaccacca tccagctcag ctggagccgt ggcttcgaca accacagccc catcgctaag |
| 2101 | tacaccctgc aagctcgcac tccacctgca gggaagtgga agcaggttcg gaccaatcct |
| 2161 | gcaaacatcg agggcaatgc cgagactgca caggtgctgg gcctcacccc ctggatggac |
| 2221 | tatgagttcc gggtcatagc cagcaacatt ctgggcactg gggagcctag tgggccctcc |
| 2281 | agcaaaatcc ggaccaggga agcagccccc tcggtggcac cctcaggact cagcggagga |
| 2341 | ggtggagccc ccggagagct catcgtcaac tggacgccca tgtcacggga gtaccagaac |
| 2401 | ggagacggct tcggctacct gctgtccttc cgcaggcagg gcagcactca ctggcagacc |
| 2461 | gcccgggtgc ctggcgccga tgcccagtac tttgtctaca gcaacgagag cgtccggccc |
| 2521 | tacacgccct ttgaggtcaa gatccgcagc tacaaccgcc gcggggatgg gcccgagagc |
| 2581 | ctcactgcac tcgtgtactc agctgaggaa gagcccaggg tggcccctac caaggtgtgg |
| 2641 | gccaaagggg tctcatcctc agagatgaac gtgacctggg aacccgtgca gcaggacatg |
| 2701 | aatggtatcc tcctggggta tgagatccgc tactggaaag ctggggacaa agaagcagct |
| 2761 | gcggaccgag tgaggacagc agggctggac accagtgccc gagtcagcgg cctgcatccc |
| 2821 | aacaccaagt accatgtgac cgtgagggcc tacaaccggg ctggcactgg gcctgccagc |
| 2881 | ccttctgcca acgccacgac catgaagccc cctccgcggc gacctcctgg caacatctcc |
| 2941 | tggactttct caagctctag tcttagcatt aagtgggacc ctgtggtccc tttccgaaat |
| 3001 | gagtctgcag tcaccggcta taagatgctg taccagaatg acttacacct gactcccacg |
| 3061 | ctccacctca ccggcaagaa ctggatagaa atcccagtgc ctgaagacat tggccatgcc |
| 3121 | ctggtacaaa ttcggaccac agggcccgga ggggatggga tccctgcaga agtccacatc |
| 3181 | gtgaggaatg gaggcacaag catgatggtg gagaacatgg cagtccgccc agcaccacac |
| 3241 | cctggcaccg tcatttccca ctccgtggcg atgctgatcc tcataggctc cctggagctc |
| 3301 | tgatcctgga acccctccct ctgcgccgca gctggacgcc acctccgacg gacacagcca |
| 3361 | gccccttcct gctgccaagg tggcctgaca ctgtgccaga gagtggctgg ttttaaatac |
| 3421 | ctactttaaa cagtgccctt tttgtaggag gtaggatatt ttatattctg ccgcaggata |
| 3481 | gaacccacgc aaggattttc tttaaattga gaggcaccag gcagtaactt ccatgatgac |
| 3541 | actgacgcct atacctgagc tctaggctgc ctggagggaa ggaacaggcc catgggaaga |
| 3601 | agggggtttt aaaaacatgt cttcaactca gcagagatgg ccctctggga ccctatacgg |
| 3661 | actccgccac ttgagagcag tcctaggccc ggcaggaaca ccagacatga acaggttgaa |
| 3721 | gaactggagc gaagtgcaca cctcaccatc cttcagtcta aggaagaagg gcaagccctg |
| 3781 | ggaccaagag ctctcccgcc ttctccctcg agcagcagca aggaccctga cgctgtcccc |
| 3841 | gataactccc taggggctcc tgcctgccca agcggctgag aaccagcgcc ccgatgcctg |
| 3901 | aggctgggag cctgagcccc ttcagctttg aggggggtga tactccaggc tgtttggggt |
| 3961 | gggagccaaa aagagttgag aggccagggc ccttggtgga aaggggcacc agccttggtc |
| 4021 | tgagatagtc acaacccagg tgacgatgcc ctctcagcca acactgccaa cctgaccctg |
| 4081 | tcatcccgat tgacagcgcc acttcaggtg gctgggtgac taaagggctt gtcttggtgg |
| 4141 | ggtctcccac ccctccaaga cccattctgc acagtccctc cagggtttgg gcaggagatg |
| 4201 | gccaatcatg cgcccacctc tccagtgctg cctgcagtca gctcggcctc cccgacctgc |
| 4261 | agccccagac tctgctctcc cagcactgac tcactcctgc ctgggagggg aatgcagcat |
| 4321 | tcatgctgtg tgtcctggta ttgggaggtt tctgggaagg gcagaggata aatgtggccc |
| 4381 | tgcctgctcc caggtatacc taggaccacc tggccagatc cgctcccaga cggccttgga |
| 4441 | ctgcttgcat ttccccggag aaaaaggggt taataaatgg gccatccttt cctgagctct |
| 4501 | gggtatacta ccagtcacag aacgtcagag ctggaagaag ccttagagct caacttcttc |
| 4561 | aagcccctca ctttacagat gaggaaatgg aggtggtcca gagagggtct gggattccca |
| 4621 | aggtcacaca gcccagaaga gatggggctg ggttaagaac tcgagtcttc cacctttctg |
| 4681 | ttcaaggctg tttgtctacc cagaggaagg aggcactgct gaatggctat ggcctggcta |
| 4741 | agaaggtgat tagtcagtag ggtgtgaaaa ttctacttca aggggttcgg attggtgatc |
| 4801 | atggggattg gcatggctgg gttcccgtcc aaggtgtggg cagagcttct accaaacttc |
| 4861 | aacatggagg gctgacttga agctccctgt ccccctcact cttgccccaa gaaaagaggc |
| 4921 | caaagcaaga gcagattccc taggcaagag cagcagcaca actaggaaac cccaaagccc |
| 4981 | atgctccgac aggtggccct tcacaggggg cagcgggaca ggcatcttga agggcatatg |
| 5041 | tcctcggaag ctccgagcct gttttctgta gtttatagtt agagctctat tttgttatgg |
| 5101 | ttttttaaac ttttaagtcc tgctctattt tcctgggcag gtttatgttg atgtttaccc |
| 5161 | actacaattt tttaaaaata taagctcaca tgccttttcc ctgccacagc caaaccccca |
| 5221 | ctgcacccta cccacccacc cctagcccag gtcagctttc ctggagctgg ctaatgaaag |
| 5281 | cctcctcacc tcttcccaac ccttacaagc aagggtgcta ggggctcagc tatacgacca |
| 5341 | ttctccctga cagggagtcc aaacttggcc tagcatccct cctggccccc ctctggccac |
| 5401 | gacttggcct gtgcctggtt ctctatcaga aaggggatgc tgaacaaaac ctccttccaa |
| 5461 | gttttatcca attcgttcct cattgcctcg ggctgcgtca ggggaagcag gggacaggtg |
| 5521 | tccagttgct gggccgaggg aggagctggt ttggcatagg acctaaccag tgaagctaga |
| 5581 | ggctacagcc actaaacttg cttcaggcca acgatagtta ctcacaagta agtaccttaa |
| 5641 | tgctaatgag gtccactaaa aaggggagga aggcagacct cctgggagac ccacgaaggg |
| 5701 | tttttagcca gggaaaactg agccccagga aaacctaacc actgggcagg cagaatttgt |
| 5761 | ttgagggata gaacgacaac aaaataaatg ttcctgcagc ctgagatttc aggtagagta |
| 5821 | ctgactaagg tttaataaga caataggtga cctgaggaca tgcaagcttg taaaatgcaa |
| 5881 | cagcctcctg ctagagtgac ttgtacatga gcttgcttgc agaagactag attagatgtt |
| 5941 | tctcaggatc ccctcctgcg caggggttct ctgattttcg tgttctctgc ccagatgggc |
| 6001 | tgggggagtt gagagtgtgc ttattttcac tgcgatcatg agaccacagt tctgggttat |
| 6061 | ctcctctcat acatcaagcc ccagaggagg cggcaagagg aacagccaca aacaagtact |
| 6121 | ttaccccaca gcttagtggc cagtaaacac cctggggact aggaaaagga accaactgta |
| 6181 | ggcacctctc cagggcctag ggagacaagt gtcctctctt ctgcatacat ttgggctccc |
| 6241 | cttacagagc cctttgccct ggctctctgg tccttgttgc tctaacagtc cagatgtaca |
| 6301 | cccagcctca gggggaaggc agctctctcc agacagagtc tcagggccca gcaaggtcag |
| 6361 | gttatctgct ttcattcagg gcaacaaatg atacaaatgg tgccagggag tggcaaggcc |
| 6421 | atgggggtag gtgggggtgt ctttttcttt tcataaagta acaacagacg agactgaggt |
| 6481 | taaacatcag aaaaaaacct ctggaatgac cttcctcatt ccaggaggcc ctggaataag |
| 6541 | gaagaggctt ctttctgagg gagctttgag gaattttgac agctgttgac atgggatttg |
| 6601 | ggaaaggtga agctgtgact ggaggggcag gagatggtcc aagtgtccat ccagagatga |
| 6661 | gactcttaga atcaaagtgt tcagcccagg aagtcttgga gatcccacct tctgtggccc |
| 6721 | tgcaccttat gggaagccat taagggggct catctaggaa ttctggttac agcccagtgc |
| 6781 | tcatcccagc gtatgctgcc tctttagggc agccccaagg gccagccagc ctgtactctg |
| 6841 | ggcaagagcc caaaatggct aggaatgttt gactccctta atctcttccc cagctacaga |
| 6901 | ggaatctttt ctctgcctgg tctcagaatg ggactgccaa ctggctcatt ggtgggagac |
| 6961 | acagtatcct caaacctgtg gccactggca tgacagtggt gctctgtctc cctgggtgac |
| 7021 | acccacccta ggcttcctcc tggatgtgat ggggattgcc agagaggctc ttagcataaa |
| 7081 | aggcattagg tgggcatttt tctgtgtgcc cccaaaaagc tccatggaaa caggcacctg |
| 7141 | gtagctgcgg aacacccgtg gacttgtgta tatggtcata ggctttggga agacaggacg |
| 7201 | taaaggaaaa tgagagaaac aaaatgggtc agatagcttt ggccacagcc ccaggcagcc |
| 7261 | tttggggcct atgacactta gtgcccttag atgggataca tcttgcctcg gccccaagac |
| 7321 | tcctccaact tacccgtccc atccagggcc tgcacagctt agagaggctc acagcttggc |
| 7381 | aaatgctagg gcttcatcag accactgact tgactcagtg tttgttaaaa tggaaccact |
| 7441 | cccgttggcc tactgtttct ctcctgtact tcttgtaatg atagttattt attgactctg |
| 7501 | gtagcaggca gttcttaaat aaagatggtt tctcaacctg ttggggaagc tggcacgagc |
| 7561 | tgtgggtgtt atcatcaggc taaagcatac ctccttgtcc tgttcttcac tccagaggct |
| 7621 | tttatccaag aatttcttta ccccccacac agtgaaatat aagtaagtta caacatacag |
| 7681 | tctatattgc ttcatccagt cccaggagga gggaaaaatt aggccaagat cttaaggcag |
| 7741 | gtccaagcag agcacaggca ggcatttggc tagaatcagt tggctttacc taatacagtg |
| 7801 | gcagtaaaca atgcttatgt gatggta |

SEQ ID NO: 16 Human Contactin-2 Precursor Amino Acid Sequence (NP_001333012.1)

| | |
|---|---|
| 1 | mgtatrrkph lllvaavalv sssawssalg sqttfgpvfe dqplsvlfpe esteeqvlla |
| 61 | crarasppat yrwkmngtem klepgsrhql vggnlvimnp tkaqdagvyq clasnpvgtv |
| 121 | vsreailrfg flqefskeer dpvkahegwg vmlpcnppah ypglsyrwll nefpnfiptd |
| 181 | grhfvsqttg nlyiartnas dlgnysclat shmdfstksv fskfaqlnla aedtrlfaps |
| 241 | ikarfpaety alvgqqvtle cfafgnpvpr ikwrkvdgsl spqwttaept lqipsvsfed |
| 301 | egtyeceaen skgrdtvqgr iivqaqpewl kvisdteadi gsnlrwgcaa agkprptvrw |
| 361 | lrngeplasq nrvevlagdl rfsklsleds gmyqcvaenk hgtiyasael avqalapdfr |
| 421 | lnpvrrlipa arggeilipc qpraapkavv lwskgteilv nssrvtvtpd gtliirnisr |
| 481 | sdegkytcfa enfmgkanst gilsvrdatk itlapssadi nlgdnltlqc hashdptmdl |
| 541 | tftwtlddfp idfdkpgghy rrtnvketig dltilnaqlr hggkytcmaq tvvdsaskea |
| 601 | tvlvrgppgp pggvvvrdig dttiqlswsr gfdnhspiak ytlqartppa gkwkqvrtnp |
| 661 | aniegnaeta qvlgltpwmd yefrviasni lgtgepsgps skirtreaap svapsglsgg |
| 721 | ggapgelivn wtpmsreyqn gdgfgyllsf rrqgsthwqt arvpgadaqy fvysnesvrp |
| 781 | ytpfevkirs ynrrgdgpes ltalvysaee eprvaptkvw akgvsssemn vtwepvqqdm |
| 841 | ngillgyeir ywkagdkeaa adrvrtagld tsarvsglhp ntkyhvtvra ynragtgpas |
| 901 | psanattmkp pprrppgnis wtfsssslsi kwdpvvpfrn esavtgykml yqndlhltpt |
| 961 | lhltgknwie ipvpedigha lvqirttgpg gdgipaevhi vrnggtsmmv enmavrpaph |
| 1021 | pgtvishsva mliligslel |

SEQ ID NO: 17 Homo sapiens paired box 2 (PAX2) (NM_000278)

| | |
|---|---|
| 1 | agtctccggc cgagtcttct cgcagccgca acccacctgg ggccagccca gagctgccag |
| 61 | cgccgctcgg ctccctccct ccctcccggc ccttcggccg cggcggcgtg cgcctgcctt |
| 121 | ttccgggggc gggggcctgg cccgcgcgct cccctcccgc aggcgccacc tcggacatcc |
| 181 | ccgggattgc tacttctctg ccaacttcgc caactcgcca gcacttggag aggcccggct |
| 241 | cccctcccgg cgccctctga ccgcccccgc cccgcgcgct ctccgaccac cgcctctcgg |
| 301 | atgaccaggt tccaggggag ctgagcgagt cgcctccccc gcccagcttc agccctggct |
| 361 | gcagctgcag cgcgagccat gcgcccccag tgcaccccgg cccggcccac cgccccgggg |
| 421 | ccattctgct gaccgcccag ccccgagccc cgacagtggc aagttgcggc tactgcagtt |
| 481 | gcaagctccg gccaacccgg aggagcccca gcggggagcg cagtgctgcg ccccccgccc |
| 541 | ccgcgcgccc cgcagcagcc gggcgttcac tcatcctccc tcccccaccg tccctccctt |
| 601 | ttctcctcaa gtcctgaagt tgagtttgag aggcgacacg gcggcggcgg ccgcgctgct |
| 661 | cccgctcctc tgcctcccca tggatatgca ctgcaaagca gaccccttct ccgcgatgca |
| 721 | cccagggcac gggggtgtga accagctcgg gggggtgttt gtgaacggcc ggcccctacc |
| 781 | cgacgtggtg aggcagcgca tcgtggagct ggcccaccag ggtgtgcggc cctgtgacat |
| 841 | ctcccggcag ctgcgggtca gccacggctg tgtcagcaaa atcctgggca ggtactacga |
| 901 | gaccggcagc atcaagccgg gtgtgatcgg tggctccaag cccaaagtgg cgacgcccaa |
| 961 | agtggtggac aagattgctg aatacaaacg acagaacccg actatgttcg cctgggagat |
| 1021 | tcgagaccgg ctcctggccg agggcatctg tgacaatgac acagtgccca gcgtctcttc |
| 1081 | catcaacaga atcatccgga ccaaagttca gcagcctttc cacccaacgc cggatggggc |
| 1141 | tgggacagga gtgaccgccc ctggccacac cattgttccc agcacggcct cccctcctgt |
| 1201 | ttccagcgcc tccaatgacc cagtgggatc ctactccatc aatgggatcc tggggattcc |
| 1261 | tcgctccaat ggtgagaaga ggaaacgtga tgaagatgtg tctgagggct cagtccccaa |
| 1321 | tggagattcc cagagtggtg tggacagttt gcggaagcac ttgcgagctg acaccttcac |
| 1381 | ccagcagcag ctggaagctt tggatcgggt ctttgagcgt ccttcctacc ctgacgtctt |
| 1441 | ccaggcatca gagcacatca aatcagaaca ggggaacgag tactccctcc cagccctgac |
| 1501 | ccctgggctt gatgaagtca agtcgagtct atctgcatcc accaaccctg agctgggcag |
| 1561 | caacgtgtca ggcacacaga catacccagt tgtgactggt cgtgacatgg cgagcaccac |
| 1621 | tctgcctggt tacccccctc acgtgccccc cactggccag ggaagctacc ccacctccac |
| 1681 | cctggcagga atggtgcctg ggagcgagtt ctccggcaac ccgtacagcc acccccagta |
| 1741 | cacggcctac aacgaggctt ggagattcag caaccccgcc ttactaagtt ccccttatta |
| 1801 | ttatagtgcc gccccccggg gctccgcccc tgccgctgct gccgctgcct atgaccgcca |
| 1861 | ctagttaccg cggggaccac atcaagcttc aggccgacag cttcggcctc cacatcgtcc |
| 1921 | ccgtctgacc ccaccccgga gggagggagg accgacgcga cgcgatgcct cccggccacc |
| 1981 | gccccagcct caccccatcc cacgaccccc gcaacccttc acatcacccc cctcgaaggt |
| 2041 | cggacaggac gggtggagcc gtgggcggga ccctcaggcc cgggcccgcc gcccccagcc |
| 2101 | ccgcctgccg cccctccccg cctgcctgga ctgcgcggcg ccgtgagggg gattcggccc |
| 2161 | agctcgtccc ggcctccacc aagccagccc cgaagcccgc cagccaccct gccggactcg |
| 2221 | ggcgcgacct gctggcgcgc gccggatgtt tctgtgacac acaatcagcg cggaccgcag |
| 2281 | cgcggcccag ccccgggcac ccgcctcgga cgctcgggcg ccaggaggct tcgctggagg |
| 2341 | ggctgggcca aggagattaa gaagaaaacg actttctgca ggaggaagag cccgctgccg |
| 2401 | aatccctggg aaaaattctt ttcccccagt gccagccgga ctgccctcgc cttccgggtg |
| 2461 | tgccctgtcc cagaagatgg aatgggggtg tgggggtccg gctctaggaa cgggctttgg |
| 2521 | gggcgtcagg tctttccaag gttgggaccc aaggatcggg gggcccagca gcccgcaccg |
| 2581 | atcgagccgg actctcggct cttcactgct cctcctggcc tgcctagttc cccagggccc |
| 2641 | ggcacctcct gctgcgagac ccggctctca gccctgcctt gcccctacct cagcgtctct |
| 2701 | tccacctgct ggcctcccag tttcccctcc tgccagtcct tcgcctgtcc cttgacgccc |
| 2761 | tgcatcctcc tccctgactc gcagccccat cggacgctct cccgggaccg ccgcaggacc |
| 2821 | agtttccata gactgcggac tggggtcttc ctccagcagt tacttgatgc cccctccccc |
| 2881 | gacacagact ctcaatctgc cggtggtaag aaccggttct gagctggcgt ctgagctgct |
| 2941 | gcggggtgga agtggggggc tgcccactcc actcctccca tcccctccca gcctcctcct |
| 3001 | ccggcaggaa ctgaacagaa ccacaaaaag tctacattta tttaatatga tggtctttgc |
| 3061 | aaaaaggaac aaaacaacac aaaagcccac caggctgctg ctttgtggaa agacggtgtg |
| 3121 | tgtcgtgtga aggcgaaacc cggtgtacat aacccctccc cctccgcccc gccccgcccg |
| 3181 | gccccgtaga gtccctgtcg cccgccggcc ctgcctgtag atacgccccg ctgtctgtgc |
| 3241 | tgtgagagtc gccgctcgct gggggggaag ggggggacac agctacacgc ccattaaagc |
| 3301 | acagcacgtc ctgggggagg ggggcatttt ttatgttaca aaaaaaaatt acgaaagaaa |
| 3361 | agaaatctct atgcaaaatg acgaacatgg tcctgtggac tcctctggcc tgttttgttg |
| 3421 | gctctttctc tgtaattccg tgttttcgct ttttcctccc tgcccctctc tccctctgcc |
| 3481 | cctctctcct ctccgcttct ctccccctct gtctctgtct ctctccgtct ctgtcgctct |
| 3541 | tgtctgtctg tctctgctct ttcctcggcc tctctcccca gacctggccc ggccgccctg |
| 3601 | tctccgcagg ctagatccga ggtggcagct ccagcccccg ggctcgcccc ctcgcgggcg |
| 3661 | tgccccgcgc gccccgggcg gccgaaggcc gggccgcccc gtcccgcccc gtagttgctc |
| 3721 | tttcggtagt ggcgatgcgc cctgcatgtc tcctcacccg tggatcgtga cgactcgaaa |
| 3781 | taacagaaac aaagtcaata aagtgaaaat aaataaaaat ccttgaacaa atccgaaaag |
| 3841 | gcttggagtc ctcgcccaga tctctctccc ctgcgagccc tttttatttg agaaggaaaa |
| 3901 | agagaaaaga gaatcgttta agggaacccg gcgcccagcc aggctccagt ggcccgaacg |
| 3961 | gggcggcgag ggcggcgagg gcgccgaggt ccggcccatc ccagtcctgt ggggctggcc |
| 4021 | gggcagagac cccggaccca ggcccaggcc taacctgcta aatgtccccg gacggttctg |
| 4081 | gtctcctcgg ccactttcag tgcgtcggtt cgttttgatt ctttttttt tgtgcacata |
| 4141 | agaaataaat aataataata aataaagaat aaaattttgt atgtcactc |

SEQ ID NO 18: Homo sapiens aldehyde dehydrogenase 1 family member A3 (ALDH1A3)
(NM_000693)

| | |
|---|---|
| 1 | agcgggctgc gcagtgtccg ggccgagccg gtgcgccgca gactagggcg cctcgggcca |
| 61 | gggagcgcgg aggagccatg gccaccgcta acggggccgt ggaaaacggg cagccggaca |
| 121 | ggaagccgcc ggccctgccg cgccccatcc gcaacctgga ggtcaagttc accaagatat |
| 181 | ttatcaacaa tgaatggcac gaatccaaga gtgggaaaaa gtttgctaca tgtaaccctt |
| 241 | caactcggga gcaaatatgt gaagtggaag aaggagataa gcccgacgtg gacaaggctg |
| 301 | tggaggctgc acaggttgcc ttccagaggg gctcgccatg gcgccggctg gatgccctga |
| 361 | gtcgtgggcg gctgctgcac cagctggctg acctggtgga gagggaccgc gccaccttgg |
| 421 | ccgccctgga gacgatggat acagggaagc catttcttca tgcttttttc atcgacctgg |
| 481 | agggctgtat tagaaccctc agatactttg cagggtgggc agacaaaatc cagggcaaga |
| 541 | ccatccccac agatgacaac gtcgtgtgct tcaccaggca tgagcccatt ggtgtctgtg |
| 601 | gggccatcac tccatggaac ttccccctgc tgatgctggt gtggaagctg gcacccgccc |
| 661 | tctgctgtgg gaacaccatg gtcctgaagc ctgcggagca gacacctctc accgcccttt |
| 721 | atctcggctc tctgatcaaa gaggccgggt tccctccagg agtggtgaac attgtgccag |
| 781 | gattcgggcc cacagtggga gcagcaattt cttctcaccc tcagatcaac aagatcgcct |
| 841 | tcaccggctc cacagaggtt ggaaaactgg ttaaagaagc tgcgtcccgg agcaatctga |
| 901 | agcgggtgac gctggagctg ggggggaaga acccctgcat cgtgtgtgcg gacgctgact |
| 961 | tggacttggc agtggagtgt gcccatcagg gagtgttctt caaccaaggc cagtgttgca |
| 1021 | cggcagcctc cagggtgttc gtggaggagc aggtctactc tgagtttgtc aggcggagcg |
| 1081 | tggagtatgc caagaaacgg cccgtgggag accccttcga tgtcaaaaca gaacaggggc |
| 1141 | ctcagattga tcaaaagcag ttcgacaaaa tcttagagct gatcgagagt gggaagaagg |
| 1201 | aaggggccaa gctggaatgc gggggctcag ccatggaaga caaggggctc ttcatcaaac |
| 1261 | ccactgtctt ctcagaagtc acagacaaca tgcggattgc caaagaggag attttcgggc |
| 1321 | cagtgcaacc aatactgaag ttcaaaagta tcgaagaagt gataaaaaga gcgaatagca |
| 1381 | ccgactatgg actcacagca gccgtgttca caaaaaatct cgacaaagcc ctgaagttgg |
| 1441 | cttctgcctt agagtctgga acggtctgga tcaactgcta caacgccctc tatgcacagg |
| 1501 | ctccatttgg tggctttaaa atgtcaggaa atggcagaga actaggtgaa tacgctttgg |
| 1561 | ccgaatacac agaagtgaaa actgtcacca tcaaacttgg cgacaagaac ccctgaagga |
| 1621 | aaggcggggc tccttcctca aacatcggac ggcggaatgt ggcagatgaa atgtgctgga |
| 1681 | ggaaaaaaat gacatttctg accttcccgg gacacattct tctggaggct ttacatctac |
| 1741 | tggagttgaa tgattgctgt tttcctctca ctctcctgtt tattcaccag actggggatg |
| 1801 | cctataggtt gtctgtgaaa tcgcagtcct gcctggggag ggagctgttg gccatttctg |
| 1861 | tgtttccctt taaaccagat cctggagaca gtgagatact cagggcgttg ttaacaggga |
| 1921 | gtggtatttg aagtgtccag cagttgcttg aaatgctttg ccgaatctga ctccagtaag |
| 1981 | aatgtgggaa aaccccctgt gtgttctgca agcagggctc ttgcaccagc ggtctcctca |
| 2041 | gggtggacct gcttacagag caagccacgc ctctttccga ggtgaaggtg ggaccattcc |
| 2101 | ttgggaaagg attcacagta aggttttttg gtttttgttt tttgttttct tgtttttaaa |
| 2161 | aaaaggattt cacagtgaga aagttttggt tagtgcatac cgtggaaggg cgccagggtc |
| 2221 | tttgtggatt gcatgttgac attgaccgtg agattcggct tcaaaccaat actgcctttg |
| 2281 | gaatatgaca gaatcaatag cccagagagc ttagtcaaag acgatatcac ggtctacctt |
| 2341 | aaccaaggca ctttcttaag cagaaaatat tgttgaggtt acctttgctg ctaaagatcc |
| 2401 | aatcttctaa cgccacaaca gcatagcaaa tcctaggata attcacctcc tcatttgaca |
| 2461 | aatcagagct gtaattcgct ttaacaaatt acgcatttct atcacgttca ctaacagctt |
| 2521 | atgataagtc tgtgtagtct tccttttctc cagttctgtt acccaattta gattagtaaa |
| 2581 | gcgtacacaa ctggaaagac tgctgtaata acacagcctt gttattttta agtcctattt |
| 2641 | tgatattaat ttctgattag ttagtaaata acacctggat tctatggagg acctcggtct |
| 2701 | tcatccaagt ggcctgagta tttcactggc aggttgtgaa tttttctttt cctctttggg |
| 2761 | gatccaaatg atgatgtgca atttcatgtt ttaacttggg aaactgaaag tgttcccata |
| 2821 | tagcttcaaa aacaaaaaca aatgtgttat ccgacggata cttttatggt tactaactag |
| 2881 | tactttccta attgggaaag tagtgcttaa gtttgcaaat taagttgggg agggcaataa |
| 2941 | taaaatgagg gcccgtaaca gaaccagtgt gtgtataacg aaaaccatgt ataaaatggg |
| 3001 | cctatcaccc ttgtcagaga tataaattac cacatttgcc ttcccttcat cagctaacac |
| 3061 | ttatcactta tactaccaat aacttgttaa atcaggattt ggcttcatac actgaatttt |
| 3121 | cagtatttta tctcaagtag atatagacac taaccttgat agtgatacgt tagagggttc |
| 3181 | ctattcttcc attgtacgat aatgtcttta atatgaaatg ctacattatt tataattggt |
| 3241 | agagttattg tatcttttta tagttgtaag tacacagagg tggtatattt aaacttctgt |
| 3301 | aatatactgt atttagaaat ggaaatatat atagtgttag gtttcacttc ttttaaggtt |
| 3361 | tacccctgtg gtgtggttta aaaatctata ggcctgggaa ttccgatcct agctgcagat |
| 3421 | cgcatcccac aatgcgagaa tgataaaata aaattggata tttgagaaa |

Included in Table 1 are RNA nucleic acid molecules (e.g., thymines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.
Included in Table 1 are orthologs of the proteins, such as in human, mouse, monkey, etc., as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.

### Detection, Isolation, and Purification of Cells (e.g., RGCs)

Methods of characterizing organoids of the present disclosure as well as methods of detecting, isolating, and purifying specific cell types from said organoids include, without limitation, immunofluorescence-based methods, including those employed in cell sorting (e.g., magnetic activated cell sorting (MACS), a cell separation technique that uses antibodies conjugated to the surface of a paramagnetic bead and fluorescence activated cell sorting (FACS)), immunofluorescence staining, and the like. Regarding MACS, cells of interest that express biomarkers on their surfaces can be bound by the antibody-magnetic bead and column purified using magnets (e.g., placing a column in a magnetic field (e.g., between two magnets).

Alternatively or concurrently, the antibodies may be labeled with a detectable marker (e.g., a fluorescent moiety). Thus, cells of interested recognized by the antibody can be fluorescently labeled, allowing visual analysis/detection of the cells. The cells can be sorted using fluorescence assisted cell sorting or simply visually inspected using fluorescence microscopy.

Other marker detection methods include, but are not limited to, biochip arrays, fluorescence assays (e.g., sandwich immunoassay), surface plasmon resonance, ellipsometry and atomic force microscopy. Expression levels of markers (e.g., polynucleotides or polypeptides) are compared by procedures well-known in the art, such as RT-PCR, Northern blotting, Western blotting, mass spectrometry, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays.

In some embodiments of the methods of the present disclosure, multiple markers are measured. For example, in some embodiments, developing human RGCs express markers ATOH7, POU4F2, ONCUT2, and TUBB3. These cells may also express cell surface protein marker CNTN2. The use of multiple markers increases the predictive value of the test and provides greater utility in sample characterization, diagnosis, toxicology, patient stratification, and patient monitoring.

### Binding Agents

### Antigen-Binding Proteins & Antibodies

Antigen-binding proteins and/or antibodies that specifically bind a marker (e.g., of a cell in an organoid of the present disclosure or precursor thereof) are useful in various methods, including detection, isolation, and purification methods. In particular embodiments, the disclosure provides methods of detecting surface markers of cells in or derived from an organoid of the present disclosure (e.g., RGCs) by contacting the cells with antibodies that specifically bind these markers. The markers can be, but are not limited to, the markers in Table 1.

As used herein, an antigen-binding protein encompasses any polypeptide that bind specifically to any one of the antigens described herein. In some embodiments, an antigen-binding protein comprises an antibody. For example, in some embodiments, an antigen-binding protein comprises an intact antibody. In some embodiments, an antigen-binding protein comprises a fragment of an antibody. In some embodiments, an antigen-binding protein comprises an antigen-binding portion (e.g., one or more CDRs) of an antibody. Accordingly an antigen-binding protein of the present disclosure may take any natural or engineered form known in the art. In various embodiments, the antigen-binding protein and/or the antibody of the present disclosure further comprises a label for detection or purification (e.g., biotin, a histidine tag (e.g., comprising at least 3 histidines), a myc tag, a HA tag, a flag tag, a fluorescent moiety, an enzyme (e.g., horseradish peroxidase), or any marker known in the art).

Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. As used herein, the term “antibody” means not only intact antibody molecules, but also fragments of antibody molecules that retain immunogen-binding ability. Such fragments are also well known in the art and are regularly employed both in vitro and in vivo. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments.

Methods of preparing antibodies are well known to those of ordinary skill in the science of immunology. Antibodies can be made by any of the methods known in the art utilizing a soluble polypeptide, or immunogenic fragment thereof, as an immunogen. Nucleic acid sequences encoding polypeptides or immunogenic fragments thereof, can be provided to the host in a delivery vehicle that is taken up by immune cells of the host. The cells will in turn express the polypeptide thereby generating an immunogenic response in the host. Alternatively, nucleic acid sequences encoding human polypeptides or immunogenic fragments thereof, can be expressed in cells in vitro, followed by isolation of the polypeptide and administration of the polypeptide to a suitable host in which antibodies are raised.

### Aptamers

Aptamers are another class of binding agent or capture reagent that can be used to target cells in or isolated from an organoid of the present disclosure (e.g., RGCs or RGC biomarkers). Aptamers are nucleic acid-based molecules that bind specific ligands. Aptamers that specifically bind a marker of the cell (e.g., an RGC biomarker) are useful in the methods of the invention. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

### Nucleic Acid Detection

Nucleic acid molecules (i.e., mRNA) encoding biomarkers can be detected to determine if a biomarker is expressed in a cell. Methods of detecting nucleic acids are well known in the art. In some embodiments, oligonucleotides having substantial sequence identity to a target nucleic acid sequence hybridizes to the nucleic acid molecule encoding the biomarker to aid in detection. The oligonucleotide may be labeled (e.g., a fluorescent label) to further enable the detection of the target nucleic acid molecule. In some embodiments, the oligonucleotide is a primer suitable for use in an amplification reaction, whereby the target nucleic acid is amplified to detectable levels. Oligonucleotides that hybridize to target sequences in the nucleic acid sequences encoding any of the biomarker nucleic acid sequences listed in Table 1 are contemplated herein.

### Generation of a Telencephalon-Eye Organoid

The organoids disclosed herein comprise concentric zones of anterior ectodermal progenitors (a “CONCEPT” organoid). CONCEPT organoids are an ex vivo cell-based system that can comprise single cells of a particular type, sub-type or state, or a combination of cells of the same or differing type, sub-type, or state. CONCEPT organoids may be a model for screening perturbations to better understand the underlying biology or to identify putative targets for treating a disease or for screening putative therapeutics. In some embodiments, CONCEPT organoids are developed to be implanted into a living organism as a therapeutic or to further study a disease or condition. Thus, an ex vivo cell-based system may also be a cell-based therapeutic for delivery to an organism to treat disease, or an implant meant to restore or regenerate damaged tissue.

Accordingly, one aspect of the present disclosure provides a method of generating a CONCEPT organoid, especially a telencephalon-eye organoid that can produce retinal ganglion cells (RGCs). In some embodiments, the organoids are generated from primary cells, for example, from primary human cells. “Primary,” to a cell that is genetically substantially identical to an in vivo cell. For example, a primary cell could be a cell taken directly from a patient or subject. In some embodiments, a primary cell has been obtained from and/or propagated in a cell culture (e.g., a typical cell culture or an organoid). Accordingly, the organoids or cells derived from said organoids may be autologous or allogeneic to the subject. The rate of accumulation of mutations in the primary cells (e.g., obtained from a subject or from culture) is substantially the same as the rate of accumulation of mutations observed in in vivo cells. The CONCEPT organoids can be generated from stem cells (e.g., adult stem cells). In some embodiments, stem cells (e.g., human embryonic stem cells (hESC)) or inducible progenitor stem cells (iPSCs) are cultured, followed by detaching the cells from the culture dish or vessel. The cells can then be added to an extracellular matrix (decellularized or engineered). In some embodiments, an engineered matrix (e.g., Matrigel® (Corning Inc., Corning, NY), Geltrex® (ThermoFisher, Waltham, MA)), is used to provide a scaffolding for the maturing cultured cells.

To generate CONCEPT organoids, pre-organoid cell matrix clumps can gently be dispersed and cultured in a suspension medium. In some embodiments, culturing occurs in a low-adherent vessel (e.g., flask, plate, dish, etc.). Cysts can develop in the cultures, and those cysts with a single lumen can be selected for additional processing. In some embodiments, the cysts are passaged at a low density into another culture vessel. This matures into a functional CONCEPT organoid. The CONCEPT organoid can be screened for properties (e.g., physiological, functional, or characteristic (e.g., marker expression, concentric zones of anterior ectodermal progenitors) to ensure the organoid is suitable for its desired purpose. To confirm the presence of concentric patterns, the organoid can be subjected to methods of detecting certain biomarkers to confirm the cellular content of the organoid. For example, in some embodiments human RGCs are POU4F2+ that grow TUBB3+ directional long axons in a stem cell-derived organoid in which FOXG1+ telencephalon progenitors, PAX2+ optic stalk progenitor cells, PAX2+ optic disc progenitor cells, VSX2+ neuroretinal progenitors, and PAX6+ multi-lineage ocular progenitors form concentric zones.

In some embodiments, the retinal organoid is derived from cells obtained from a normal control (e.g., a subject who does not have and is not at risk of getting a disease or condition). A “normal” organoid mimics the genotype and phenotype of a normal, healthy organ. However, a “normal” organoid may also be generated from stem cells of a diseased subject or a subject who is at risk of getting a disease, as long as the subject does not have a genetic mutation that causes a loss of an RGC. In some embodiments, RGCs generated from the stem cells of a diseased subject without a genetic mutation may be preferred as it is autologous to the subject and does not trigger immune response to the transplantation/grafting/injection of the RGCs of the present disclosure to the subject. Alternatively, the organoids disclosed herein can be “disease” organoids (e.g., harboring a mutation). Similarly to “normal” organoids, disease organoids mimic the in vivo disease genotype and phenotype as it may comprise a mutation (e.g., in inherited optic neuropathy). In certain cases, such organoids are derived from in vivo cells with disease phenotypes. Disease organoids can also be generated by mutation of a normal organoid. In some embodiments, the organoids mimic an organ affected by a disease or condition.

In some embodiments and in contrast to previous studies, the CONCEPT organoids described herein produce human RGCs that grow directional long axons, and the organoids exhibit coordinated development of telencephalon and ocular tissues. In some embodiments, these RGCs express biomarkers that include, but are not limited to, ATOH7, POU4F2, ONECUT2, and TUBB3, as well as cell surface protein CNTN2. Fluorescent imaging can be used to investigate axonal grow in RGCs in CONCEPT organoids. Specifically, fluorescently labeled antibodies that specifically bind RGC biomarkers can be used to label RGCs for visual inspection or for isolation and or purification. In some embodiments, the RGCs are bound by an antibody against CNTN2 and isolated or purified using MACS. In some embodiments, the RGCs are bound by an antibody against CNTN2 and isolated or purified using FACS. Compared to methods using engineered tags for immuno-purification, human RGC isolation described herein is based on a native cell surface protein (CNTN2) that is specific for developing human RGCs in a native condition. This property has tremendous value in researching and understanding retinal cells, drug discovery/screening, and therapeutic applications. In some embodiments, POU4F2+ RGCs grow directional TUBB3+ long axons in CONCEPT organoids. In some embodiments, CNTN2 and TUBB3 exhibit very similar expression patterns in CONCEPT organoids. In general, RGCs isolated from CONCEPT organoids exhibit typical neuronal morphology.

In some aspects, methods are provided for isolating RGCs from organoids including, but not limited to, CONCEPT organoids. In some embodiments, the method comprises contacting the organoid with an antibody that specifically binds to CNTN2, capturing an anti-CNTN2 antibody-bound RGC, and removing the antibody from the antibody-bound RGC, thereby isolating the RGC from the organoid.

### Cellular Compositions

Compositions of the invention include cellular and pharmaceutical compositions comprising CONCEPT organoids and cells derived therefrom (e.g., RGCs), and a pharmaceutically acceptable carrier. In some embodiments, the cells in the cellular composition are isolated from one or more CONCEPT organoids and. Administration can be autologous or allogeneic. For example, can be obtained from one subject, and administered to the same subject or a different, compatible subject.

CONCEPT organoids and cells derived therefrom can be administered via localized injection. When administering a therapeutic cellular composition of the present invention (e.g., a pharmaceutical composition), it will generally be formulated in a unit dosage injectable form.

Cellular compositions of the present disclosure can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

The compositions can be isotonic, i.e., they can have the same osmotic pressure as ocular fluid or blood. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

One consideration concerning the therapeutic use of CONCEPT organoids and cells derived therefrom of the invention is the quantity of organoids and/or cells necessary to achieve an optimal effect. The quantity of cells to be administered will vary for the subject being treated. In some embodiments, between 10⁴ to 10⁸ cells from CONCEPT organoids derived therefrom of the invention can be administered to a human subject.

CONCEPT organoids and cells derived therefrom of the invention can express markers and have functional activities consistent with the organ and/or cells that make up the organ that the organoid is designed to mimic. Those skilled in the art can readily determine the percentage of CONCEPT organoids and cells derived therefrom in a population using various well-known methods, such as fluorescence activated cell sorting (FACS). Ranges of purity of cells in populations comprising cells derived from the CONCEPT organoids may be about 70 to about 75%, about 75 to about 80%, about 80 to about 85%, about 85 to about 90%, about 90 to about 95%, or about 95 to about 100%. Ranges of purity of cells in populations comprising cells derived from CONCEPT organoids may be at least about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Purity of cells derived from CONCEPT organoids can be determined according to the marker profile within a population. Dosages can be readily adjusted by those skilled in the art (e.g., a decrease in purity may require an increase in dosage).

The skilled artisan can readily determine the amount of cells and optional additives, vehicles, and/or carrier in compositions and to be administered in methods of the invention. Typically, any additives (in addition to the active CONCEPT organoids and/or cells derived therefrom and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Additional disclosures regarding pharmaceutical compositions comprising the cells or agents that treat diseases are presented below.

### Administration of Cellular Compositions

Compositions comprising cells of the present disclosure can be administered to (introduced into) a mammal according to methods known to those practiced in the art. In one embodiment, the cells are administered by injection or implantation. In some embodiments, CONCEPT organoids and/or cells derived therefrom (e.g., RGCs) are administered in a medium suitable for injection, such as phosphate buffered saline, into a mammal.

The purified CONCEPT organoids and cells derived therefrom used in the methods of the present invention can be obtained from a mammal to whom they will be returned or from another/different mammal of the same or different species (donor) and introduced into a recipient mammal. For example, the cells can be obtained from a pig and administered to a human. In some embodiments of particular interest, the recipient mammal is a human patient.

The present disclosure provides methods of treating diseases and/or disorders or symptoms thereof that comprise administering a therapeutically effective amount of a pharmaceutical composition comprising a cell of the disclosure to a subject (e.g., a mammal, such as a human). Thus, in some embodiments, provided herein is a method of treating a subject afflicted with or suffering from or susceptible to an eye disease or disorder or symptom thereof. The method includes administering to the mammal a therapeutic amount of cells described herein sufficient to treat the disease or disorder or symptom thereof, under conditions such that the disease or disorder is treated.

The methods herein include administering to the subject (including a subject identified as in need of such treatment) an effective amount of a cell described herein, or a cellular composition described herein to produce such effect. A health care professional can rely on her judgment to form a subject opinion of whether a subject is in need of such treatment. Alternatively, objective standards (e.g. measurable by a test or diagnostic method) can be used to identify such a subject.

In other embodiments, provided herein is a method of monitoring treatment progress. The method includes a step of determining a level of a diagnostic marker (e.g., any target delineated herein modulated by a compound herein or diagnostic measurement (e.g., a screen or assay) in a subject suffering from or susceptible to a disorder or symptoms thereof associated with muscle disease, in which the subject has been administered a therapeutic amount of a compound disclosed herein sufficient to treat the disease or symptoms thereof. The level of a marker determined in the method can be compared to known levels of the marker in either healthy normal controls or in other afflicted patients to establish the subject's disease status. In some embodiments, a second level of the marker in the subject is determined at a time point later than the determination of the first level, and the two levels are compared to monitor the course of disease or the efficacy of the therapy. In some embodiments, a pre-treatment level of the marker in the subject is determined prior to beginning treatment according to this disclosure; this pre-treatment level of the marker can then be compared to the level of the marker in the subject after the treatment commences to determine the efficacy of the treatment.

### Drug Discovery

The CONCEPT organoids disclosed herein are suitable for use in drug discovery and/or screening. Accordingly, CONCEPT organoids provide an ex vivo platform to which potential therapeutic agents can be applied and changes in organoid physiology, function, and viability (among other factors) can be observed. For example, novel therapeutics for glaucoma can be administered to a CONCEPT organoid. In such a case, modifications to the RGCs (i.e., axon) can be observed and classified (e.g., as beneficial, neutral, or detrimental). In some embodiments, changes in the organoid's size or its cellular makeup can indicate that the candidate therapeutic may affect the retina or cells therein in a subject (e.g., a human subject).

CONCEPT organoids that have concentric zones of anterior ectodermal progenitors produce RGC's. As these play a role in the maturation of the cell, changes to the distribution of the anterior ectodermal progenitor cells or in the size or location concentric zones may provide insight into the putative therapeutic agent's safety and/or efficacy. In some embodiments, CONCEPT organoids can be used to determine how the organoid or RGCs react to a therapeutic agent currently being administered to a subject. If the organoid and/or RGC show no adverse effects, the organoid and/or RGC can be a candidate cellular therapeutic. In some embodiments, the CONCEPT organoid to which the therapeutic agent is applied is derived from a cell obtained from the subject that will receive the cellular therapy.

In some embodiments, the CONCEPT organoids are assessed post-administration of a candidate therapeutic to determine changes to marker expression. In some embodiments, the markers assayed are one or more of the markers listed in Table 1. CONCEPT organoids can also be monitored for signs or apoptosis or the upregulation of genes involved in responding to genetic changes or exposure to toxins.

### Therapeutic Methods

The present invention provides methods of treating disease and/or disorders or symptoms thereof. These methods comprise administering a therapeutically effective amount of a pharmaceutical composition comprising a cell (e.g., RGC, lens cells, telencephalic cells) or an organoid herein to a subject (e.g., a mammal such as a human) sufficient to treat the disease or disorder or symptom thereof, under conditions such that the disease or disorder is treated.

Identifying a subject in need of such treatment can be in the judgment of a subject or a health care professional and can be subjective (e.g. opinion) or objective (e.g. measurable by a test or diagnostic method). In some instances, methods can include selection of a human subject who has or had a condition or disease and who exhibits or exhibited a positive immune response towards the condition or disease. In some instances, suitable subjects include, for example, subjects who have or had a condition or disease but that resolved the disease or an aspect thereof, present reduced symptoms of disease (e.g., relative to other subjects (e.g., the majority of subjects) with the same condition or disease), and/or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease).

In some instances, subject selection can include obtaining a sample from a subject (e.g., a candidate subject) and testing the sample for an indication that the subject is suitable for selection. In some instances, the subject can be confirmed or identified, e.g. by a health care professional, as having had or having a condition or disease. In some instances, exhibition of a positive immune response towards a condition or disease can be made from patient records, family history, and/or detecting an indication of a positive immune response. In some instances multiple parties can be included in subject selection. For example, a first party can obtain a sample from a candidate subject and a second party can test the sample. In some instances, subjects can be selected and/or referred by a medical practitioner (e.g., a general practitioner). In some instances, subject selection can include obtaining a sample from a selected subject and storing the sample and/or using the in the methods disclosed herein. Samples can include, for example, cells or populations of cells.

Provided herein are methods for treating and/or preventing diseases and disorders of the eye in a subject comprising administering to the subject a therapeutically effective amount of CONCEPT organoids and cells derived therefrom (e.g., RGCs, lens cells, telencephalic cells), or a pharmaceutical composition comprising CONCEPT organoids and cells derived therefrom (e.g., RGCs, lens cells, telencephalic cells). The disease can be any disease, disorder, or condition mediated by RGC dysfunction and/or RGC structural abnormality and/or associated with RGC dysfunction and/or RGC structural abnormality. In some embodiments, the subject has been diagnosed as having glaucoma.

The methods disclosed herein can be applied to a wide range of species, e.g., humans, non-human primates (e.g., monkeys), horses, cattle, pigs, sheep, deer, elk, goats, dogs, cats, mustelids, rabbits, guinea pigs, hamsters, rats, and mice.

By way of example for the prevention or treatment of an eye disease, e.g., a loss of an RGC, e.g., glaucoma, a therapeutically effective amount of retinal organoids or cells derived therefrom (e.g., RGCs, lens cells, telencephalic cells) reduces ocular pressure. In the example of glaucoma, the normal range for ocular pressure is between about 12-22 mm Hg, whereas glaucoma is generally diagnosed when ocular pressure exceeds 20 mm Hg, which is the on the high-end of normal pressure. Thus, a therapeutically effective amount of CONCEPT organoids and cells derived therefrom (e.g., RGCs) reduces ocular pressure by at least about 5%, more preferably by at least about 10%, even more preferably by at least about 15%, and still more preferably by at least about 20% or more relative to a subject's ocular pressure pre-treatment.

In general, methods include selecting a subject at risk for or with a condition or disease. In some instances, the subject's condition or disease can be treated with a pharmaceutical or cellular composition disclosed herein. For example, in some instances, methods include selecting a subject with an eye disease, e.g., a loss of an RGC, e.g., glaucoma, e.g., wherein the glaucoma can be treated by administering CONCEPT organoids and cells derived therefrom (e.g., RGCs, lens cells, telencephalic cells) to the subject.

In some instances, treatments methods can include a single administration, multiple administrations, and repeating administration as required for the prophylaxis or treatment of the disease or condition from which the subject is suffering. In some instances treatment methods can include assessing a level of disease in the subject prior to treatment, during treatment, and/or after treatment. In some instances, treatment can continue until a decrease in the level of disease in the subject is detected.

For example, dosage regimens are adjusted to provide the optimum desired response (e.g., a therapeutic response).

Following administration, the subject can be evaluated to detect, assess, or determine their level of disease. In some instances, treatment can continue until a change (e.g., reduction) in the level of disease in the subject is detected.

In some instances, the CONCEPT organoids or cells derived therefrom (e.g., RGCs, lens cells, telencephalic cells) described herein can be administered in combination with compounds, drugs, and/or agents used for the treatment of a disease (e.g., glaucoma). For example, the CONCEPT organoids or cells derived therefrom (e.g., RGCs, lens cells, telencephalic cells) disclosed herein may be administered in combination with at least one drug (e.g., a glaucoma drug), such as prostaglandin analogs (e.g., Xalatan® (latanoprost), Lumigan® (bimatoprost), Travatan Z® (travoprost), and Zioptan™ (tafluprost), and Vyzulta™ (latanoprostene bunod)); beta blockers (e.g., timolol); alpha agonists (e.g., Alphagan®P (brimonidine), Iopidine® (apraclonidine)); carbonic anhydrase inhibitors (CAIs, e.g., Trusopt® (dorzolamide), Azopt® (brinzolamide) Diamox® (acetazolamide), and Neptazane® (methazolamide)); rho kinase inhibitors (Rhopressa® (netarsudil)); or combinations thereof. In some instances, therapeutic methods disclosed herein can include administration of one or more (e.g., one, two, three, four, five, or less than ten) compounds.

In addition to administering at least one pharmaceutical agent in combination with the cellular therapies described herein, the CONCEPT organoids and cells (e.g., RGCs, lens cells, telencephalic cells) derived therefrom can be administered to a subject in need before or after laser treatment or surgery to further repair or restore a subject's eyesight, reduce pain and pressure associated with glaucoma, and/or inhibit a progression of the disease.

### Exemplary Stem Cells

### Embryonic Stem Cell

Embryonic stem cells (ES cells or ESCs) are pluripotent stem cells derived from the inner cell mass of a blastocyst, an early-stage pre-implantation embryo. Human embryos reach the blastocyst stage 4-5 days post fertilization, at which time they consist of 50-150 cells. Isolating the embryoblast, or inner cell mass (ICM) results in destruction of the blastocyst. ESCs are distinguished by their ability to differentiate into any embryonic cell type and by their ability to self-renew. ESCs have a normal karyotype, maintain high telomerase activity, and exhibit remarkable long-term proliferative potential.

Embryonic stem cells of the inner cell mass are pluripotent, meaning they are able to differentiate to generate primitive ectoderm, which ultimately differentiates during gastrulation into all derivatives of the three primary germ layers: ectoderm, endoderm, and mesoderm. These germ layers generate each of the more than 220 cell types in the adult human body. When provided with the appropriate signals, ESCs initially form precursor cells that in subsequently differentiate into the desired cell types. Pluripotency distinguishes embryonic stem cells from adult stem cells, which are multipotent and can only produce a limited number of cell types.

Due to their plasticity and potentially unlimited capacity for self-renewal, embryonic stem cell therapies have been proposed for regenerative medicine and tissue replacement after injury or disease. Pluripotent stem cells have shown promise in treating a number of varying conditions, including but not limited to: spinal cord injuries, age related macular degeneration, diabetes, neurodegenerative disorders (such as Parkinson's disease), AIDS, etc. In addition to their potential in regenerative medicine, embryonic stem cells provide a possible alternative source of tissue/organs which serves as a possible solution to the donor shortage dilemma.

Induced Pluripotent Stem Cell (iPSCs)

Induced pluripotent stem cells (also known as iPS cells or iPSCs) are a type of pluripotent stem cell that can be generated directly from a somatic cell. The iPSC technology was pioneered by Shinya Yamanaka's lab in Kyoto, Japan, who showed in 2006 that the introduction of four specific genes (named Myc, Oct3/4, Sox2 and Klf4), collectively known as Yamanaka factors, encoding transcription factors could convert somatic cells into pluripotent stem cells. He was awarded the 2012 Nobel Prize along with Sir John Gurdon “for the discovery that mature cells can be reprogrammed to become pluripotent.”

Pluripotent stem cells hold promise in the field of regenerative medicine. Because they can propagate indefinitely, as well as give rise to every other cell type in the body (such as neurons, heart, pancreatic, and liver cells), they represent a single source of cells that could be used to replace those lost to damage or disease.

Since iPSCs can be derived directly from adult tissues, they not only bypass the need for embryos, but can be made in a patient-matched manner, which means that each individual could have their own pluripotent stem cell line. These unlimited supplies of autologous cells could be used to generate transplants without the risk of immune rejection. The iPSCs are readily being used in personalized drug discovery efforts and understanding the patient-specific basis of disease.

### Exemplary Diseases

The compositions, cells, and methods of the present disclosure are particularly useful in preventing and/or treating a subject having or suspected of having a loss of a retinal ganglion cell (RGC). As used herein, the term “loss of an RGC” encompasses a loss of at least one RGC in a subject's eye. In some embodiments, the loss of an RGC comprises the loss of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the RGCs in the subject's eye.

The loss of an RGC encompasses a loss or a decrease in the function and/or activity of an RGC. In some embodiments, the loss of an RGC means a loss or decrease in the function and/or activity of an RGC by at least about 1%, 2%, 3%, 4%, 5%, 6%, 7%, 8%, 9%, 10%, 11%, 12%, 13%, 14%, 1%, 1⁶%, 1⁷%, 1⁸%, 1⁹%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the RGC function and/or activity of a healthy subject. The loss or a decrease in the function and/or activity of an RGC can be determined using the methods described herein or those known in the art (see below; see also Kim et al. (2021) Front Neurol. 12:661938, which is incorporated herein by reference).

In some embodiments, the loss of an RGC is a loss of its function and/or activity without the loss of the cell. In other embodiments, the loss of an RGC (e.g., a loss in function and/or activity) is due to degeneration of RGC. In some embodiments, the loss is due to a death (e.g., apoptotic death) of an RGC. In some embodiments, the loss is due to an injury to the RGC (e.g., injury to the eye).

Various conditions are known to result in the loss of an RGC. Inherited and acquired optic neuropathies are important causes of loss of RGCs. Treatment options remain limited, and when available, they mostly slow down or prevent further loss of RGCs. Visual loss is usually irreversible although in some cases, spontaneous visual recovery can occur owing to the functional recovery of RGCs that have not undergone apoptosis. Exemplary conditions/diseases that result from the loss of an RGC are described below.

### Inherited Optic Neuropathies

The minimum prevalence of inherited optic neuropathies has been estimated at 1 in 10,000. This group of disorders is genetically heterogeneous with disease-causing mutations occurring in both mitochondrial and nuclear DNA. Remarkably, all genes identified to date encode proteins that are either directly or indirectly involved in regulating mitochondrial function. The generation of ATP by the mitochondrial respiratory chain is central to cell survival and mitochondria also regulate other key pathways, including the level of reactive oxygen species and the tight control of apoptosis. An intriguing aspect of inherited optic neuropathies is the preferential vulnerability of RGCs compared with other neuronal populations despite the ubiquitous expression of the genes involved. There have been limited post mortem studies on the pattern of RGC loss in inherited optic neuropathies owing to the lack of access to diseased human tissues. Nevertheless, useful insight has been obtained with the application of high-resolution optical coherence tomography (OCT) imaging and psychophysical evaluation of patients at different stages of the disease process. The two best studied inherited optic neuropathies are Leber hereditary optic neuropathy (LHON) and autosomal dominant optic atrophy (ADOA).

### (1) Leber Hereditary Optic Neuropathy (LHON)

LHON is a primary mitochondrial DNA (mtDNA) disorder and ˜90% of cases are due to one of three mtDNA point mutations, namely, m.3460G>A (MT-ND1), m.11778G>A (MT-ND4), and m.14484T>C (MT-ND6) (158, 159). The peak age of onset is from 15 to 35 years old and the majority of patients are men (80-90%). Although bilateral simultaneous onset can occur in some patients, sequential involvement of the second eye within a few months is more typical. LHON is characterised by severe visual loss with dyschromatopsia and a dense central or cecocentral scotoma on visual field testing. OCT initially shows swelling of the RNFL, follows by marked thinning of retinal nerve fibre layer (RNFL), especially in the temporal quadrant corresponding to the papillomacular bundle. Childhood-onset LHON and the m.14484T>C mutation are associated with a more favorable visual outcome. Most patients with LHON are registered legally blind with <20% of patients carrying the m.11778G>A mutation experiencing some visual recovery.

In LHON, RGCs with the smallest calibre axons, which have smaller mitochondrial reserve per energy requirement, are preferentially affected and these are predominantly located within the papillomacular bundle. The peripapillary RNFL is swollen in the acute stage of LHON, as demonstrated by OCT, with subsequent thinning occurring as the disease progresses into the chronic stage. Measurement of ganglion cell and inner plexiform layer (GC-IPL) thickness in the macular area indicate that pathological thinning is already evident in the pre-symptomatic stage about 6 weeks before the onset of visual loss in the fellow eye. These findings suggest that midget RGCs, which are a major component of the papillomacular bundle, could be more vulnerable to the underlying mtDNA mutation. Selective attenuation of four of the six layers in the LGN that are connected to the parvocellular pathway have been reported, but this feature is controversial as the magnocellular pathway is known to be also affected in LHON.

The ipRGC subtype is relatively preserved in LHON, explaining why the pupillary light reflex is maintained even in severely affected patients. The mechanisms that account for this enhanced resilience of ipRGCs remain unclear, although several hypotheses have been proposed. From an anatomical perspective, ipRGCs are predominantly located in the parafoveal area and at the far end of the nasal hemiretina, rather than feeding into the papillomacular bundle. In a post mortem study of a patient carrying the m.3460G>A mtDNA mutation, the pupillary fibres in the pretectum were found to be preserved. It is possible that ipRGCs are protected because of their higher concentration of mitochondrial cytochrome c oxidase and a greater abundance of mitochondria. Several protective factors such as PI3K and pituitary adenylate cyclase-activating polypeptide (PACAP) may further reinforce the survival of ipRGCs under certain conditions.

### (2) Autosomal Dominant Optic Atrophy (ADOA)

ADOA is the most common inherited optic neuropathy with an estimated prevalence of 1 in 25,000 in the general population. Mutations in the nuclear gene OPA1 (3q28-q29) account for ˜70% of all cases of ADOA. The classical clinical features of ADOA are progressive bilateral visual loss starting in early childhood, dyschromatopsia, a central or cecocentral scotoma, and optic disc pallor that is more prominent temporally due to the preferential involvement of the papillomacular bundle. There is a marked variability in disease severity with visual acuity ranging from 6/6 to light perception, and variable rates of disease progression even within the same family. OCT typically shows RNFL thinning, which is more marked temporally, with gradual loss of RGCs occurring over time. The disease process is thought to start in utero with OPA1 carriers having a reduced number of RGCs at birth compared with normal healthy individuals.

In ADOA, midget RGCs, parasol RGCs and small bistratified RGCs are all affected, impairing sensitivity to high spatial frequencies, long- and middle-wave colour discrimination, sensitivity to high temporal frequencies, and short-wave sensitivity. The S-cone-related losses showed a significant deterioration with increasing patient age and could therefore prove useful biomarkers of disease progression in ADOA. The S-cone chromatic response and koniocellular pathway are impaired in the early stage of the disease, which suggest a vulnerability of small bistratified RGCs. Although tritanopia has been reported as the characteristic colour vision defect in ADOA, only 7.5% of patients with ADOA had exclusively tritanopia in one study, with the most common colour defect in 81.2% of patients being of the mixed type.

As in LHON, the pupillary response in ADOA is relatively preserved, indicating that ipRGCs in mitochondrial optic neuropathies appear to be more resistant to the underlying mitochondrial dysfunction compared with other RGC subtypes. Studies using chromatic pupillometry also reported preservation of ipRGCs in ADOA patients with severe visual loss and optic atrophy.

### Acquired Optic Neuropathies

There is a long list of aetiological factors that can result in RGC injury and optic nerve degeneration. Compared with inherited optic neuropathies, fewer studies have focused specifically on RGC pathophysiology in acquired optic neuropathies. More work is, therefore, needed to elucidate subtype selectivity, if any, of RGC loss in ischemic, compressive, inflammatory, autoimmune and paraneoplastic optic neuropathies. However, we know that most toxic optic neuropathies have an underlying mitochondrial aetiology. There is a growing body of evidence that mitochondrial dysfunction plays a prominent pathophysiological role in glaucoma, demyelinating optic neuritis and toxic optic neuropathies. This aetiological link is relevant and comparing the pattern of RGC loss between these acquired optic neuropathies and classical monogenic optic neuropathies could reveal common pathways amenable to therapeutic intervention.

### (1) Glaucoma

Glaucoma is a leading cause of irreversible blindness affecting 3-5% of the population over the age of 70 years. Extrafoveal RGCs usually deteriorate in the early stages resulting in arcuate scotomas in the visual field. Traditional anatomical studies reported greater loss of axons of large diameter, corresponding to the magnocellular pathway (parasol cells), and the magnocellular LGN layers were more affected compared with the parvocellular LGN layers. However, there are rarer types of retinal ganglion cells with large axons and further investigations are needed to evaluate the changes of these cells in glaucoma. The relative vulnerability of large axons in glaucoma may simply reflect the anatomical location of the affected ganglion cells. Glaucoma patients have poor response to high temporal frequency light stimuli that correspond to the magnocellular pathway. In a primate study using immunohistochemistry, a decrease in large RGCs was observed after elevating IOP. This specific vulnerability was ascribed to calcium-permeable receptors, the relative proximity of RGCs and their dendrites to blood supply in the IPL layer, and the differing metabolic requirements of these particular large cell types. However, other studies suggested no predilection for a specific pathway. Compared with inherited optic neuropathies, the ipRGCs are vulnerable in both patients with confirmed glaucoma and glaucoma suspects. In contrast, ocular hypertension does not seem to result in significant loss of ipRGCs.

### (2) Demyelinating Optic Neuritis

Inflammatory demyelination resulting in optic neuritis is a major manifestation of multiple sclerosis. Inflammation of the retinal vascular endothelium can precede demyelination and perivascular cuffing and oedema of the optic nerve sheath leads to breakdown of myelin. Idiopathic demyelinating optic neuritis leads to visual loss with minimal axonal loss.

Optic neuritis is associated with alteration of both the parvocellular and magnocellular pathways. Viret et al. suggested that the more heavily myelinated magnocellular axons are more vulnerable in patients with optic neuritis because low spatial frequencies, which are transmitted by the magnocellular pathway, are affected predominantly 1 month after the acute phase of the optic neuritis episode. Despite the recovery of visual acuity, the magnocellular pathway did not fully normalise. In contrast, a significant loss at high spatial frequencies has been reported in the affected eye and the parvocellular pathway was more impaired in patients with resolved optic neuritis who had 20/20 visual acuity after recovery. Fallowfield and Krauskopf suggested that chromatic discrimination is more severely impaired than luminance discrimination in the demyelinating diseases. This discrepancy might be due to differences in the timing and severity of optic neuritis. Consequently, it is still unclear which pathway is more vulnerable in the context of demyelinating optic neuritis. Both red-green and tritan defects have been reported in optic neuritis. Characteristics of colour deficiency may change over time as assessed with the FM 100-hue test, with blue-yellow defects being more common in the acute stage and red-green changes being predominant in the chronic stage. It is possible, of course, that the variability of symptoms in optic neuritis reflects immunologically distinct conditions that differentially affect different types of RGCs.

### (3) Toxic Optic Neuropathy

Various substances such as ethambutol, isoniazid, linezolid, chloramphenicol and methanol can cause optic nerve dysfunction, probably through acquired mitochondrial dysfunction. As in inherited optic neuropathies, the papillomacular bundle is selectively vulnerable and this typical feature can be confirmed by optical coherence tomography, which shows a profound decrease in temporal RNFL thickness. The parvocellular pathway within the papillomacular bundle is affected extensively likely secondary to a number of factors, including smaller and more thinly myelinated nerve fibres and a faster firing response with higher average rates of action potentials. However, there is a lack of evidence on whether this is simply because the parvocellular neurons predominate in the papillomacular bundle, or whether the midget cells are the primary target of the triggering toxic substances.

Clinical Relevance and Future Work The physiological features of the major RGC subtypes (mRGCs, pRGCs, and sbRGCs) are well-known, but the role and characteristics of other RGCs require further study. An in-depth characterisation of the chronological structural and functional changes occurring within the RGC layer in optic nerve disorders, including inherited and acquired optic neuropathies, are important to inform the future design of clinical trials. Understanding which RGC subtypes are selectively affected will help optimise outcome measures in natural history studies and trials of experimental therapies. As mentioned earlier, the FDT test is used for the early detection of glaucoma because the magnocellular pathway is more vulnerable. Given that a common variant in the SIX6 gene (rs33912345) is strongly associated with primary open-angle glaucoma (POAG) and the fact that this gene is highly expressed in midget RGCs, tests that evaluate this particular cell type could prove to be a useful sensitive biomarker of disease progression.

The remarkable advances in gene delivery and editing technology have led to an increasing number of clinical trials for optic neuropathies, in particular gene replacement therapy for monogenic inherited optic neuropathies. Gene therapy using adeno-associated viral vectors is currently favoured and there is now cumulative evidence of its long-term safety and efficacy in delivering gene constructs to retinal cells. Promising results have been obtained with allotopic expression of the MT-DN4 gene in patients with LHON treated within 1 year of disease onset. Genomic editing, such as the CRISPR-Cas system, and stem cell therapy is an exciting development that has the potential to revolutionise the treatment of ophthalmological disorders given the eye's relative ease of anatomical access and its relative immune privilege.

### Exemplary Methods for Determining the Loss of an RGC

Retinal ganglion cells (RGCs) are the bridging neurons that connect the retinal input to the visual processing centres within the central nervous system.

### Structural Quantification of RGCs

### Optical Coherence Tomography

Optical coherence tomography (OCT) is a non-invasive imaging technique that uses low-coherence light waves to capture a cross-section of various tissues. Major advances have led to the development of spectral domain OCT, which can produce a segmentation of ten layers of retina, including the retinal nerve fibre layer (RNFL) and ganglion cell layer. OCT has become a standard tool to investigate changes with RGCs as it is non-invasive, rapid, highly reproducible.

The RNFL can be measured in both the peripapillary and the macular areas. Several studies suggest that changes can be detected earlier by assessing the thickness of the RNFL in the macula compared with the peripapillary RNFL, owing to the latter's thickness. There is a good correlation between RNFL thickness and both visual acuity and visual field changes, offering an objective structural parameter for assessing glaucoma and other optic neuropathies. However, to avoid misinterpretation of OCT, several factors need to be considered: segmentation errors can occur particularly in the presence of a tilted optic disc; and RNFL thickness is also affected by age as well as by refractive error and axial length. In addition, there is lag time before any changes in the thickness of the RNFL can be detected after disease onset, and the thickness can be overestimated in the presence of optic disc or RNFL swelling.

In addition, RNFL thickness exhibits a floor effect that must be considered in advanced optic neuropathies. RNFL thinning reaches a trough at a certain level owing to residual tissues such as vessels and glial cells. Furthermore, RNFL loss usually signifies irreversible damage and functional tests (as described below) might be needed to identify ganglion cell dysfunction at a potentially reversible stage. It is well-established that visual acuity and visual fields can recover despite extensive RGC layer thinning.

Microcysts in the inner nuclear layer have been reported on macular OCT imaging in some patients with advanced loss of macular RGCs. These are thought to arise from retrograde transsynaptic degeneration and/or vitreous traction in the presence of RGC and RNFL loss. They do not seem to be specific to a particular aetiology, having been reported in patients with inherited optic neuropathies, demyelinating optic neuritis, compressive and nutritional optic neuropathies, endemic optic neuropathy and advanced glaucoma. It is not clear why these microcysts develop in only a subgroup of patients. They are seen more often in younger patients who may have a more adherent vitreous surface and ILM tension has been implicated as part of the pathophysiology. However, microcysts have also been reported as a long-term consequence associated with RGC loss in patients with silicon oil-related visual loss. These patients have undergone prior removal of the vitreous suggesting that simple vitreous traction may not be sufficient to explain the development of these microcysts.

### Detection of Apoptosing Retinal Cells

The detection of apoptosing retinal cells (DARC) is a new technique that enables visualisation of real-time RGC apoptosis using fluorescently-labelled annexin A5. This 36 kDa protein is expressed in humans and it is a well-established indicator of apoptosis. DARC has the advantage of early detection of RGC loss before visual deterioration has occurred, and it being considered for the evaluation of optic neuropathies, including glaucoma disease progression.

### Functional Evaluation of RGCs

A number of psychophysical measurements can be used to investigate changes in RGC function.

### Visual Acuity Tests

Visual acuity has been defined as the “spatial resolving capacity” of the visual system and it is the most common primary outcome measure in clinical trials. Although Snellen charts are widely used, the LogMAR scale based on the Early Treatment Diabetic Retinopathy Study (ETDRS) chart is the gold standard for clinical trials, overcoming many of the limitations of Snellen charts. However, as visual acuity tests central foveal function, patients can have widespread ganglion cell loss with preserved central visual acuity.

Spatial and Temporal Contrast Sensitivity Tests Achromatic stimuli of low and high spatial frequencies can be used to differentiate responses from the magnocellular and parvocellular systems. The magnocellular pathway has lower spatial resolution and responds to higher temporal frequencies than the parvocellular pathway. However, this difference is relatively small and the two pathways have a degree of overlap.

### Colour Vision Tests

Colour vision impairment is a frequent feature of ganglion cell pathology, but outer retinal dysfunction can also affect colour vision, such as anomalies of the cone photoreceptors. Congenital stationary red-green colour deficiencies commonly affect men, owing to loss or alteration of the long or medium wavelength opsin genes on the X-chromosome. Rarely, abnormalities in the same genetic region can give rise to S-cone monochromacy. Congenital tritan anomalies, arising from abnormalities in S-cones are also rare. Progressive or later onset cone or macular dystrophies, or congenital achromatopsia, will also affect colour vision, but in these conditions visual acuity is also usually impaired. In acquired ganglion cell pathology, however, visual acuity can be preserved with colour vision being preferentially affected. Many optic neuropathies affect red-green discrimination, although glaucoma commonly affects the blue-yellow axis.

Colour vision tests are widely used to screen patients with congenital colour vision defects and to investigate acquired pathology. There are three broad types of colour vision tests in practice. Pseudoisochromatic tests, such as the Ishihara, the Hardy-Rand-Rittler (HRR), and the Standard Pseudoisochromatic Plates (SPP), the Colour Vision Testing Made Easy (CVTME), and the Cambridge Colour Test are widely used. In arrangement tests, such as the Farnsworth-Munsell (FM) Dichotomous D-15 tests and 100-hue test, the patient is required to arrange a set of colours in order. The FM 100-hue test is highly sensitive, but time-consuming. Lastly, anomaloscopes are based on colour-matching where the observer adjusts a mixture of red and green lights to match a monochromatic orange light.

As congenital anomalies of colour perception more commonly affect red-green discrimination, many standard tests such as the Ishihara plates and the Nagel anomaloscope do not probe for tritan disorders, which are common in acquired pathologies. Tritan defects can be identified readily by other tests, including the D-15 and FM 100-hue, the Cambridge Colour Test, and the HRR plates. In addition, more specialised psychophysical methods, including measurement of the three primary colour vision mechanisms, colour adaptometry, and colour perimetry can be applied. Among them, SWAP, a specialised type of perimetry, can also be considered a colour vision test, as the targets are short-wave and the field is of long wavelength and high intensity (in order to adapt the long- and middle-wave cones).

### Visual Field Tests

In addition to conventional visual field testing, short wavelength automated perimetry (SWAP) probes the small bistratified ganglion cells and the konioceullar pathway, and high-pass resolution (ring) perimetry tests the parvocellular pathway, whereas flicker perimetry, motion perimetry, and frequency doubling technology (FDT) target the magnocellular pathway. Among these tests, SWAP and FDT are available as commercial products.

### (1) Frequency Doubling Technology (FDT)

FDT has the advantage of greater sensitivity, potentially detecting RGC damage earlier than standard automated perimetry (SAP). Modern FDT uses targets of low spatial frequency that flicker at a high temporal frequency and that predominantly stimulate the magnocellular pathway, which corresponds to motion detection and flicker detection. FDT has been put forward for the early detection of glaucoma on the basis that the magnocellular pathway is more vulnerable in glaucoma. However, there is evidence that both the parvocellular and magnocellular pathways are affected early in glaucoma with no significant differences between these two pathways in terms of their vulnerability. Furthermore, a recent study indicated that FDT is neither sensitive nor specific as a screening tool for glaucoma. Further studies are, therefore, needed to evaluate the role of FDT in the early detection of glaucoma.

### (2) Short Wavelength Automated Perimetry (SWAP)

Unlike standard visual field testing, which uses a white stimulus on a white background, SWAP employs a blue stimulus on a yellow background. Several studies suggested that SWAP is more sensitive for the early detection of glaucomatous changes compared with standard visual field testing. There is, however, no definitive evidence that the small bistratified ganglion cells (short-wave response) are more vulnerable in glaucoma. SWAP was reported to be 10-20 times more sensitive than standard perimetry in patients with ADOA (134). As a result, SWAP was able to differentiate between normal tension glaucoma with or without OPA1 polymorphism. However, SWAP has some limitations as it is time-consuming, it needs a higher level of cooperation, and it has lower reproducibility compared with standard perimetry.

### Chromatic Pupillometry

The primate pupil responds to signals from ipRGCs, which additionally receive input derived from cone responses. Chromatic pupillometry uses selective wavelengths to quantify pupil size before, during, and after a light stimulus has been applied. Comparison of pupillary responses to short-wavelength and long-wavelength light can selectively probe the function of outer retinal photoreceptors or the intrinsic response of ipRGCs. The ipRGCs are blue light sensitive and maximally sensitive to wavelengths that lie between the peak sensitivities of the rods and S-cones. Several studies using chromatic pupillometry in experimental animal models have shown that the light sensitive ipRGCs were spared in retinitis pigmentosa characterised by marked photoreceptor loss. Generally, the ipRGCs are relatively preserved in mitochondrial optic neuropathies, such as LHON and ADOA, but affected in other optic neuropathies such as glaucoma, non-arteritic anterior ischemic optic neuropathy and demyelinating optic neuritis. Bichromatic pupillometry has been used to differentiate between mitochondrial and non-mitochondrial optic neuropathies.

### Electrophysiological Tests

Electrophysiology allows direct objective assessment of electrical responses in vivo. The visual evoked potential (VEP), recorded over the visual cortex, has long been used as a means of assessing the function of the visual pathway, as well as demonstrating developmental abnormalities, such as the misrouting of ganglion cell axons in albinism. In addition, the electroretinogram (ERG), which represents the summed electrical response of the retina to light stimuli, can be recorded non-invasively. The pattern ERG (PERG), arising from stimulation of the macula, is largely derived from responses in the macular RGCs. In contrast, the full-field ERG, which is generated from the stimulation of the whole retina, is usually used to evaluate responses from photoreceptors and bipolar cells. However, a late component, the photopic negative response (PhNR) has been shown to arise in ganglion cells.

### (1) Pattern Electroretinogram

The PERG is recorded in response to a patterned stimulus (typically a checkerboard pattern reversing 4 times per second), which stimulates the central 15 degrees of the retina. The PERG comprises a cornea-positive wave at 50 ms (termed P50) and a negative wave at 95 ms (termed N95). The test is performed in photopic conditions with undilated pupils and it requires optimal refraction. The response is driven by the macular cone photoreceptors, but it appears to arise largely from the macular RGCs, whose signals appear to give rise to the N95 component and the majority of the P50 component. Various optic neuropathies that affect the ganglion cells within the retina (either as the primary site of impairment or from retrograde degeneration from an optic nerve lesion), for example demyelinating optic neuritis, ischemic optic neuropathy, compressive optic neuropathy, toxic optic neuropathy, and inherited optic neuropathies can result in a reduction of the N95 and P50 amplitudes, with N95 being reduced more than P50, and a shortening of the P50 peak time. Whilst the PERG is sensitive to macular RGC dysfunction, precise correlation with RGC subtype is not known, and the test will not detect extramacular RGC impairment.

### (2) Photopic Negative Response

The PhNR is a negative wave of long latency that follows the b-wave of the photopic cone-driven ERG and it arises in RGCs. Whilst it can be detected in standard white-on-white flash responses, specific chromatic protocols can be used to optimise the PhNR signal. As with the PERG, the amplitude of the PhNR decreases in optic nerve disorders. Unlike in PERG recordings, optimal refraction is not needed, but the pupils need to be dilated. In addition, a hand-held mini-Ganzfeld stimulator is available to test PhNR. The flashes stimulate the retina as a whole so the PhNR can be indicative of global RGC function.

Focal PhNR recordings can be performed to assess RGCs over a particular region (typically the macula). The PhNR can be used to examine the parvocellular pathway whereas the steady-state PERG is focused on the magnocellular pathway in glaucoma. Although the PERG and PhNR can detect glaucoma, there is no significant correlation between PhNR ratio and PERG ratio values.

### Pharmaceutical Compositions

The compositions of the present disclosure and/or additional therapeutic agent can be incorporated into pharmaceutical compositions suitable for administration to a subject.

For pharmaceutical compositions comprising the cells of the present disclosure, cells (e.g., RGCs, lens cells, telencephalic cells, cells derived from the organoids described herein) can be administered at a dose of at least about 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, or more, or any range in between or any value in between, cells.

The number of cells transplanted may be adjusted based on the desired level of engraftment in a given amount of time. Generally, from about 1×10² to about 1×10¹⁰ cells, from about 1×10⁴ to about 1×10⁸ cells, from about 1×10⁶ to about 1×10⁸ cells, or about 1×10⁷ cells, or more cells, as necessary, may be transplanted. In some embodiments, transplantation of at least about 100, 1000, 10,000, 0.1×10⁶, 0.5×10⁶, 1.0×10⁶, 2.0×10⁶, 3.0×10⁶, 4.0×10⁶, or 5.0×10⁶ total cells is effective.

Pharmaceutical compositions comprising the cells of the present disclosure (e.g., RGCs, lens cells, telencephalic cells, cells of the organoids described herein) may be transplanted into a subject more than once. For example, the pharmaceutical composition comprising the cells of the present disclosure may be transplanted into a subject repeatedly until the condition of the subject improves.

Pharmaceutical compositions of the present disclosure, e.g., comprising cells and/or a therapeutic agent(s), may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, subcutaneous, intradermal, transdermal, transmucosal, intraosseous infusion (the process of injecting directly into the marrow of a bone), rectal, oral, nasal, transdermal, topical, or intramuscular administration. In preferred embodiments, the pharmaceutical compositions comprising cells are transplanted in the eye of the patient.

For example, subjects of interest may be engrafted, infused, or transplanted with the cells of the present disclosure by various routes known in the art. In preferred embodiments, the pharmaceutical compositions comprising cells are administration to a specific tissue (e.g., focal transplantation, e.g., injection into the eye). Cells may be administered in one injection, or through successive injections over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

Engraftment or transplantation of cells may be assessed by any of various methods, such as, but not limited to, biomarker levels, time of administration, increase in cell number and/or function at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, or 28 days can signal the time for assessing the desired effect. Any such metrics are variables that can be adjusted according to well-known parameters in order to determine the effect of the variable on a response to the pharmaceutical compositions comprising the cells of the present disclosure. In addition, the transplanted cells can be co-transplanted with other agents, such as cytokines, extracellular matrices, cell culture supports, and the like.

Pharmaceutical compositions comprising the cells of the present disclosure can also be administered before, concurrently with, or after, other therapeutic agent(s).

Pharmaceutical compositions comprising the cells of the present disclosure and/or a therapeutic agent(s) typically comprise a pharmaceutically acceptable carrier and/or diluent (see below). As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

Pharmaceutical compositions of the present disclosure are formulated to be compatible with its intended route of administration.

Solutions or suspensions (e.g., comprising cells and/or a therapeutic agent(s)) used for parenteral, intradermal, or subcutaneous application can include the following components: a sterile diluent (pharmaceutically acceptable diluent) such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions may be co-administered with enzyme inhibitors or in an appropriate carrier such as liposomes. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

Depending on the route of administration, the active compound can be coated in a material to protect the compound from the action of enzymes, acids and other natural conditions which may inactivate the compound. For example, for administration of agents by other than parenteral administration (esp. for oral administration, e.g., an agent other than cells that treats the condition), it may be desirable to coat the agent with, or co-administer the agent with, a material to prevent its inactivation.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds including, e.g., viral particles are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

### Kits for Detection of CONCEPT Organoids

In another aspect, the invention provides kits for aiding in the detection CONCEPT organoids or cells derived therefrom (e.g., RGCs). In some embodiments, the kit comprises agents that specifically recognize biomarkers for CONCEPT organoids and/or cells derived therefrom. In related embodiments, the agents are antibodies. The kit may contain 1, 2, 3, 4, 5, or more different antibodies that each specifically recognize biomarkers for CONCEPT organoids or cells derived therefrom.

In some embodiments, the kit comprises a magnetic or paramagnetic bead having an antibody conjugated to its surface that specifically binds markers expressed by the cells in the CONCEPT organoids or cells derived therefrom. The magnetic or paramagnetic bead can be used in MACS to isolate or purify a CONCEPT organoid or cells derived therefrom. In alternative embodiments, the kit comprises reagents to isolate or purify a CONCEPT organoid or cells derived therefrom using FACS. In some embodiments, the kit comprises a solid support, such as a chip, a microtiter plate, or a bead or resin having capture reagents attached thereon, wherein the capture reagents bind the biomarkers of the invention.

The kit can also comprise a washing solution or instructions for making a washing solution, in which the combination of the capture reagent and the washing solution allows capture of the biomarker or biomarkers on the solid support for subsequent detection.

In a further embodiment, such a kit can comprise instructions for use in any of the methods described herein. In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

In one embodiment, the kits described herein can be used in assessing the safety and efficacy of a candidate therapeutic in an ex vivo environment.

### Exemplary Embodiments

1. An organoid comprising at least two concentric zones of telencephalic and ocular progenitors.

2. The organoid of 1, further comprising FOXG1+ telencephalic cells.

3. The organoid of 1 or 2, further comprising PAX6+ multi-lineage ocular cells.

4. The organoid of any one of 1-3, further comprising VSX2+ retinal cells.

5. The organoid of any one of 1-4, further comprising PAX2+ optic disc and optic stalk cells.

6. The organoid of any one of 1-5, further comprising a lens cell.

7. The organoid of any one of 1-6, further comprising a retinal ganglion cell (RGC).

8. The organoid of 7, wherein the RGC expresses at least one cell surface marker selected from ATOH7, POU4F2, ONECUT2, and TUBB3.

9. The organoid of 7 or 8, wherein the RGC comprises a TUBB3+ axon.

10. The organoid of any one of 7-9, wherein the RGC comprises an axon that grows in a long, directional manner.

11. The organoid of any one of 7-10, wherein the RGC expresses CNTN2 on the cell surface.

12. The organoid of any one of 1-11, wherein the organoid is of a mammal, optionally a mouse or a human.

13. The organoid of 12, wherein the organoid is of a human.

14. A method of producing the organoid of any one of 1-13, the method comprising:
(a) inducing pluripotent stem cells to form a cyst showing an epithelial structure;
(b) plating a cyst at a low density; and
(c) growing the cyst as an adherent colony.

15. The method of 14, wherein the cyst is produced by culturing Matrigel-embedded stem cell sheets in a suspension culture.

16. The method of 14 or 15, wherein
(a) the cyst is formed in a medium comprising the N2 and B27 supplements; and/or
(b) the cyst is plated in a medium comprising the N2 and B27 supplements.

17. The method of any one of 14-16, wherein the adherent colony of the cyst is grown in a medium comprising the KnockOut® Serum Replacement.

18. The method of any one of 14-17, wherein the pluripotent stem cells are embryonic stem cells (ESCs) or induced pluripotent stem cells (iPSCs).

19. The method of any one of 14-18, wherein the pluripotent stem cells are of a mammal, optionally a mouse or a human.

20. An organoid produced by the method of any one of 14-19.

21. A method of producing a lens cell, the method comprising
(a) producing an organoid according to the method of any one of 14-19;
(b) detaching and partially dissociating the organoid; and
(c) optionally growing the partially dissociated organoid in a suspension culture.

22. The method of 21, wherein partially dissociating the organoid separates the lens cells from other types of cells but leaves the lens cells as clusters.

23. The method of 21 or 22, wherein the lens cells express CRYAA and/or beta crystalline.

24. The method of any one of 21-24, further comprising isolating the lens cell by manually picking crystal-like cell clusters comprising the lens cell from the suspension culture.

25. A lens cell produced according to the method of any one of 21-24; and/or a lens cell derived from the organoid of any one of 1-13 and 20.

26. A method of producing a telencephalic cell and/or a retinal ganglion cell (RGC), the method comprising producing an organoid comprising the telencephalic cell and RGC according to the method of any one of 14-19.

27. A telencephalic cell produced according to the method of 26; and/or a telencephalic cell derived from the organoid of any one of 1-13 and 20.

28. A retinal ganglion cell (RGC) produced according to the method of 26; and/or an RGC derived from the organoid of any one of 1-13 and 20.

29. The RGC of 28, wherein the RGC is CNTN2+.

30. A method of isolating a retinal ganglion cell (RGC) from an organoid, the method comprising:
contacting a cell from the organoid with an antigen-binding protein that specifically binds to CNTN2;
capturing an antigen-binding protein-RGC complex; and
removing the antibody from the antigen-binding protein-RGC complex, thereby isolating the RGC from the organoid.

31. The method of 30, wherein the organoid is the organoid of any one of 1-13 and 20; and/or the organoid is produced using the method of any one of 14-19.

32. The method of 30 or 31, wherein the antigen-binding protein is conjugated to a paramagnetic bead.

33. The method of any one of 30-32, wherein capturing the antigen-binding protein-RCC complex comprises performing magnetic activated cell sorting (MACS).

34. The method of any one of 30-33, wherein the antigen-binding protein comprises a label.

35. The method of 34, wherein the label is selected from biotin, a histidine tag, a myc tag, a HA tag, a flag tag, a fluorescent moiety, and an enzyme.

36. The method of any one of 30-35, wherein capturing the antigen-binding protein-RCC complex comprises performing fluorescence activated cell sorting.

37. The method of any one of 30-36, wherein the antigen-binding protein is an antibody.

38. A retinal ganglion cell (RGC) isolated by the method of any one of 30-37.

39. A pharmaceutical composition comprising a lens cell of 25, a telencephalic cell of 27, an RGC of any one of 28, 29, and 38, or any combination thereof.

40. A method of treating a subject having or suspected of having a loss of a retinal ganglion cell, the method comprising:
administering to the subject a composition comprising the RGC of any one of 28, 29, and 38; or the pharmaceutical composition of 39.

41. The method of 40, wherein the RGC expresses at least one cell surface marker selected from ATOH7, POU4F2, ONECUT2, and TUBB3.

42. The method of 40 or 41, wherein the RGC comprises a TUBB3+ axon.

43. The method of any one of 40-42, wherein the RGC comprises an axon that grows in a long, directional manner.

44. The method of any one of 40-43, wherein the RGC expresses CNTN2 on the cell surface.

45. The method of any one of 40-44, wherein the subject is administered with at least about 104 RGCs and/or between 104 to 10⁸ RGCs.

46. The method of any one of 40-45, wherein the subject has an optic neuropathy, demyelinating optic neuritis, toxic optic neuropathy, glaucoma, an age-related loss of RGCs, optic neuritis, autosomal dominant optic atrophy, Leber hereditary optic neuropathy, or an eye injury.

47. The method of any one of 40-46, wherein the subject has glaucoma.

48. The method of any one of 40-47, further comprising administering to the subject at least one agent that prevents or treats the loss of an RGC.

49. The method of 48, wherein the at least one agent is selected from prostaglandin analogs (e.g., Xalatan® (latanoprost), Lumigan® (bimatoprost), Travatan Z® (travoprost), and Zioptan™ (tafluprost), and Vyzulta™ (latanoprostene bunod)); beta blockers (e.g., timolol); alpha agonists (e.g., Alphagan®P (brimonidine), Iopidine® (apraclonidine)); carbonic anhydrase inhibitors (CAIs, e.g., Trusopt® (dorzolamide), Azopt® (brinzolamide) Diamox® (acetazolamide), and Neptazane® (methazolamide)); rho kinase inhibitors (Rhopressa® (netarsudil)); and a combination thereof.

50. The method of any one of 40-49, wherein the RGC or a pharmaceutical composition is administered intraorbitally.

51. The method of any one of 40-50, wherein the RGC or a pharmaceutical composition is administered more than once.

52. The method of any one of 40-51, wherein the RGC or a pharmaceutical composition comprising the RGC is autologous or allogeneic to the subject.

53. The method of any one of 40-52, wherein the subject is a mammal, optionally a mouse or a human.

54. The method of any one of 40-53, wherein the subject is a human.

EXAMPLES

The invention is further described in the following examples, which do not limit the scope of the invention described in the claims.

### Example 1: Generation of a Telencephalon-Eye Organoid that is Composed of Concentric Zones of Anterior Ectodermal Progenitors (“CONCEPT”)

H1 hESCs or iPSCs were passaged using ReLeSR three days before the experiment. On day 0, cells were detached using dispase and suspended in ice-cold Matrigel. After gelling at 37° C. for 15 minutes, the hESC/Matrigel clump was gently dispersed in a N2B27 Medium for suspension culture in a low-adherent 24-well plate. Cysts with a single lumen formed on day 1. To generate CONCEPT organoids, cysts were passaged to 24-well plates at a low density on day 3-5 and grown as attached colonies (FIG. 1A-FIG. 1L). Telencephalon marker FOXG1 (FIG. 1A), neuroretinal progenitor marker VSX2 (FIG. 1), and multi-lineage retinal maker PAX6 (FIG. 1C) exhibit concentric patterns in CONCEPT organoids (FIG. 1D-FIG. 1L).

### Example 2: Cell Cluster Analysis of CONCEPT Organoids

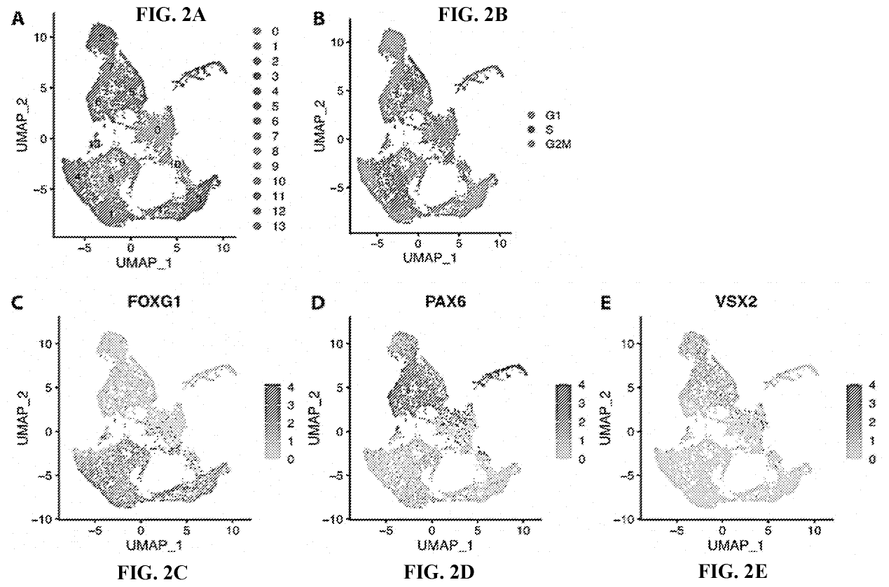

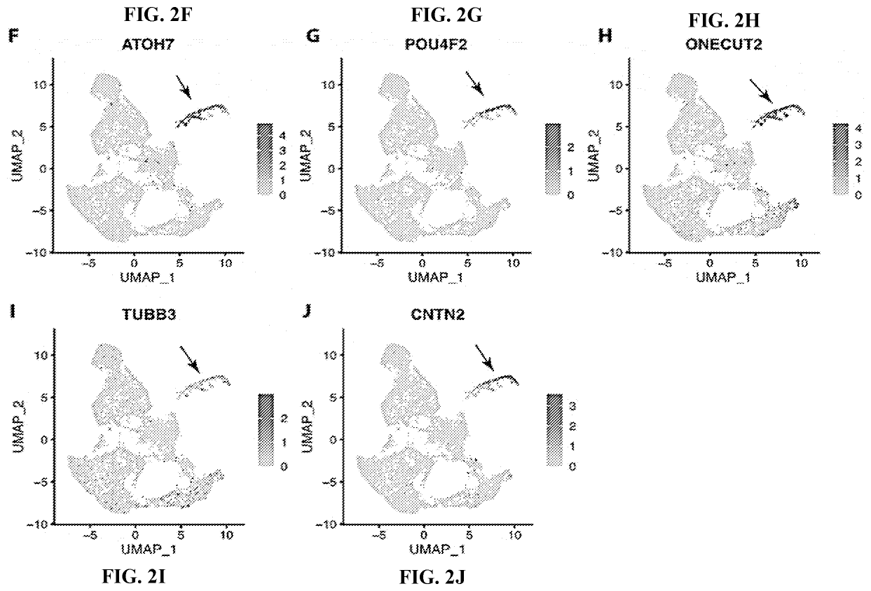

Cell cluster analysis of CONCEPT organoids at day 24 identified a cell surface protein, CNTN2, that is specifically expressed in retinal ganglion cells. CONCEPT organoids at day 24 were dissociated into singles and then captured for single-cell RNA sequencing on 10× (Pleasanton, CA) platform. Referring to FIG. 2A, cell cluster analysis showed 14 distinct cell populations and G1, S, and G2M stages of the cell cycle. Cell clustering analysis (Seurat 3) confirmed that CONCEPT organoids were composed of FOXG1+ telencephalon cells and PAX6+ and/or VSX2+ retinal cells. Cell cluster 11 specifically expressed RGC markers ATOH7, POU4F2, ONECUT2, and TUBB3, as well as cell surface protein CNTN2.

### Example 3: Human RGCs in CONCEPT Organoids Grow Directional Long Axons Along a Path Defined by Tissues Reminiscent of the Optic Disc and/or Optic Stalk

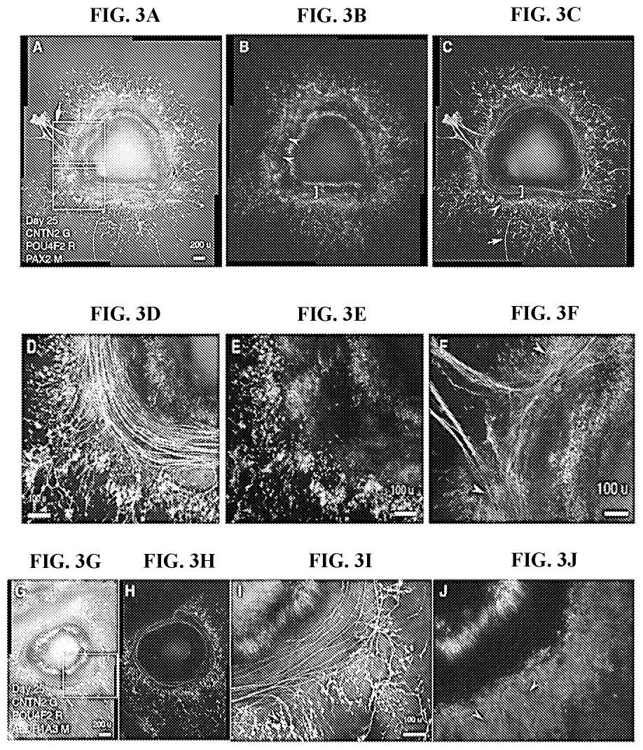

FIG. 3A-3F show concentric patterns of immunostaining for RGC nuclear marker POU4F2 (red), RGC axon marker CNTN2+(green), and optic disc and optic stalk marker PAX2 (magenta) in CONCEPT organoids at day 25. Composite color images of three and two channels at low and high magnification are shown. POU4F2+ RGC nucleus (red) are mostly abundant adjacent to a PAX2+ tissue (magenta at moderate levels, bracketed areas in FIG. 3B, FIG. 3C). RGCs grow axons along a path that is defined by the adjacent PAX2+ cell population (FIG. 3A-FIG. 3E), reminiscent of RGC axon growth towards the optic disc in vivo. In areas where there is a gap in the PAX2+ cell population, RGC axons exit the circular path and grow centrifugally (regions between the two arrowheads in FIG. 3C, FIG. 3F), further indicating that the PAX2+ cell population play critical roles in RGCs axon growth. The PAX2^{High} cell population in the inner circle appears to be a border of the path for RGC axon growth. FIG. 3G-FIG. 3J show that the path for RGC axon growth is within regions that do not express optic stalk marker ALDH1A3, reminiscent of RGC axon growth in the path that is confined by the optic stalk in vivo.

### Example 4: Retinal Ganglion Cells (RGCs) Derived from CONCEPT Organoids

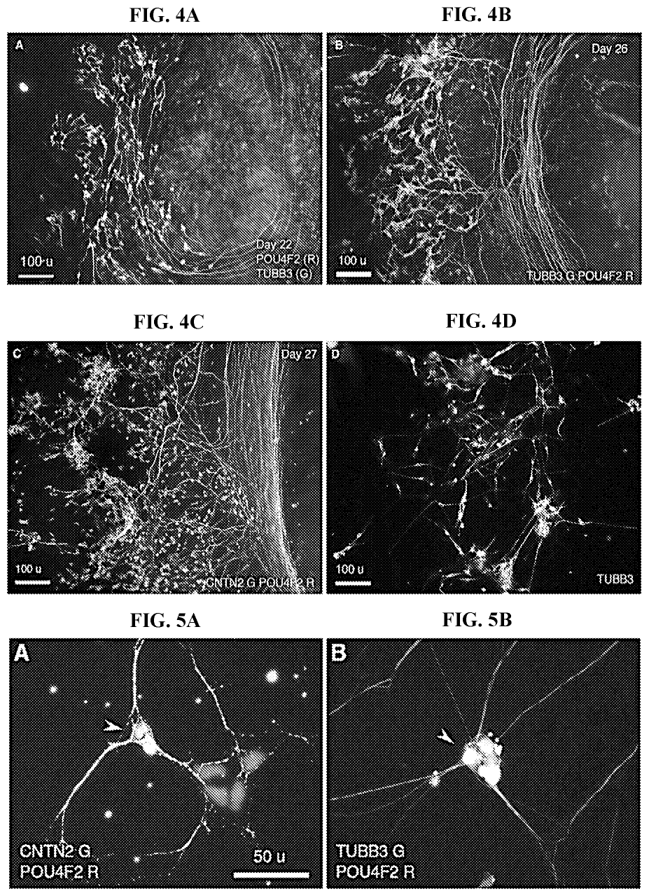

Fluorescent imaging was used to investigate axonal grow in RGCs in CONCEPT organoids. Specifically, fluorescently labeled antibodies that specifically bind POU4F2 and TUBB3 were used to label RGCs purified using magnetic activated cell sorting with an antibody against CNTN2. Results show that RGCs grow directional axons in CONCEPT organoids and can be efficiently purified using cell sorting with a CNTN2 antibody. POU4F2+ RGCs grow directional TUBB3+ long axons in CONCEPT organoids (FIG. 4A and FIG. 4B). CNTN2 and TUBB3 exhibit very similar expression patterns in CONCEPT organoids (FIG. 4B, FIG. 4C). Isolated RGCs exhibit typical neuronal morphology and expressed TUBB3. Compared to directional axon outgrowth in CONCEPT organoids (FIG. 4A-FIG. 4C), axon growth of isolated human RGCs appeared to be in random directions (FIG. 4D).

### Example 5: Further Validation of Isolated Human RGCs

FIG. 5A shows that isolated human RGCs express both CNTN2 and POU4F2. FIG. 5B shows that isolated human RGCs express both TUBB3 and POU4F2.

### Example 6: Materials and Methods for Examples 1-15

Maintenance of hESCs

ESCRO and TRB committees at AECOM approved the use of hESCs in this project. Undifferentiated H1 hESCs (WiCell WA01) or hiPSCs (Corriell Institute AICS 0023) were grown on Matrigel-coated 6-well plates in mTeSR1 medium and passaged using ReLeSR (STEMCELL technologies) following manufacturer instructions.

### Retinal Cell Differentiation

CONCEPT telencephalon-eye organoids were generated as follows. A humidified incubator at 37° C. with 5% C02 was used for cell culture. H1 hESCs or iPSCs that were passaged using ReLeSR two or three days before experiments were detached using Dispase (GIBCO 17105041) and then harvested by centrifugation. After that, the cell pellets were suspended in ice-cold Matrigel. After gelling at 37° C. for 15-20 minutes, the hESC/Matrigel clump was gently dispersed in a N2B27 Medium (DMEM/F12+GlutaMAX (GIBCO):Neurobasal medium (GIBCO)=1:1, 0.5×B27 supplement (GIBCO), 0.5×N2 supplement (GIBCO), 0.1 mM β-mercaptoethanol, and 0.2 mM L-GlutaMax) for floating culture. With the starting day of cell differentiation designated as day 0, cysts with a single lumen formed on day 1. On days 3-5, individual cysts were manually picked using a curved Pasteur pipets under an inverted microscope and then seeded onto Matrigel-coated 24-well plates at a density of 2-6 cysts per well. Cysts spontaneously attached to the culture surface and grew. From a time during days 13-16, attached cell colonies were grown in a KSR medium (GMEM medium supplemented with 10% KnockOut® serum replacement (ThermoFisher Scientific), 1 mM sodium pyruvate, 0.1 mM non-essential amino acids, 2 mM 1-glutamine, and 55 μM 2-mercaptoethanol; all the culture reagents were from Life Technologies). Culture mediums were changed every two or three days.

Inhibition of FGF Signaling in CONCEPT Telencephalon-Eye Organoids with FGFR and MEK Inhibitors

To inactivate FGF signaling in CONCEPT organoids, FGFR1/2/3 inhibitor PD 161570 (1 μM; Tocris) and MEK1/2 inhibitor PD 0325901 (2 μM; Tocris) were supplemented to the culture medium staring on day 15. DMSO was used as a control. Treated CONCEPT organoids were harvested on day 24.

### Magnetic-Activated Cell Sorting (MACS) of Developing Human RGCs

Retinal organoids in suspension culture were generated using the established method. For each MACS experiment, 84-140 retinal organoids at stages of day 41-70 were dissociated into single cells using Accutase (GIBCO A1110501). Non-retinal cells were trimmed if there were any. Dissociated single cells were harvested using centrifugation and then incubated for 25 minutes at room temperature with MagnaBind goat anti-mouse IgG (ThermoScientific 21354) beads that were previously coupled with a CNTN2 antibody (DSHB 4D7) following manufacturer instructions. Cells bound to the beads were isolated using a magnetic rack and then washed one time with the KSR medium supplemented with antibiotic:Antimycotic (GEMINI 400101) while the tube was still against the magnetic rack. After the wash, the cells were released from the beads via Accutase digestion for 30 minutes and then harvested using centrifugation. The isolated cells were plated onto a chamber slide (ibidi 80826, ibidiTreat μ-Slide 8 Well, coated with poly-ornithine and Matrigel, 30,000-50,000 cells/200 μl/well) in BrainPhys neuronal medium (Stem Cell Technology 05790) supplemented with N2 and B27 (GIBCO 17502001, A3582801). From 100 retinal organoids on day 41 to 48, around 385,000 RGCs were isolated. After 10 days of culture in chamber slides, RGCs were fixed in 4% paraformaldehyde (PFA) for 10 minutes and then processed for immunostaining.

### Immunostaining, Antibodies, and Light Microscopy

CONCEPT telencephalon-eye organoids were fixed in 4% PFA for 15-30 minutes at room temperature and processed for immunostaining. These primary antibodies were used: FOXG1 (Abcam, ab18259, 1:500), TUBB3 (Covance MMS-435P, 1:1000), FGF8 (1:500, R&D MAB323), RBPMS (1:200, PhosphoSolution 1830-RBPMS), ISL1 (1:500, DSHB 40.2D6), SNCG (1:200, Abcam ab55424), PAX2 (Invitrogen, 716000, 1:200), alpha A crystallin (Santa Cruz sc 22743, 1:500, shown as CRYAA in figure panels), beta crystallin (Santa Cruz sc-22745, 1:100, shown as CRY B in figure panels), CNTN2 (DSHB 4D7, 1:100), ALDH1A3 (Invitrogen, PA529188, 1:500), VAX1/2 (Santa Cruz sc-98613, 1:200), PAX6 (1:500, Covance PRB-278P), POU4F2 (Santa Cruz, SC-6026, 1:200), SIX3 (1:500, Rockland), and VSX2 (1:500, Millipore AB9016). Primary antibodies were visualized using Alexa Fluor 488-, 568-, and 647-conjugated secondary antibodies and imaged using a Zeiss AxioObserver Z1 microscope. When the sample did not fit in one image, multiple images were stitched to obtain an overview. In dual-color immunohistochemistry for PAX2 and FGF8, FGF8 was visualized using AP-conjugated anti mouse secondary antibody (Invitrogen A16038), and PAX2 was detected by biotin-conjugated anti rabbit secondary antibody (Invitrogen B2770) followed by H1RP-conjugated streptavidin (ThermoScientific 21130).

### In Situ Hybridization

DIG-labeled anti-sense RNA probes for in situ hybridization were generated via in vitro transcription using a DIG RNA labeling kit (Millipore Sigma-Aldrich 11175025910). DNA templates for in vitro transcription were generated using PCR with the cDNA of CONCEPT telencephalon-eye organoids at day 24 as a template. The sequence of T7 promoter was added to reverse primers for in vitro transcription. In situ hybridization was performed as previously described. Images were taken using a Leica stereomicroscope.

PCR primers are as follows:

BMP4: forward,
CGGAAGCTAGGTGAGTGTGG,
reverse,
GAGtaatacgactcactatagggGGAAGCCCCTTTCCCAATCA;
BMP7: forward,
gaggtccctctccattccct,
reverse,
GAGtaatacgactcactatagggtgcacccatcagacctccta;
FGF8: forward,
GTTGCACTTGCTGGTCCTCT,
reverse,
GAGtaatacgactcactatagggTTGAGTTTTGGGTGCCCTAC;
PAX2: forward,
gctgtctgtgctgtgagagt,
reverse,
GAGtaatacgactcactatagggccggggacatttagcaggtt;
SEMA5A: forward,
CAGAGGCTCAGGCACAATGA,
reverse,
GAGtaatacgactcactatagggTCCGTGTCTACCCAGGACTT;
CYP1B1: forward,
cccagcggttcttcatgagt,
reverse,
GAGtaatacgactcactataggggcacacttggttgcgttagt;
LEFTY2: forward,
agccctctaactgaacgtgtg,
reverse,
GAGtaatacgactcactatagggtcttctgagtatctacattcaattgct;
EMX2: forward,
ACCGAGAAAGGGAGAGGGAA,
reverse,
GAGtaatacgactcactatagggTCGGCCAATTTCTCCAACCA;
FGF9: forward,
GTCCGCTATGAACCTGTGGT,
reverse,
GAGtaatacgactcactatagggATAGTCTCGCTTGCCCAAGG;
FGF2: forward,
aacaccgaaatgctggaggt,
reverse,
GAGtaatacgactcactataggggagaccacatgtacacgcca.

To assess the in vivo expression of DEGs identified by single-cell RNA sequencing, in situ hybridization images of their mouse orthologs in E14.5 mouse brains were downloaded from a public database (World Wide Web at gp3.mpg.de/) with permission and then assembled in the figures.

### Electron Microscopy (EM)

EM was performed by Analytical Imaging Facility in Albert Einstein College of Medicine with a standard method. Lens cell clusters were fixed in 0.1M Cacodylate buffer containing 2% paraformaldehyde and 2.5% glutaraldehyde for 60 minutes at room temperature and then processed for EM.

### Single-Cell RNA Sequencing

CONCEPT telencephalon-eye organoids at day 24 from one culture well were dissociated into single cells using activated Papain (Worthington Biochemical Corporation LS003126) following manufacturer's instructions. Then, 10,000 dissociated cells were captured using Chromium Controller (10× Genomics), followed by library preparation using Single Cell 3′ version 3.1 kit (10× Genomics) following manufacturer's instructions. The library was sequenced in one lane of Illumina HiSeq (2×150 bp) in GeneWIZ company.

Fastq sequences were mapped to the human genome (GRCh38-3.0.0) using CellRanger (3.1.0) to generate a count matrix, which was then analyzed using the Seurat Package (v3.2.0). Sequenced cells were filtered (nFeature_RNA >200 & nFeature_RNA <6000 & percent.mt<20), resulting in 10218 cells with high quality. Dimension reduction and clustering were performed using the following functions: NormalizeData, FindVariableFeatures, ScaleData, RunPCA, ElbowPlot, FindNeighbors (dims=1:17), and FindClusters (resolution=0.5). Differentially expressed genes were identified using the function FindAllMarkers (only.pos=FALSE, min.pct=0.25, logfc.threshold=0.25).

### Statistical Analysis

Statistical analysis in DEG identification was performed using the Seurat Package (v3.2.0). Adjusted p-values were shown.

### Data Availability

Single-cell RNA sequencing data of CONCEPT telencephalon-eye organoids at day 24 is available in Gene Expression Omnibus (World Wide Web at ncbi.nlm.nih.gov/geo) using the accession number GSExxx.

### Whole-Cell Patch Clamp Recordings of Isolated RGCs in Culture.

RGCs were isolated from retinal organoids on day 48 using MACS via a CNTN2 antibody and then grown on polymer coverslips in chamber slides (ibidi 80826) for 20-27 days. At the time of whole-cell patch clamping recordings, the polymer coverslips carrying RGCs were carefully cut out with a scalpel blade and then placed in a recording chamber under an upright microscope (Zeiss Examiner A1), containing artificial cerebrospinal fluid (aCSF) composed of (in mM): NaCl (140), MgCl2 (1), KCl (5), CaCl2) (2), Hepes (10), Glucose (10). Osmolarity and pH were adjusted to 300 mOsm and 7.3 respectively. Whole-cell patch-clamp recordings in voltage and current-clamp mode were obtained at room temperature using an Optopatch amplifier (Cairn Research, UK) and acquired with WinWCP 5.2 freeware (John Dempster, SIPBS, University of Strathclyde, UK). Patch pipettes (3-4MΩ when filled with corresponding solution) were pulled from borosilicate capillaries using a horizontal puller (Sutter P97, USA) and coated with dental wax to reduce the pipette capacitance. For current clamp recordings, patch pipettes were filled with a solution containing (in mM): K-gluconate (130), Na-gluconate (10), NaCl (4), Hepes (10), Phosphocreatin (10), MgATP (4), Na2GTP (0.3). Osmolarity and pH were adjusted at 295mOsm and 7.3 respectively. Resting membrane potential was obtained from averaging membrane potential recorded for one minute in I=0 mode immediately after breaking the cell membrane. In current clamp mode, with the cell hyperpolarized to −70 mV, current steps of 100 to 500 pA were made to explore whether the cells were excitable. For voltage-clamp recordings, cells were held at clamped potential of −80 mV, and series resistance was monitored and compensated (>80%). Membrane potentials were corrected for the liquid junction potential, calculated at +5.4 mV (https://swharden.com/LJPcalc). Patch pipettes were filled with a solution containing (in mM): KCl (140), MgCl2 (5), CaCl2) (2.5), Hepes (10), MgATP (4), Na2GTP (0.3). Osmolarity and pH were adjusted at 295mOsm and 7.3 respectively. A −P/4 subtraction protocol was used to isolate voltage-gated currents by removing linear leak current and capacitance artifacts. In all cases, voltage and current recordings were low pass filtered at 3 kHz and digitized at 10-20 kHz (Axon Digidata 1550b, Molecular Device, USA). Tetraethylammonium-chloride (TEA, Sigma-Aldrich, USA) and Tetrodotoxin-citrate (TTX, Fisher Scientific) stocks were diluted in aCSF to reach 20 mM and 1 μM respectively. All recordings were analyzed with WinWcp 5.2 and custom scripts and routines written in Python 3.9. Statistical tests were performed with corresponding function from scipy.stats package (v1.8).

### Example 7: Generation of a Telencephalon-Eye Organoid that is Composed of Concentric Zones of Anterior Ectodermal Progenitors (CONCEPT)

The telencephalon and eye in mammals are developed from adjacent fields in the anterior neural plate through coordinated cell specification. Early specification of telencephalic and ocular tissues in humans is not well understood. We recently generated an epithelial structure—the cyst—from human pluripotent stem cells through suspension culture of hESC sheets/Matrigel clumps. Cysts were used for efficient generation of retinal organoids. Despite these advances, developmental potentials of cysts have not been fully characterized.

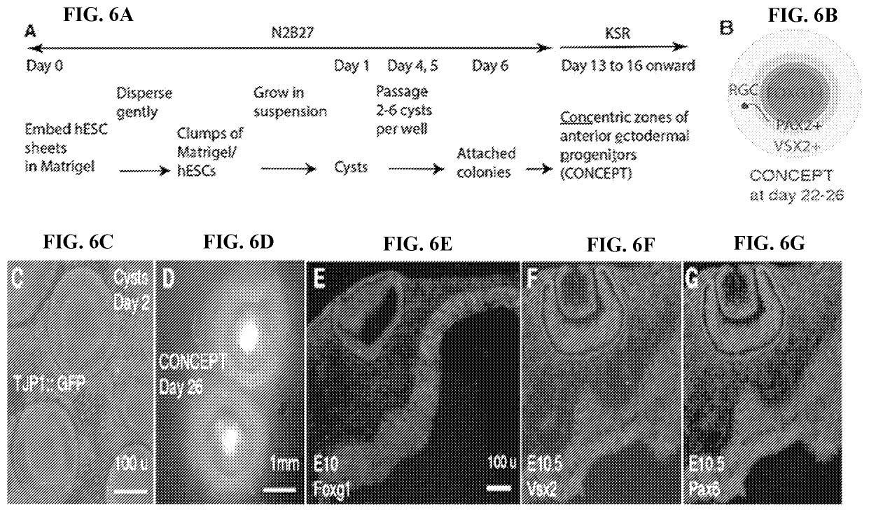

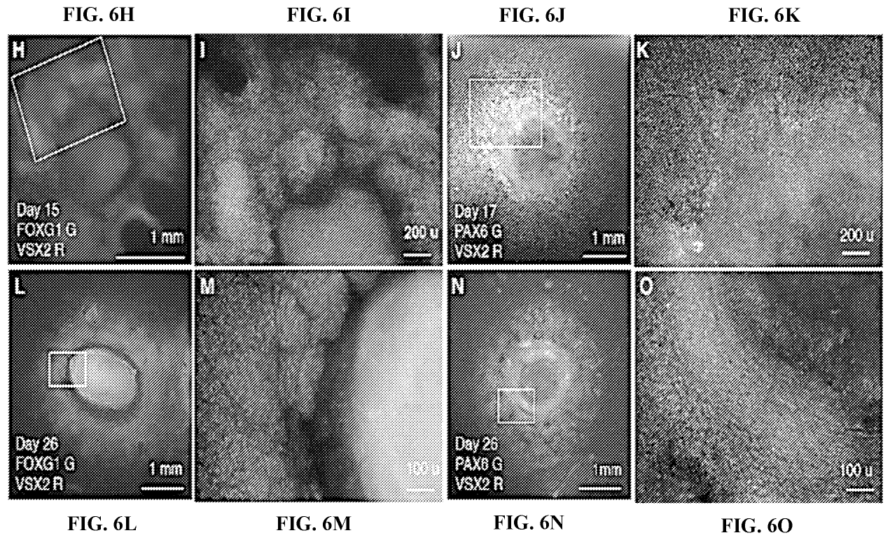

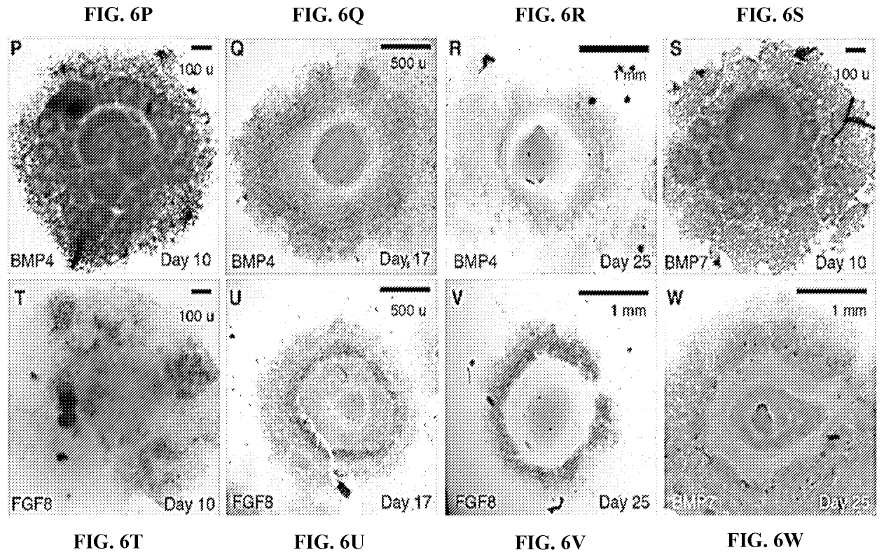

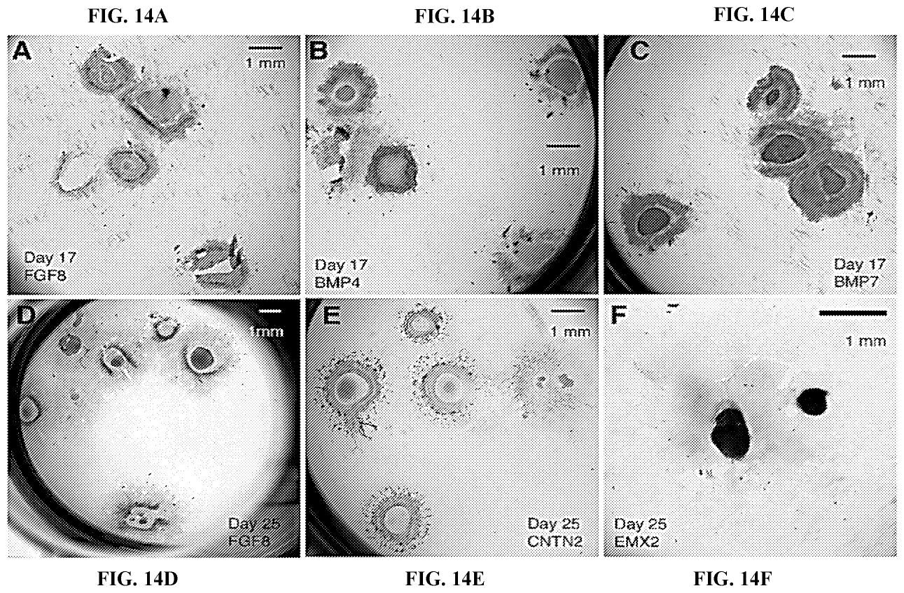

Using the cysts, herein we generated a telencephalon-eye organoid that is composed of concentric zones of anterior ectodermal progenitors (CONCEPT) (FIG. 6A, 6B; see also Example 6 for a method of generating the CONCEPT organoids). The epithelial structure of cysts was demonstrated by apically localized TJP1::GFP reporter at the lumen (FIG. 6C), consistent with our previous findings. To assess developmental potentials of cysts, individual cysts were manually picked and then seeded onto the Matrigel-coated surface at low densities (FIG. 6A). After attaching to the culture surface, cysts initially grew as dome-shaped individual colonies. Subsequent culture of the colonies in a KSR medium led to the self-formation of concentric zones of anterior ectodermal progenitors: an elevated central zone surrounded by multiple zones; this morphology was distinguishable under a steoromicroscope (FIG. 6D) or an inverted microscope. CONCEPT structures were highly reproducible, which was demonstrated by consistent gene expression profiles of multiple CONCEPT organoids in whole culture wells (FIG. 14). If multiple cysts were fused together or cysts for seeding were too big or small, CONCEPT structures were affected or incomplete. CONCEPT structures expressed gene markers for telencephalon and eye in concentric patterns. In mice, Foxg1 is specifically expressed in the E10 telencephalon at high levels and in the retina and lens at lower levels (FIG. 6E); Vsx2 is specifically expressed in the E10.5 neuroretina (FIG. 6F); Pax6 is specifically expressed in the E10.5 neuroretina, RPE, lens vesicles, and surface ectoderm (FIG. 6G). In CONCEPT structures, the expression of FOXG1, VSX2, and PAX6 generally exhibited concentric patterns spanning from the center to the periphery (FIG. 6H-6O). Similar to FOXG1, telencephalon marker EMX2 was also expressed at the central region (FIG. 14F). PAX6 was expressed in multiple concentric zones at distinct levels, mimicking its expression patterns in multiple ocular tissues in E10.5 mouse eyes (FIG. 6G, 6J, 6K, 6N, 6O). Therefore, the CONCEPT structure is a telencephalon-eye organoid mimicking telencephalic and ocular tissues in the aspects of cell types and patterning at early stages.

Morphogens FGFs and BMPs play crucial roles in patterning the forebrain and eye in vivo. In CONCEPT telencephalon-eye organoids, FGF8, BMP4, and BMP7 were expressed in attached cell colonies starting from early stages (FIG. 6P, 6S, 6T) and subsequently exhibited concentric patterns (FIG. 6R, 6V, 6W). At day 10, the expression of BMP4 and BMP7 delineated multiple rings, with a big ring mostly at the center surrounded by numerous small rings (FIG. 6P, 6S). These observations suggest that the attachment of a single-lumen cyst to the culture surface caused differences in cell behaviors: some cells separated from the original cyst and migrated peripherally; some of the separated cells formed small ring-like structures; the cells that remained at the center formed a big ring-like structure. At day 17, concentric patterns of BMP4 and FGF8 emerged (FIG. 6Q, 6U). At day 25, BMP4, FGF8, and BMP7 clearly exhibited concentric patterns (FIG. 6R, 6V, 6W). Hence, FGFs and BMPs spontaneously formed concentric gradients, which dictated coordinated cell specification in CONCEPT telencephalon-eye organoids.

### Example 8: Early Differentiated RGCs Grow Directional Long Axons Toward and Along a Path Defined by PAX2+ Cell Populations in CONCEPT Telencephalon-Eye Organoids

To assess cell differentiation in CONCEPT telencephalon-eye organoids, we examined marker expression for RGCs, the first type of cells that differentiate in the neuroretina. In mice, transcription factor Pou4f2 is expressed in the early differentiated RGCs and required for the development of a large set of RGCs. Tubb3 is expressed in the somas and axons of differentiating RGCs.

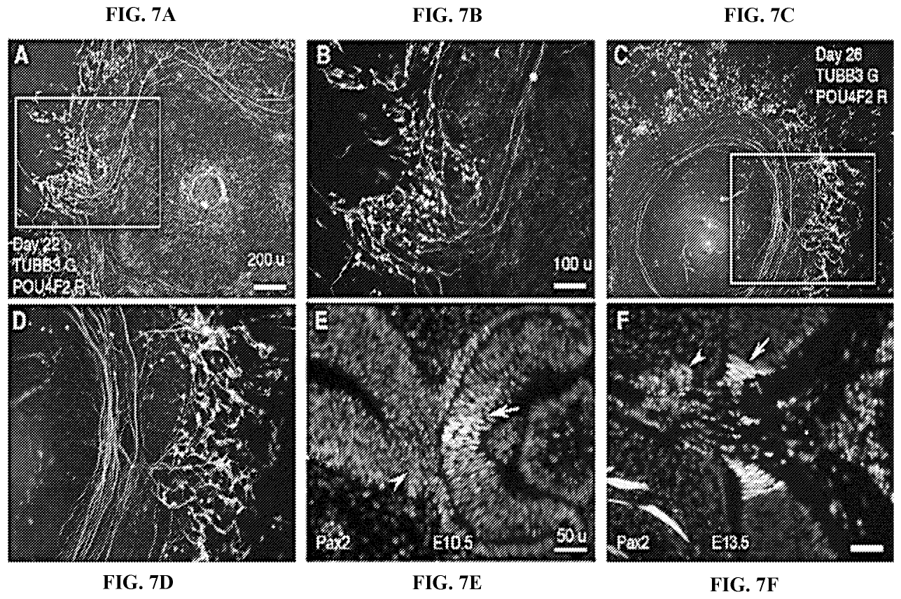

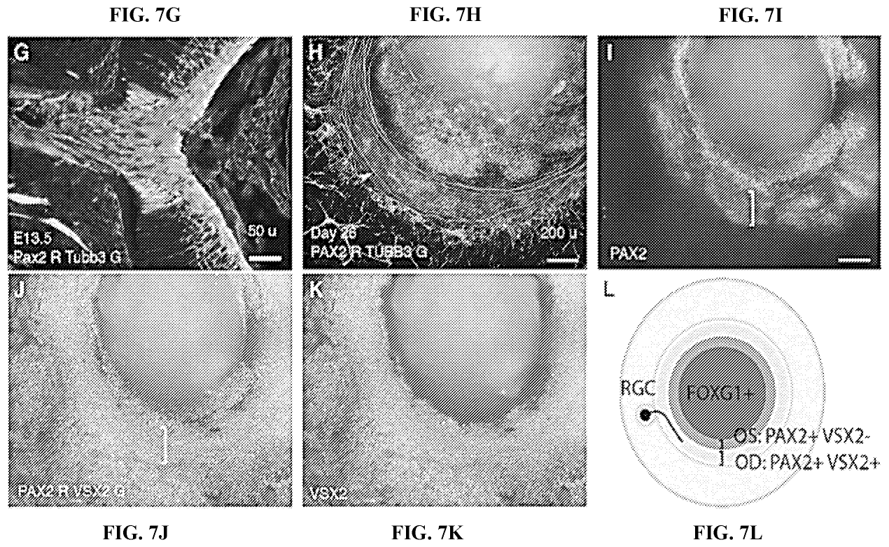

In CONCEPT telencephalon-eye organoids, POU4F2 and TUBB3 were detectable as early as day 17 and increased to higher levels at day 22. RGC somas were marked by POU4F2 and TUBB3 co-expression; RGC axons were marked by TUBB3 expression. Interestingly, RGCs grew directional long axons that followed a circular path (FIG. 7A, 7B). The circular path of RGC axon outgrowth became more evident in the organoids at day 26 (FIG. 7C, 7D). In mice, the initially differentiated RGCs are adjacent to the optic disc; their axons grow towards the optic disc to exit the eye and then navigate within the optic stalk to reach their targets in the brain; the optic disc and stalk specifically express Pax2 at distinct levels (FIG. 7E-7G); presumptive optic disc cells expressed Vsx2 whereas optic stalk cells do not (FIG. 6F; 7E). In CONCEPT organoids, there were two PAX2+ cell populations that formed two adjacent rings (FIG. 7H-7L). POU4F2+ RGCs grew TUBB3+ axons toward and then navigated along the adjacent PAX2+VSX2+ cell population (FIG. 7H-7L), mimicking axon growth from the initial RGCs toward the PAX2+ optic disc in vivo. Meanwhile, PAX2+VSX2− cell population at the inner zone set up an inner boundary of the path for RGC axon growth, mimicking PAX2+ optic stalk that spatially confines RGC axon growth in vivo. Based on these findings, we designated PAX2+VSX2+ cells and PAX2+VSX2− cells as the optic disc and optic stalk, respectively. Taken together, our findings demonstrated that RGCs grow directional axons toward and then along a path defined by PAX2+ cell populations in CONCEPT telencephalon-eye organoids.

### Example 9: CONCEPT Telencephalon-Eye Organoids Contain Lens Cells that Undergo Terminal Differentiation

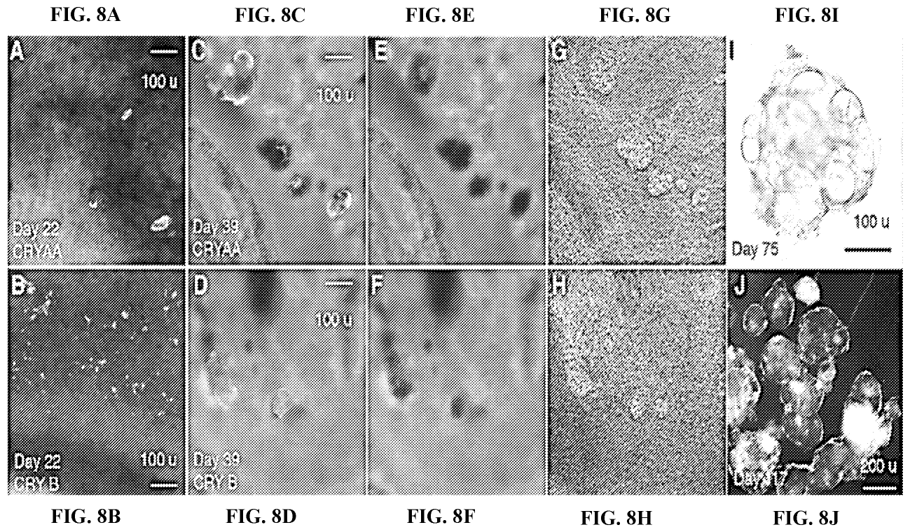

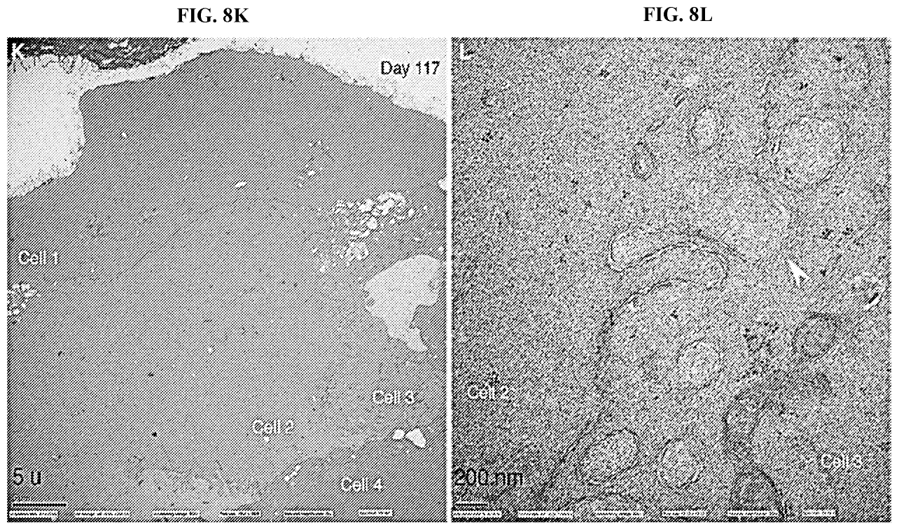

CONCEPT telencephalon-eye organoids at stages around day 25 contained transparent structures reminiscent of the ocular lens. To determine their cell identity, we performed immunostaining. Starting at day 22, lens markers CRYAA and beta crystalline (shown as CRY B in FIG. 8B) were found (FIG. 8A, 8B) and continuously expressed in the transparent cell structures (FIG. 8C, 8D). Interestingly, these transparent structures were not stained by DAPI (FIG. 8C-8F), indicating denucleation in these lens cells. In contrast, lentoid bodies generated in a previous study do not exhibit denucleation. When CONCEPT telencephalon-eye organoids were detached using Dispase at around day 28 and continuously grown as suspension cultures, crystal-like clusters with fused transparent spheres were found, and they continuously survived for months (FIG. 8I, 8J). In mice, terminally-differentiated lens fiber cells are free of organelles and featured by specialized interlocking cell membrane domains shown as ball-and-sockets and protrusions. Using transmission electron microscopy, we found that the crystal-like lens clusters were free of organelles and exhibited ball-and-socket structures (FIG. 8K, 8L). Taken together, our findings demonstrated that CONCEPT telencephalon-eye organoids contain lens cells that undergo terminal differentiation; FGFs in the organoids likely promote terminal lens differentiation as seen in other settings.

### Example 10: Single-Cell RNA Sequencing Analysis Identifies Telencephalic and Ocular Cell Populations in CONCEPT Telencephalon-Eye Organoids

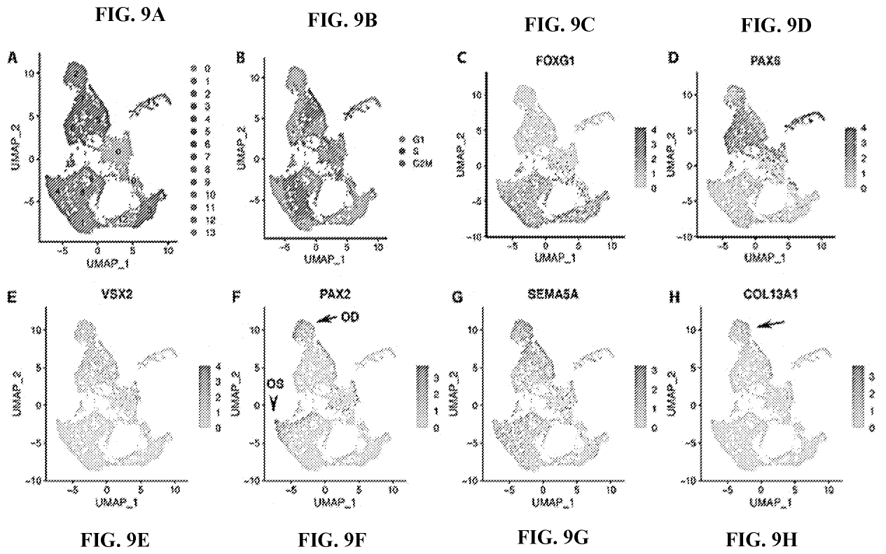

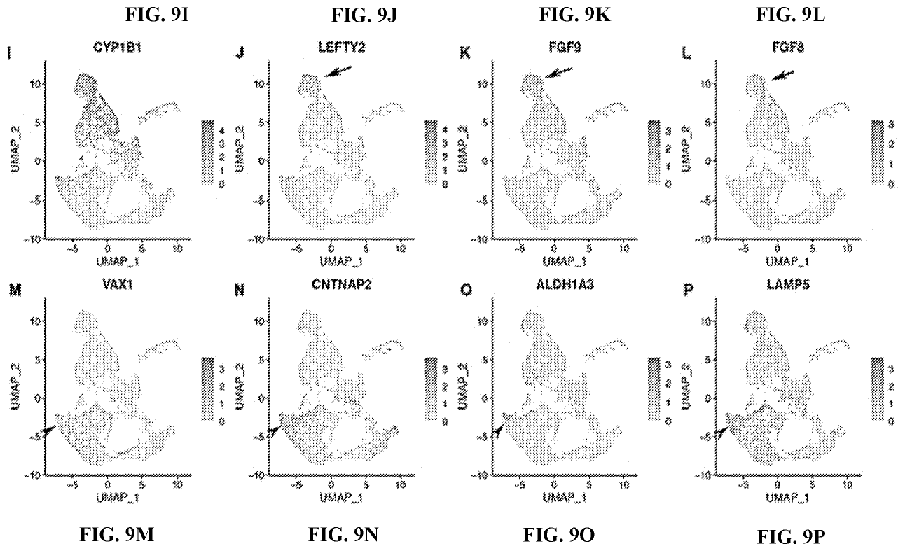

To fully characterize cell populations in CONCEPT organoids and the mechanisms underlying RGC axon pathfinding, we performed single-cell RNA sequencing of CONCEPT telencephalon-eye organoids at day 24, around the stage when RGCs grew long axons toward and along the PAX2+VSX2+ cell population. In the 10× single-cell RNA sequencing assay, 11158 single cells were captured and then sequenced at a depth of 27,842 reads and 2,967 genes per cell. After that, the dataset was analyzed using the Seurat (v3.2.0) package. Sequenced cells were filtered (nFeature_RNA >200 & nFeature_RNA <6000 & percent.mt<20), resulting in 10218 cells with high quality. Cell clustering grouped cells into 14 clusters (FIG. 9A). In addition, cell cycle scores were plotted using an established method (FIG. 9B). Several cell clusters, e.g., clusters 4, 8, 9, were separated along cell cycle scores (FIG. 9A, 9B), indicating the differences in cell cycle phases across these cell clusters.

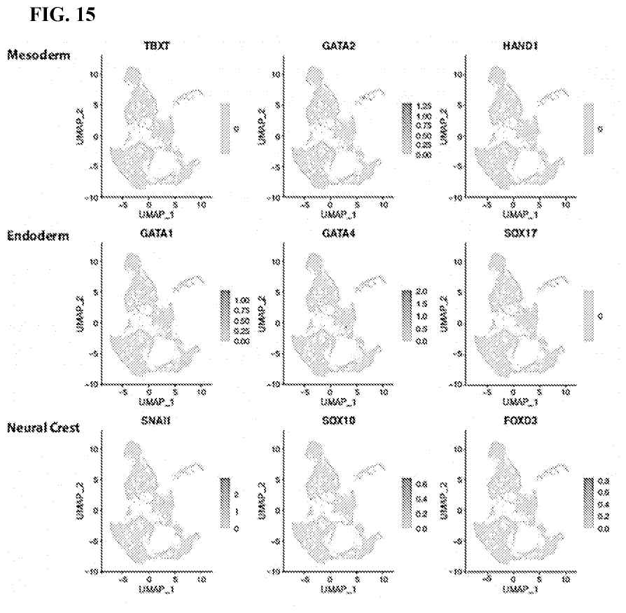

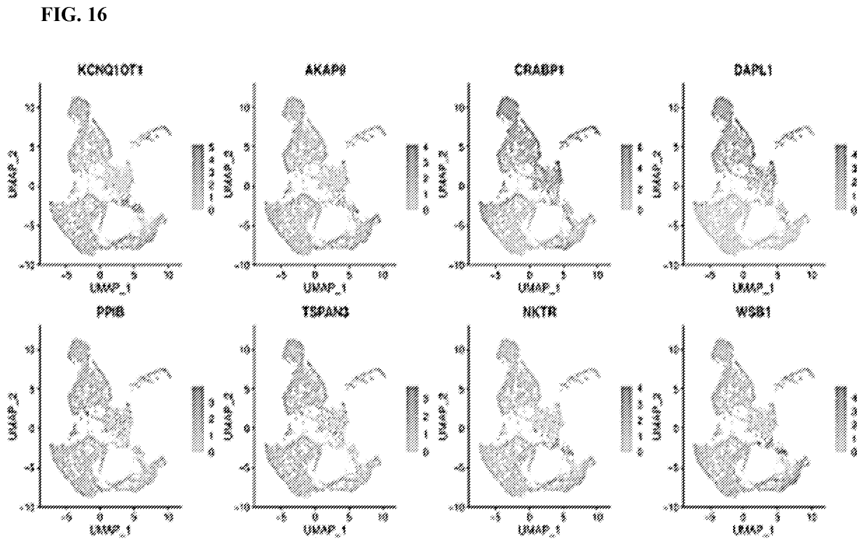

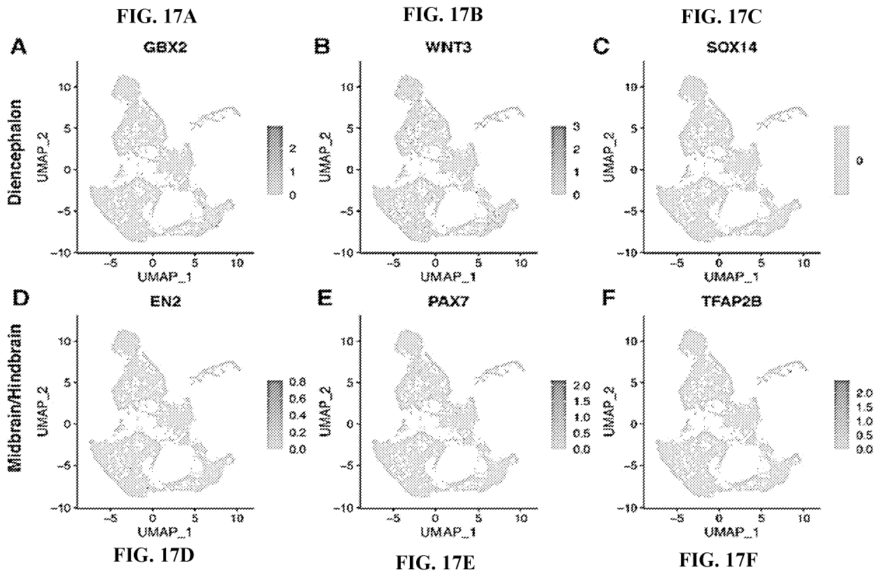

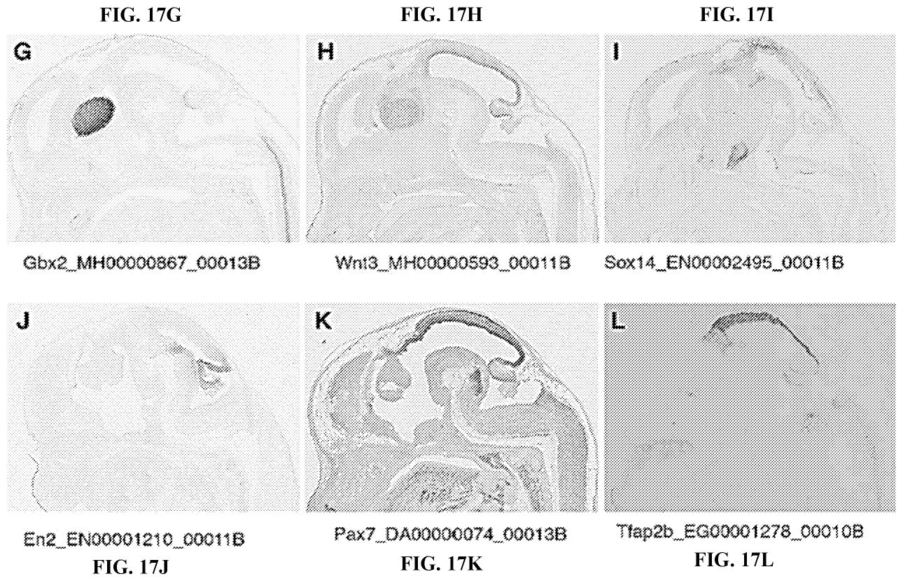

We next assessed the cell identities of CONCEPT telencephalon-eye organoids at day 24 using known markers. Mesoderm, endoderm, and neural crest are identified by a group of gene markers. In CONCEPT organoids, gene markers for mesoderm (TBXT, GATA2, HAND1), endoderm (GATA1, GATA4, SOX17), and neural crest (SNAI1, SOX10, FOXD3) were not expressed (FIG. 15). In contrast, gene markers for the anterior neuroectoderm were widely expressed. Telencephalic progenitor marker FOXG1 was expressed in clusters 10, 3, 12, 1, 4, 8, 9, 13; retinal progenitor markers PAX6 and VSX2 were expressed in clusters 2, 7, 5, 6, 11 (FIG. 6; FIG. 9C-9E). Cluster 0 was composed of both FOXG1+ telencephalic cells and PAX6+ and/or VSX2+ retinal cells; cluster 0 was defined by a group of negative markers (FIG. 16). In contrast to telencephalon and retinal markers, diencephalon markers (GBX2, WNT3, SOX14) and midbrain/hindbrain markers (EN2, PAX7, TFAP2B) were rarely expressed (FIG. 17). Lens markers CRYAA and FOXE3 were expressed in a very small cell population; lens cells did not form a separate cluster probably due to their low abundance. Taken together, these findings indicate that CONCEPT telencephalon-eye organoids at day 24 are mostly composed of FOXG1+ telencephalic cells and PAX6+ and/or VSX2+ retinal cells.

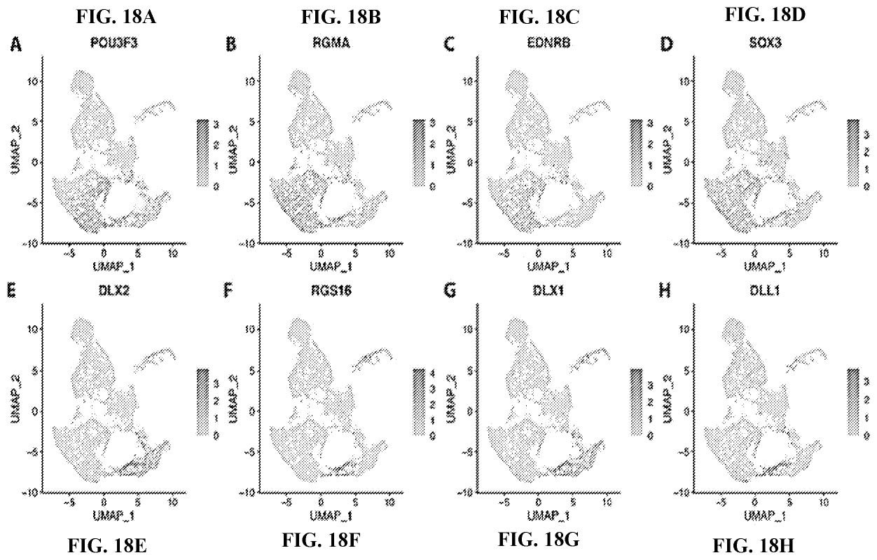

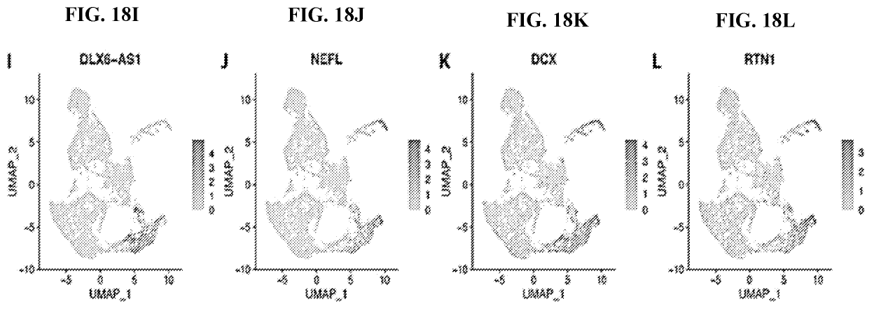

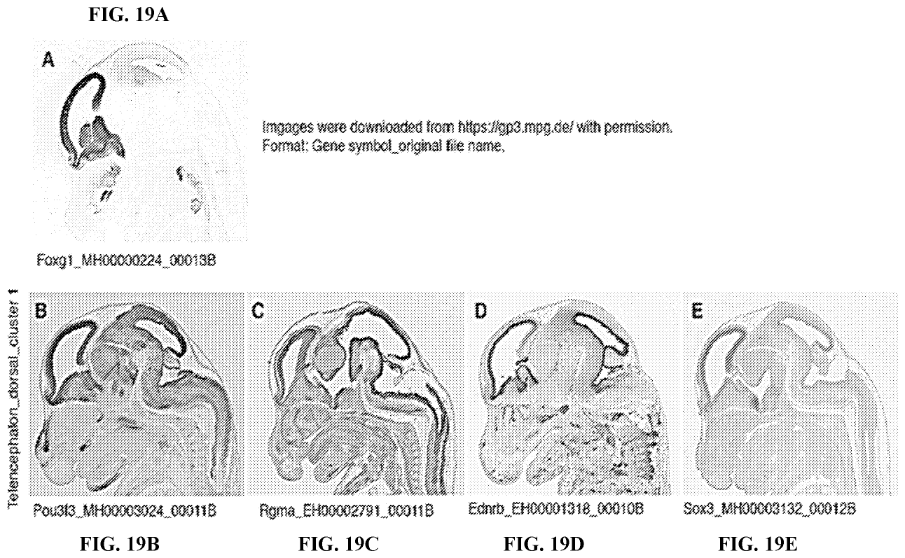

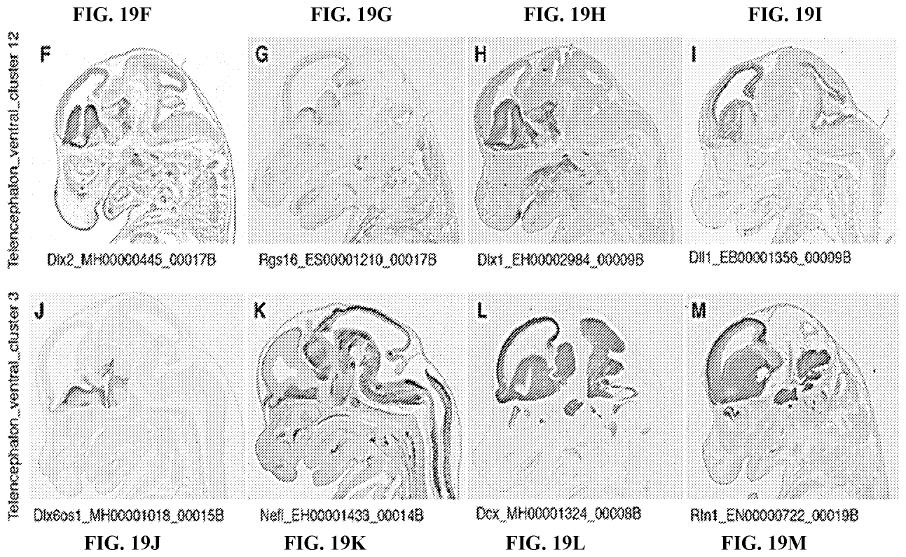

We next characterized the FOXG1+ telencephalic cell population via assessing the profiles of differentially expressed genes (DEGs). DEGs in clusters 4, 8, 9, 1 included POU3F3, RGMA, EDNRB, and SOX3, which orthologs in mice are expressed in the pallium. DEGs in clusters 12, 3, 10 included DLX2, RGS16, DLX1, DLL1, DLX6-AS1, NEFL, DCX, and RTN1, which orthologs in mice are expressed in the subpallium (FIG. 18-FIG. 19). Therefore, FOXG1+ telencephalic cell population in CONCEPT telencephalon-eye organoids at day 24 comprise multiple cell clusters in both the pallium and subpallium.

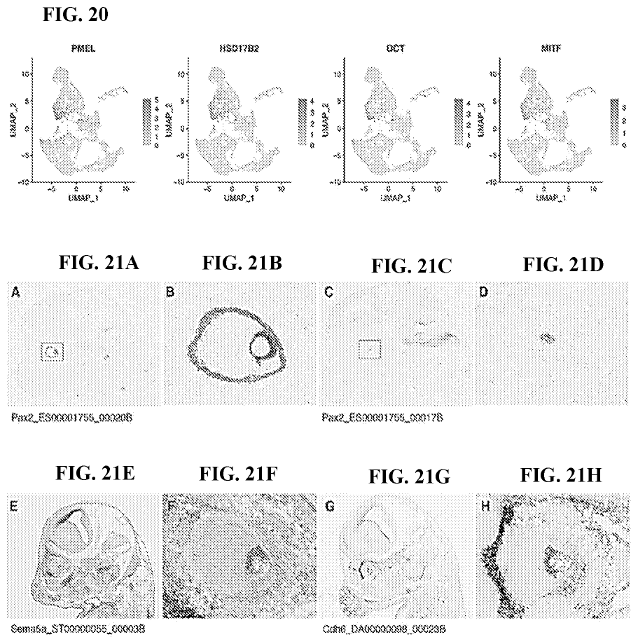

PAX6+ and/or VSX2+ retinal cell populations were also grouped into several clusters. VSX2 was expressed in clusters 2, 7, 5, and 0, with cluster 2 at G1 phase, cluster 7 in G1 and S phases, and cluster 5 in S and G2M phases (FIGS. 9A, 9B, 9E). Cluster 6 did not VSX2 but expressed PAX6 and was at the G1 phase, suggesting cells in cluster 6 exited the retinal progenitor cell state and underwent cell differentiation (FIGS. 9A, 9B, 9D, 9E). Consistently, cluster 6 differentially expressed RPE markers, e.g., PMEL, HSD17B2, DCT, and MITF (FIG. 20), indicating they were mostly differentiating RPE. Cluster 11 did not express VSX2; instead, it expressed PAX6 and was at the G1 phase (FIGS. 9A, 9B, 9D, 9E), suggesting that cells in cluster 11 exited the retinal progenitor cell state and underwent cell differentiation.

Taken together, our single-cell RNA sequencing analysis of CONCEPT telencephalon-eye organoids at day 24 confirmed their telencephalic and ocular identities, establishing a valuable transcriptomic dataset for mechanistic studies.

### Example 11: Identification of Two PAX2+ Cell Populations that Mimic the Optic Disc and Optic Stalk, Respectively, in the scRNA-Seq Dataset of CONCEPT Telencephalon-Eye Organoids

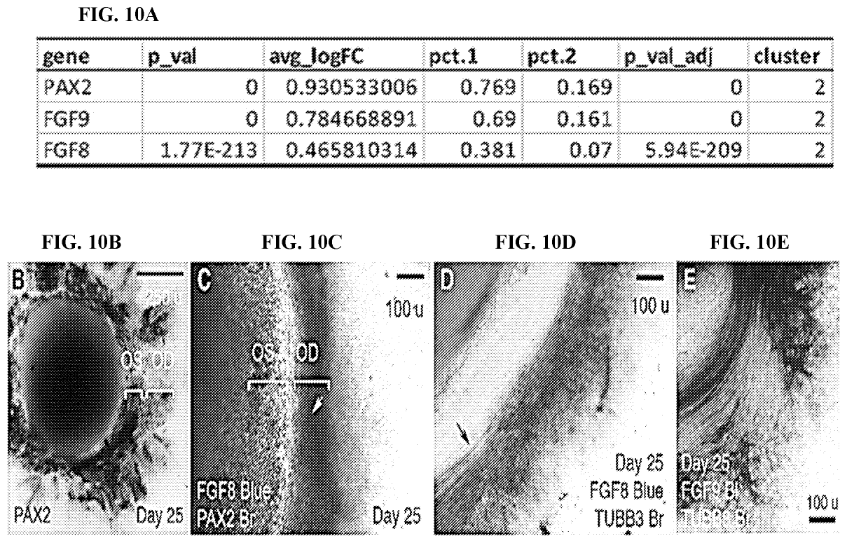

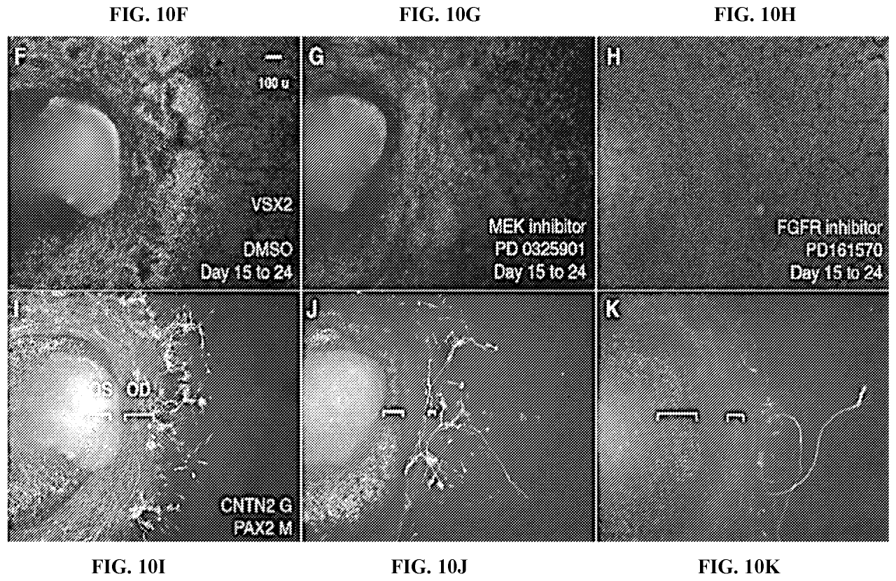

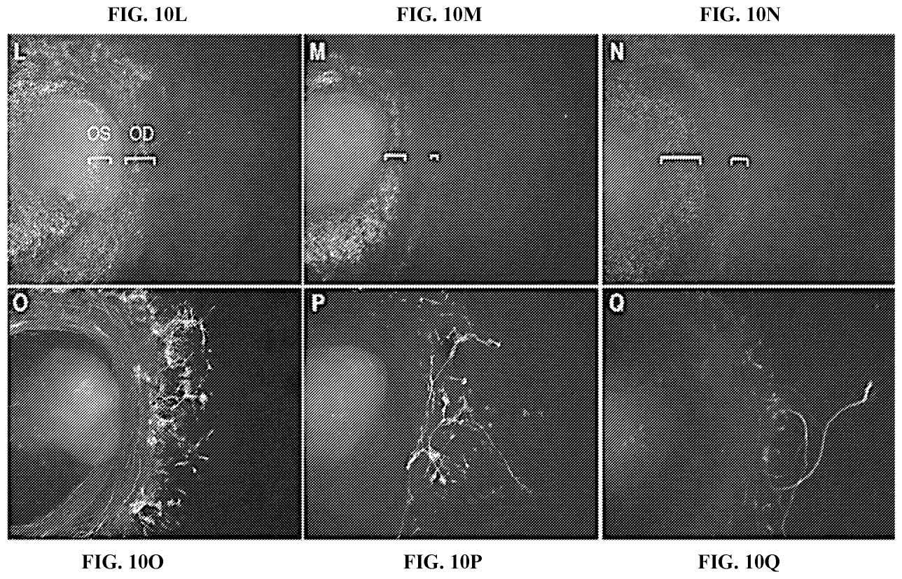

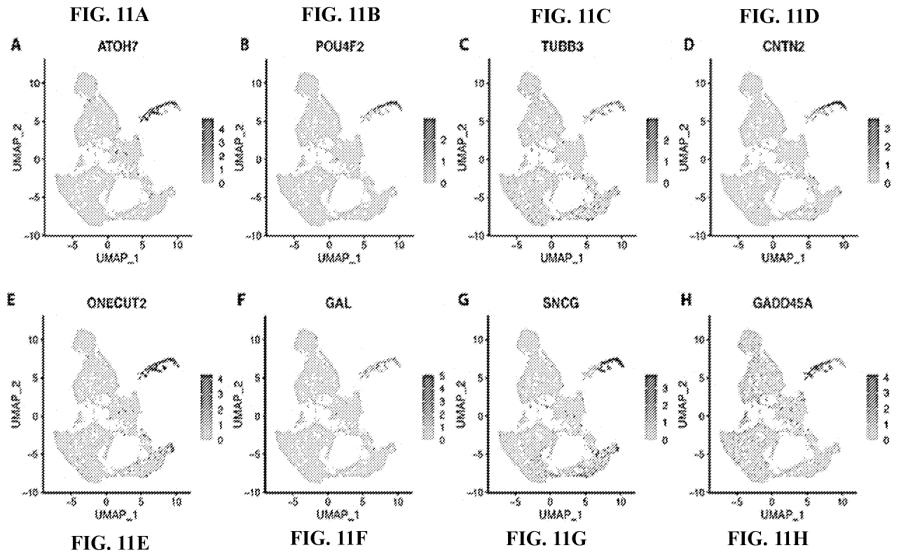

To identify PAX2+ cell populations that defined the path for RGC axon outgrowth, we examined PAX2 expression in the single-cell RNA sequencing dataset. PAX2 was mostly expressed in cluster 2 and subsets of clusters 7, 5, 0, 4, 8, and 9 (FIGS. 9A, 9F). Since clusters 2, 7, 5, and 0 expressed VSX2 (FIGS. 9E, 9F), we deduced that PAX2+VSX2+ cells in cluster 2 and subsets of clusters 7, 5, and 0 corresponded to those PAX2+VSX2+ cells that defined the path for RGC axon growth (FIG. 7H-7L). Therefore, these PAX2+VSX2+ cells in the dataset of single-cell RNA sequencing were assigned as the optic disc (OD) (FIG. 9F). PAX2+VSX2+ cells also expressed optic disc and optic stalk marker SEMA5A (FIGS. 9F, 9G). We particularly focused on cluster 2 since PAX2+VSX2+ cells were mostly found in this cluster. Interestingly, COL13A1 was differentially expressed in cluster 2 (FIG. 9H; FIG. 23); in situ hybridization indicates that COL13A1 is specifically expressed in the optic disc of human fetal retinas (Sandberg-Lall et al., 2000, Exp Eye Res 70, 401-410). These findings firmly demonstrate that PAX2+VSX2+ cells in CONCEPT telencephalon-eye organoids corresponded to optic disc cells in human fetal retinas. Cluster 2 also differentially expressed glaucoma gene CYP1B1, signaling molecules LEFTY2, FGF9, and FGF8 (FIG. 9I-9L; FIG. 23). The expression of PAX2, SEMA5A, CYP1B1, LEFTY2, FGF9, and FGF8 in CONCEPT organoids was validated using in situ hybridization (FIG. 6T-6V; FIG. 10B-10E; FIG. 23).

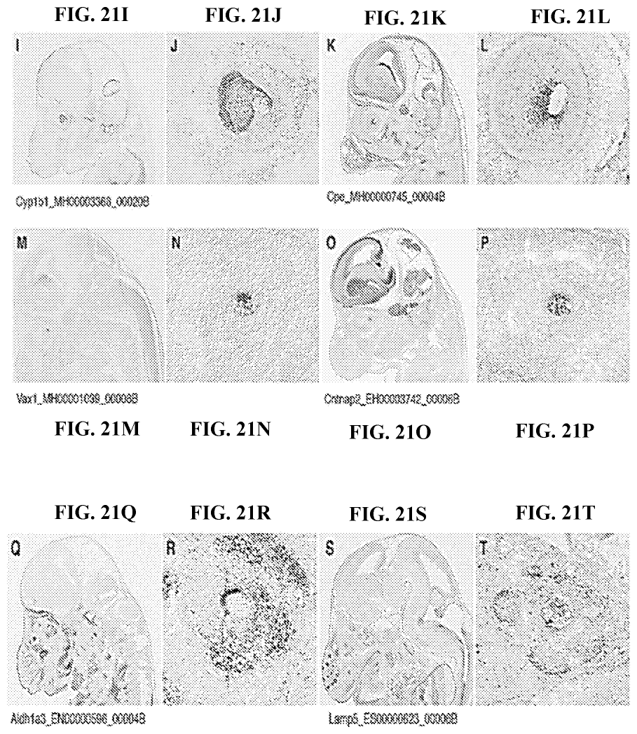

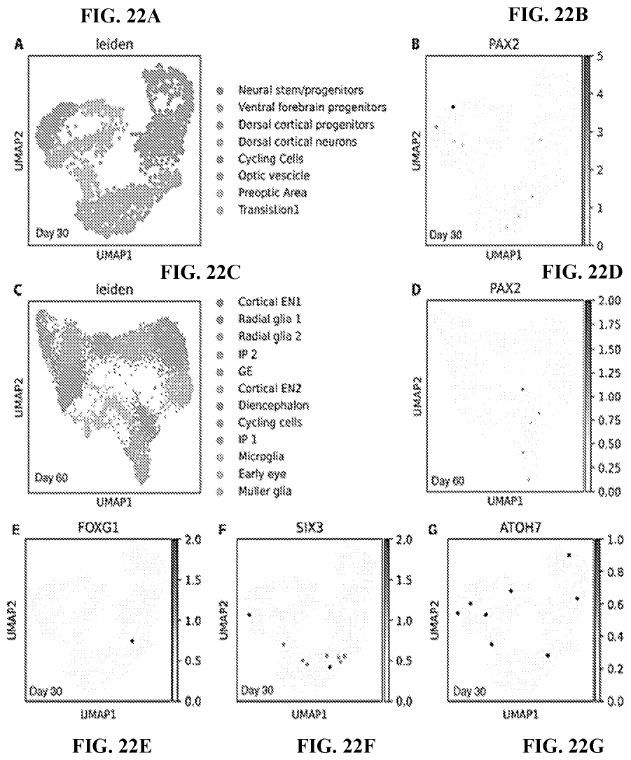

In contrast, PAX2+VSX2− cells found in subsets of clusters 4, 8, 9 (FIGS. 9A, 9E, 9F) expressed the optic stalk marker VAX1 (FIG. 9F, 9M); these cells corresponded to those PAX2+VSX2− cells that set up the inner boundary of the path for RGC axon growth in CONCEPT organoids (FIG. 7H-7L). Therefore, PAX2+VSX2− cells in subsets of clusters 4, 8, 9 were assigned as the optic stalk (OS) (FIG. 9F). PAX2+VSX2− optic stalk cells also differentially expressed CNTNAP2, ALDHA3, and LAMP5 (FIG. 9N-9P), which orthologs in mice are expressed in the optic stalk/nerve (FIG. 21). Compared to PAX2+VSX2+ optic disc cells, PAX2+VSX2− optic stalk cells were closer to telencephalic cells in both the UMAP plot (FIG. 9M-9P; FIG. 18A-18D) and their positioning in CONCEPT organoids (FIG. 7H-7L), mimicking relative positions of telencephalon, optic stalk, and optic disc along the midline-periphery axis in E12.5-E13.5 mouse embryos. Consistently, several markers for PAX2+VSX2− optic stalk cells were also expressed in the telencephalon (FIG. 21). Notably, our CONCEPT telencephalon-eye organoids were substantially different from Gabriel et al.'s brain organoids (Gabriel et al. (2021) Cell Stem Cell, 28(10):1740-1757) in which PAX2+ cells, FOXG1+ cells, SIX3+ cells, and VSX2+ cells are extremely rare (FIG. 22). Collectively, we identified two PAX2+ cell populations that mimic the optic disc and optic stalk, respectively, in the scRNA-seq dataset of CONCEPT telencephalon-eye organoids.

### Example 12: Inhibition of FGF Signaling Prior to RGC Differentiation with FGFR and MEK Inhibitors Drastically Decreases PAX2+ Optic Disc Cells, RGCs, and Directional RGC Axon Growth

Directional RGC axon growth toward and then along PAX2+ optic disc cells in CONCEPT organoids could be mediated by signaling molecules secreted from PAX2+ optic disc cells. scRNA-seq analysis identified expression signatures of cluster 2, the major component of PAX2+ optic disc cells (FIG. 23). Interestingly, FGF8 and FGF9 were differentially expressed in cluster 2 (FIG. 9K, FIG. 9L, FIG. 10A). To assess the roles of FGF8 and FGF9 in CONCEPT organoids, we validated that FGF8 and FGF9 were expressed in PAX2+ optic disc cells of CONCEPT organoids (FIG. 10B-10E). TUBB3+ RGC axons grew towards and then along the regions with high-level FGF8 and FGF9 (FIG. 10D, 10E), suggesting that FGF8 and FGF9 may attract RGC axon growth. FGFR1, FGFR2, FGFR3, MAP2K1, and MAP2K2 were expressed in multiple types of cells in CONCEPT organoids. In RGCs, FGFR1 and MAP2K2 were clearly expressed (FIG. 24), indicating that RGCs expressed the components that transduce FGF signaling. Since FGF8 and FGF9 are probably redundant and it is challenging to inactivate both FGF8 and FGF9 in CONCEPT organoids, we decided to inactivate FGF signaling with FGFR1/2/3 inhibitor PD 161570 and MEK1/2 inhibitor PD 0325901 (MEK1 and MEK2 are also known as MAP2K1 and MAP2K2, respectively). When MEK was inhibited from day 15, a stage shortly before RGC differentiation, neuroretinal progenitors marked by VSX2 expression regions largely remained (FIG. 10G), so did optic stalk cells that expressed high-level PAX2 at the inner zone (FIGS. 10J, 10M). In contrast, optic disc cells marked by moderate-level PAX2 expression at the outer regions were nearly absent (FIGS. 10J, 10M), and RGC somas and directional axon growth marked by CNTN2 expression were drastically reduced (FIGS. 10J, 10P). When FGFR was inhibited, VSX2 in retinal progenitors (FIG. 10H) and PAX2 in optic stalk cells (FIG. 10K, 10N) were reduced, PAX2 in optic disc cells was severely reduced (FIGS. 10K, 10N), RGC somas and directional axon growth marked by CNTN2 expression were drastically reduced or nearly absent (FIG. 10K, 10Q). These findings indicated that a) PAX2+ optic disc cells differentially expressed FGF8 and FGF9; b) inhibition of FGF signaling prior to RGC differentiation with FGFR and MEK inhibitors drastically decreases PAX2+ optic disc cells, RGCs, and directional RGC axon growth.

### Example 13: Identification of Glycosylphosphatidylinositol (GPI)-Anchored Cell Membrane Protein CNTN2 as a Specific Marker for Developing Human RGCs

To identify RGCs in the dataset of single-cell RNA sequencing, we checked the expression of RGC markers. We found that RGC markers ATOH7, POU4F2, ONECUT2, GAL, SNCG, GADD45A, TUBB3, and CNTN2 were specifically expressed in cluster 11, indicating its RGC identity (FIG. 9A, FIG. 11). CNTN2 caught our attention because it is a GPI-anchored cell membrane protein, which may be used as a native marker for RGC isolation. Cntn2 is specifically expressed in developing RGCs in mice and chicks.

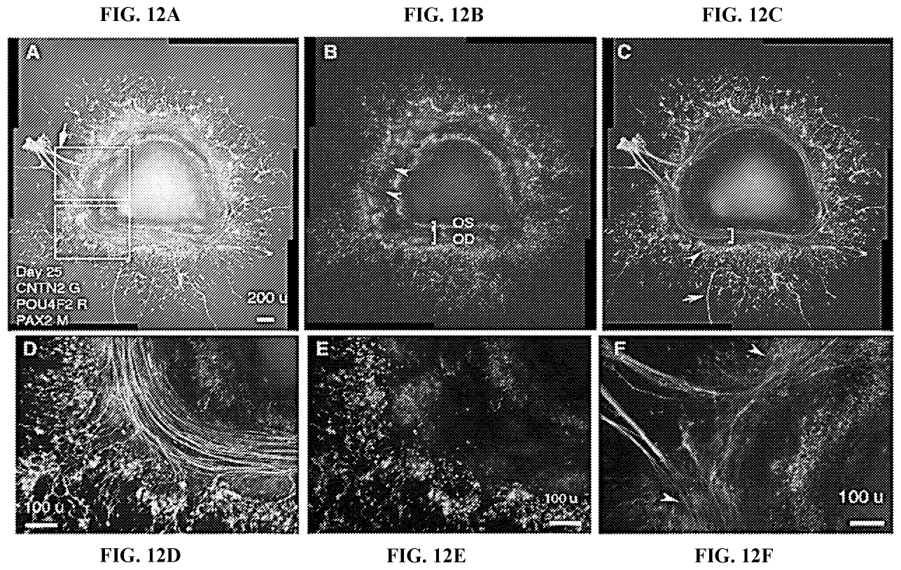

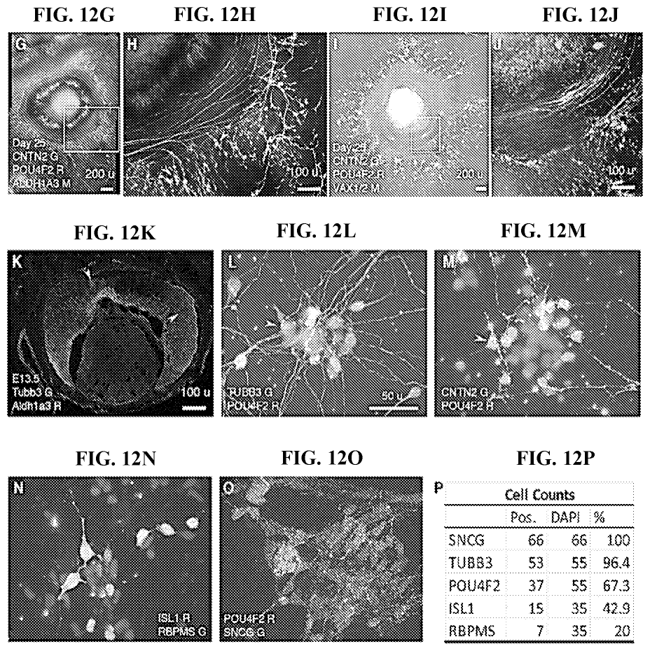

In CONCEPT telencephalon-eye organoids at day 25, CNTN2 exhibited an expression pattern very similar to that of TUBB3 (FIG. 7, FIG. 12). CNTN2 was found in the cell membrane of RGCs that expressed POU4F2 in the cell nucleus (FIG. 12A, 12D). PAX2+ cells formed inner and outer concentric zones, mimicking the optic stalk and optic disc, respectively. POU4F2+ RGCs formed a dense circular zone adjacent to PAX2+ optic disc cells; POU4F2+ RGCs were sparse in more peripheral areas (FIG. 12B). These findings indicate that early differentiated RGCs were adjacent to PAX2+ optic disc cells. Interestingly, RGCs in the dense POU4F2+ zone grew CNTN2+ axons toward and along the path that was defined by adjacent PAX2+ optic disc cells (FIG. 12A-12E). In areas where there were a gap in PAX2+ optic disc cells, CNTN2+ axons exited the circular path (diamond arrow in FIG. 12A; a gap between two arrowheads in FIG. 12B, 12F). In regions where POU4F2+ RGCs were a few hundreds of micrometers away from PAX2+ optic disc cells, RGCs grew axons in centrifugal directions (arrow in FIG. 12C). Very similar findings were found using hiPSCs (FIG. 25), indicating the reproducibility of CONCEPT organoids in multiple cell lines. PAX2+ optic disc cells did not express ALDH1A3; instead, the path for RGC axon growth was bordered by two cell populations that highly expressed ALDH1A3 (FIG. 12G-12H). In mice, Aldh1a3 expression was low in differentiating RGCs in the central retina but high in peripheral retinal progenitors and optic stalk (FIG. 12K), consistent with ALDH1A3 expression in CONCEPT organoids (FIG. 12G, 12H). Cells that set up the inner boundary for RGC axon growth also expressed the optic stalk marker VAX1/2 (FIGS. 12I, 12J; the antibody recognizes both VAX1 and VAX2). Taken together, our data demonstrated that CNTN2 is a specific cell surface marker for differentiating human RGCs; RGCs grow axons toward and along a path that is defined by adjacent PAX2+VSX2+ ALDH1A3− optic disc cells.

### Example 14: One-Step Isolation of Developing Human RGCs Via Native Marker CNTN2

Since cell surface protein CNTN2 was specifically expressed in developing human RGCs, we sought to test whether CNTN2 can be used as a biomarker for isolating human RGCs under a native condition. To that goal, retinal organoids in suspension culture on days 41-70 were dissociated using Accutase to generate a single cell suspension, which was then subject to magnetic-activated cell sorting (MACS) with an antibody against CNTN2. From 100 retinal organoids on day 41 to 48, around 385,000 RGCs were isolated. Isolated RGCs were plated onto Matrigel-coated chamber slides for 10-day adherent culture. These cells exhibited neuronal morphology and widely expressed RGC markers TUBB3 and POU4F2 (FIG. 12L). Isolated RGCs had a tendency to form clusters in culture. In contrast to directional axon growth found in CONCEPT telencephalon-eye organoids, isolated RGCs grew neurites in random directions (FIG. 12L), indicating the differences in axon pathfinding cues between CONCEPT telencephalon-eye organoids and isolated RGC cultures. RGC neurites were also marked by CNTN2 expression (FIG. 12M). A portion of isolated RGCs expressed POU4F2 but not CNTN2 (FIG. 12M), indicating that CNTN2 was downregulated at some RGCs. Isolated RGCs also expressed RGC markers ISL1, RBPMS, and SNCG (FIG. 12N-12P). Collectively, we have developed a one-step method for the isolation of differentiating human RGCs in a native condition.

### Example 15: Isolated RGCs Exhibit Electrophysiological Signature of Excitable Cells

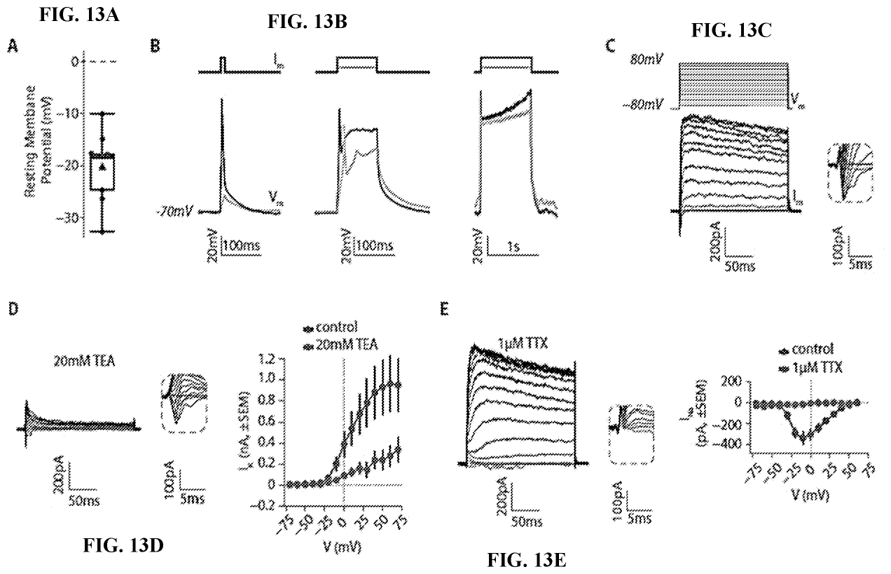

In order to determine the functional properties of isolated RGCs in culture, we examined their electrophysiological properties using whole-cell patch clamp recordings (FIG. 13, see Methods in Example 6). Using current-clamp configuration, we found that RGCs displayed had a hyperpolarized resting membrane potential (mean±SD: −20.1±6.4 mV, FIG. 13A). When cells were held at −70 mV by current injection, in most cases (6/9), depolarizing currents steps could trigger an action potential, which were often followed by a depolarization plateau if the current was injected for more than 10 ms (FIG. 13B). In voltage clamp recordings we examined the nature of the voltage-gated conductances (see Methods in Example 6). From a holding membrane potential of −80 mV, both inward and outward currents (Im) were observed in response to depolarizing voltage steps (Vm, FIG. 13C). Outward currents were primarily mediated by voltage-gated potassium channels, as application of 20 mM TEA significantly reduced their amplitude (mean IK±SD at 60 mV; 964±537 pA in control; 279±201 pA in TEA, U=24.0; p=0.019, two-sided MannWhitneyU test, FIG. 13D). Conversely, 1 μM TTX abolished all inward currents, demonstrating that they were mediated by activation of voltage-gated sodium channels (mean INa+SD at −10 mV; −338.2±121 in control; −18.3±13 pA in TTX; U=0.0; p=0.035, two-sided MannWhitneyU test, FIG. 13E). Therefore, RGCs in our preparation showed comparable electrophysiological features to M1 melanopsin-expressing RGCs recorded from mouse retinal explants. Taken together, these results indicate that isolated RGCs exhibit functional features traditionally found in excitable cells, such as in neurons.

In this study, we report the self-formation of concentric zones of telencephalic and ocular tissues in CONCEPT telencephalon-eye organoids from human pluripotent stem cells, establishing a model for studying the early development of telencephalic and ocular tissues in humans. RGCs grew axons toward and along a path defined by PAX2+ cell populations, setting up a model for studying RGC axon pathfinding. We identified expression signatures of cell clusters in CONCEPT organoids using single-cell RNA sequencing. Lastly, we established a one-step method for the isolation of human RGCs via CNTN2 under a native condition. Our studies not only provide deeper insight into coordinated specification of telencephalic and ocular tissues for RGC axon growth in humans, but also generate useful models and tools for therapeutic studies of RGC-related diseases such as glaucoma.

### Cysts is a Radially-Symmetric Epithelium Mimicking the Anterior Ectoderm

Cysts are hollow spheres composed of homogeneous columnar epithelial cells. They are induced from pluripotent stem cells via embedding small sheets of hESCs into Matrigel and subsequent growth either as a solid thin film on the cell culture surface or as a suspension culture. Cyst growth as a suspension culture is cost-effective and scalable. When small cell sheets of hESCs were directly embedded into Matrigel, the apical surface of cells managed to avoid its contact with the basal cues in Matrigel, leading to its internalization at the lumen of cysts. The formation of cysts induced by Matrigel mimics the epithelization of the epiblast by the extracellular matrix (ECM) in the blastocyst.

Our studies indicate that using the methods of the present disclosure, individual cysts efficiently generate telencephalic and ocular tissues in the absence of any extrinsic factors, indicating their default cell fates of the anterior ectoderm. Our single-cell RNA sequencing indicates that the tissues spontaneously differentiated from individual cysts were mostly the anterior neuroectodermal tissues and a small amount of the anterior surface ectodermal tissues such as lens. Anterior neural tissues are generated from re-aggregated single pluripotent stem cells through dual inhibition of Smad signaling or inhibition of Wnt/ß-catenin signaling. In our methods, we did not supplement any extrinsic factors; surpringly and unexpectedly, we generated tissues of the anterior ectoderm efficiently. The initial epithelial structure of cysts and subsequent adherent growth of individual cysts at a low density made a difference in neural induction and differentiation since pluripotent stem cells that are continuously kept as adherent culture in the same differentiation medium would have much less efficiency in differentiation toward the anterior ectoderm. Collectively, the cyst mimics the anterior ectoderm in the aspects of the epithelial structure and cell fates.

### Pattern Formation and Coordinated Cell Differentiation in CONCEPT Telencephalon-Eye Organoids

Tissue patterning is fundamental for the formation of a body plan. The overall body plan is defined by the anteroposterior, dorsoventral, and left-right axes. The telencephalic field and eye field are the most anterior structures; the telencephalon and retina are outpouchings of the secondary prosencephalon. After the outpouching, the telencephalon, optic stalk, and optic vesicle are arranged in a midline-periphery axis; the optic disc subsequently forms between the optic stalk and neuroretina. In CONCEPT telencephalon-eye organoids on days 22-26, FOXG1+ telencephalon, PAX2+ optic stalk, PAX2+ optic disc, and VSX2+ neuroretinas are positioned along the center-periphery axis, mimicking the relative positions of those tissues in E12.5-13.5 mouse embryos. We estimate these mouse embryonic stages based on the timing of early RGC differentiation in both systems. Notably, our CONCEPT telencephalon-eye organoids are substantially different from Gabriel et al.'s organoids: PAX2+, FOXG1+, and VSX2+ cell populations are extremely rare in Gabriel et al.'s organoids (FIG. 21). Collectively, our CONCEPT telencephalon-eye organoids represent the early formation of telencephalic and ocular tissues in humans.

Tissue patterning in CONCEPT telencephalon-eye organoids is originated from the attachment of a radially-symmetric epithelium to the culture surface followed by growth as an anchored colony. When individual cysts were kept for suspension culture, no apparent tissue patterning was observed. The attachment of individual cysts to the culture surface followed by growth as colonies caused the initial homogeneous cell population in cysts to form multiple cell populations along the center-peripheral axis. When a floating cyst initially contacted the culture surface, the contact resulted in ECM-cell adhesions, which later became the central region of a CONCEPT telencephalon-eye organoid. The initial ECM-cell adhesions caused additional ECM-cell contacts in neighboring regions, resulting in the flattening and spreading of a cyst onto the culture surface. Since the cyst is a radially symmetric sphere with an epithelial structure, ECM-cell adhesions between the cyst and the culture surface formed sequentially along the central-peripheral axis in a concentric manner. Remodeling of cell-cell and ECM-cell adhesions and self-organization underlay the tissue patterning. Molecular events in a concentric pattern during the attachment of a cyst onto the culture surface were eventually translated to concentric gradients of morphogens that specify cell fates. The radially symmetric-epithelial structure of cysts is important for the formation of a concentric pattern since such pattern would not be generated when an amorphous embryoid body is attached to the culture surface and grow as a colony. Timed BMP4 treatment is shown to promote neuroretinal differentiation from pluripotent stem cells, and FGF8 promotes telencephalic and eye development. In our system, BMP4 and FGF8, along with BMP7 and other FGFs, were highly expressed starting at early stages and gradually formed concentric morphogen gradients, which would dictate tissue patterning, resulting in coordinated specification of telencephalic, optic stalk, optic disc, and neuroretinal tissues along the center-periphery axis in CONCEPT telencephalon-eye organoids.

Concentric patterns of stem cell-derived cultures are reported in a few other experiments. When dissociated single pluripotent stem cells are grown in micropatterned culture surface at certain cell densities in a differentiation medium supplemented with BMP4, concentric zones of progenitors expressing markers for trophectoderm, endoderm, mesoderm, and ectoderm are found. Cell density and colony geometry dictate cell fate specification from pluripotent stem cells. A concentric gradient of BMP4 activity regulates the patterning. When dissociated single pluripotent stem cells are grown in pre-patterned geometrically confined culture surface in a differentiation medium supplemented with dual inhibitors for TGF-β and BMP4, concentric zones of progenitors expressing the markers for neural plate and neural plate border are observed. Morphogenetic cues-cell shape and cytoskeletal contractile force-dictate the patterning of the neural plate and neural plate border via BMP-SMAD signaling. When dissociated single pluripotent stem cells are grown as individual colonies in the StemFit medium and later in a differentiating medium supplemented by KnockOut® serum replacement, multiple zones of ectodermal cells autonomously form. In all three experiments, dissociated single cells were used to generate cell colonies through either cell re-aggregation or proliferation.

In our differentiation system, adherent culture of a radially-symmetric epithelium—cyst—was used to generate CONCEPT telencephalon-eye organoids. Therefore, the starting cells in the aforementioned experiments and our experiments differ in developmental potentials.

### RGC Axon Pathfinding Cues in CONCEPT Telencephalon-Eye Organoids

In the mouse retina, multiple RGC axon guidance cues are concentrically organized around the optic disc, regulating RGC axon growth and exit from the eye through the optic stalk. Early differentiated RGCs are in a short distance from the nascent optic disc, and axons of later differentiated RGCs in more peripheral retinal regions follow the path of the initial axons. It is accepted that the optic disc regions provide growth-promoting guidance cues whereas peripheral retinal regions provide inhibitory guidance cues. Pax2 is specifically expressed in the ventral optic stalk, optic vesicles, central neuroretina, optic disc, and optic stalk; Pax2 is essential for optic stalk and nerve development in mice.

In CONCEPT telencephalon-eye organoids, coordinated specification of telencephalic and ocular tissues leads to the generation of PAX2+ cell populations that define the path for RGC axon growth. To this date, directional RGC axon growth guided by intrinsic cues within organoids has not been reported. In our organoids, there were two PAX2+ cell populations that formed two adjacent concentric zones, mimicking those cells in the optic disc and optic stalk, respectively. The two PAX2+ cell populations differed in VSX2 expression and in their roles in defining the path for RGC axon growth. In CONCEPT organoids, initially differentiated RGCs grew their axons toward the adjacent PAX2+VSX2+ cell population and navigated along them, mimicking axon growth from the initial RGCs toward the optic disc in vivo. The PAX2+VSX2− cell population at the inner region set up an inner boundary of RGC axon growth, mimicking the optic stalk cells that spatially confine RGC axon growth in vivo. Additionally, the path for RGC axon growth was also marked by the absence of ALDH1A3, and the inner boundary was also delineated by VAX1/2 in CONCEPT organoids. Collectively, restricted expression of PAX2, VAX1/2, and ALDH1A3 in CONCEPT telencephalon-eye organoids define the path for RGC axon growth.

Single-cell RNA sequencing of CONCEPT telencephalon-eye organoids identified the expression signatures of PAX2+ cell populations that mimic the optic disc and optic stalk, respectively. Interestingly, FGF8 and FGF9 were differentially expressed in PAX2+ optic disc cells; inhibition of FGF signaling prior to RGC differentiation with FGFR and MEK inhibitors drastically decreases PAX2+ optic disc cells, RGCs, and directional RGC axon growth. These findings indicate that both the maintenance of PAX2+ optic cells and initial RGC differentiation require FGFR and MEK signaling. In addition, FGF8 and FGF9 secreted from PAX2+ optic disc cells may attract RGC axon growth since remaining RGCs did not grew directional axons after FGFR and MEK inhibition. Alternatively, the loss of directional RGC axon growth caused by FGFR and MEK inhibitors may be due to the severe reduction of PAX2+ optic disc cells. Besides FGF8 and FGF9, single-cell RNA sequencing of CONCEPT organoids identified additional expression signatures of PAX2+ optic disc cells, establishing candidate molecules for functional studies on the mechanisms of RGC axon growth and pathfinding in humans.

### One-Step Isolation of Developing Human RGCs Under a Native Condition

RGCs are degenerated in glaucoma, a major cause of vision impairment in developed countries. Disease modeling and drug discovery to suppress RGC death will have a huge impact on saving vision. The use of human RGC models is critical for therapeutic studies since humans and rodents differ significantly in RGCs. Additionally, cell replacement therapies for glaucoma are extensively evaluated. Therefore, efficient isolation of human RGCs in a native condition will have a substantial impact on therapeutic studies of RGC-related diseases such as glaucoma. In literature, cell surface marker Thy1 is used for the isolation of adult mouse RGCs, but it is unsuccessful in the isolation of stem cell-derived human RGCs. Tagging human RGCs with an engineered marker Thy1.2 leads to efficient isolation of human RGCs. However, tagged human RGCs are unsuitable for clinical uses. Using single-cell RNA sequencing, we identified cell surface protein CNTN2 as a specific marker for developing human RGCs. We isolated human RGCs using MACS with an antibody against CNTN2. Therefore, we establish a one-step method for isolating human RGCs under a native condition, facilitating therapeutic treatment for RGC-related retinal diseases such as glaucoma.

### REFERENCES

1. T. Inoue, S. Nakamura, N. Osumi, Fate mapping of the mouse prosencephalic neural plate. Dev Biol 219, 373-383 (2000).
2. S. F. Oster, M. Deiner, E. Birgbauer, D. W. Sretavan, Ganglion cell axon pathfinding in the retina and optic nerve. Semin Cell Dev Biol 15, 125-136 (2004).
3. L. Erskine, E. Herrera, The retinal ganglion cell axon's journey: insights into molecular mechanisms of axon guidance. Dev Biol 308, 1-14 (2007).
4. L. Erskine, E. Herrera, Connecting the retina to the brain. ASN Neuro 6 (2014).
5. S. W. Wilson, J. L. Rubenstein, Induction and dorsoventral patterning of the telencephalon. Neuron 28, 641-651 (2000).
6. S. M. Danesh, A. Villasenor, D. Chong, C. Soukup, O. Cleaver, BMP and BMP receptor expression during murine organogenesis. Gene Expr Patterns 9, 255-265 (2009).
7. Y. Furuta, B. L. Hogan, BMP4 is essential for lens induction in the mouse embryo. Genes Dev 12, 3764-3775 (1998).
8. A. T. Dudley, E. J. Robertson, Overlapping expression domains of bone morphogenetic protein family members potentially account for limited tissue defects in BMP7 deficient embryos. Dev Dyn 208, 349-362 (1997).
9. M. J. Solloway et al., Mice lacking Bmp6 function. Dev Genet 22, 321-339 (1998).
10. J. Morcillo et al., Proper patterning of the optic fissure requires the sequential activity of BMP7 and SHH. Development 133, 3179-3190 (2006).
11. K. Shimamura, J. L. Rubenstein, Inductive interactions direct early regionalization of the mouse forebrain. Development 124, 2709-2718 (1997).
12. E. E. Storm et al., Dose-dependent functions of Fgf8 in regulating telencephalic patterning centers. Development 133, 1831-1844 (2006).
13. J. R. Martinez-Morales et al., Differentiation of the vertebrate retina is coordinated by an FGF signaling center. Dev Cell 8, 565-574 (2005).
14. A. Vogel-Hopker et al., Multiple functions of fibroblast growth factor-8 (FGF-8) in chick eye development. Mech Dev 94, 25-36 (2000).
15. C. Soukkarieh, E. Agius, C. Soula, P. Cochard, Pax2 regulates neuronal-glial cell fate choice in the embryonic optic nerve. Dev Biol 303, 800-813 (2007).
16. S. Xuan et al., Winged helix transcription factor BF-1 is essential for the development of the cerebral hemispheres. Neuron 14, 1141-1152 (1995).
17. M. E. Zuber, G. Gestri, A. S. Viczian, G. Barsacchi, W. A. Harris, Specification of the vertebrate eye by a network of eye field transcription factors. Development 130, 5155-5167 (2003).
18. T. Marquardt et al., Pax6 is required for the multipotent state of retinal progenitor cells. Cell 105, 43-55 (2001).
19. R. Ashery-Padan, T. Marquardt, X. Zhou, P. Gruss, Pax6 activity in the lens primordium is required for lens formation and for correct placement of a single retina in the eye. Genes Dev 14, 2701-2711 (2000).
20. N. Baumer et al., Retinal pigmented epithelium determination requires the redundant activities of Pax2 and Pax6. Development 130, 2903-2915 (2003).
21. M. Torres, E. Gomez-Pardo, P. Gruss, Pax2 contributes to inner ear patterning and optic nerve trajectory. Development 122, 3381-3391 (1996).
22. R. Macdonald et al., The Pax protein Noi is required for commissural axon pathway formation in the rostral forebrain. Development 124, 2397-2408 (1997).
23. J. R. Martinez-Morales, M. Signore, D. Acampora, A. Simeone, P. Bovolenta, Otx genes are required for tissue specification in the developing eye. Development 128, 2019-2030 (2001).
24. M. Schwarz et al., Spatial specification of mammalian eye territories by reciprocal transcriptional repression of Pax2 and Pax6. Development 127, 4325-4334 (2000).
25. S. H. Mui, J. W. Kim, G. Lemke, S. Bertuzzi, Vax genes ventralize the embryonic eye. Genes Dev 19, 1249-1259 (2005).
26. M. Take-uchi, J. D. Clarke, S. W. Wilson, Hedgehog signalling maintains the optic stalk-retinal interface through the regulation of Vax gene activity. Development 130, 955-968 (2003).
27. D. J. Horsford et al., Chx10 repression of Mitf is required for the maintenance of mammalian neuroretinal identity. Development 132, 177-187 (2005).
28. S. Rowan, C. M. Chen, T. L. Young, D. E. Fisher, C. L. Cepko, Transdifferentiation of the retina into pigmented cells in ocular retardation mice defines a new function of the homeodomain gene Chx10. Development 131, 5139-5152 (2004).
29. W. Liu, O. Lagutin, E. Swindell, M. Jamrich, G. Oliver, Neuroretina specification in mouse embryos requires Six3-mediated suppression of Wnt8b in the anterior neural plate. J Clin Invest 120, 3568-3577 (2010).
30. K. Bharti et al., A regulatory loop involving PAX6, MITF, and WNT signaling controls retinal pigment epithelium development. PLoS Genet 8, e1002757 (2012).
31. H. Li et al., A retinoic acid synthesizing enzyme in ventral retina and telencephalon of the embryonic mouse. Mech Dev 95, 283-289 (2000).
32. M. Yahyavi et al., ALDH1A3 loss of function causes bilateral anophthalmia/microphthalmia and hypoplasia of the optic nerve and optic chiasm. Hum Mol Genet 22, 3250-3258 (2013).
33. M. A. Lancaster, J. A. Knoblich, Organogenesis in a dish: modeling development and disease using organoid technologies. Science 345, 1247125 (2014).
34. M. Eiraku et al., Self-organizing optic-cup morphogenesis in three-dimensional culture. Nature 472, 51-56 (2011).
35. J. S. Meyer et al., Optic vesicle-like structures derived from human pluripotent stem cells facilitate a customized approach to retinal disease treatment. Stem Cells 29, 1206-1218 (2011).
36. T. Nakano et al., Self-formation of optic cups and storable stratified neural retina from human ESCs. Cell Stem Cell 10, 771-785 (2012).
37. A. Lowe, R. Harris, P. Bhansali, A. Cvekl, W. Liu, Intercellular Adhesion-Dependent Cell Survival and ROCK-Regulated Actomyosin-Driven Forces Mediate Self-Formation of a Retinal Organoid. Stem Cell Reports 6, 743-756 (2016).
38. X. Zhong et al., Generation of three-dimensional retinal tissue with functional photoreceptors from human iPSCs. Nat Commun 5, 4047 (2014).
39. S. Reichman et al., From confluent human iPS cells to self-forming neural retina and retinal pigmented epithelium. Proc Natl Acad Sci USA 111, 8518-8523 (2014).
40. C. S. Cowan et al., Cell Types of the Human Retina and Its Organoids at Single-Cell Resolution. Cell 182, 1623-1640 e1634 (2020).
41. C. M. Fligor et al., Extension of retinofugal projections in an assembled model of human pluripotent stem cell-derived organoids. Stem Cell Reports 10.1016/j.stemcr.2021.05.009 (2021).
42. S. Kim et al., Generation, transcriptome profiling, and functional validation of cone-rich human retinal organoids. Proc Natl Acad Sci USA 116, 10824-10833 (2019).
43. K. B. Langer et al., Retinal Ganglion Cell Diversity and Subtype Specification from Human Pluripotent Stem Cells. Stem Cell Reports 10, 1282-1293 (2018).
44. C. M. Fligor et al., Three-Dimensional Retinal Organoids Facilitate the Investigation of Retinal Ganglion Cell Development, Organization and Neurite Outgrowth from Human Pluripotent Stem Cells. Sci Rep 8, 14520 (2018).
45. P. Teotia et al., Generation of Functional Human Retinal Ganglion Cells with Target Specificity from Pluripotent Stem Cells by Chemically Defined Recapitulation of Developmental Mechanism. Stem Cells 35, 572-585 (2017).
46. M. A. Lancaster et al., Cerebral organoids model human brain development and microcephaly. Nature 501, 373-379 (2013).
47. T. Kadoshima et al., Self-organization of axial polarity, inside-out layer pattern, and species-specific progenitor dynamics in human ES cell-derived neocortex. Proc Natl Acad Sci USA 110, 20284-20289 (2013).
48. J. Mariani et al., Modeling human cortical development in vitro using induced pluripotent stem cells. Proc Natl Acad Sci USA 109, 12770-12775 (2012).
49. X. Qian, H. N. Nguyen, F. Jacob, H. Song, G. L. Ming, Using brain organoids to understand Zika virus-induced microcephaly. Development 144, 952-957 (2017).
50. S. Velasco et al., Individual brain organoids reproducibly form cell diversity of the human cerebral cortex. Nature 570, 523-527 (2019).
51. E. Gabriel et al., Human brain organoids assemble functionally integrated bilateral optic vesicles. Cell Stem Cell 10.1016/j.stem.2021.07.010 (2021).
52. C. Redies, L. Puelles, Modularity in vertebrate brain development and evolution. Bioessays 23, 1100-1111 (2001).
53. Y. Suda et al., The same enhancer regulates the earliest Emx2 expression in caudal forebrain primordium, subsequent expression in dorsal telencephalon and later expression in the cortical ventricular zone. Development 137, 2939-2949 (2010).
54. L. Gan et al., POU domain factor Brn-3b is required for the development of a large set of retinal ganglion cells. Proc Natl Acad Sci USA 93, 3920-3925 (1996).
55. E. A. Bassett et al., AP-2alpha knockout mice exhibit optic cup patterning defects and failure of optic stalk morphogenesis. Hum Mol Genet 19, 1791-1804 (2010).
56. C. Yang et al., Efficient generation of lens progenitor cells and lentoid bodies from human embryonic stem cells in chemically defined conditions. FASEB J24, 3274-3283 (2010).
57. S. K. Biswas, J. E. Lee, L. Brako, J. X. Jiang, W. K. Lo, Gap junctions are selectively associated with interlocking ball-and-sockets but not protrusions in the lens. Mol Vis 16, 2328-2341 (2010).
58. F. J. Lovicu, J. W. McAvoy, FGF-induced lens cell proliferation and differentiation is dependent on MAPK (ERK1/2) signalling. Development 128, 5075-5084 (2001).
59. T. Stuart et al., Comprehensive Integration of Single-Cell Data. Cell 177, 1888-1902 e1821 (2019).
60. I. Tirosh et al., Dissecting the multicellular ecosystem of metastatic melanoma by single-cell RNA-seq. Science 352, 189-196 (2016).
61. Z. Yao et al., A Single-Cell Roadmap of Lineage Bifurcation in Human ESC Models of Embryonic Brain Development. Cell Stem Cell 20, 120-134 (2017).
62. Y. C. Poh et al., Generation of organized germ layers from a single mouse embryonic stem cell. Nat Commun 5, 4000 (2014).
63. B. V. Varga et al., Signal requirement for cortical potential of transplantable human neuroepithelial stem cells. biorxiv World Wide Web at doi.org/10.1101/2021.03.27.437311 (2021).
64. M. Simoes-Costa, M. E. Bronner, Establishing neural crest identity: a gene regulatory recipe. Development 142, 242-257 (2015).
65. M. Chatterjee, J. Y. Li, Patterning and compartment formation in the diencephalon. Front Neurosci 6, 66 (2012).
66. A. Martinez-Ferre, S. Martinez, Molecular regionalization of the diencephalon. Front Neurosci 6, 73 (2012).
67. S. F. Oster, M. O. Bodeker, F. He, D. W. Sretavan, Invariant Sema5A inhibition serves an ensheathing function during optic nerve development. Development 130, 775-784 (2003).
68. M. Sandberg-Lall, P. O. Hagg, I. Wahlstrom, T. Pihlajaniemi, Type XIII collagen is widely expressed in the adult and developing human eye and accentuated in the ciliary muscle, the optic nerve and the neural retina. Exp Eye Res 70, 401-410 (2000).
69. E. Chatzopoulou et al., Structural requirement of TAG-1 for retinal ganglion cell axons and myelin in the mouse optic nerve. J Neurosci 28, 7624-7636 (2008).
70. M. Yamagata, J. R. Sanes, Expanding the Ig superfamily code for laminar specificity in retina: expression and role of contactins. J Neurosci 32, 14402-14414 (2012).
71. J. A. Lucas, T. M. Schmidt, Cellular properties of intrinsically photosensitive retinal ganglion cells during postnatal development. Neural Dev 14, 8 (2019).
72. T. M. Schmidt, P. Kofuji, Functional and morphological differences among intrinsically photosensitive retinal ganglion cells. J Neurosci 29, 476-482 (2009).
73. Y. Zhu et al., Three-dimensional neuroepithelial culture from human embryonic stem cells and its use for quantitative conversion to retinal pigment epithelium. PLoS One 8, e54552 (2013).
74. I. Bedzhov, M. Zernicka-Goetz, Self-organizing properties of mouse pluripotent cells initiate morphogenesis upon implantation. Cell 156, 1032-1044 (2014).
75. E. Coucouvanis, G. R. Martin, Signals for death and survival: a two-step mechanism for cavitation in the vertebrate embryo. Cell 83, 279-287 (1995).
76. S. M. Chambers et al., Highly efficient neural conversion of human ES and iPS cells by dual inhibition of SMAD signaling. Nat Biotechnol 27, 275-280 (2009).
77. R. Argelaguet et al., Multi-omics profiling of mouse gastrulation at single-cell resolution. Nature 576, 487-491 (2019).
78. V. Tropepe et al., Direct neural fate specification from embryonic stem cells: a primitive mammalian neural stem cell stage acquired through a default mechanism. Neuron 30, 65-78 (2001).
79. I. Munoz-Sanjuan, A. H. Brivanlou, Neural induction, the default model and embryonic stem cells. Nat Rev Neurosci 3, 271-280 (2002).
80. A. Kuwahara et al., Generation of a ciliary margin-like stem cell niche from self-organizing human retinal tissue. Nat Commun 6, 6286 (2015).
81. F. Etoc et al., A Balance between Secreted Inhibitors and Edge Sensing Controls Gastruloid Self-Organization. Dev Cell 39, 302-315 (2016).
82. K. T. Minn et al., High-resolution transcriptional and morphogenetic profiling of cells from micropatterned human ESC gastruloid cultures. Elife 9 (2020).
83. X. Xue et al., Mechanics-guided embryonic patterning of neuroectoderm tissue from human pluripotent stem cells. Nat Mater 17, 633-641 (2018).
84. R. Hayashi et al., Co-ordinated ocular development from human iPS cells and recovery of corneal function. Nature 531, 376-380 (2016).
85. R. Shirasaki, J. W. Lewcock, K. Lettieri, S. L. Pfaff, FGF as a target-derived chemoattractant for developing motor axons genetically programmed by the LIM code. Neuron 50, 841-853 (2006).
86. V. M. Sluch et al., Enhanced Stem Cell Differentiation and Immunopurification of Genome Engineered Human Retinal Ganglion Cells. Stem Cells Transl Med 6, 1972-1986 (2017).
87. R. Diacou, P. Bhansali, W. Liu, Three-Dimensional Culture of Mouse Eyecups. Methods Mol Biol 2092, 35-43 (2020).
88. A. Visel, C. Thaller, G. Eichele, GenePaint.org: an atlas of gene expression patterns in the mouse embryo. Nucleic Acids Res 32, D552-556 (2004).

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. An organoid comprising at least two concentric zones of telencephalic and ocular progenitors.

2. The organoid of claim 1, further comprising FOXG1+ telencephalic cells, PAX6+ multi-lineage ocular cells, VSX2+ retinal cells, PAX2+ optic disc and optic stalk cells, a lens cell, and/or a retinal ganglion cell (RGC).

3-7. (canceled)

8. The organoid of claim 2, wherein the RGC expresses at least one cell surface marker selected from ATOH7, POU4F2, ONECUT2, and TUBB3.

9. The organoid of claim 2, wherein the RGC comprises a TUBB3+ axon.

10. (canceled)

11. The organoid of claim 2, wherein the RGC expresses CNTN2 on the cell surface.

12-13. (canceled)

14. A method of producing the organoid of claim 1, the method comprising:
(a) inducing pluripotent stem cells to form a cyst showing an epithelial structure;
(b) plating a cyst at a low density; and
(c) growing the cyst as an adherent colony.

15. The method of claim 14, wherein the cyst is produced by culturing Matrigel-embedded stem cell sheets in a suspension culture.

16. The method of claim 14, wherein
(a) the cyst is formed in a medium comprising the N2 and B27 supplements; and/or
(b) the cyst is plated in a medium comprising the N2 and B27 supplements.

17. The method of claim 14, wherein the adherent colony of the cyst is grown in a medium comprising the KnockOut® Serum Replacement.

18. The method of claim 14, wherein the pluripotent stem cells are embryonic stem cells (ESCs) or induced pluripotent stem cells (iPSCs).

19. (canceled)

20. An organoid produced by the method of claim 14.

21. A method of producing a lens cell, the method comprising
(a) producing an organoid according to the method of claim 14;
(b) detaching and partially dissociating the organoid; and
(c) optionally growing the partially dissociated organoid in a suspension culture.

22. The method of claim 21, wherein partially dissociating the organoid separates the lens cells from other types of cells but leaves the lens cells as clusters.

23. The method of claim 21, wherein the lens cells express CRYAA and/or beta crystalline.

24. (canceled)

25. A lens cell produced according to the method of claim 21.

26. A method of producing a telencephalic cell and/or a retinal ganglion cell (RGC), the method comprising producing an organoid comprising the telencephalic cell and RGC according to the method of claim 14.

27-29. (canceled)

30. A method of isolating a retinal ganglion cell (RGC) from an organoid, the method comprising:
contacting a cell from the organoid with an antigen-binding protein that specifically binds to CNTN2;
capturing an antigen-binding protein-RGC complex; and
removing the antibody from the antigen-binding protein-RGC complex, thereby isolating the RGC from the organoid.

31-37. (canceled)

38. A retinal ganglion cell (RGC) isolated by the method of claim 30.

39. (canceled)

40. A method of treating a subject having or suspected of having a loss of a retinal ganglion cell, the method comprising:
administering to the subject a therapeutically effective amount of a pharmaceutical composition comprising the RGC produced according to the method of claim 26.

41. The method of claim 40, wherein the RGC expresses at least one cell surface marker selected from ATOH7, POU4F2, ONECUT2, and TUBB3.

42-54. (canceled)
